(19) United States
(12) Patent Application Publication
(10) Pub. No.: US 2026/0049364 A1
(43) Pub. Date: Feb. 19, 2026

(54) 2K SNP LIQUID CHIP OF AVENA SATIVA AND APPLICATIONS THEREOF

(71) Applicant: INNER MONGOLIA AGRICULTURAL UNIVERSITY, Hohhot (CN)

(72) Inventors: Bing HAN, Hohhot (CN); Jinsheng NAN, Hohhot (CN); Ting WANG, Hohhot (CN); Huiyan LIU, Hohhot (CN); Erniu HAN, Hohhot (CN); Daxiao ZHANG, Hohhot (CN); Ziyang WANG, Hohhot (CN)

(21) Appl. No.: 18/926,896

(22) Filed: Oct. 25, 2024

(30) Foreign Application Priority Data
Aug. 19, 2024 (CN) 202411133186.8

(51) Int. Cl. C12Q 1/6895; G16B 20/20

(52) U.S. Cl. CPC C12Q 1/6895; G16B 20/20; C12Q 2600/13; C12Q 2600/156

(57) ABSTRACT

A 2K SNP liquid chip of Avena sativa and applications thereof are provided, and relates to the technical field of molecular biology. High throughput 2K SNP liquid chip for Avena sativa genotyping is developed based on the targeted capture sequencing technology, and the liquid chip is determined by multi-point and multi-sample experiments for many years.

### CROSS-REFERENCE TO RELATED APPLICATIONS

This application claims priority to Chinese Patent Application No. 202411133186.8, filed on Aug. 19, 2024, the contents of which are hereby incorporated by reference.

### TECHNICAL FIELD

The disclosure relates to the technical field of molecular biology, and in particular to a 2K SNP liquid chip of Avena sativa and applications thereof.

### BACKGROUND

Avena sativa used in agricultural production belongs to the genus Avena, graminae family (2n=6x=42; AACCDD genome), and Avena sativa is a kind of food and feed crops planted all over the world, which has important economic value. Grains of Avena sativa play an important role in lowering blood sugar and preventing cardiovascular diseases with rich dietary fiber, antioxidants, minerals and vitamins, and have long been cherished by consumers. Cultivated Avena sativa has large genome (about 11G) and high repetitive sequence content (about 87%). With the rapid development of high-throughput sequencing technology and genome assembly technology, the genome sequence information of high-quality diploid, tetraploid and hexaploid Avena sativa is released, which improves the understanding of Avena sativa basic biology and is very important for genetics, genomics and molecular breeding research.

Variety cultivation is the foundation for the development of Avena sativa seed industry, and at the same time, variety molecular identifying is the inevitable requirement for the healthy development of seed industry. Molecular identifying of Avena sativa varieties is capable of being applied to variety approval and variety right protection, so that the domestic and international seed market and breeding utilization may be legally protected. At present, there are a large number of seed business entities in China, fake and shoddy seeds are banned, and the problem of counterfeit infringement is prominent, which seriously infringes on the rights and interests of farmers and legitimate business enterprises. Molecular identifying technology of varieties may be used in market supervision to quickly identify authenticity, crack down on market violations and effectively maintain fair market order. Therefore, it is very necessary to establish the technical standard for the identifying of Avena sativa varieties by SNP liquid chip. Since the implementation of “Seed Law of the People's Republic of China”, the Ministry of Agriculture and Rural Affairs of China aims to improve the examination and approval of crop varieties to meet the needs of modern agricultural development. The establishment of a national unified variety information database and supervision system enables to enhance the supervision of variety authenticity, strengthen spot checks in markets and enterprises, crack down on counterfeit card-making, and protect seed quality and farmers' interests.

In the aspect of variety authenticity identifying, the technology has developed from the initial electron microscope scanning, physical and chemical identifying and protein electrophoresis technology to the hyperspectral image technology and deep learning image processing technology at the image level, and now to the molecular level identifying. Common molecular identifying methods include simple sequence repeats (SSR) markers, kompetitive allele specific PCR (KASP), single nucleotide polymorphism (SNP) identifying methods and the application of multiple nucleotide polymorphism (MNP) molecular markers. As the most widely distributed genetic marker in the genome, SNP has the characteristics of high density, high genetic stability and easy automatic analysis, which makes efficient and low-cost SNP genotype detection technology the first choice for the development of sharing technology and platform. At present, gene chip method is the most commonly used method for SNP marker genotyping, including SNP solid chip and SNP liquid chip. High-throughput SNP liquid chip is an important tool to carry out molecular breeding research such as high-throughput genotyping, germplasm identifying, true and false seed identifying and fingerprint construction. Identifying of crop varieties by SNP molecular markers has become one of the important bases for the protection of new varieties. The “Seed Law of the People's Republic of China” stipulates that the reinspection shall not adopt the same detection method. Therefore, the combined use of multiple detection methods will meet the needs of rapid detection in the future. At present, there is no relevant technical regulations for the identifying of Avena sativa germplasm resources, variety authenticity and purity at home and abroad, which shows that there is still a lot of room for development in this field. At present, there is no relevant technology at home and abroad for the identifying of Avena sativa germplasm resources, variety authenticity and purity. Developing an economical, high-density and high-throughput Avena sativa genotyping chip for Avena sativa variety identifying is beneficial to better serve the industrialized application of Avena sativa.

### SUMMARY

The objective of the present disclosure is to provide a 2K SNP liquid chip of Avena sativa and applications thereof, so as to solve the problems existing in the prior art. The 2K liquid chip of Avena sativa developed based on the targeted capture sequencing technology have the advantages of uniformly covering the genome by the loci, strong representativeness, high polymorphism, high specificity and strong universality, and are suitable for the authenticity identifying of Avena sativa varieties.

In order to achieve the above objectives, provided is the following technical scheme.

Provided is an SNP loci combination for Avena sativa variety identifying, including SNP loci located in reference genomes sanfensan and OT3098 as shown in Table 1.

The disclosure also provides an application of the SNP loci combination in variety identifying, seed purity analysis, hybrid breeding and fingerprint construction of Avena sativa.

The disclosure also provides an application of the SNP loci combination in preparing 2K SNP liquid chip for Avena sativa variety identifying.

Also provided is a 2K SNP liquid chip of Avena sativa, including a probe combination used for identifying a genotype of each SNP locus in the SNP loci combination.
also provided is an application of the 2K SNP liquid chip of Avena sativa in variety identifying, seed purity analysis, hybrid breeding and fingerprint construction of Avena sativa.

Also provided is a variety identifying method of Avena sativa including following steps.
(1) obtaining genomic DNAs of samples to be detected;
(2) constructing a sequencing library based on the genomic DNAs;
(3) performing a probe hybridization reaction on the sequencing library and the 2K SNP liquid chip of Avena sativa above;
(4) extracting genotyping information after sequence sequencing captured by the liquid chip to form a genotyping file; and
(5) constructing fingerprint data of 2429 SNP loci of the samples to be detected; comparing the fingerprints of the samples to be detected in pairs, and performing statistical comparison on a total number of loci and a number of difference loci to calculate similarity of loci of the samples to be detected.

The disclosure has the following technical effects.

First, the disclosure adopts the liquid phase probe capture technology to design and synthesize a set of liquid phase chips containing 2K SNP loci information to detect the liquid phase chips of Avena sativa background loci. The liquid phase chips may quickly and effectively track the genetic material of Avena sativa, which is beneficial to accelerating the Avena sativa breeding process and improving the breeding efficiency of new varieties.

Second, material background of data related to SNP loci involved in the liquid chip of the disclosure covers a wider variety of Avena sativa, with high representativeness and rich diversity information.

Third, SNP loci involved in the liquid chip of the disclosure are uniformly distributed on chromosomes.

Fourth, compared with other Avena sativa chips in the prior art, the disclosure has the advantage of low cost for variety authenticity identifying.

Fifth, the chip of the present disclosure is a liquid chip, and compared with the solid chips, the liquid chip has high detection accuracy, more effective information and high flexibility and diversity, and may effectively reduce the detection cost and improve the efficiency of scientific research and breeding.

Sixth, the 2K liquid chip developed by the disclosure may be applied to the authenticity identifying of Avena sativa varieties, seed purity analysis, hybrid breeding and fingerprint construction, which is beneficial to accelerating the process of Avena sativa research and breeding.

Seventh, screening of SNP markers: this technology uses SNP markers to identify Avena sativa varieties instead of traditional molecular markers such as SSR or amplified fragment length polymorphism (AFLP). SNP markers are highly stable and abundant, widely exist in the genomes, and have high genetic diversity, so they may identify Avena sativa varieties more accurately.

Eighth, data analysis: the technical specification for identifying of Avena sativa variety authenticity by SNP liquid chip also includes data analysis of SNP genotyping results. By analyzing and comparing the genotype combinations of different SNP loci, the Avena sativa varieties may be determined. This data analysis method may identify Avena sativa varieties more quickly and accurately and improve the identifying efficiency.

To sum up, the high throughput SNP 2K liquid chip for Avena sativa genotyping is developed based on the targeted capture sequencing technology, and the liquid chip is determined by multi-point and multi-sample experiments for many years, with good repeatability and stability and convenient popularization and application; the liquid chip may be effectively applied to the authenticity identifying of Avena sativa varieties, seed purity analysis, hybrid breeding and fingerprint construction; the liquid chip provides scientific and technological support for Avena sativa variety management, quality inspection and intellectual property protection, and lays a technical foundation for Avena sativa germplasm resources analysis, which is conducive to accelerating the breeding of excellent Avena sativa varieties.

### BRIEF DESCRIPTION OF THE DRAWINGS

In order to explain the embodiments of the present disclosure or the technical scheme in the prior art more clearly, the drawings needed in the embodiments will be briefly introduced below. Obviously, the drawings described below are only some embodiments of the present disclosure, and other drawings may be obtained according to these drawings without creative work for ordinary people in the field.

FIG. 1 is a schematic diagram showing the distribution of SNP loci in different chromosomes of 2K SNP chips of Avena sativa.

FIG. 2 shows a uniform distribution schematic diagram of SNP loci.

FIG. 3 is a statistical chart of MAF distribution.

FIG. 4 is a statistical diagram of annotation information of SNP loci in genomes.

FIG. 5 is a genetic distance cluster analysis diagram of 49 germplasm resources.

### DETAILED DESCRIPTION OF THE EMBODIMENTS

A number of exemplary embodiments will be described in detail, and this detailed description should not be considered as a limitation of the present disclosure, but as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the disclosure. The description and example of that disclosure are exemplary only.

The terms “comprising”, “including”, “having” and “containing” used in this application are all open terms, which means including but not limited to.

### Embodiment 1

The disclosure provides a 2K SNP liquid chip of Avena sativa, which includes 2K SNP loci probes of Avena sativa, where the 2K SNP loci probes of Avena sativa are single-stranded DNA synthesized according to 2K SNP molecular markers of Avena sativa, and the 2K SNP loci of Avena sativa include SNP molecular markers shown in Table 1. The 5′ end of each probe has a biotin group, and the probe is coupled to the fluorescent microsphere through C12 molecular arm and amino modification, and each fluorescent microsphere is coupled with a probe.

The preparation process of the 2K SNP liquid chip of Avena sativa has following steps.
1. The 2K SNP loci of Avena sativa are screened, specifically, the 2K SNP loci of Avena sativa are obtained by the following methods.
1.1 According to 285 Avena sativa 15X resequencing data, 659 GBS data and other information, the genome of Avena sativa sanfensan (GenBank: GCA_023646675.1) is used as the main reference genome for loci screening, and the SNP loci on the genome are detected by BWA. The program automatically screens the loci, and the parameters are as follows: MQ≥55; 30 bp/60 bp is uniquely compared on the genome; GC 35-65; LD filtration; 120 bp SNP≤3; covering windows evenly with a length of 10M. A total of 2200 SNP loci are screened out.
1.2 Avena sativa OT3098 genome (GenBank: GCA_916181665.1) is used as reference genome, supplementary design is carried out, and GAP region probes are added. Screening is performed according to the selection method in step 1.1, ensuring that the probe covers more than 98% of the region in both genes. A total of 229 SNP loci are screened.
2. The 2K SNP loci probe of Avena sativa are designed and prepared, and the process is as follows.
2.1 Determining the probe design principles

The probe length is 120 bp, the GC content of the probe is between 25% and 49%, the average GC content is about 45%, and the number of homologous regions is ≤3. The selected region does not contain SSR and GAP regions to the greatest extent; the priority is: gene region>gene promoter region (2 Kb)>gene downstream adjacent region (2 Kb)>gene spacer region, combined with the principle of uniform chromosome distribution.
2.2 Taking the SNP locus obtained in step 1 as the center, the 120 bp nucleotide sequence with the GC content closest to 45% between left and right 120 bp is screened as a probe.
2.3 According to the 120 bp nucleotide sequence screened in step 2.2, the single-stranded DNA with biotin group modification at the 5′ end is synthesized, which is called 2K SNP loci probe of Avena sativa.
3. The probes prepared in step 2 are coupled to the fluorescent microspheres, and the targeted capture reagent is added to obtain the 2K SNP liquid chip of Avena sativas.

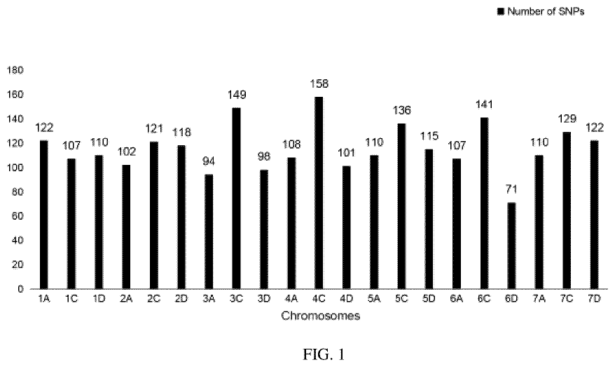

The distribution of SNP loci on different chromosomes of the 2K SNP liquid chip of Avena sativas obtained in this embodiment is shown in FIG. 1. As can be seen from FIG. 1, the probes of the present disclosure are uniformly designed on different chromosomes, so that the capture loci cover the whole genome.

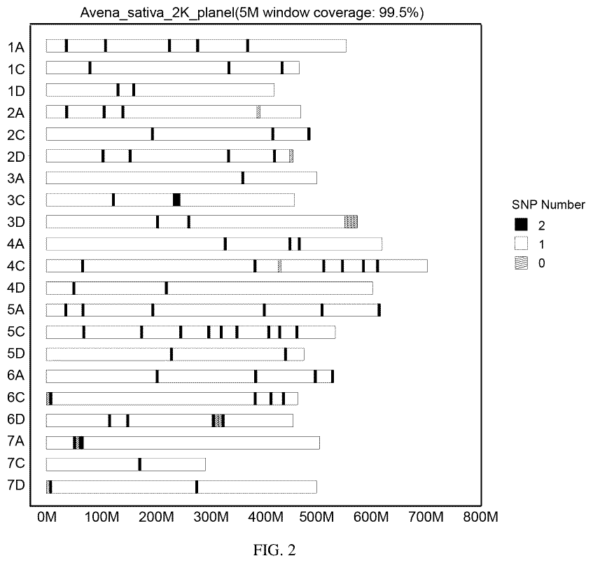

The uniform distribution diagram of SNP loci of the 2K liquid chip of Avena sativa is shown in FIG. 2. As observed from FIG. 2, the 5 Mb window coverage rate of the genome is 99.5%, and the overall coverage of the genome is good.

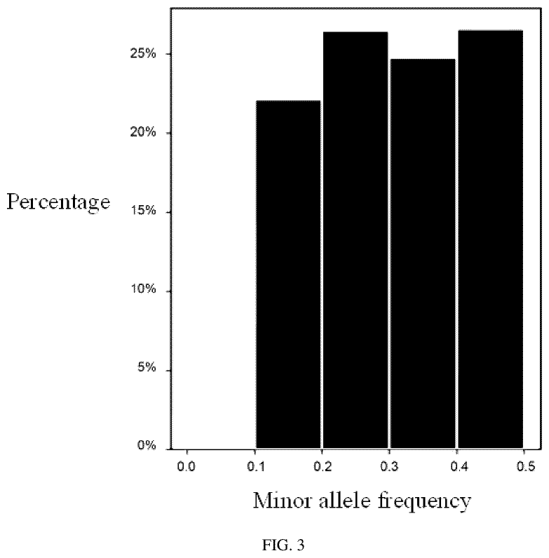

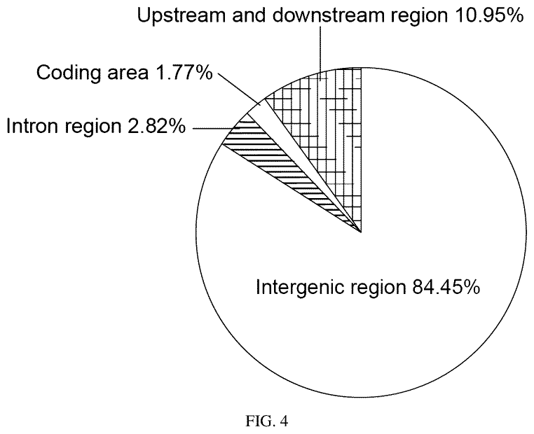

The minor allele frequency (MAF) distribution of the 2K Avena sativa liquid chip is shown in FIG. 3, and it may be seen that the MAF value is between 0.1 and 0.5. The statistical diagram of SNP annotation information in the genome is shown in FIG. 4.

TABLE 1
SNP loci of 2K liquid chip of Avena sativa

| Chromo- | Physical | Allelic | Reference | Chromo- | Physical | Allelic | Reference | Chromo- | Physical | Allelic | Reference |
|---|---|---|---|---|---|---|---|---|---|---|---|
| some | location | variation | genome | some | location | variation | genome | some | location | variation | genome |
| 1A | 4473517 | C, A | sanfensan | 3C | 488844690 | A, C | sanfensan | 6A | 108982250 | C, T | sanfensan |
| 1A | 9349344 | G, A | sanfensan | 3C | 494056611 | C, T | sanfensan | 6A | 109794291 | G, A | sanfensan |
| 1A | 14898617 | C, T | sanfensan | 3C | 499140906 | G, A | sanfensan | 6A | 113440903 | T, G | sanfensan |
| 1A | 19788850 | T, C | sanfensan | 3C | 504946938 | C, A | sanfensan | 6A | 118377169 | C, G | sanfensan |
| 1A | 24134605 | A, C | sanfensan | 3C | 505015277 | T, G | sanfensan | 6A | 124172048 | A, G | sanfensan |
| 1A | 25204308 | G, A | sanfensan | 3C | 513611369 | G, A | sanfensan | 6A | 125350773 | A, C | sanfensan |
| 1A | 34911838 | T, A | sanfensan | 3C | 515368248 | C, T | sanfensan | 6A | 130843545 | T, C | sanfensan |
| 1A | 35091182 | A, G | sanfensan | 3C | 520707894 | G, A | sanfensan | 6A | 135683529 | T, C | sanfensan |
| 1A | 39926834 | G, A | sanfensan | 3C | 529897647 | C, T | sanfensan | 6A | 143547400 | A, T | sanfensan |
| 1A | 40243841 | C, A | sanfensan | 3C | 534373018 | A, G | sanfensan | 6A | 147670247 | A, G | sanfensan |
| 1A | 46676991 | T, C | sanfensan | 3C | 536903652 | C, T | sanfensan | 6A | 151304761 | T, C | sanfensan |
| 1A | 50477703 | C, T | sanfensan | 3C | 540735781 | A, C | sanfensan | 6A | 157973297 | G, C | sanfensan |
| 1A | 55467045 | T, C | sanfensan | 3C | 545800871 | T, C | sanfensan | 6A | 158294119 | A, C | sanfensan |
| 1A | 61641698 | C, A | sanfensan | 3C | 551522255 | A, C | sanfensan | 6A | 163354468 | G, C | sanfensan |
| 1A | 65935052 | C, A | sanfensan | 3C | 556937242 | T, C | sanfensan | 6A | 165693464 | T, C | sanfensan |
| 1A | 71753358 | C, T | sanfensan | 3C | 561945091 | C, A | sanfensan | 6A | 173487971 | G, A | sanfensan |
| 1A | 76746486 | T, C | sanfensan | 3C | 565260776 | C, T | sanfensan | 6A | 176286584 | G, A | sanfensan |
| 1A | 82946413 | T, C | sanfensan | 3C | 572949143 | C, G | sanfensan | 6A | 183958426 | C, T | sanfensan |
| 1A | 85796042 | G, A | sanfensan | 3C | 577651667 | G, A | sanfensan | 6A | 186274335 | G, A | sanfensan |
| 1A | 92427340 | G, A | sanfensan | 3C | 580350958 | C, T | sanfensan | 6A | 194312081 | C, T | sanfensan |
| 1A | 98303200 | A, C | sanfensan | 3C | 589499247 | A, G | sanfensan | 6A | 198052953 | G, A | sanfensan |
| 1A | 100886423 | T, C | sanfensan | 3C | 593401324 | A, G | sanfensan | 6A | 203626460 | T, G | sanfensan |
| 1A | 105665342 | A, G | sanfensan | 3C | 596059950 | G, A | sanfensan | 6A | 208551969 | G, A | sanfensan |
| 1A | 106585584 | T, C | sanfensan | 3C | 602749897 | C, G | sanfensan | 6A | 212325078 | A, G | sanfensan |
| 1A | 110857330 | A, C | sanfensan | 3C | 606686603 | A, G | sanfensan | 6A | 216393559 | G, A | sanfensan |
| 1A | 119266034 | T, C | sanfensan | 3C | 612645249 | A, G | sanfensan | 6A | 222443511 | C, G | sanfensan |
| 1A | 120319599 | T, G | sanfensan | 3C | 615205024 | T, A | sanfensan | 6A | 227146163 | G, A | sanfensan |
| 1A | 128479703 | G, A | sanfensan | 3D | 3451014 | A, G | sanfensan | 6A | 232355131 | C, A | sanfensan |
| 1A | 131129360 | A, G | sanfensan | 3D | 6304975 | C, G | sanfensan | 6A | 236306595 | G, A | sanfensan |
| 1A | 135686787 | G, A | sanfensan | 3D | 8431856 | G, A | sanfensan | 6A | 242551165 | C, T | sanfensan |
| 1A | 140893664 | A, G | sanfensan | 3D | 12739908 | G, A | sanfensan | 6A | 248683146 | T, A | sanfensan |
| 1A | 149729422 | A, G | sanfensan | 3D | 17626366 | T, C | sanfensan | 6A | 254218064 | C, T | sanfensan |
| 1A | 154018712 | A, G | sanfensan | 3D | 22243586 | T, C | sanfensan | 6A | 256311166 | G, A | sanfensan |
| 1A | 156986726 | T, C | sanfensan | 3D | 29901822 | T, C | sanfensan | 6A | 263945630 | T, C | sanfensan |
| 1A | 160810612 | G, C | sanfensan | 3D | 34199181 | T, A | sanfensan | 6A | 265291617 | G, A | sanfensan |
| 1A | 168045464 | T, G | sanfensan | 3D | 38215402 | C, T | sanfensan | 6A | 272551424 | T, C | sanfensan |
| 1A | 174968050 | A, G | sanfensan | 3D | 40736696 | C, T | sanfensan | 6A | 276146901 | A, C | sanfensan |
| 1A | 177054360 | C, T | sanfensan | 3D | 45922953 | C, T | sanfensan | 6A | 283068354 | T, C | sanfensan |
| 1A | 184641750 | G, A | sanfensan | 3D | 54465933 | T, C | sanfensan | 6A | 287654071 | G, A | sanfensan |
| 1A | 186347220 | A, G | sanfensan | 3D | 59996227 | A, G | sanfensan | 6A | 291439784 | A, C | sanfensan |
| 1A | 190161955 | T, C | sanfensan | 3D | 63704529 | G, A | sanfensan | 6A | 296813413 | T, A | sanfensan |
| 1A | 197891296 | A, G | sanfensan | 3D | 66789700 | C, G | sanfensan | 6A | 304331839 | T, C | sanfensan |
| 1A | 202332235 | G, C | sanfensan | 3D | 70216974 | C, T | sanfensan | 6A | 309571759 | G, C | sanfensan |
| 1A | 206929081 | A, G | sanfensan | 3D | 78492763 | A, G | sanfensan | 6A | 311843254 | A, C | sanfensan |
| 1A | 213307997 | A, C | sanfensan | 3D | 82560649 | G, A | sanfensan | 6A | 318306732 | G, A | sanfensan |
| 1A | 215902606 | A, C | sanfensan | 3D | 89278480 | C, T | sanfensan | 6A | 324491687 | A, C | sanfensan |
| 1A | 223276058 | G, T | sanfensan | 3D | 94976535 | T, A | sanfensan | 6A | 326644360 | G, A | sanfensan |
| 1A | 225939090 | G, A | sanfensan | 3D | 99870793 | C, A | sanfensan | 6A | 330034066 | C, T | sanfensan |
| 1A | 229684564 | T, C | sanfensan | 3D | 102284469 | G, A | sanfensan | 6A | 335513362 | C, T | sanfensan |
| 1A | 230432457 | A, G | sanfensan | 3D | 105085292 | G, T | sanfensan | 6A | 339809705 | C, G | sanfensan |
| 1A | 239890900 | C, T | sanfensan | 3D | 112272049 | A, G | sanfensan | 6A | 343191906 | T, C | sanfensan |
| 1A | 243567990 | G, A | sanfensan | 3D | 117725865 | G, A | sanfensan | 6A | 349219222 | G, A | sanfensan |
| 1A | 245948969 | G, A | sanfensan | 3D | 124920499 | T, C | sanfensan | 6A | 351759840 | A, G | sanfensan |
| 1A | 250969537 | G, C | sanfensan | 3D | 125085251 | G, A | sanfensan | 6A | 358242236 | G, T | sanfensan |
| 1A | 257803247 | T, G | sanfensan | 3D | 130241097 | G, T | sanfensan | 6A | 362754991 | T, C | sanfensan |
| 1A | 263812100 | A, G | sanfensan | 3D | 136299812 | G, A | sanfensan | 6A | 367473345 | G, A | sanfensan |
| 1A | 268553550 | T, C | sanfensan | 3D | 140453087 | G, T | sanfensan | 6A | 371831728 | G, A | sanfensan |
| 1A | 271645348 | T, G | sanfensan | 3D | 149490075 | G, A | sanfensan | 6A | 377260484 | T, C | sanfensan |
| 1A | 283816950 | G, A | sanfensan | 3D | 154494846 | C, T | sanfensan | 6A | 382654181 | C, T | sanfensan |
| 1A | 286799892 | T, C | sanfensan | 3D | 155736382 | A, G | sanfensan | 6A | 389448736 | A, G | sanfensan |
| 1A | 292498165 | C, A | sanfensan | 3D | 160258419 | T, G | sanfensan | 6A | 394354567 | T, C | sanfensan |
| 1A | 298383411 | A, G | sanfensan | 3D | 165382934 | G, C | sanfensan | 6A | 395689953 | A, G | sanfensan |
| 1A | 300811687 | A, G | sanfensan | 3D | 174233842 | A, G | sanfensan | 6A | 402143190 | A, G | sanfensan |
| 1A | 307060043 | T, G | sanfensan | 3D | 178210807 | A, G | sanfensan | 6A | 409084859 | A, C | sanfensan |
| 1A | 314835969 | G, A | sanfensan | 3D | 183045672 | C, T | sanfensan | 6A | 414438357 | A, G | sanfensan |
| 1A | 315910953 | A, G | sanfensan | 3D | 185605470 | A, G | sanfensan | 6A | 415355947 | T, C | sanfensan |
| 1A | 321206313 | T, G | sanfensan | 3D | 191181967 | T, C | sanfensan | 6A | 421445718 | A, C | sanfensan |
| 1A | 329104188 | A, C | sanfensan | 3D | 199127188 | C, A | sanfensan | 6A | 422458226 | G, A | sanfensan |
| 1A | 333516925 | G, T | sanfensan | 3D | 201408393 | T, C | sanfensan | 6A | 425035666 | C, A | sanfensan |
| 1A | 335522334 | G, A | sanfensan | 3D | 208325818 | C, A | sanfensan | 6A | 433725430 | A, G | sanfensan |
| 1A | 342234031 | G, A | sanfensan | 3D | 213296950 | G, T | sanfensan | 6A | 437585163 | T, C | sanfensan |
| 1A | 348750824 | C, A | sanfensan | 3D | 217423350 | T, C | sanfensan | 6A | 444959115 | G, C | sanfensan |
| 1A | 354606484 | C, T | sanfensan | 3D | 223809923 | G, C | sanfensan | 6A | 445547963 | C, T | sanfensan |
| 1A | 355044825 | G, A | sanfensan | 3D | 226483167 | G, A | sanfensan | 6A | 454994988 | A, G | sanfensan |
| 1A | 361899430 | G, A | sanfensan | 3D | 231132075 | C, T | sanfensan | 6C | 4740123 | A, C | sanfensan |
| 1A | 365228574 | T, G | sanfensan | 3D | 237287427 | T, A | sanfensan | 6C | 7818111 | T, G | sanfensan |
| 1A | 370165053 | A, G | sanfensan | 3D | 241417679 | G, A | sanfensan | 6C | 10608939 | A, T | sanfensan |
| 1A | 371882871 | T, C | sanfensan | 3D | 248327064 | G, A | sanfensan | 6C | 17502954 | A, C | sanfensan |
| 1A | 379396033 | A, C | sanfensan | 3D | 250955048 | C, T | sanfensan | 6C | 21919209 | C, T | sanfensan |
| 1A | 380814167 | C, T | sanfensan | 3D | 257222395 | C, T | sanfensan | 6C | 27489398 | T, C | sanfensan |
| 1A | 385881054 | T, G | sanfensan | 3D | 263680149 | G, A | sanfensan | 6C | 32883642 | T, G | sanfensan |
| 1A | 394799443 | C, G | sanfensan | 3D | 268114489 | C, A | sanfensan | 6C | 35200287 | C, A | sanfensan |
| 1A | 396233056 | A, T | sanfensan | 3D | 273619731 | A, C | sanfensan | 6C | 38472337 | T, C | sanfensan |
| 1A | 400131929 | A, G | sanfensan | 3D | 276710058 | C, T | sanfensan | 6C | 43196331 | A, G | sanfensan |
| 1A | 408599208 | C, T | sanfensan | 3D | 284403130 | G, C | sanfensan | 6C | 47589408 | A, G | sanfensan |
| 1A | 410583439 | T, C | sanfensan | 3D | 288065151 | G, A | sanfensan | 6C | 51088171 | A, G | sanfensan |
| 1A | 417767787 | G, A | sanfensan | 3D | 294630726 | C, T | sanfensan | 6C | 59096246 | G, A | sanfensan |
| 1A | 423934932 | G, T | sanfensan | 3D | 297227408 | C, T | sanfensan | 6C | 60407986 | C, T | sanfensan |
| 1A | 427534714 | A, G | sanfensan | 3D | 300199691 | T, C | sanfensan | 6C | 65757372 | C, T | sanfensan |
| 1A | 430802770 | T, C | sanfensan | 3D | 306425893 | C, A | sanfensan | 6C | 66609935 | G, A | sanfensan |
| 1A | 439615284 | A, G | sanfensan | 3D | 314722897 | A, G | sanfensan | 6C | 72292296 | A, G | sanfensan |
| 1A | 443388417 | C, T | sanfensan | 3D | 315890257 | A, T | sanfensan | 6C | 75973462 | A, T | sanfensan |
| 1A | 447639906 | G, A | sanfensan | 3D | 323402379 | G, A | sanfensan | 6C | 81974276 | C, T | sanfensan |
| 1A | 453204191 | T, C | sanfensan | 3D | 325861485 | A, G | sanfensan | 6C | 88129794 | G, C | sanfensan |
| 1A | 457075413 | G, A | sanfensan | 3D | 330563924 | A, G | sanfensan | 6C | 94193475 | T, C | sanfensan |
| 1A | 463117262 | G, A | sanfensan | 3D | 335625837 | G, A | sanfensan | 6C | 95536874 | T, C | sanfensan |
| 1A | 465118330 | G, T | sanfensan | 3D | 340772061 | G, A | sanfensan | 6C | 101717822 | C, T | sanfensan |
| 1A | 472500626 | T, C | sanfensan | 3D | 348100481 | T, G | sanfensan | 6C | 107891612 | C, T | sanfensan |
| 1A | 475569735 | T, G | sanfensan | 3D | 352819049 | C, T | sanfensan | 6C | 112851715 | A, T | sanfensan |
| 1A | 481563062 | G, A | sanfensan | 3D | 358682575 | T, C | sanfensan | 6C | 116496712 | C, T | sanfensan |
| 1A | 486246321 | T, C | sanfensan | 3D | 361158177 | G, A | sanfensan | 6C | 121111029 | T, C | sanfensan |
| 1A | 490249212 | G, A | sanfensan | 3D | 369050731 | T, C | sanfensan | 6C | 125320836 | G, A | sanfensan |
| 1A | 495026085 | C, T | sanfensan | 3D | 373598604 | C, T | sanfensan | 6C | 132900758 | T, A | sanfensan |
| 1A | 500157268 | G, A | sanfensan | 3D | 378481165 | T, C | sanfensan | 6C | 135785484 | G, C | sanfensan |
| 1A | 505420346 | T, C | sanfensan | 3D | 381350490 | G, A | sanfensan | 6C | 143030880 | C, A | sanfensan |
| 1A | 513237629 | C, A | sanfensan | 3D | 388861217 | T, C | sanfensan | 6C | 148851945 | G, A | sanfensan |
| 1A | 516943173 | A, G | sanfensan | 3D | 389878476 | T, G | sanfensan | 6C | 154103608 | A, C | sanfensan |
| 1A | 520725745 | C, G | sanfensan | 3D | 391163814 | T, C | sanfensan | 6C | 156034908 | T, G | sanfensan |
| 1A | 529117339 | C, T | sanfensan | 3D | 397016298 | C, T | sanfensan | 6C | 162091462 | A, C | sanfensan |
| 1A | 532148235 | T, C | sanfensan | 3D | 403496100 | A, G | sanfensan | 6C | 165806162 | A, T | sanfensan |
| 1A | 538845292 | C, T | sanfensan | 3D | 406924921 | A, G | sanfensan | 6C | 172155825 | G, A | sanfensan |
| 1A | 540384422 | G, C | sanfensan | 3D | 410226093 | G, A | sanfensan | 6C | 175590346 | C, T | sanfensan |
| 1A | 546023901 | C, G | sanfensan | 3D | 417099879 | A, G | sanfensan | 6C | 180731171 | G, A | sanfensan |
| 1A | 551040971 | C, T | sanfensan | 3D | 419164521 | C, G | sanfensan | 6C | 185475790 | A, G | sanfensan |
| 1C | 4866196 | A, C | sanfensan | 3D | 423904272 | T, C | sanfensan | 6C | 194315523 | G, A | sanfensan |
| 1C | 8009186 | G, A | sanfensan | 3D | 426092994 | A, G | sanfensan | 6C | 196088194 | G, A | sanfensan |
| 1C | 12114531 | T, C | sanfensan | 3D | 433821318 | G, A | sanfensan | 6C | 196851358 | C, T | sanfensan |
| 1C | 17916692 | G, A | sanfensan | 3D | 436973559 | T, G | sanfensan | 6C | 200122712 | A, C | sanfensan |
| 1C | 22093157 | T, C | sanfensan | 3D | 439655087 | A, G | sanfensan | 6C | 207092401 | A, G | sanfensan |
| 1C | 25463149 | C, T | sanfensan | 3D | 441290663 | T, C | sanfensan | 6C | 213333184 | T, G | sanfensan |
| 1C | 32954922 | G, T | sanfensan | 3D | 448791478 | T, C | sanfensan | 6C | 217526046 | G, A | sanfensan |
| 1C | 38255313 | G, A | sanfensan | 3D | 451012282 | C, G | sanfensan | 6C | 222420550 | C, T | sanfensan |
| 1C | 40225359 | C, T | sanfensan | 3D | 456635851 | G, A | sanfensan | 6C | 227353579 | G, A | sanfensan |
| 1C | 47044975 | C, T | sanfensan | 3D | 460647997 | A, T | sanfensan | 6C | 234060684 | A, G | sanfensan |
| 1C | 54713053 | C, T | sanfensan | 4A | 167665 | A, G | sanfensan | 6C | 235857775 | G, A | sanfensan |
| 1C | 57342838 | G, T | sanfensan | 4A | 5170696 | A, G | sanfensan | 6C | 244931366 | T, C | sanfensan |
| 1C | 64447286 | C, T | sanfensan | 4A | 12321972 | G, A | sanfensan | 6C | 246070168 | G, A | sanfensan |
| 1C | 68342251 | C, G | sanfensan | 4A | 19482740 | T, C | sanfensan | 6C | 253789587 | C, T | sanfensan |
| 1C | 73736931 | T, C | sanfensan | 4A | 22870941 | T, C | sanfensan | 6C | 257684248 | G, C | sanfensan |
| 1C | 75426247 | G, A | sanfensan | 4A | 27380296 | T, C | sanfensan | 6C | 260070968 | C, T | sanfensan |
| 1C | 81261715 | C, T | sanfensan | 4A | 30862932 | G, T | sanfensan | 6C | 266510278 | C, T | sanfensan |
| 1C | 89052462 | A, C | sanfensan | 4A | 36420099 | T, G | sanfensan | 6C | 272777330 | C, T | sanfensan |
| 1C | 93949401 | C, T | sanfensan | 4A | 38087452 | T, G | sanfensan | 6C | 275890891 | G, A | sanfensan |
| 1C | 97665480 | T, A | sanfensan | 4A | 44284137 | A, C | sanfensan | 6C | 284003530 | C, T | sanfensan |
| 1C | 104414265 | C, T | sanfensan | 4A | 45337009 | T, C | sanfensan | 6C | 289057124 | C, T | sanfensan |
| 1C | 107889124 | C, T | sanfensan | 4A | 50385302 | T, C | sanfensan | 6C | 292273925 | G, A | sanfensan |
| 1C | 112840945 | A, G | sanfensan | 4A | 58613149 | T, C | sanfensan | 6C | 295852267 | A, C | sanfensan |
| 1C | 117607567 | C, A | sanfensan | 4A | 62632618 | T, C | sanfensan | 6C | 301581063 | G, A | sanfensan |
| 1C | 123766670 | G, C | sanfensan | 4A | 69360237 | A, C | sanfensan | 6C | 308002832 | T, C | sanfensan |
| 1C | 124698034 | C, T | sanfensan | 4A | 72548515 | T, C | sanfensan | 6C | 310533434 | G, A | sanfensan |
| 1C | 125624389 | G, A | sanfensan | 4A | 78860501 | A, G | sanfensan | 6C | 316651599 | G, C | sanfensan |
| 1C | 132272583 | G, T | sanfensan | 4A | 83477002 | A, G | sanfensan | 6C | 320349680 | G, A | sanfensan |
| 1C | 137221231 | G, A | sanfensan | 4A | 86718114 | A, C | sanfensan | 6C | 325082414 | T, C | sanfensan |
| 1C | 140336177 | G, A | sanfensan | 4A | 90186259 | T, C | sanfensan | 6C | 331834200 | C, T | sanfensan |
| 1C | 148863724 | C, T | sanfensan | 4A | 99717884 | C, G | sanfensan | 6C | 335435627 | T, C | sanfensan |
| 1C | 152576285 | T, G | sanfensan | 4A | 103226101 | C, T | sanfensan | 6C | 341601920 | C, A | sanfensan |
| 1C | 158557656 | G, C | sanfensan | 4A | 108841089 | C, A | sanfensan | 6C | 348384420 | T, C | sanfensan |
| 1C | 161866271 | C, A | sanfensan | 4A | 109097229 | G, A | sanfensan | 6C | 354770875 | T, C | sanfensan |
| 1C | 166850618 | G, A | sanfensan | 4A | 110362403 | T, C | sanfensan | 6C | 355787593 | T, C | sanfensan |
| 1C | 171246538 | C, A | sanfensan | 4A | 119524477 | G, C | sanfensan | 6C | 363981785 | C, T | sanfensan |
| 1C | 177121539 | G, A | sanfensan | 4A | 124335960 | C, T | sanfensan | 6C | 367279098 | C, G | sanfensan |
| 1C | 184861598 | C, T | sanfensan | 4A | 129987141 | C, T | sanfensan | 6C | 372097294 | G, T | sanfensan |
| 1C | 187896025 | C, A | sanfensan | 4A | 132188846 | A, G | sanfensan | 6C | 376086340 | G, A | sanfensan |
| 1C | 192704844 | C, T | sanfensan | 4A | 138010184 | A, G | sanfensan | 6C | 382987219 | C, T | sanfensan |
| 1C | 196342850 | G, A | sanfensan | 4A | 141248665 | T, C | sanfensan | 6C | 385017932 | A, G | sanfensan |
| 1C | 203152613 | A, G | sanfensan | 4A | 141974369 | A, G | sanfensan | 6C | 393722890 | C, T | sanfensan |
| 1C | 206698681 | G, A | sanfensan | 4A | 146462646 | G, A | sanfensan | 6C | 396489334 | T, C | sanfensan |
| 1C | 212808498 | G, A | sanfensan | 4A | 151979768 | T, C | sanfensan | 6C | 402999061 | G, A | sanfensan |
| 1C | 219911347 | A, G | sanfensan | 4A | 158451505 | G, A | sanfensan | 6C | 404034553 | G, A | sanfensan |
| 1C | 223588927 | G, A | sanfensan | 4A | 163350428 | A, G | sanfensan | 6C | 407439238 | G, C | sanfensan |
| 1C | 228818861 | G, T | sanfensan | 4A | 167565220 | A, G | sanfensan | 6C | 412317336 | C, T | sanfensan |
| 1C | 231062523 | C, T | sanfensan | 4A | 173953101 | T, G | sanfensan | 6C | 416156258 | T, C | sanfensan |
| 1C | 244308309 | G, A | sanfensan | 4A | 178883204 | G, C | sanfensan | 6C | 422586122 | G, C | sanfensan |
| 1C | 244949293 | G, C | sanfensan | 4A | 184596923 | C, T | sanfensan | 6C | 429700897 | C, T | sanfensan |
| 1C | 247382592 | G, A | sanfensan | 4A | 189201669 | A, C | sanfensan | 6C | 430532023 | A, G | sanfensan |
| 1C | 251563472 | C, G | sanfensan | 4A | 193547898 | G, A | sanfensan | 6C | 436366975 | G, A | sanfensan |
| 1C | 258583411 | G, C | sanfensan | 4A | 199508298 | A, G | sanfensan | 6C | 440291570 | T, G | sanfensan |
| 1C | 264870633 | C, T | sanfensan | 4A | 201195563 | C, T | sanfensan | 6C | 447368142 | A, G | sanfensan |
| 1C | 269866051 | G, A | sanfensan | 4A | 207185396 | C, A | sanfensan | 6C | 454158229 | C, T | sanfensan |
| 1C | 270041152 | T, C | sanfensan | 4A | 213373498 | T, C | sanfensan | 6C | 457431878 | G, A | sanfensan |
| 1C | 279714245 | T, C | sanfensan | 4A | 216222538 | A, G | sanfensan | 6C | 464408570 | A, G | sanfensan |
| 1C | 281426354 | G, A | sanfensan | 4A | 221795376 | C, T | sanfensan | 6C | 466196657 | G, A | sanfensan |
| 1C | 289269367 | C, G | sanfensan | 4A | 228345642 | A, T | sanfensan | 6C | 470467140 | G, T | sanfensan |
| 1C | 290192142 | C, A | sanfensan | 4A | 231330723 | A, C | sanfensan | 6C | 475226773 | G, A | sanfensan |
| 1C | 295651800 | G, T | sanfensan | 4A | 235514032 | G, A | sanfensan | 6C | 484622983 | G, A | sanfensan |
| 1C | 301166258 | G, C | sanfensan | 4A | 242193423 | C, T | sanfensan | 6C | 489733584 | G, T | sanfensan |
| 1C | 305159269 | G, T | sanfensan | 4A | 247945600 | C, T | sanfensan | 6C | 491124804 | T, C | sanfensan |
| 1C | 311692892 | C, T | sanfensan | 4A | 252369025 | T, C | sanfensan | 6C | 496637243 | G, A | sanfensan |
| 1C | 318201468 | T, G | sanfensan | 4A | 259286784 | G, C | sanfensan | 6C | 502828531 | C, T | sanfensan |
| 1C | 322797850 | G, A | sanfensan | 4A | 261531070 | A, G | sanfensan | 6C | 505002174 | G, A | sanfensan |
| 1C | 329636395 | C, T | sanfensan | 4A | 268127413 | G, T | sanfensan | 6C | 507736024 | C, T | sanfensan |
| 1C | 331069160 | G, A | sanfensan | 4A | 273296067 | A, G | sanfensan | 6C | 512237599 | C, T | sanfensan |
| 1C | 338302628 | G, A | sanfensan | 4A | 276596131 | T, C | sanfensan | 6C | 516058707 | C, T | sanfensan |
| 1C | 343573672 | A, G | sanfensan | 4A | 280307793 | C, T | sanfensan | 6C | 522649701 | C, G | sanfensan |
| 1C | 346562089 | G, A | sanfensan | 4A | 289509128 | C, T | sanfensan | 6C | 529463088 | G, T | sanfensan |
| 1C | 350268981 | C, T | sanfensan | 4A | 293451571 | G, A | sanfensan | 6C | 532838127 | C, T | sanfensan |
| 1C | 359261478 | T, C | sanfensan | 4A | 299169983 | T, G | sanfensan | 6C | 538744714 | C, T | sanfensan |
| 1C | 361793363 | G, A | sanfensan | 4A | 300515091 | T, C | sanfensan | 6C | 541894652 | T, C | sanfensan |
| 1C | 366795285 | C, G | sanfensan | 4A | 305599904 | G, A | sanfensan | 6C | 548890274 | A, G | sanfensan |
| 1C | 373361346 | G, A | sanfensan | 4A | 311085314 | A, C | sanfensan | 6C | 550597198 | C, T | sanfensan |
| 1C | 377285872 | C, T | sanfensan | 4A | 316042196 | C, T | sanfensan | 6C | 555553384 | T, C | sanfensan |
| 1C | 384514418 | T, G | sanfensan | 4A | 322946253 | T, C | sanfensan | 6C | 563333574 | G, T | sanfensan |
| 1C | 388701553 | G, A | sanfensan | 4A | 325478648 | G, C | sanfensan | 6C | 566938058 | T, C | sanfensan |
| 1C | 391109953 | C, T | sanfensan | 4A | 330347981 | A, G | sanfensan | 6C | 574826495 | A, C | sanfensan |
| 1C | 398781194 | A, C | sanfensan | 4A | 337443775 | C, T | sanfensan | 6C | 576805876 | G, A | sanfensan |
| 1C | 404904380 | A, C | sanfensan | 4A | 340462080 | A, G | sanfensan | 6C | 581584319 | G, C | sanfensan |
| 1C | 409167422 | A, G | sanfensan | 4A | 347382275 | A, G | sanfensan | 6C | 585343852 | C, T | sanfensan |
| 1C | 410239696 | G, A | sanfensan | 4A | 354497490 | T, G | sanfensan | 6C | 590741273 | T, C | sanfensan |
| 1C | 418466563 | A, G | sanfensan | 4A | 359050714 | C, A | sanfensan | 6C | 599908901 | G, A | sanfensan |
| 1C | 424408198 | C, T | sanfensan | 4A | 362340167 | C, T | sanfensan | 6C | 601571029 | C, T | sanfensan |
| 1C | 426142267 | T, C | sanfensan | 4A | 367751846 | G, A | sanfensan | 6C | 605547418 | C, T | sanfensan |
| 1C | 434346817 | G, A | sanfensan | 4A | 370622530 | G, T | sanfensan | 6D | 2777295 | C, T | sanfensan |
| 1C | 435126767 | A, G | sanfensan | 4A | 379177676 | T, C | sanfensan | 6D | 6267655 | C, A | sanfensan |
| 1C | 444951853 | C, T | sanfensan | 4A | 381166365 | T, C | sanfensan | 6D | 13107420 | C, T | sanfensan |
| 1C | 446632589 | T, C | sanfensan | 4A | 385406079 | C, G | sanfensan | 6D | 16132609 | C, A | sanfensan |
| 1C | 451346048 | G, A | sanfensan | 4A | 398015147 | A, C | sanfensan | 6D | 23982220 | A, C | sanfensan |
| 1C | 458628576 | C, T | sanfensan | 4A | 401416507 | G, A | sanfensan | 6D | 26754468 | T, G | sanfensan |
| 1D | 4748051 | C, T | sanfensan | 4A | 406634471 | A, G | sanfensan | 6D | 32172782 | C, T | sanfensan |
| 1D | 9972757 | C, T | sanfensan | 4A | 413012139 | A, G | sanfensan | 6D | 35628869 | T, C | sanfensan |
| 1D | 14428103 | T, C | sanfensan | 4A | 418384264 | C, T | sanfensan | 6D | 44217561 | A, G | sanfensan |
| 1D | 17910637 | T, G | sanfensan | 4A | 424141863 | C, T | sanfensan | 6D | 45240610 | T, G | sanfensan |
| 1D | 22886105 | A, C | sanfensan | 4A | 426462945 | A, T | sanfensan | 6D | 50128337 | G, C | sanfensan |
| 1D | 26189931 | T, C | sanfensan | 4A | 434286106 | G, T | sanfensan | 6D | 55030113 | A, G | sanfensan |
| 1D | 34096819 | T, G | sanfensan | 4A | 435134503 | C, T | sanfensan | 6D | 64553482 | T, C | sanfensan |
| 1D | 36812285 | T, C | sanfensan | 4A | 440807368 | C, G | sanfensan | 6D | 66247035 | T, C | sanfensan |
| 1D | 44167411 | T, C | sanfensan | 4A | 445324027 | C, T | sanfensan | 6D | 73309196 | A, G | sanfensan |
| 1D | 48753097 | C, T | sanfensan | 4A | 450745492 | T, C | sanfensan | 6D | 77363272 | A, G | sanfensan |
| 1D | 52943769 | G, A | sanfensan | 4A | 458822974 | A, G | sanfensan | 6D | 84655953 | T, C | sanfensan |
| 1D | 59943693 | C, T | sanfensan | 4A | 462418830 | A, C | sanfensan | 6D | 89015326 | T, C | sanfensan |
| 1D | 61299591 | T, A | sanfensan | 4A | 466738641 | T, C | sanfensan | 6D | 91516090 | A, G | sanfensan |
| 1D | 67828154 | C, T | sanfensan | 4C | 3595818 | G, A | sanfensan | 6D | 98584233 | A, G | sanfensan |
| 1D | 74812678 | T, C | sanfensan | 4C | 6028133 | A, G | sanfensan | 6D | 104170504 | T, G | sanfensan |
| 1D | 76558850 | A, G | sanfensan | 4C | 13049345 | T, A | sanfensan | 6D | 108654428 | T, C | sanfensan |
| 1D | 83489940 | C, T | sanfensan | 4C | 19972099 | A, G | sanfensan | 6D | 114351697 | C, G | sanfensan |
| 1D | 85424309 | G, A | sanfensan | 4C | 20771307 | T, C | sanfensan | 6D | 118475182 | G, A | sanfensan |
| 1D | 90502825 | G, T | sanfensan | 4C | 25886686 | G, C | sanfensan | 6D | 120657246 | A, G | sanfensan |
| 1D | 99173014 | C, A | sanfensan | 4C | 32211660 | C, A | sanfensan | 6D | 128950046 | A, G | sanfensan |
| 1D | 101866462 | A, G | sanfensan | 4C | 36873418 | G, C | sanfensan | 6D | 132670154 | T, C | sanfensan |
| 1D | 105176621 | G, T | sanfensan | 4C | 40449202 | C, G | sanfensan | 6D | 139598945 | A, G | sanfensan |
| 1D | 112466846 | A, G | sanfensan | 4C | 47158151 | C, A | sanfensan | 6D | 142833751 | A, C | sanfensan |
| 1D | 116735117 | T, C | sanfensan | 4C | 51926597 | T, C | sanfensan | 6D | 146878955 | C, T | sanfensan |
| 1D | 120724766 | T, G | sanfensan | 4C | 57722231 | G, A | sanfensan | 6D | 150804995 | G, A | sanfensan |
| 1D | 125083241 | A, G | sanfensan | 4C | 61365967 | G, A | sanfensan | 6D | 159442646 | T, C | sanfensan |
| 1D | 134949445 | T, C | sanfensan | 4C | 66290570 | C, T | sanfensan | 6D | 161979848 | G, A | sanfensan |
| 1D | 136722735 | T, C | sanfensan | 4C | 69406750 | G, A | sanfensan | 6D | 166007399 | C, T | sanfensan |
| 1D | 140036387 | C, T | sanfensan | 4C | 71656189 | G, A | sanfensan | 6D | 171110144 | T, C | sanfensan |
| 1D | 149197821 | G, A | sanfensan | 4C | 75202185 | A, G | sanfensan | 6D | 178157758 | C, T | sanfensan |
| 1D | 154586415 | G, T | sanfensan | 4C | 80202847 | G, T | sanfensan | 6D | 181144200 | A, C | sanfensan |
| 1D | 156993466 | T, G | sanfensan | 4C | 86069619 | A, G | sanfensan | 6D | 181398145 | T, G | sanfensan |
| 1D | 160591807 | T, G | sanfensan | 4C | 92181840 | C, T | sanfensan | 6D | 188963826 | T, C | sanfensan |
| 1D | 169373396 | G, A | sanfensan | 4C | 95153438 | G, A | sanfensan | 6D | 190320583 | G, A | sanfensan |
| 1D | 172161852 | G, T | sanfensan | 4C | 101779057 | T, C | sanfensan | 6D | 198738426 | G, A | sanfensan |
| 1D | 178558164 | T, C | sanfensan | 4C | 106362411 | C, T | sanfensan | 6D | 204695478 | C, T | sanfensan |
| 1D | 182138470 | A, G | sanfensan | 4C | 113373499 | T, G | sanfensan | 6D | 209111889 | A, C | sanfensan |
| 1D | 188332946 | T, C | sanfensan | 4C | 116319458 | C, T | sanfensan | 6D | 213388210 | T, C | sanfensan |
| 1D | 191338972 | T, C | sanfensan | 4C | 122530727 | A, G | sanfensan | 6D | 217365606 | G, A | sanfensan |
| 1D | 197805002 | G, A | sanfensan | 4C | 128925968 | T, G | sanfensan | 6D | 220497638 | C, T | sanfensan |
| 1D | 201653641 | A, C | sanfensan | 4C | 130658845 | T, C | sanfensan | 6D | 226248655 | G, T | sanfensan |
| 1D | 205231760 | T, C | sanfensan | 4C | 139094233 | A, G | sanfensan | 6D | 230357776 | T, C | sanfensan |
| 1D | 213849974 | A, G | sanfensan | 4C | 141439434 | G, T | sanfensan | 6D | 239771210 | A, G | sanfensan |
| 1D | 218342762 | G, C | sanfensan | 4C | 149617745 | A, C | sanfensan | 6D | 240712867 | T, A | sanfensan |
| 1D | 221295907 | A, C | sanfensan | 4C | 151871212 | T, C | sanfensan | 6D | 245616215 | G, A | sanfensan |
| 1D | 225627642 | A, G | sanfensan | 4C | 159944864 | C, T | sanfensan | 6D | 254910351 | T, C | sanfensan |
| 1D | 233309591 | C, T | sanfensan | 4C | 164691088 | T, C | sanfensan | 6D | 257401569 | T, C | sanfensan |
| 1D | 234707599 | G, A | sanfensan | 4C | 169970304 | C, A | sanfensan | 6D | 263701170 | T, G | sanfensan |
| 1D | 239408154 | C, T | sanfensan | 4C | 170331200 | A, C | sanfensan | 6D | 268768437 | A, G | sanfensan |
| 1D | 241284677 | T, G | sanfensan | 4C | 178692748 | C, T | sanfensan | 6D | 270928208 | C, A | sanfensan |
| 1D | 248921833 | T, G | sanfensan | 4C | 184841316 | C, T | sanfensan | 6D | 278371196 | C, T | sanfensan |
| 1D | 250623142 | A, T | sanfensan | 4C | 185887772 | T, C | sanfensan | 6D | 284570630 | A, G | sanfensan |
| 1D | 258844136 | C, T | sanfensan | 4C | 193734961 | C, T | sanfensan | 6D | 285173852 | A, G | sanfensan |
| 1D | 262320123 | A, C | sanfensan | 4C | 197024216 | G, A | sanfensan | 6D | 290592914 | T, A | sanfensan |
| 1D | 265785336 | T, C | sanfensan | 4C | 202625381 | G, A | sanfensan | 7A | 556988 | G, A | sanfensan |
| 1D | 270470362 | C, T | sanfensan | 4C | 209016901 | A, G | sanfensan | 7A | 9387452 | T, C | sanfensan |
| 1D | 275515735 | A, G | sanfensan | 4C | 213200350 | C, T | sanfensan | 7A | 10624667 | T, C | sanfensan |
| 1D | 284647168 | G, A | sanfensan | 4C | 215000144 | C, T | sanfensan | 7A | 16247028 | G, A | sanfensan |
| 1D | 288750391 | A, G | sanfensan | 4C | 224645115 | C, T | sanfensan | 7A | 24258432 | T, A | sanfensan |
| 1D | 293777596 | A, G | sanfensan | 4C | 229625246 | A, G | sanfensan | 7A | 29212189 | T, C | sanfensan |
| 1D | 298973779 | C, A | sanfensan | 4C | 234547399 | T, C | sanfensan | 7A | 32277248 | T, G | sanfensan |
| 1D | 304978046 | T, C | sanfensan | 4C | 236119582 | A, C | sanfensan | 7A | 38743380 | T, C | sanfensan |
| 1D | 306430734 | T, G | sanfensan | 4C | 241687009 | G, A | sanfensan | 7A | 42755867 | A, G | sanfensan |
| 1D | 313322569 | T, C | sanfensan | 4C | 245369143 | G, T | sanfensan | 7A | 46387061 | C, T | sanfensan |
| 1D | 317482376 | T, C | sanfensan | 4C | 252253086 | T, A | sanfensan | 7A | 51855350 | A, G | sanfensan |
| 1D | 321285642 | T, C | sanfensan | 4C | 255164714 | C, T | sanfensan | 7A | 57885922 | C, A | sanfensan |
| 1D | 328096417 | A, G | sanfensan | 4C | 260860063 | G, A | sanfensan | 7A | 63281807 | T, C | sanfensan |
| 1D | 334194238 | G, A | sanfensan | 4C | 267013553 | G, A | sanfensan | 7A | 65480794 | G, A | sanfensan |
| 1D | 336604011 | G, A | sanfensan | 4C | 273797546 | G, A | sanfensan | 7A | 71137163 | T, C | sanfensan |
| 1D | 342605207 | A, C | sanfensan | 4C | 277862984 | C, T | sanfensan | 7A | 75733143 | T, A | sanfensan |
| 1D | 348951141 | A, C | sanfensan | 4C | 282087726 | G, A | sanfensan | 7A | 82158130 | G, C | sanfensan |
| 1D | 352790704 | A, G | sanfensan | 4C | 287337582 | G, A | sanfensan | 7A | 85385227 | C, T | sanfensan |
| 1D | 355455291 | G, C | sanfensan | 4C | 292238812 | C, T | sanfensan | 7A | 91146020 | C, T | sanfensan |
| 1D | 364550878 | A, G | sanfensan | 4C | 296562693 | C, T | sanfensan | 7A | 99518702 | T, C | sanfensan |
| 1D | 369719851 | A, G | sanfensan | 4C | 301499481 | T, C | sanfensan | 7A | 100100809 | C, A | sanfensan |
| 1D | 372379876 | A, C | sanfensan | 4C | 308695483 | G, A | sanfensan | 7A | 108559177 | C, T | sanfensan |
| 1D | 379801584 | A, C | sanfensan | 4C | 311224923 | G, A | sanfensan | 7A | 110159568 | G, T | sanfensan |
| 1D | 380728291 | A, G | sanfensan | 4C | 316095744 | G, A | sanfensan | 7A | 119859092 | T, C | sanfensan |
| 1D | 388559569 | T, C | sanfensan | 4C | 324951331 | C, T | sanfensan | 7A | 120329198 | C, A | sanfensan |
| 1D | 394958911 | T, C | sanfensan | 4C | 325766510 | T, G | sanfensan | 7A | 125074054 | T, C | sanfensan |
| 1D | 395282752 | A, G | sanfensan | 4C | 331060999 | G, A | sanfensan | 7A | 131168556 | C, T | sanfensan |
| 1D | 403815045 | T, C | sanfensan | 4C | 339044922 | C, T | sanfensan | 7A | 139918960 | A, G | sanfensan |
| 1D | 409759489 | T, C | sanfensan | 4C | 342159259 | G, A | sanfensan | 7A | 143266476 | T, C | sanfensan |
| 1D | 412133901 | T, C | sanfensan | 4C | 345139072 | G, A | sanfensan | 7A | 149300335 | G, A | sanfensan |
| 1D | 418421166 | C, G | sanfensan | 4C | 350531907 | G, T | sanfensan | 7A | 154734171 | T, C | sanfensan |
| 1D | 423994932 | T, C | sanfensan | 4C | 357426076 | G, A | sanfensan | 7A | 159313052 | C, T | sanfensan |
| 1D | 429511063 | A, G | sanfensan | 4C | 362889043 | G, A | sanfensan | 7A | 160075815 | C, G | sanfensan |
| 1D | 433441263 | A, G | sanfensan | 4C | 369798229 | G, A | sanfensan | 7A | 167272883 | C, T | sanfensan |
| 1D | 435135220 | T, A | sanfensan | 4C | 372472310 | C, T | sanfensan | 7A | 171591003 | T, G | sanfensan |
| 1D | 442087046 | G, A | sanfensan | 4C | 378265862 | T, C | sanfensan | 7A | 177018083 | G, T | sanfensan |
| 1D | 442124461 | G, A | sanfensan | 4C | 381319655 | G, A | sanfensan | 7A | 184807238 | C, A | sanfensan |
| 1D | 449745797 | G, C | sanfensan | 4C | 385095261 | T, G | sanfensan | 7A | 186333580 | A, G | sanfensan |
| 1D | 450598659 | A, G | sanfensan | 4C | 387723697 | C, T | sanfensan | 7A | 190961467 | T, C | sanfensan |
| 1D | 459581711 | T, C | sanfensan | 4C | 392326058 | T, C | sanfensan | 7A | 197600313 | G, A | sanfensan |
| 1D | 460387603 | C, G | sanfensan | 4C | 395186922 | T, C | sanfensan | 7A | 201074764 | C, A | sanfensan |
| 1D | 468646779 | G, A | sanfensan | 4C | 404320541 | A, C | sanfensan | 7A | 208272139 | T, G | sanfensan |
| 1D | 470605204 | C, T | sanfensan | 4C | 407798339 | C, G | sanfensan | 7A | 212498087 | C, T | sanfensan |
| 2A | 1294852 | A, G | sanfensan | 4C | 414198946 | A, G | sanfensan | 7A | 219185506 | G, T | sanfensan |
| 2A | 8354341 | G, A | sanfensan | 4C | 418786637 | C, T | sanfensan | 7A | 222607909 | A, G | sanfensan |
| 2A | 10205647 | T, G | sanfensan | 4C | 420987303 | G, C | sanfensan | 7A | 226206685 | T, C | sanfensan |
| 2A | 17133037 | A, G | sanfensan | 4C | 425274444 | A, T | sanfensan | 7A | 234382860 | A, T | sanfensan |
| 2A | 22147985 | C, G | sanfensan | 4C | 437039125 | T, C | sanfensan | 7A | 235670912 | C, T | sanfensan |
| 2A | 25577776 | C, A | sanfensan | 4C | 442594181 | C, T | sanfensan | 7A | 242252774 | A, G | sanfensan |
| 2A | 33113184 | G, T | sanfensan | 4C | 446643052 | G, T | sanfensan | 7A | 246670561 | A, C | sanfensan |
| 2A | 38311226 | G, A | sanfensan | 4C | 451884828 | C, A | sanfensan | 7A | 250206642 | T, C | sanfensan |
| 2A | 43688833 | A, C | sanfensan | 4C | 455850824 | G, A | sanfensan | 7A | 259731844 | C, T | sanfensan |
| 2A | 45363179 | C, T | sanfensan | 4C | 463494104 | C, T | sanfensan | 7A | 261123685 | T, C | sanfensan |
| 2A | 54582062 | A, G | sanfensan | 4C | 467165660 | C, T | sanfensan | 7A | 267061512 | G, C | sanfensan |
| 2A | 58712383 | A, G | sanfensan | 4C | 472240988 | T, G | sanfensan | 7A | 273875056 | A, T | sanfensan |
| 2A | 60354747 | A, G | sanfensan | 4C | 475026617 | T, C | sanfensan | 7A | 279451537 | C, G | sanfensan |
| 2A | 69920614 | A, G | sanfensan | 4C | 481585885 | G, A | sanfensan | 7A | 283880271 | G, A | sanfensan |
| 2A | 74714222 | T, C | sanfensan | 4C | 487240271 | G, T | sanfensan | 7A | 289470204 | T, C | sanfensan |
| 2A | 78592686 | A, C | sanfensan | 4C | 493243160 | C, T | sanfensan | 7A | 294405482 | T, C | sanfensan |
| 2A | 79665300 | C, A | sanfensan | 4C | 499531487 | C, A | sanfensan | 7A | 296729065 | G, T | sanfensan |
| 2A | 83491335 | A, G | sanfensan | 4C | 504518607 | G, A | sanfensan | 7A | 304733749 | T, C | sanfensan |
| 2A | 88992475 | A, C | sanfensan | 4C | 509955623 | G, A | sanfensan | 7A | 306515921 | T, C | sanfensan |
| 2A | 91196574 | C, T | sanfensan | 4C | 511514837 | A, G | sanfensan | 7A | 310678817 | G, A | sanfensan |
| 2A | 98762331 | C, G | sanfensan | 4C | 514138096 | G, A | sanfensan | 7A | 317998480 | C, T | sanfensan |
| 2A | 102839151 | T, C | sanfensan | 4C | 519111243 | A, T | sanfensan | 7A | 324083519 | A, G | sanfensan |
| 2A | 105115673 | C, T | sanfensan | 4C | 520487689 | C, T | sanfensan | 7A | 326015574 | A, G | sanfensan |
| 2A | 113143954 | G, A | sanfensan | 4C | 526516206 | A, G | sanfensan | 7A | 334096551 | G, A | sanfensan |
| 2A | 115778719 | C, T | sanfensan | 4C | 530500162 | C, T | sanfensan | 7A | 337503568 | G, A | sanfensan |
| 2A | 123726669 | G, C | sanfensan | 4C | 537644622 | T, C | sanfensan | 7A | 342389640 | T, C | sanfensan |
| 2A | 125030830 | G, A | sanfensan | 4C | 540730312 | G, A | sanfensan | 7A | 349080210 | A, G | sanfensan |
| 2A | 133605969 | C, A | sanfensan | 4C | 545836115 | G, T | sanfensan | 7A | 352615237 | T, C | sanfensan |
| 2A | 136067584 | A, C | sanfensan | 4C | 549932188 | A, G | sanfensan | 7A | 355460008 | G, A | sanfensan |
| 2A | 142891390 | C, T | sanfensan | 4C | 550915489 | A, T | sanfensan | 7A | 361858462 | T, C | sanfensan |
| 2A | 148925271 | C, T | sanfensan | 4C | 558054888 | C, T | sanfensan | 7A | 363873233 | C, T | sanfensan |
| 2A | 150242064 | A, G | sanfensan | 4C | 562963997 | A, G | sanfensan | 7A | 367416741 | A, G | sanfensan |
| 2A | 159722254 | T, C | sanfensan | 4C | 569467277 | A, G | sanfensan | 7A | 370900705 | C, A | sanfensan |
| 2A | 161633504 | G, A | sanfensan | 4C | 572657475 | T, C | sanfensan | 7A | 377494585 | C, T | sanfensan |
| 2A | 166801852 | G, A | sanfensan | 4C | 575327801 | A, G | sanfensan | 7A | 380620357 | T, C | sanfensan |
| 2A | 172884455 | G, C | sanfensan | 4C | 584368593 | T, G | sanfensan | 7A | 387542205 | A, G | sanfensan |
| 2A | 177892881 | A, G | sanfensan | 4C | 588543504 | A, G | sanfensan | 7A | 393193897 | T, C | sanfensan |
| 2A | 180161463 | A, C | sanfensan | 4C | 589445451 | A, G | sanfensan | 7A | 397890567 | A, G | sanfensan |
| 2A | 187191668 | G, A | sanfensan | 4C | 593669777 | A, C | sanfensan | 7A | 402356276 | A, T | sanfensan |
| 2A | 194352120 | C, A | sanfensan | 4C | 599151537 | A, G | sanfensan | 7A | 408034177 | T, G | sanfensan |
| 2A | 195938867 | T, C | sanfensan | 4C | 600053663 | T, C | sanfensan | 7A | 411938390 | C, T | sanfensan |
| 2A | 202623332 | G, T | sanfensan | 4C | 607995668 | A, G | sanfensan | 7A | 415820845 | C, T | sanfensan |
| 2A | 209957751 | G, T | sanfensan | 4C | 610178903 | T, C | sanfensan | 7A | 421650338 | A, G | sanfensan |
| 2A | 210875267 | T, G | sanfensan | 4C | 614566248 | G, C | sanfensan | 7A | 429369869 | A, G | sanfensan |
| 2A | 215784372 | C, T | sanfensan | 4C | 616610794 | C, T | sanfensan | 7A | 433335698 | G, A | sanfensan |
| 2A | 224379638 | G, C | sanfensan | 4C | 624701501 | T, C | sanfensan | 7A | 438899194 | T, C | sanfensan |
| 2A | 227479068 | C, T | sanfensan | 4C | 626629588 | G, A | sanfensan | 7A | 442020982 | T, C | sanfensan |
| 2A | 231405711 | A, C | sanfensan | 4C | 631097435 | A, C | sanfensan | 7A | 449206870 | A, G | sanfensan |
| 2A | 237629147 | C, T | sanfensan | 4C | 635505874 | C, A | sanfensan | 7A | 452644138 | A, C | sanfensan |
| 2A | 240260139 | T, C | sanfensan | 4C | 642622496 | T, C | sanfensan | 7A | 455480446 | T, G | sanfensan |
| 2A | 248913514 | G, C | sanfensan | 4C | 645881564 | A, C | sanfensan | 7A | 463118331 | A, G | sanfensan |
| 2A | 251101436 | C, T | sanfensan | 4C | 653135330 | G, T | sanfensan | 7A | 467735097 | C, T | sanfensan |
| 2A | 258858124 | C, T | sanfensan | 4C | 655135466 | A, G | sanfensan | 7A | 471902717 | A, G | sanfensan |
| 2A | 262168685 | G, A | sanfensan | 4C | 660684760 | G, A | sanfensan | 7A | 476107323 | A, G | sanfensan |
| 2A | 269432173 | A, C | sanfensan | 4C | 665124776 | G, C | sanfensan | 7A | 482021904 | C, T | sanfensan |
| 2A | 272066634 | T, C | sanfensan | 4C | 674949846 | A, G | sanfensan | 7A | 485131125 | A, G | sanfensan |
| 2A | 275191428 | C, T | sanfensan | 4C | 675185316 | A, G | sanfensan | 7A | 493482291 | G, A | sanfensan |
| 2A | 282263456 | T, G | sanfensan | 4C | 683318163 | T, C | sanfensan | 7A | 495121727 | T, C | sanfensan |
| 2A | 288156444 | C, G | sanfensan | 4C | 689664776 | T, C | sanfensan | 7C | 4614858 | T, G | sanfensan |
| 2A | 291947063 | A, C | sanfensan | 4C | 694624379 | C, T | sanfensan | 7C | 5317219 | C, T | sanfensan |
| 2A | 298698676 | T, C | sanfensan | 4C | 695265876 | A, G | sanfensan | 7C | 14340861 | A, G | sanfensan |
| 2A | 302165782 | G, A | sanfensan | 4C | 701273830 | A, G | sanfensan | 7C | 19478034 | G, A | sanfensan |
| 2A | 307413298 | T, C | sanfensan | 4D | 3022923 | A, G | sanfensan | 7C | 24565676 | G, A | sanfensan |
| 2A | 314041736 | C, G | sanfensan | 4D | 7849878 | C, T | sanfensan | 7C | 29930105 | T, C | sanfensan |
| 2A | 319115852 | C, T | sanfensan | 4D | 10672344 | A, G | sanfensan | 7C | 32494696 | G, A | sanfensan |
| 2A | 324309925 | C, T | sanfensan | 4D | 16331251 | T, G | sanfensan | 7C | 39005353 | C, T | sanfensan |
| 2A | 325423488 | A, C | sanfensan | 4D | 23073905 | A, G | sanfensan | 7C | 41317427 | A, G | sanfensan |
| 2A | 332013465 | T, C | sanfensan | 4D | 28279824 | T, G | sanfensan | 7C | 47961168 | A, C | sanfensan |
| 2A | 336329704 | T, G | sanfensan | 4D | 32649688 | A, G | sanfensan | 7C | 50182911 | G, C | sanfensan |
| 2A | 337567230 | G, T | sanfensan | 4D | 37038379 | A, C | sanfensan | 7C | 55925952 | A, G | sanfensan |
| 2A | 340385583 | C, T | sanfensan | 4D | 43180451 | T, C | sanfensan | 7C | 61239403 | C, G | sanfensan |
| 2A | 347331178 | A, G | sanfensan | 4D | 45472604 | A, G | sanfensan | 7C | 67047337 | T, G | sanfensan |
| 2A | 354110393 | C, T | sanfensan | 4D | 50482410 | A, G | sanfensan | 7C | 71975946 | G, T | sanfensan |
| 2A | 355224874 | C, T | sanfensan | 4D | 58490879 | G, A | sanfensan | 7C | 72522696 | C, T | sanfensan |
| 2A | 361450337 | A, G | sanfensan | 4D | 62917919 | G, A | sanfensan | 7C | 79287234 | A, C | sanfensan |
| 2A | 365973983 | T, C | sanfensan | 4D | 65612418 | G, A | sanfensan | 7C | 84848962 | G, A | sanfensan |
| 2A | 374152559 | T, C | sanfensan | 4D | 71637400 | C, T | sanfensan | 7C | 88219749 | C, T | sanfensan |
| 2A | 378774926 | A, G | sanfensan | 4D | 76187635 | C, T | sanfensan | 7C | 94995046 | A, G | sanfensan |
| 2A | 380652237 | C, T | sanfensan | 4D | 82058789 | T, C | sanfensan | 7C | 97771126 | T, C | sanfensan |
| 2A | 386433427 | G, A | sanfensan | 4D | 88887772 | G, A | sanfensan | 7C | 104891291 | G, A | sanfensan |
| 2A | 391759319 | C, T | sanfensan | 4D | 92323177 | A, G | sanfensan | 7C | 108552236 | A, G | sanfensan |
| 2A | 397676516 | C, G | sanfensan | 4D | 97430755 | A, G | sanfensan | 7C | 113384152 | G, T | sanfensan |
| 2A | 404431299 | T, C | sanfensan | 4D | 102821671 | C, G | sanfensan | 7C | 118141656 | A, G | sanfensan |
| 2A | 406323117 | T, C | sanfensan | 4D | 106018930 | T, G | sanfensan | 7C | 123007101 | G, C | sanfensan |
| 2A | 414368443 | A, G | sanfensan | 4D | 110865436 | A, G | sanfensan | 7C | 129029468 | T, C | sanfensan |
| 2A | 418701849 | C, A | sanfensan | 4D | 116039043 | C, T | sanfensan | 7C | 134291990 | T, C | sanfensan |
| 2A | 421611597 | T, C | sanfensan | 4D | 118774001 | C, G | sanfensan | 7C | 137256580 | T, C | sanfensan |
| 2A | 429392443 | C, A | sanfensan | 4D | 121609230 | A, G | sanfensan | 7C | 140877690 | G, A | sanfensan |
| 2A | 432088076 | C, A | sanfensan | 4D | 128046905 | T, C | sanfensan | 7C | 145949568 | G, C | sanfensan |
| 2A | 432433952 | T, G | sanfensan | 4D | 134025258 | T, C | sanfensan | 7C | 151007290 | G, A | sanfensan |
| 2A | 437058043 | T, C | sanfensan | 4D | 137854184 | C, G | sanfensan | 7C | 157054447 | C, T | sanfensan |
| 2A | 442675069 | G, C | sanfensan | 4D | 141417395 | A, G | sanfensan | 7C | 163909424 | G, A | sanfensan |
| 2A | 447978464 | A, G | sanfensan | 4D | 148636319 | A, G | sanfensan | 7C | 166612341 | A, C | sanfensan |
| 2A | 452816108 | C, A | sanfensan | 4D | 151219343 | C, T | sanfensan | 7C | 171906105 | A, G | sanfensan |
| 2A | 456332224 | T, C | sanfensan | 4D | 153032013 | T, C | sanfensan | 7C | 177293559 | T, C | sanfensan |
| 2A | 460704136 | T, C | sanfensan | 4D | 159949170 | T, C | sanfensan | 7C | 179283192 | T, C | sanfensan |
| 2C | 3755642 | C, G | sanfensan | 4D | 163913258 | A, G | sanfensan | 7C | 180628837 | C, T | sanfensan |
| 2C | 6523209 | T, C | sanfensan | 4D | 165337682 | T, A | sanfensan | 7C | 185998111 | A, G | sanfensan |
| 2C | 14666414 | G, A | sanfensan | 4D | 173510202 | T, G | sanfensan | 7C | 191666515 | A, G | sanfensan |
| 2C | 18219538 | G, A | sanfensan | 4D | 179301600 | C, G | sanfensan | 7C | 197044982 | A, G | sanfensan |
| 2C | 20598121 | C, T | sanfensan | 4D | 184426060 | G, A | sanfensan | 7C | 204791800 | A, C | sanfensan |
| 2C | 25528699 | G, A | sanfensan | 4D | 187340496 | G, T | sanfensan | 7C | 206297454 | T, C | sanfensan |
| 2C | 34351879 | G, A | sanfensan | 4D | 193603755 | A, G | sanfensan | 7C | 214665049 | T, C | sanfensan |
| 2C | 37480085 | G, A | sanfensan | 4D | 196382892 | T, C | sanfensan | 7C | 216739159 | A, G | sanfensan |
| 2C | 40282520 | C, T | sanfensan | 4D | 202074844 | C, T | sanfensan | 7C | 220606602 | T, C | sanfensan |
| 2C | 47255505 | G, A | sanfensan | 4D | 206447702 | G, A | sanfensan | 7C | 225604579 | C, A | sanfensan |
| 2C | 52177917 | C, T | sanfensan | 4D | 212380635 | C, T | sanfensan | 7C | 232971786 | G, C | sanfensan |
| 2C | 55006635 | C, A | sanfensan | 4D | 215885690 | T, C | sanfensan | 7C | 236226644 | C, T | sanfensan |
| 2C | 62911833 | A, C | sanfensan | 4D | 220033123 | T, G | sanfensan | 7C | 242089348 | C, G | sanfensan |
| 2C | 69182434 | C, T | sanfensan | 4D | 227048389 | A, G | sanfensan | 7C | 247003405 | C, T | sanfensan |
| 2C | 73235690 | G, A | sanfensan | 4D | 232039750 | C, T | sanfensan | 7C | 249178004 | T, G | sanfensan |
| 2C | 75663383 | C, T | sanfensan | 4D | 236815937 | T, A | sanfensan | 7C | 250146710 | C, T | sanfensan |
| 2C | 80698781 | C, T | sanfensan | 4D | 242091618 | C, T | sanfensan | 7C | 257100796 | G, A | sanfensan |
| 2C | 88439094 | G, C | sanfensan | 4D | 245101303 | C, A | sanfensan | 7C | 261648943 | C, A | sanfensan |
| 2C | 93854384 | C, T | sanfensan | 4D | 250407222 | A, G | sanfensan | 7C | 268903706 | T, C | sanfensan |
| 2C | 97362350 | G, A | sanfensan | 4D | 256019130 | T, C | sanfensan | 7C | 273820692 | C, G | sanfensan |
| 2C | 104745256 | C, T | sanfensan | 4D | 260909900 | T, C | sanfensan | 7C | 278686128 | T, C | sanfensan |
| 2C | 108205218 | C, T | sanfensan | 4D | 267887871 | A, G | sanfensan | 7C | 280947265 | G, A | sanfensan |
| 2C | 110557690 | G, A | sanfensan | 4D | 271126060 | C, A | sanfensan | 7C | 286809973 | T, C | sanfensan |
| 2C | 119588975 | C, T | sanfensan | 4D | 277162174 | T, G | sanfensan | 7C | 291012394 | G, C | sanfensan |
| 2C | 122248235 | C, T | sanfensan | 4D | 283660950 | G, A | sanfensan | 7C | 296841339 | A, C | sanfensan |
| 2C | 129849765 | C, T | sanfensan | 4D | 285824135 | G, T | sanfensan | 7C | 301552328 | T, C | sanfensan |
| 2C | 134962790 | G, A | sanfensan | 4D | 294845699 | G, A | sanfensan | 7C | 304378578 | A, G | sanfensan |
| 2C | 135013067 | T, G | sanfensan | 4D | 299632398 | G, A | sanfensan | 7C | 306040659 | G, A | sanfensan |
| 2C | 140602512 | C, T | sanfensan | 4D | 303350038 | T, C | sanfensan | 7C | 314412301 | G, T | sanfensan |
| 2C | 145478940 | T, C | sanfensan | 4D | 307597358 | T, A | sanfensan | 7C | 318527191 | C, T | sanfensan |
| 2C | 150692118 | G, A | sanfensan | 4D | 314228425 | C, T | sanfensan | 7C | 320205502 | A, G | sanfensan |
| 2C | 156314693 | G, A | sanfensan | 4D | 314253165 | G, A | sanfensan | 7C | 320293039 | G, T | sanfensan |
| 2C | 160485851 | C, T | sanfensan | 4D | 315607825 | A, C | sanfensan | 7C | 327857134 | C, A | sanfensan |
| 2C | 167462260 | G, A | sanfensan | 4D | 321533893 | G, T | sanfensan | 7C | 331712162 | T, C | sanfensan |
| 2C | 173642372 | C, T | sanfensan | 4D | 325136857 | A, T | sanfensan | 7C | 339499335 | T, C | sanfensan |
| 2C | 179174429 | C, A | sanfensan | 4D | 326786150 | A, T | sanfensan | 7C | 340372302 | C, T | sanfensan |
| 2C | 182456426 | G, A | sanfensan | 4D | 332125743 | A, G | sanfensan | 7C | 349292850 | C, T | sanfensan |
| 2C | 188223122 | C, T | sanfensan | 4D | 335136720 | A, G | sanfensan | 7C | 350022239 | G, A | sanfensan |
| 2C | 190626883 | C, T | sanfensan | 4D | 340382269 | C, T | sanfensan | 7C | 350993553 | A, G | sanfensan |
| 2C | 196959827 | G, A | sanfensan | 4D | 348383382 | A, G | sanfensan | 7C | 358188563 | T, C | sanfensan |
| 2C | 201765496 | G, T | sanfensan | 4D | 352477479 | T, C | sanfensan | 7C | 363492217 | C, T | sanfensan |
| 2C | 207043151 | C, T | sanfensan | 4D | 356651761 | A, G | sanfensan | 7C | 369432993 | G, A | sanfensan |
| 2C | 208215965 | C, T | sanfensan | 4D | 363724413 | C, G | sanfensan | 7C | 371369521 | T, G | sanfensan |
| 2C | 213490479 | G, A | sanfensan | 4D | 366480345 | A, G | sanfensan | 7C | 377634589 | A, G | sanfensan |
| 2C | 217643681 | G, A | sanfensan | 4D | 374238192 | T, C | sanfensan | 7C | 384171632 | C, T | sanfensan |
| 2C | 222136297 | G, A | sanfensan | 4D | 378149215 | T, C | sanfensan | 7C | 386369415 | G, A | sanfensan |
| 2C | 226477864 | C, T | sanfensan | 4D | 384366440 | G, A | sanfensan | 7C | 392363537 | C, T | sanfensan |
| 2C | 231505039 | C, T | sanfensan | 4D | 389105351 | A, C | sanfensan | 7C | 396114953 | A, G | sanfensan |
| 2C | 237675775 | C, T | sanfensan | 4D | 390762487 | A, G | sanfensan | 7C | 400812310 | C, T | sanfensan |
| 2C | 240667277 | G, A | sanfensan | 4D | 398109909 | C, G | sanfensan | 7C | 405139125 | A, G | sanfensan |
| 2C | 246770167 | A, C | sanfensan | 4D | 402374421 | T, C | sanfensan | 7C | 413833654 | A, C | sanfensan |
| 2C | 252643615 | G, A | sanfensan | 4D | 407356318 | G, A | sanfensan | 7C | 414551856 | G, T | sanfensan |
| 2C | 256169319 | G, A | sanfensan | 4D | 413268611 | T, C | sanfensan | 7C | 416262975 | C, T | sanfensan |
| 2C | 261833087 | G, A | sanfensan | 4D | 418031647 | G, A | sanfensan | 7C | 420923703 | T, C | sanfensan |
| 2C | 262787175 | A, T | sanfensan | 4D | 420702529 | G, A | sanfensan | 7C | 426911439 | C, A | sanfensan |
| 2C | 267745937 | G, A | sanfensan | 4D | 426669740 | A, G | sanfensan | 7C | 430613225 | G, A | sanfensan |
| 2C | 271735685 | G, A | sanfensan | 4D | 432669116 | G, A | sanfensan | 7C | 432461759 | G, A | sanfensan |
| 2C | 277919032 | G, A | sanfensan | 4D | 437532533 | G, T | sanfensan | 7C | 435550002 | C, T | sanfensan |
| 2C | 281899341 | C, T | sanfensan | 4D | 443034471 | C, T | sanfensan | 7C | 444070312 | G, T | sanfensan |
| 2C | 287576313 | C, T | sanfensan | 4D | 445235008 | A, C | sanfensan | 7C | 445489155 | G, A | sanfensan |
| 2C | 294884196 | G, A | sanfensan | 4D | 451513413 | A, G | sanfensan | 7C | 452005889 | A, T | sanfensan |
| 2C | 298708412 | T, A | sanfensan | 5A | 2255953 | A, C | sanfensan | 7C | 457711724 | T, C | sanfensan |
| 2C | 304070232 | A, G | sanfensan | 5A | 6260213 | C, A | sanfensan | 7C | 462688367 | G, A | sanfensan |
| 2C | 305356521 | G, T | sanfensan | 5A | 13803621 | C, T | sanfensan | 7C | 464956118 | A, G | sanfensan |
| 2C | 312681417 | G, A | sanfensan | 5A | 15561310 | G, C | sanfensan | 7C | 465254207 | G, T | sanfensan |
| 2C | 317317802 | T, C | sanfensan | 5A | 24452734 | T, C | sanfensan | 7C | 471154727 | G, A | sanfensan |
| 2C | 324693110 | G, T | sanfensan | 5A | 27224463 | G, A | sanfensan | 7C | 476266406 | C, A | sanfensan |
| 2C | 329639427 | C, G | sanfensan | 5A | 34502392 | A, G | sanfensan | 7C | 480020947 | C, T | sanfensan |
| 2C | 334559533 | C, A | sanfensan | 5A | 36710142 | G, A | sanfensan | 7C | 485814933 | A, G | sanfensan |
| 2C | 335512805 | C, T | sanfensan | 5A | 44344522 | G, A | sanfensan | 7C | 491712274 | T, C | sanfensan |
| 2C | 342249614 | G, A | sanfensan | 5A | 49562544 | T, C | sanfensan | 7C | 496402779 | G, C | sanfensan |
| 2C | 349065985 | C, T | sanfensan | 5A | 53433795 | T, C | sanfensan | 7C | 500349440 | G, A | sanfensan |
| 2C | 354184855 | C, T | sanfensan | 5A | 56109353 | G, A | sanfensan | 7C | 505836662 | C, T | sanfensan |
| 2C | 358792181 | C, T | sanfensan | 5A | 64589225 | C, T | sanfensan | 7C | 514393934 | A, G | sanfensan |
| 2C | 361602927 | A, C | sanfensan | 5A | 68491563 | T, C | sanfensan | 7C | 518087154 | C, G | sanfensan |
| 2C | 365574905 | T, C | sanfensan | 5A | 70531911 | C, T | sanfensan | 7C | 520313196 | A, G | sanfensan |
| 2C | 372578823 | A, T | sanfensan | 5A | 79896986 | T, C | sanfensan | 7C | 528181463 | G, C | sanfensan |
| 2C | 377682611 | C, T | sanfensan | 5A | 83113930 | T, C | sanfensan | 7C | 530421340 | G, C | sanfensan |
| 2C | 383773493 | G, A | sanfensan | 5A | 86401636 | T, C | sanfensan | 7D | 2445778 | G, A | sanfensan |
| 2C | 388264545 | G, T | sanfensan | 5A | 94264887 | T, C | sanfensan | 7D | 9742047 | A, G | sanfensan |
| 2C | 392647080 | T, A | sanfensan | 5A | 95056890 | T, C | sanfensan | 7D | 9847518 | A, G | sanfensan |
| 2C | 399910470 | G, A | sanfensan | 5A | 102064390 | A, G | sanfensan | 7D | 13815389 | A, T | sanfensan |
| 2C | 402251417 | T, G | sanfensan | 5A | 106428331 | G, A | sanfensan | 7D | 18736551 | G, T | sanfensan |
| 2C | 405239508 | A, G | sanfensan | 5A | 110419074 | G, A | sanfensan | 7D | 20505929 | C, T | sanfensan |
| 2C | 412396338 | A, G | sanfensan | 5A | 116006562 | G, A | sanfensan | 7D | 25511353 | G, A | sanfensan |
| 2C | 416260936 | G, A | sanfensan | 5A | 121002541 | A, T | sanfensan | 7D | 30499092 | G, A | sanfensan |
| 2C | 423413408 | G, A | sanfensan | 5A | 127363246 | T, C | sanfensan | 7D | 36614804 | T, G | sanfensan |
| 2C | 429152113 | C, T | sanfensan | 5A | 132453075 | T, C | sanfensan | 7D | 40700604 | G, C | sanfensan |
| 2C | 433743437 | G, A | sanfensan | 5A | 137069112 | A, C | sanfensan | 7D | 46604719 | C, T | sanfensan |
| 2C | 438694947 | G, A | sanfensan | 5A | 142368295 | C, T | sanfensan | 7D | 50727705 | C, G | sanfensan |
| 2C | 441372926 | G, A | sanfensan | 5A | 147275003 | T, G | sanfensan | 7D | 57700439 | C, T | sanfensan |
| 2C | 446094456 | T, G | sanfensan | 5A | 152777713 | C, T | sanfensan | 7D | 60169262 | C, A | sanfensan |
| 2C | 451391070 | A, G | sanfensan | 5A | 156493283 | C, T | sanfensan | 7D | 69063876 | A, C | sanfensan |
| 2C | 456022723 | A, C | sanfensan | 5A | 160244662 | A, G | sanfensan | 7D | 71993254 | T, C | sanfensan |
| 2C | 462451470 | G, A | sanfensan | 5A | 166708997 | C, G | sanfensan | 7D | 79211559 | A, G | sanfensan |
| 2C | 469973109 | G, A | sanfensan | 5A | 174872733 | T, C | sanfensan | 7D | 82904994 | C, T | sanfensan |
| 2C | 473490736 | G, A | sanfensan | 5A | 176197051 | A, G | sanfensan | 7D | 85615821 | A, G | sanfensan |
| 2C | 476541629 | C, T | sanfensan | 5A | 181213909 | T, C | sanfensan | 7D | 91224657 | T, C | sanfensan |
| 2C | 482708999 | T, G | sanfensan | 5A | 188859714 | T, C | sanfensan | 7D | 98952743 | G, A | sanfensan |
| 2C | 488486085 | C, G | sanfensan | 5A | 190215196 | C, T | sanfensan | 7D | 100841898 | C, T | sanfensan |
| 2C | 492299240 | T, G | sanfensan | 5A | 195549254 | T, C | sanfensan | 7D | 108477777 | G, A | sanfensan |
| 2C | 497502793 | C, T | sanfensan | 5A | 198785103 | A, G | sanfensan | 7D | 110334702 | C, T | sanfensan |
| 2C | 504463768 | G, A | sanfensan | 5A | 202377152 | T, C | sanfensan | 7D | 116643732 | C, T | sanfensan |
| 2C | 506288307 | G, A | sanfensan | 5A | 207723478 | G, A | sanfensan | 7D | 123307950 | T, C | sanfensan |
| 2C | 511067660 | T, C | sanfensan | 5A | 212325208 | A, G | sanfensan | 7D | 127161912 | A, G | sanfensan |
| 2C | 515199747 | G, T | sanfensan | 5A | 216205965 | A, G | sanfensan | 7D | 133410614 | T, G | sanfensan |
| 2C | 520028290 | C, G | sanfensan | 5A | 221258102 | C, T | sanfensan | 7D | 138641756 | C, T | sanfensan |
| 2C | 526639651 | C, T | sanfensan | 5A | 225234818 | G, T | sanfensan | 7D | 141652553 | T, G | sanfensan |
| 2C | 530779807 | G, A | sanfensan | 5A | 230456063 | A, T | sanfensan | 7D | 146207944 | C, T | sanfensan |
| 2C | 537336195 | A, G | sanfensan | 5A | 238470416 | T, C | sanfensan | 7D | 151573471 | G, A | sanfensan |
| 2C | 540940586 | T, C | sanfensan | 5A | 240660477 | G, C | sanfensan | 7D | 156040797 | C, A | sanfensan |
| 2C | 547202871 | G, A | sanfensan | 5A | 245072572 | A, G | sanfensan | 7D | 161660385 | A, G | sanfensan |
| 2C | 552060518 | G, C | sanfensan | 5A | 250830642 | G, C | sanfensan | 7D | 167061192 | A, G | sanfensan |
| 2D | 4090001 | G, A | sanfensan | 5A | 259737745 | A, G | sanfensan | 7D | 170724248 | A, G | sanfensan |
| 2D | 8884387 | G, A | sanfensan | 5A | 261185047 | G, A | sanfensan | 7D | 178681077 | A, C | sanfensan |
| 2D | 13578443 | C, G | sanfensan | 5A | 269804753 | A, G | sanfensan | 7D | 183800740 | A, C | sanfensan |
| 2D | 17675396 | A, G | sanfensan | 5A | 273021787 | T, C | sanfensan | 7D | 186691354 | A, G | sanfensan |
| 2D | 23696567 | G, C | sanfensan | 5A | 276143054 | T, C | sanfensan | 7D | 192032150 | G, A | sanfensan |
| 2D | 29926609 | C, A | sanfensan | 5A | 282716721 | A, C | sanfensan | 7D | 197240877 | G, A | sanfensan |
| 2D | 31795322 | C, G | sanfensan | 5A | 289601566 | A, C | sanfensan | 7D | 203331302 | A, G | sanfensan |
| 2D | 38891629 | C, T | sanfensan | 5A | 290520143 | A, T | sanfensan | 7D | 206895614 | A, G | sanfensan |
| 2D | 43755471 | A, C | sanfensan | 5A | 297192869 | T, G | sanfensan | 7D | 214500259 | A, T | sanfensan |
| 2D | 49839859 | T, C | sanfensan | 5A | 301832004 | T, C | sanfensan | 7D | 216095702 | C, A | sanfensan |
| 2D | 52853593 | T, G | sanfensan | 5A | 308399750 | G, T | sanfensan | 7D | 223545306 | G, A | sanfensan |
| 2D | 59369523 | A, G | sanfensan | 5A | 311247256 | G, A | sanfensan | 7D | 226229762 | G, A | sanfensan |
| 2D | 64844533 | C, T | sanfensan | 5A | 317923031 | C, A | sanfensan | 7D | 232450357 | G, C | sanfensan |
| 2D | 67973342 | G, C | sanfensan | 5A | 321801333 | T, C | sanfensan | 7D | 237413151 | T, C | sanfensan |
| 2D | 72762945 | G, T | sanfensan | 5A | 327241850 | C, T | sanfensan | 7D | 243355850 | T, C | sanfensan |
| 2D | 77924703 | G, A | sanfensan | 5A | 333717258 | A, G | sanfensan | 7D | 247929240 | G, T | sanfensan |
| 2D | 82246788 | C, G | sanfensan | 5A | 335041321 | G, A | sanfensan | 7D | 254107993 | C, G | sanfensan |
| 2D | 85545627 | A, G | sanfensan | 5A | 341600328 | C, T | sanfensan | 7D | 255422386 | T, C | sanfensan |
| 2D | 93391163 | T, C | sanfensan | 5A | 347629159 | C, T | sanfensan | 7D | 264369755 | A, C | sanfensan |
| 2D | 97511713 | T, C | sanfensan | 5A | 350824983 | T, C | sanfensan | 7D | 267080055 | G, C | sanfensan |
| 2D | 103508833 | A, G | sanfensan | 5A | 356271802 | A, G | sanfensan | 7D | 272102782 | T, C | sanfensan |
| 2D | 105773491 | C, T | sanfensan | 5A | 363526707 | C, T | sanfensan | 7D | 276724318 | T, C | sanfensan |
| 2D | 112039326 | T, C | sanfensan | 5A | 369477353 | T, C | sanfensan | 7D | 278615324 | G, C | sanfensan |
| 2D | 118313919 | A, G | sanfensan | 5A | 370529828 | C, G | sanfensan | 7D | 283701259 | T, G | sanfensan |
| 2D | 124707738 | G, A | sanfensan | 5A | 375579309 | T, C | sanfensan | 7D | 285490224 | C, T | sanfensan |
| 2D | 127418722 | T, C | sanfensan | 5A | 383503810 | C, T | sanfensan | 7D | 290974761 | C, T | sanfensan |
| 2D | 134051671 | A, G | sanfensan | 5A | 387182267 | G, C | sanfensan | 7D | 299205902 | T, C | sanfensan |
| 2D | 139923233 | T, C | sanfensan | 5A | 390102471 | A, G | sanfensan | 7D | 302081398 | G, A | sanfensan |
| 2D | 141313127 | G, T | sanfensan | 5A | 398649985 | C, A | sanfensan | 7D | 305799487 | A, G | sanfensan |
| 2D | 147473787 | C, A | sanfensan | 5A | 404206934 | A, G | sanfensan | 7D | 310083219 | A, G | sanfensan |
| 2D | 153131701 | A, G | sanfensan | 5A | 409217956 | G, A | sanfensan | 7D | 318265612 | C, T | sanfensan |
| 2D | 155782705 | A, C | sanfensan | 5A | 411203550 | C, T | sanfensan | 7D | 324554035 | C, A | sanfensan |
| 2D | 161543833 | T, G | sanfensan | 5A | 415301859 | A, G | sanfensan | 7D | 328162737 | A, G | sanfensan |
| 2D | 168209065 | T, C | sanfensan | 5A | 416065809 | T, G | sanfensan | 7D | 332476238 | C, T | sanfensan |
| 2D | 171707336 | G, A | sanfensan | 5A | 421891077 | A, G | sanfensan | 7D | 336554421 | T, C | sanfensan |
| 2D | 175227041 | C, G | sanfensan | 5A | 428858871 | C, T | sanfensan | 7D | 342546157 | T, C | sanfensan |
| 2D | 183727141 | C, A | sanfensan | 5A | 434243983 | A, G | sanfensan | 7D | 348921802 | A, G | sanfensan |
| 2D | 186245952 | T, C | sanfensan | 5A | 436126819 | A, G | sanfensan | 7D | 352024567 | A, C | sanfensan |
| 2D | 191571350 | T, C | sanfensan | 5A | 441185565 | C, T | sanfensan | 7D | 358950077 | C, T | sanfensan |
| 2D | 197886074 | C, A | sanfensan | 5A | 447327633 | G, T | sanfensan | 7D | 362820207 | G, A | sanfensan |
| 2D | 201218813 | A, G | sanfensan | 5A | 452417280 | C, G | sanfensan | 7D | 368623531 | T, C | sanfensan |
| 2D | 206194560 | G, T | sanfensan | 5A | 458701937 | C, G | sanfensan | 7D | 371406914 | G, T | sanfensan |
| 2D | 209315382 | T, C | sanfensan | 5A | 460085569 | A, G | sanfensan | 7D | 379681421 | T, C | sanfensan |
| 2D | 214621383 | T, G | sanfensan | 5A | 468161069 | T, G | sanfensan | 7D | 382815770 | C, T | sanfensan |
| 2D | 217094666 | T, C | sanfensan | 5A | 474226937 | C, G | sanfensan | 7D | 389570780 | G, A | sanfensan |
| 2D | 224088866 | C, T | sanfensan | 5A | 478302848 | A, G | sanfensan | 7D | 390101972 | G, A | sanfensan |
| 2D | 228951287 | A, G | sanfensan | 5A | 483995877 | C, A | sanfensan | 7D | 399143078 | C, T | sanfensan |
| 2D | 233694297 | A, C | sanfensan | 5A | 488177553 | G, T | sanfensan | 7D | 400807921 | C, T | sanfensan |
| 2D | 237373003 | T, C | sanfensan | 5A | 488401160 | A, T | sanfensan | 7D | 406112763 | T, G | sanfensan |
| 2D | 240374430 | G, T | sanfensan | 5C | 3489579 | C, T | sanfensan | 7D | 411104681 | A, G | sanfensan |
| 2D | 247507939 | G, A | sanfensan | 5C | 6268971 | C, T | sanfensan | 7D | 415490064 | G, A | sanfensan |
| 2D | 253371162 | C, T | sanfensan | 5C | 12203702 | G, A | sanfensan | 7D | 423220521 | A, G | sanfensan |
| 2D | 259426340 | T, C | sanfensan | 5C | 16396954 | A, G | sanfensan | 7D | 426049443 | T, C | sanfensan |
| 2D | 262144878 | T, C | sanfensan | 5C | 22928378 | C, A | sanfensan | 7D | 432545626 | A, G | sanfensan |
| 2D | 268152175 | C, T | sanfensan | 5C | 26060685 | T, C | sanfensan | 7D | 438677802 | A, T | sanfensan |
| 2D | 271878835 | A, C | sanfensan | 5C | 31503941 | C, T | sanfensan | 7D | 442595402 | T, C | sanfensan |
| 2D | 278388739 | G, A | sanfensan | 5C | 39150034 | T, C | sanfensan | 7D | 447536505 | C, T | sanfensan |
| 2D | 281916083 | G, A | sanfensan | 5C | 44163319 | T, G | sanfensan | 7D | 453173144 | T, A | sanfensan |
| 2D | 289003292 | T, C | sanfensan | 5C | 47044843 | C, T | sanfensan | 7D | 456025113 | C, T | sanfensan |
| 2D | 293861011 | T, C | sanfensan | 5C | 50558717 | C, A | sanfensan | 7D | 463196054 | A, T | sanfensan |
| 2D | 297746679 | G, A | sanfensan | 5C | 53047612 | G, A | sanfensan | 7D | 466703278 | A, G | sanfensan |
| 2D | 301635985 | T, C | sanfensan | 5C | 56146722 | T, G | sanfensan | 7D | 474383321 | G, T | sanfensan |
| 2D | 307441210 | A, G | sanfensan | 5C | 63376645 | G, A | sanfensan | 7D | 478751163 | T, C | sanfensan |
| 2D | 313997317 | C, T | sanfensan | 5C | 69905284 | T, C | sanfensan | 7D | 481249084 | C, G | sanfensan |
| 2D | 317780757 | T, C | sanfensan | 5C | 74387265 | T, C | sanfensan | 7D | 486113092 | A, T | sanfensan |
| 2D | 324471320 | G, C | sanfensan | 5C | 77896048 | C, T | sanfensan | 7D | 491181988 | G, T | sanfensan |
| 2D | 325394234 | C, T | sanfensan | 5C | 81012623 | C, T | sanfensan | 7D | 497047184 | A, G | sanfensan |
| 2D | 332243555 | G, A | sanfensan | 5C | 86566325 | A, G | sanfensan | 1A | 106635350 | G, C | OT3098 |
| 2D | 336723088 | C, T | sanfensan | 5C | 94254331 | G, C | sanfensan | 1A | 304418445 | G, A | OT3098 |
| 2D | 344502614 | T, C | sanfensan | 5C | 95909941 | T, C | sanfensan | 1A | 338092374 | C, T | OT3098 |
| 2D | 348558097 | G, A | sanfensan | 5C | 101827763 | A, G | sanfensan | 1A | 348499239 | C, T | OT3098 |
| 2D | 354230586 | G, A | sanfensan | 5C | 109797422 | C, A | sanfensan | 1A | 369374878 | A, G | OT3098 |
| 2D | 356506435 | C, T | sanfensan | 5C | 114362886 | G, T | sanfensan | 1A | 383912262 | C, T | OT3098 |
| 2D | 361372636 | C, A | sanfensan | 5C | 119134297 | A, C | sanfensan | 1A | 397750385 | A, G | OT3098 |
| 2D | 367508960 | T, C | sanfensan | 5C | 121784178 | G, A | sanfensan | 1A | 512105703 | G, A | OT3098 |
| 2D | 374623643 | C, T | sanfensan | 5C | 125633452 | C, A | sanfensan | 1C | 40349872 | G, T | OT3098 |
| 2D | 376574690 | G, A | sanfensan | 5C | 132720505 | G, A | sanfensan | 1C | 80742591 | G, T | OT3098 |
| 2D | 381350972 | A, G | sanfensan | 5C | 138374966 | A, G | sanfensan | 1C | 104538708 | A, G | OT3098 |
| 2D | 385713492 | C, T | sanfensan | 5C | 143890607 | C, T | sanfensan | 1C | 119650459 | G, T | OT3098 |
| 2D | 389191706 | C, A | sanfensan | 5C | 145866226 | G, A | sanfensan | 1C | 120468329 | T, G | OT3098 |
| 2D | 393186784 | T, C | sanfensan | 5C | 150225588 | G, A | sanfensan | 1C | 181195253 | G, A | OT3098 |
| 2D | 398053878 | C, T | sanfensan | 5C | 156966699 | A, G | sanfensan | 1C | 242119623 | C, T | OT3098 |
| 2D | 402113963 | G, A | sanfensan | 5C | 160214876 | G, C | sanfensan | 1C | 265897428 | C, T | OT3098 |
| 2D | 407714293 | C, T | sanfensan | 5C | 168811131 | T, C | sanfensan | 1C | 280066160 | G, A | OT3098 |
| 2D | 413300157 | C, G | sanfensan | 5C | 174809389 | G, C | sanfensan | 1C | 291282749 | A, T | OT3098 |
| 2D | 416994083 | C, T | sanfensan | 5C | 177125115 | A, C | sanfensan | 1C | 319005689 | C, A | OT3098 |
| 2D | 422486122 | A, G | sanfensan | 5C | 182633756 | G, A | sanfensan | 1C | 342223334 | G, A | OT3098 |
| 2D | 429086006 | C, T | sanfensan | 5C | 189642953 | T, C | sanfensan | 1C | 362511327 | C, T | OT3098 |
| 2D | 430932257 | T, G | sanfensan | 5C | 193221965 | A, G | sanfensan | 1C | 403231603 | G, T | OT3098 |
| 2D | 435517110 | T, C | sanfensan | 5C | 199654787 | C, T | sanfensan | 1D | 28155193 | G, A | OT3098 |
| 2D | 440681525 | G, T | sanfensan | 5C | 201058413 | C, T | sanfensan | 1D | 105612570 | C, T | OT3098 |
| 2D | 446278114 | T, C | sanfensan | 5C | 208411390 | T, C | sanfensan | 1D | 114754382 | T, A | OT3098 |
| 2D | 452744585 | A, G | sanfensan | 5C | 214681134 | T, C | sanfensan | 1D | 153648523 | C, T | OT3098 |
| 2D | 458588987 | G, A | sanfensan | 5C | 216349665 | C, T | sanfensan | 1D | 168181978 | G, A | OT3098 |
| 2D | 460269576 | T, G | sanfensan | 5C | 220308120 | C, T | sanfensan | 1D | 178507460 | T, C | OT3098 |
| 2D | 466552364 | C, T | sanfensan | 5C | 223909826 | T, C | sanfensan | 1D | 186530691 | C, G | OT3098 |
| 2D | 473536427 | A, C | sanfensan | 5C | 227213415 | T, C | sanfensan | 1D | 199381843 | C, T | OT3098 |
| 2D | 478638656 | T, G | sanfensan | 5C | 234663041 | C, T | sanfensan | 1D | 213294278 | C, A | OT3098 |
| 2D | 484074145 | G, A | sanfensan | 5C | 236099260 | A, C | sanfensan | 1D | 234770750 | A, G | OT3098 |
| 2D | 486599496 | C, T | sanfensan | 5C | 244246397 | C, A | sanfensan | 1D | 249534977 | G, A | OT3098 |
| 2D | 492020467 | C, T | sanfensan | 5C | 246386706 | C, A | sanfensan | 1D | 336367249 | T, C | OT3098 |
| 2D | 498876308 | C, T | sanfensan | 5C | 252990985 | A, T | sanfensan | 1D | 458302223 | A, G | OT3098 |
| 2D | 499971233 | T, C | sanfensan | 5C | 255616974 | C, A | sanfensan | 2A | 288229046 | G, T | OT3098 |
| 2D | 501118019 | C, T | sanfensan | 5C | 261629346 | G, A | sanfensan | 2A | 298965546 | A, G | OT3098 |
| 2D | 509719761 | G, T | sanfensan | 5C | 266182002 | C, T | sanfensan | 2A | 312180949 | G, A | OT3098 |
| 2D | 511708692 | G, A | sanfensan | 5C | 271236736 | C, A | sanfensan | 2A | 348182068 | C, T | OT3098 |
| 2D | 515022757 | T, A | sanfensan | 5C | 275627914 | A, G | sanfensan | 2A | 374638766 | A, G | OT3098 |
| 2D | 523356028 | C, A | sanfensan | 5C | 283728720 | C, T | sanfensan | 2A | 399111341 | G, A | OT3098 |
| 2D | 525941172 | A, G | sanfensan | 5C | 285163996 | T, C | sanfensan | 2C | 28799948 | T, C | OT3098 |
| 2D | 529724172 | A, C | sanfensan | 5C | 292163090 | G, A | sanfensan | 2C | 42159271 | T, G | OT3098 |
| 2D | 530568058 | G, A | sanfensan | 5C | 297500311 | T, C | sanfensan | 2C | 61574030 | G, T | OT3098 |
| 3A | 349403 | C, T | sanfensan | 5C | 304040309 | T, C | sanfensan | 2C | 87111306 | T, C | OT3098 |
| 3A | 5686447 | A, G | sanfensan | 5C | 306535191 | C, T | sanfensan | 2C | 163497553 | G, A | OT3098 |
| 3A | 14820154 | G, C | sanfensan | 5C | 313032349 | T, G | sanfensan | 2C | 384573213 | A, G | OT3098 |
| 3A | 19103892 | A, G | sanfensan | 5C | 318243088 | T, G | sanfensan | 2C | 526110948 | A, G | OT3098 |
| 3A | 24307017 | G, A | sanfensan | 5C | 320862543 | G, A | sanfensan | 2C | 530336382 | G, A | OT3098 |
| 3A | 28596998 | G, A | sanfensan | 5C | 325755346 | C, T | sanfensan | 2D | 25489315 | A, G | OT3098 |
| 3A | 34850179 | A, G | sanfensan | 5C | 333233541 | T, C | sanfensan | 2D | 48295932 | C, T | OT3098 |
| 3A | 39937020 | C, T | sanfensan | 5C | 338139569 | T, C | sanfensan | 2D | 136287278 | G, A | OT3098 |
| 3A | 43520691 | A, G | sanfensan | 5C | 342626437 | A, G | sanfensan | 2D | 257949893 | T, C | OT3098 |
| 3A | 49770087 | T, C | sanfensan | 5C | 347361434 | G, A | sanfensan | 2D | 374246423 | T, G | OT3098 |
| 3A | 54743914 | A, G | sanfensan | 5C | 354441762 | C, T | sanfensan | 2D | 439808678 | A, G | OT3098 |
| 3A | 56540660 | A, C | sanfensan | 5C | 358341050 | C, G | sanfensan | 2D | 464345033 | G, A | OT3098 |
| 3A | 60015652 | C, T | sanfensan | 5C | 363432176 | T, C | sanfensan | 3A | 13251626 | G, A | OT3098 |
| 3A | 68312753 | T, G | sanfensan | 5C | 369584563 | T, C | sanfensan | 3A | 65224422 | C, T | OT3098 |
| 3A | 70210131 | A, G | sanfensan | 5C | 373117738 | G, A | sanfensan | 3A | 80331258 | G, A | OT3098 |
| 3A | 75548656 | C, T | sanfensan | 5C | 377549147 | G, A | sanfensan | 3A | 102356888 | G, A | OT3098 |
| 3A | 81960668 | G, A | sanfensan | 5C | 384699195 | G, A | sanfensan | 3A | 235151032 | C, T | OT3098 |
| 3A | 87385909 | G, A | sanfensan | 5C | 385128549 | C, T | sanfensan | 3A | 290609726 | A, G | OT3098 |
| 3A | 91466974 | T, C | sanfensan | 5C | 391966210 | C, T | sanfensan | 3A | 342060468 | A, G | OT3098 |
| 3A | 99324808 | G, A | sanfensan | 5C | 399070404 | C, T | sanfensan | 3A | 375362853 | G, T | OT3098 |
| 3A | 103702261 | A, T | sanfensan | 5C | 402179080 | A, G | sanfensan | 3C | 83743224 | C, T | OT3098 |
| 3A | 106902222 | A, G | sanfensan | 5C | 405507477 | T, C | sanfensan | 3C | 105875209 | T, A | OT3098 |
| 3A | 114734802 | C, T | sanfensan | 5C | 411038650 | A, G | sanfensan | 3C | 153371948 | G, A | OT3098 |
| 3A | 117018962 | T, G | sanfensan | 5C | 416614236 | G, C | sanfensan | 3C | 160415743 | A, G | OT3098 |
| 3A | 123014541 | G, T | sanfensan | 5C | 421435620 | T, G | sanfensan | 3C | 188382442 | C, T | OT3098 |
| 3A | 125235584 | G, A | sanfensan | 5C | 427497128 | A, G | sanfensan | 3C | 199311115 | T, C | OT3098 |
| 3A | 132736923 | G, T | sanfensan | 5C | 432532568 | A, C | sanfensan | 3C | 215249361 | C, T | OT3098 |
| 3A | 133400727 | T, C | sanfensan | 5C | 435770963 | C, T | sanfensan | 3C | 242293326 | A, C | OT3098 |
| 3A | 135881496 | A, G | sanfensan | 5C | 440258824 | A, G | sanfensan | 3C | 253359904 | T, C | OT3098 |
| 3A | 140132188 | A, C | sanfensan | 5C | 447827826 | T, C | sanfensan | 3C | 272308234 | G, T | OT3098 |
| 3A | 149216557 | G, T | sanfensan | 5C | 451307256 | C, A | sanfensan | 3C | 302216223 | G, A | OT3098 |
| 3A | 153688728 | T, C | sanfensan | 5C | 458701577 | A, C | sanfensan | 3C | 312338539 | T, C | OT3098 |
| 3A | 157365828 | T, G | sanfensan | 5C | 462771476 | G, T | sanfensan | 3C | 327416798 | T, C | OT3098 |
| 3A | 162629353 | G, A | sanfensan | 5C | 468438848 | G, A | sanfensan | 3C | 349649824 | A, C | OT3098 |
| 3A | 163249925 | C, T | sanfensan | 5C | 473285230 | T, C | sanfensan | 3C | 351983098 | G, A | OT3098 |
| 3A | 165620860 | A, G | sanfensan | 5C | 477766211 | T, C | sanfensan | 3C | 416217578 | T, A | OT3098 |
| 3A | 170114512 | T, C | sanfensan | 5C | 480911717 | C, G | sanfensan | 3C | 533296338 | T, C | OT3098 |
| 3A | 175321928 | G, A | sanfensan | 5C | 489509201 | C, G | sanfensan | 3C | 543025252 | A, G | OT3098 |
| 3A | 183395685 | C, T | sanfensan | 5C | 490176188 | G, C | sanfensan | 3C | 551825075 | C, T | OT3098 |
| 3A | 188224937 | A, C | sanfensan | 5C | 498023277 | A, G | sanfensan | 3C | 571627195 | T, C | OT3098 |
| 3A | 194044731 | G, A | sanfensan | 5C | 503467351 | T, C | sanfensan | 3C | 606797452 | C, A | OT3098 |
| 3A | 197334481 | G, A | sanfensan | 5C | 509322035 | T, C | sanfensan | 3C | 620635579 | G, A | OT3098 |
| 3A | 204024961 | T, C | sanfensan | 5C | 512872294 | A, G | sanfensan | 3D | 471246706 | C, T | OT3098 |
| 3A | 209320496 | A, G | sanfensan | 5C | 515412167 | A, G | sanfensan | 4A | 4383966 | T, C | OT3098 |
| 3A | 214269791 | G, A | sanfensan | 5C | 520108101 | T, C | sanfensan | 4A | 91672697 | A, G | OT3098 |
| 3A | 215870959 | A, G | sanfensan | 5C | 526886988 | G, A | sanfensan | 4A | 176172514 | T, C | OT3098 |
| 3A | 220058696 | A, C | sanfensan | 5C | 533665172 | G, A | sanfensan | 4A | 262281263 | T, G | OT3098 |
| 3A | 228807812 | G, A | sanfensan | 5C | 536150519 | G, A | sanfensan | 4A | 281599989 | C, T | OT3098 |
| 3A | 234527749 | A, G | sanfensan | 5C | 542459731 | T, C | sanfensan | 4A | 315467166 | T, C | OT3098 |
| 3A | 237989422 | G, A | sanfensan | 5C | 548440305 | T, C | sanfensan | 4A | 330624554 | C, T | OT3098 |
| 3A | 241256197 | C, T | sanfensan | 5C | 551331730 | A, G | sanfensan | 4A | 344502223 | G, A | OT3098 |
| 3A | 248608009 | C, T | sanfensan | 5C | 555077838 | C, G | sanfensan | 4A | 370035053 | A, G | OT3098 |
| 3A | 254861238 | T, C | sanfensan | 5C | 560097889 | T, C | sanfensan | 4A | 409954087 | G, A | OT3098 |
| 3A | 255490400 | A, G | sanfensan | 5C | 569206294 | C, T | sanfensan | 4A | 438257237 | C, G | OT3098 |
| 3A | 261499403 | T, C | sanfensan | 5C | 572903980 | A, G | sanfensan | 4A | 452257049 | G, A | OT3098 |
| 3A | 268848139 | T, G | sanfensan | 5C | 579001353 | A, G | sanfensan | 4C | 228404747 | C, T | OT3098 |
| 3A | 273512863 | T, C | sanfensan | 5C | 582680547 | C, T | sanfensan | 4C | 326801689 | T, C | OT3098 |
| 3A | 279998619 | A, G | sanfensan | 5C | 585411392 | C, T | sanfensan | 4C | 372872071 | G, A | OT3098 |
| 3A | 280635220 | A, C | sanfensan | 5C | 593970148 | C, T | sanfensan | 4C | 405897221 | T, C | OT3098 |
| 3A | 289497088 | C, T | sanfensan | 5C | 598395218 | T, C | sanfensan | 4C | 415253727 | C, G | OT3098 |
| 3A | 291617575 | G, A | sanfensan | 5C | 601401624 | C, A | sanfensan | 4C | 501843966 | T, G | OT3098 |
| 3A | 296690651 | T, C | sanfensan | 5D | 4994611 | T, G | sanfensan | 4C | 589184920 | A, T | OT3098 |
| 3A | 303858433 | G, A | sanfensan | 5D | 6622542 | G, C | sanfensan | 4C | 629827712 | C, A | OT3098 |
| 3A | 308390029 | A, G | sanfensan | 5D | 10884878 | C, G | sanfensan | 4C | 646042521 | A, T | OT3098 |
| 3A | 313163176 | C, T | sanfensan | 5D | 16839171 | G, T | sanfensan | 4C | 660379855 | G, A | OT3098 |
| 3A | 315321758 | G, T | sanfensan | 5D | 21309845 | G, A | sanfensan | 4C | 680363280 | G, A | OT3098 |
| 3A | 323449820 | C, T | sanfensan | 5D | 27535507 | T, A | sanfensan | 4C | 690482618 | G, A | OT3098 |
| 3A | 326487847 | G, T | sanfensan | 5D | 34803208 | G, A | sanfensan | 4D | 323386091 | T, C | OT3098 |
| 3A | 334646062 | C, G | sanfensan | 5D | 36778603 | T, G | sanfensan | 4D | 348557099 | G, A | OT3098 |
| 3A | 339385657 | T, G | sanfensan | 5D | 41959313 | T, C | sanfensan | 4D | 377370815 | A, C | OT3098 |
| 3A | 344905994 | C, T | sanfensan | 5D | 47178435 | T, C | sanfensan | 4D | 386553314 | C, T | OT3098 |
| 3A | 346539460 | G, A | sanfensan | 5D | 52127682 | C, T | sanfensan | 4D | 434004607 | A, G | OT3098 |
| 3A | 354806237 | C, T | sanfensan | 5D | 54096343 | T, G | sanfensan | 4D | 455273707 | C, T | OT3098 |
| 3A | 358217253 | C, T | sanfensan | 5D | 56457835 | G, A | sanfensan | 5A | 12250117 | C, T | OT3098 |
| 3A | 363057750 | C, T | sanfensan | 5D | 63243306 | A, C | sanfensan | 5A | 66713969 | G, T | OT3098 |
| 3A | 368774140 | C, T | sanfensan | 5D | 64721781 | C, T | sanfensan | 5A | 163771340 | C, T | OT3098 |
| 3A | 370439246 | C, T | sanfensan | 5D | 65173806 | A, C | sanfensan | 5A | 229347037 | A, G | OT3098 |
| 3A | 379158179 | A, G | sanfensan | 5D | 66436115 | C, T | sanfensan | 5A | 247731935 | C, T | OT3098 |
| 3A | 380572844 | T, C | sanfensan | 5D | 74642065 | T, C | sanfensan | 5A | 259020640 | C, T | OT3098 |
| 3A | 385843902 | C, T | sanfensan | 5D | 79567830 | G, A | sanfensan | 5A | 275554651 | C, T | OT3098 |
| 3A | 393548401 | C, T | sanfensan | 5D | 83382549 | G, A | sanfensan | 5A | 450812604 | G, A | OT3098 |
| 3A | 396511248 | A, G | sanfensan | 5D | 87296427 | T, C | sanfensan | 5A | 464203725 | C, T | OT3098 |
| 3A | 400231496 | G, A | sanfensan | 5D | 93053910 | T, C | sanfensan | 5C | 19684941 | G, A | OT3098 |
| 3A | 407861434 | T, C | sanfensan | 5D | 99261576 | C, T | sanfensan | 5C | 34175263 | G, T | OT3098 |
| 3A | 410745937 | A, G | sanfensan | 5D | 101623688 | C, T | sanfensan | 5C | 56717315 | T, G | OT3098 |
| 3A | 417480860 | A, C | sanfensan | 5D | 109259133 | T, C | sanfensan | 5C | 103289073 | G, T | OT3098 |
| 3C | 4934705 | T, C | sanfensan | 5D | 110187772 | C, T | sanfensan | 5C | 162396640 | T, C | OT3098 |
| 3C | 9789448 | T, C | sanfensan | 5D | 116420976 | T, C | sanfensan | 5C | 276244815 | G, A | OT3098 |
| 3C | 14114292 | A, G | sanfensan | 5D | 120360110 | G, A | sanfensan | 5C | 313968741 | G, A | OT3098 |
| 3C | 16111717 | T, C | sanfensan | 5D | 128589324 | T, C | sanfensan | 5C | 330901610 | A, G | OT3098 |
| 3C | 20060240 | T, C | sanfensan | 5D | 134480766 | G, A | sanfensan | 5C | 491868915 | T, C | OT3098 |
| 3C | 29738599 | G, T | sanfensan | 5D | 136403599 | C, T | sanfensan | 5C | 501340024 | G, A | OT3098 |
| 3C | 34251466 | G, A | sanfensan | 5D | 142232990 | C, T | sanfensan | 5C | 537051000 | T, C | OT3098 |
| 3C | 39605336 | A, C | sanfensan | 5D | 145960007 | A, G | sanfensan | 5C | 567644495 | G, A | OT3098 |
| 3C | 41571752 | T, C | sanfensan | 5D | 153395376 | G, A | sanfensan | 5C | 570184840 | A, C | OT3098 |
| 3C | 48463788 | G, T | sanfensan | 5D | 155066422 | T, C | sanfensan | 5D | 94959246 | G, A | OT3098 |
| 3C | 50619185 | G, A | sanfensan | 5D | 162991627 | T, C | sanfensan | 5D | 100600265 | G, A | OT3098 |
| 3C | 59812660 | G, A | sanfensan | 5D | 167232512 | C, T | sanfensan | 5D | 124321696 | T, C | OT3098 |
| 3C | 63059450 | T, C | sanfensan | 5D | 172000670 | T, C | sanfensan | 5D | 149532876 | G, A | OT3098 |
| 3C | 69756699 | C, T | sanfensan | 5D | 177160582 | G, A | sanfensan | 5D | 193714037 | A, G | OT3098 |
| 3C | 72007306 | T, C | sanfensan | 5D | 182553299 | C, T | sanfensan | 5D | 220790821 | A, G | OT3098 |
| 3C | 75582282 | G, C | sanfensan | 5D | 187357553 | C, T | sanfensan | 5D | 247323372 | T, C | OT3098 |
| 3C | 81453337 | T, C | sanfensan | 5D | 191107356 | A, G | sanfensan | 5D | 341143271 | A, G | OT3098 |
| 3C | 89524634 | T, C | sanfensan | 5D | 199208574 | C, T | sanfensan | 5D | 392150519 | A, C | OT3098 |
| 3C | 94729319 | C, A | sanfensan | 5D | 203029590 | A, G | sanfensan | 5D | 487408616 | A, G | OT3098 |
| 3C | 96052025 | C, T | sanfensan | 5D | 209809131 | T, G | sanfensan | 5D | 496068151 | G, T | OT3098 |
| 3C | 100397477 | A, G | sanfensan | 5D | 211555734 | A, T | sanfensan | 6A | 47261549 | G, C | OT3098 |
| 3C | 108020814 | A, G | sanfensan | 5D | 216140070 | T, G | sanfensan | 6A | 79634441 | C, A | OT3098 |
| 3C | 113507812 | A, C | sanfensan | 5D | 221499002 | A, G | sanfensan | 6A | 90176068 | T, C | OT3098 |
| 3C | 119056248 | C, T | sanfensan | 5D | 228576676 | A, G | sanfensan | 6A | 103858240 | T, C | OT3098 |
| 3C | 121539032 | G, T | sanfensan | 5D | 230582553 | G, C | sanfensan | 6A | 143659802 | T, C | OT3098 |
| 3C | 127564610 | C, T | sanfensan | 5D | 238425107 | C, T | sanfensan | 6A | 159101934 | A, T | OT3098 |
| 3C | 130874626 | G, A | sanfensan | 5D | 242649305 | G, A | sanfensan | 6A | 171391185 | C, A | OT3098 |
| 3C | 137072359 | G, A | sanfensan | 5D | 245087633 | T, C | sanfensan | 6A | 184973728 | A, G | OT3098 |
| 3C | 142064744 | G, A | sanfensan | 5D | 250750865 | G, T | sanfensan | 6A | 192005904 | G, A | OT3098 |
| 3C | 147139993 | T, C | sanfensan | 5D | 258642655 | G, A | sanfensan | 6A | 263939310 | C, T | OT3098 |
| 3C | 152287508 | G, A | sanfensan | 5D | 262006477 | A, G | sanfensan | 6A | 300620942 | A, G | OT3098 |
| 3C | 158231130 | C, T | sanfensan | 5D | 266745740 | G, A | sanfensan | 6A | 419205117 | T, A | OT3098 |
| 3C | 161849544 | C, T | sanfensan | 5D | 271633260 | G, A | sanfensan | 6C | 59229976 | C, T | OT3098 |
| 3C | 167803980 | A, G | sanfensan | 5D | 278837964 | C, T | sanfensan | 6C | 84591374 | C, A | OT3098 |
| 3C | 171441935 | T, C | sanfensan | 5D | 280282084 | T, C | sanfensan | 6C | 87587254 | G, C | OT3098 |
| 3C | 178608408 | T, G | sanfensan | 5D | 289610610 | A, T | sanfensan | 6C | 144536268 | C, A | OT3098 |
| 3C | 180422132 | T, C | sanfensan | 5D | 293820840 | C, T | sanfensan | 6C | 238362770 | C, A | OT3098 |
| 3C | 186181001 | G, A | sanfensan | 5D | 298575083 | A, T | sanfensan | 6C | 246928523 | C, T | OT3098 |
| 3C | 192589566 | T, C | sanfensan | 5D | 302837192 | T, C | sanfensan | 6C | 292396236 | T, C | OT3098 |
| 3C | 195188213 | G, A | sanfensan | 5D | 305502791 | T, C | sanfensan | 6C | 342467150 | A, G | OT3098 |
| 3C | 204778810 | C, T | sanfensan | 5D | 314628778 | A, G | sanfensan | 6C | 366301685 | G, A | OT3098 |
| 3C | 207904801 | T, C | sanfensan | 5D | 319756146 | G, A | sanfensan | 6C | 467269710 | C, T | OT3098 |
| 3C | 214065619 | T, G | sanfensan | 5D | 322226424 | C, A | sanfensan | 6C | 485083612 | C, T | OT3098 |
| 3C | 217226719 | T, C | sanfensan | 5D | 328035262 | C, T | sanfensan | 6C | 574716448 | C, T | OT3098 |
| 3C | 221780018 | G, C | sanfensan | 5D | 332326959 | C, T | sanfensan | 6C | 602967220 | T, C | OT3098 |
| 3C | 228014218 | G, A | sanfensan | 5D | 339720781 | T, A | sanfensan | 6C | 611213471 | T, C | OT3098 |
| 3C | 233526022 | G, A | sanfensan | 5D | 344363610 | A, C | sanfensan | 6D | 67717241 | G, A | OT3098 |
| 3C | 235353093 | A, C | sanfensan | 5D | 345547743 | T, C | sanfensan | 6D | 87582596 | A, G | OT3098 |
| 3C | 244162351 | G, A | sanfensan | 5D | 353592498 | G, A | sanfensan | 6D | 130251011 | A, G | OT3098 |
| 3C | 248122047 | C, T | sanfensan | 5D | 357027031 | C, T | sanfensan | 6D | 143238944 | C, T | OT3098 |
| 3C | 253823594 | C, T | sanfensan | 5D | 363095927 | T, G | sanfensan | 6D | 161653963 | A, G | OT3098 |
| 3C | 259462643 | A, G | sanfensan | 5D | 365082132 | T, C | sanfensan | 6D | 193391379 | C, T | OT3098 |
| 3C | 260199510 | T, C | sanfensan | 5D | 372087578 | A, C | sanfensan | 6D | 202580655 | G, A | OT3098 |
| 3C | 269465277 | G, T | sanfensan | 5D | 375075448 | A, G | sanfensan | 6D | 240523148 | G, C | OT3098 |
| 3C | 274931211 | G, A | sanfensan | 5D | 382109948 | T, C | sanfensan | 6D | 256237708 | G, T | OT3098 |
| 3C | 277523171 | C, A | sanfensan | 5D | 385044778 | A, G | sanfensan | 6D | 265237433 | T, C | OT3098 |
| 3C | 284109639 | G, C | sanfensan | 5D | 394858589 | C, T | sanfensan | 6D | 283670697 | G, A | OT3098 |
| 3C | 285346116 | A, G | sanfensan | 5D | 399910298 | C, T | sanfensan | 7A | 6894726 | A, G | OT3098 |
| 3C | 294939142 | A, C | sanfensan | 5D | 402227007 | G, A | sanfensan | 7A | 93976095 | A, C | OT3098 |
| 3C | 296525393 | A, G | sanfensan | 5D | 407502206 | T, C | sanfensan | 7A | 212399895 | C, G | OT3098 |
| 3C | 304406734 | T, C | sanfensan | 5D | 410635861 | A, G | sanfensan | 7A | 226861026 | A, C | OT3098 |
| 3C | 309329884 | C, T | sanfensan | 5D | 419664992 | T, C | sanfensan | 7A | 295965773 | A, G | OT3098 |
| 3C | 310447021 | T, G | sanfensan | 5D | 421256053 | A, G | sanfensan | 7A | 423933210 | T, C | OT3098 |
| 3C | 319961145 | T, C | sanfensan | 5D | 427332097 | C, G | sanfensan | 7A | 440302050 | T, C | OT3098 |
| 3C | 321458383 | T, C | sanfensan | 5D | 433626893 | G, T | sanfensan | 7A | 457458508 | A, G | OT3098 |
| 3C | 325121366 | A, G | sanfensan | 5D | 437055110 | C, G | sanfensan | 7A | 488323283 | A, G | OT3098 |
| 3C | 330206369 | A, G | sanfensan | 5D | 440529761 | C, T | sanfensan | 7C | 2133869 | C, T | OT3098 |
| 3C | 332130807 | C, T | sanfensan | 5D | 446386566 | C, T | sanfensan | 7C | 6687935 | A, G | OT3098 |
| 3C | 338543178 | T, C | sanfensan | 5D | 454896966 | T, C | sanfensan | 7C | 40731489 | G, A | OT3098 |
| 3C | 343287383 | T, C | sanfensan | 5D | 458673318 | G, A | sanfensan | 7C | 135716079 | C, A | OT3098 |
| 3C | 349175645 | T, C | sanfensan | 5D | 462992703 | G, A | sanfensan | 7C | 192742152 | C, T | OT3098 |
| 3C | 353910191 | C, T | sanfensan | 5D | 466900163 | C, T | sanfensan | 7C | 213883268 | A, G | OT3098 |
| 3C | 358917955 | T, C | sanfensan | 5D | 474924404 | C, T | sanfensan | 7C | 225224321 | T, C | OT3098 |
| 3C | 363942089 | G, A | sanfensan | 5D | 478407877 | T, C | sanfensan | 7C | 271932318 | G, A | OT3098 |
| 3C | 366028325 | A, C | sanfensan | 5D | 482793426 | T, C | sanfensan | 7C | 303692756 | A, G | OT3098 |
| 3C | 372945120 | C, T | sanfensan | 5D | 488236543 | A, C | sanfensan | 70 | 314575954 | T, C | OT3098 |
| 3C | 375489817 | C, G | sanfensan | 5D | 492418351 | A, G | sanfensan | 7C | 433548395 | T, C | OT3098 |
| 3C | 384457117 | A, G | sanfensan | 5D | 497166345 | G, T | sanfensan | 7C | 461822950 | C, T | OT3098 |
| 3C | 388958566 | A, G | sanfensan | 5D | 500430897 | T, G | sanfensan | 7C | 478807736 | A, G | OT3098 |
| 3C | 394935493 | A, T | sanfensan | 6A | 4492157 | T, C | sanfensan | 7D | 43006316 | G, A | OT3098 |
| 3C | 395752355 | G, A | sanfensan | 6A | 8762221 | G, T | sanfensan | 7D | 53808953 | G, A | OT3098 |
| 3C | 403204424 | G, A | sanfensan | 6A | 11190798 | T, C | sanfensan | 7D | 72525182 | T, A | OT3098 |
| 3C | 408346907 | A, C | sanfensan | 6A | 17691406 | C, T | sanfensan | 7D | 100996318 | C, T | OT3098 |
| 3C | 412555939 | T, C | sanfensan | 6A | 20852081 | T, C | sanfensan | 7D | 119049740 | A, G | OT3098 |
| 3C | 418959428 | A, G | sanfensan | 6A | 26524896 | C, T | sanfensan | 7D | 123197785 | C, T | OT3098 |
| 3C | 424637971 | G, A | sanfensan | 6A | 33584644 | C, T | sanfensan | 7D | 131225276 | A, G | OT3098 |
| 3C | 425426160 | G, A | sanfensan | 6A | 36710498 | A, G | sanfensan | 7D | 196173654 | T, G | OT3098 |
| 3C | 431869178 | A, C | sanfensan | 6A | 42587073 | A, G | sanfensan | 7D | 227132197 | T, C | OT3098 |
| 3C | 437116797 | T, G | sanfensan | 6A | 48196250 | G, C | sanfensan | 7D | 237053803 | T, G | OT3098 |
| 3C | 442997546 | A, G | sanfensan | 6A | 54285408 | C, G | sanfensan | 7D | 248735393 | T, C | OT3098 |
| 3C | 445451564 | C, A | sanfensan | 6A | 57328665 | T, A | sanfensan | 7D | 256172681 | C, T | OT3098 |
| 3C | 449848039 | C, T | sanfensan | 6A | 64386015 | C, A | sanfensan | 7D | 291312060 | C, T | OT3098 |
| 3C | 450156513 | C, T | sanfensan | 6A | 67285702 | C, T | sanfensan | 7D | 363340392 | T, C | OT3098 |
| 3C | 458930099 | T, C | sanfensan | 6A | 71815074 | C, A | sanfensan | 7D | 370273697 | A, G | OT3098 |
| 3C | 460938762 | C, T | sanfensan | 6A | 77438434 | C, A | sanfensan | 7D | 382112487 | G, A | OT3098 |
| 3C | 465773933 | A, G | sanfensan | 6A | 83659654 | G, A | sanfensan | 7D | 391806334 | A, G | OT3098 |
| 3C | 466127904 | C, A | sanfensan | 6A | 85235001 | C, A | sanfensan | 7D | 416042498 | C, T | OT3098 |
| 3C | 472480324 | G, T | sanfensan | 6A | 94561841 | T, G | sanfensan | 7D | 430774655 | T, C | OT3098 |
| 3C | 476852155 | T, C | sanfensan | 6A | 95323662 | A, C | sanfensan | 7D | 466485714 | C, T | OT3098 |
| 3C | 482946601 | C, T | sanfensan | 6A | 103614490 | T, C | sanfensan | | | | |

### Embodiment 2

The application method of 2K SNP liquid chip of Avena sativas includes the following steps.
(1) DNA extraction: high throughput DNA extraction kit or cetyltrimethylammonium bromide (CTAB) method is used to extract DNA from Avena sativa samples to be detected.
(2) DNA quality inspection: 1% agarose gel electrophoresis is used to analyze the purity, integrity and contamination of DNAs; accurate quantification of DNA concentration is performed by Qubit; accurate integrity detection is performed by Agilent 2100. The DNAs with qualified standard for quality inspection are DNAs with total amount not less than 4 μg, sample concentration less than 40 ng/μL, good sample integrity and no impurity pollution.
(3) Library construction: the qualified sample DNAs are randomly interrupted by an ultrasonic breaker, DNA fragments with required length are recovered by electrophoresis, and a linkers are added at the ends to form a library.
(4) Construction of sequencing library: LM-PCR is used to amplify the sample library and purification is performed, which is the sequencing library, and the sequencing library is used for probe hybridization experiments.
(5) Construction of hybridization capture library: 300 ng of sequencing library after the construction is taken, freeze-dried, 2K SNP liquid chip of Avena sativa and hybridization reagent are added, denatured and incubated at 65° C. for 6 hours to complete hybridization reaction; after washing the hybridization products with lotion, another round of PCR is carried out to complete the construction of hybridization capture library.
(6) Quality inspection of hybridization capture library: Qubit2.0 is used for preliminary quantification, and the effective concentration of the library is accurately quantified by qPCR method to ensure the quality of the library.
(7) Sequencing: sequencing is performed with illumina sequencer and related reagents.
(8) Analysis: after obtaining the off-board data, GTAK best practice process is used to carry out the standard call SNP of the data; the sequencing results are compared with the Avena sativa reference genomes, so as to obtain the genotyping data of the genomes to be tested.

### Embodiment 3

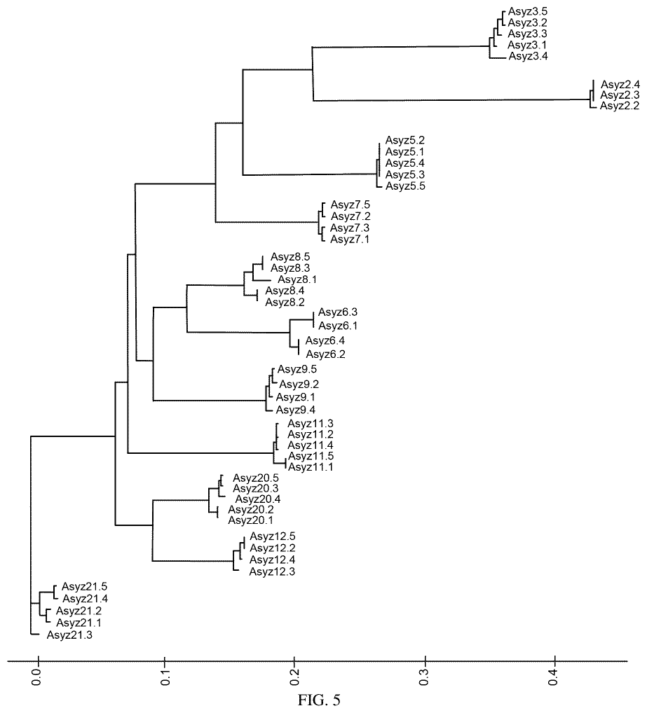

The 2K SNP liquid chip of Avena sativa obtained in Embodiment 1 has the following application cases.
(1) 49 parts of Avena sativa leaves are taken, the samples are shown in Table 2 (including different duplicate samples), and stored in the refrigerator at −20° C. for DNA extraction.
(2) DNA extraction, library construction, computer sequencing are carried out according to the application method of 2K SNP liquid chip of Avena sativas in Embodiment 2, and final SNP data is obtained.
(3) Variety authenticity identifying:
fingerprint data of 2429 SNP loci of the samples to be tested is constructed; the fingerprints of the samples to be detected are compared in pairs, statistical comparison is performed on the total number of loci and the number of difference loci, and the similarity of the samples to be detected is calculated. The calculation results of similarity are shown in Table 3-Table 8. The genetic distance cluster analysis diagram of 49 germplasm resources is shown in FIG. 5.

Similarity (%)=number of consistent loci/total number of loci×100%

According to the 49 Avena sativa varieties tested, it is determined by similarity calculation that when the similarity of loci is less than 88.00%, the two varieties are excluded as the same variety. When the similarity of loci is between 88% and 96%, it is not certain that the two varieties are the same variety; the similarity of loci is more than 96.00%, which does not rule out that the two varieties belong to the same variety.

TABLE 2
49 parts of Avena sativa samples

| | Material | | Material | | Material | | Material |
|---|---|---|---|---|---|---|---|
| No. | name | No. | name | No. | name | No. | name |
| Asyz2.2 | Xiaoyoumai | Asyz6.1 | Mengsiyan No. 7 | Asyz9.1 | Baiyan No. 8 | Asyz20.1 | Baylor |
| Asyz2.3 | Xiaoyoumai | Asyz6.2 | Mengsiyan No. 7 | Asyz9.2 | Baiyan No. 8 | Asyz2 | Baylor |
| Asyz2.4 | Xiaoyoumai | Asyz6.3 | Mengsiyan No. 7 | Asyz9.4 | Baiyan No. 8 | Asyz20.3 | Baylor |
| Asyz3.1 | 578 | Asyz6.4 | Mengsiyan No. 7 | Asyz9.5 | Baiyan No. 8 | Asyz20.4 | Baylor |
| Asyz3.2 | 578 | Asyz7.1 | Mengsiyan No. 1 | Asyz11.1 | K107722 | Asyz20.5 | Baylor |
| Asyz3.3 | 578 | Asyz7.2 | Mengsiyan No. 1 | Asyz11.2 | K107722 | Asyz21.1 | Modern |
| Asyz3.4 | 578 | Asyz7.3 | Mengsiyan No. 1 | Asyz11.3 | K107722 | Asyz21.2 | Modern |
| Asyz3.5 | 578 | Asyz7.5 | Mengsiyan No. 1 | Asyz11.4 | K107722 | Asyz21.3 | Modern |
| Asyz5.1 | Yanke No. 2 | Asyz8.1 | Huazao No. 2 | Asyz11.5 | K107722 | Asyz21.4 | Modern |
| Asyz5.2 | Yanke No. 2 | Asyz8.2 | Huazao No. 2 | Asyz12.2 | Dingyan No. 2 | Asyz21.5 | Modern |
| Asyz5.3 | Yanke No. 2 | Asyz8.3 | Huazao No. 2 | Asyz12.3 | Dingyan No. 2 | | |
| Asyz5.4 | Yanke No. 2 | Asyz8.4 | Huazao No. 2 | Asyz12.4 | Dingyan No. 2 | | |
| Asyz5.5 | Yanke No. 2 | Asyz8.5 | Huazao No. 2 | Asyz12.5 | Dingyan No. 2 | | |

TABLE 3
Similarity calculation results among 49 parts of Avena sativa samples, K107722 and Dingyan No. 2

| Material No. | Asyz11.1 | Asyz11.2 | Asyz11.3 | Asyz11.4 | Asyz11.5 | Asyz12.2 | Asyz12.3 | Asyz12.4 | Asyz12.5 |
|---|---|---|---|---|---|---|---|---|---|
| Asyz11.1 | 100.00% | 99.96% | 99.96% | 99.96% | 100.00% | 77.76% | 77.75% | 77.80% | 77.76% |
| Asyz11.2 | 99.96% | 100.00% | 100.00% | 100.00% | 99.96% | 77.76% | 77.75% | 77.80% | 77.76% |
| Asyz11.3 | 99.96% | 100.00% | 100.00% | 100.00% | 99.96% | 77.76% | 77.75% | 77.80% | 77.76% |
| Asyz11.4 | 99.96% | 100.00% | 100.00% | 100.00% | 99.96% | 77.76% | 77.75% | 77.80% | 77.76% |
| Asyz11.5 | 100.00% | 99.96% | 99.96% | 99.96% | 100.00% | 77.76% | 77.75% | 77.80% | 77.76% |
| Asyz12.2 | 77.76% | 77.76% | 77.76% | 77.76% | 77.76% | 100.00% | 99.91% | 99.96% | 100.00% |
| Asyz12.3 | 77.75% | 77.75% | 77.75% | 77.75% | 77.75% | 99.91% | 100.00% | 99.87% | 99.91% |
| Asyz12.4 | 77.80% | 77.80% | 77.80% | 77.80% | 77.80% | 99.96% | 99.87% | 100.00% | 99.96% |
| Asyz12.5 | 77.76% | 77.76% | 77.76% | 77.76% | 77.76% | 100.00% | 99.91% | 99.96% | 100.00% |
| Asyz2.2 | 55.11% | 55.16% | 55.16% | 55.16% | 55.11% | 55.05% | 55.03% | 55.01% | 55.05% |
| Asyz2.3 | 55.09% | 55.14% | 55.14% | 55.14% | 55.09% | 55.03% | 55.01% | 54.99% | 55.03% |
| Asyz2.4 | 55.05% | 55.09% | 55.09% | 55.09% | 55.05% | 54.98% | 54.96% | 54.94% | 54.98% |
| Asyz20.1 | 78.05% | 78.09% | 78.09% | 78.09% | 78.05% | 88.61% | 88.69% | 88.65% | 88.61% |
| Asyz20.2 | 78.10% | 78.15% | 78.15% | 78.15% | 78.10% | 88.66% | 88.74% | 88.70% | 88.66% |
| Asyz20.3 | 79.80% | 79.84% | 79.84% | 79.84% | 79.80% | 86.69% | 86.77% | 86.65% | 86.69% |
| Asyz20.4 | 79.48% | 79.53% | 79.53% | 79.53% | 79.48% | 87.32% | 87.40% | 87.36% | 87.32% |
| Asyz20.5 | 79.92% | 79.88% | 79.88% | 79.88% | 79.92% | 86.77% | 86.86% | 86.82% | 86.77% |
| Asyz21.1 | 81.48% | 81.53% | 81.53% | 81.53% | 81.48% | 83.24% | 83.24% | 83.29% | 83.24% |
| Asyz21.2 | 81.53% | 81.57% | 81.57% | 81.57% | 81.53% | 83.33% | 83.33% | 83.38% | 83.33% |
| Asyz21.3 | 81.62% | 81.66% | 81.66% | 81.66% | 81.62% | 83.33% | 83.33% | 83.38% | 83.33% |
| Asyz21.4 | 81.17% | 81.21% | 81.21% | 81.21% | 81.17% | 83.20% | 83.19% | 83.24% | 83.20% |
| Asyz21.5 | 81.21% | 81.26% | 81.26% | 81.26% | 81.21% | 83.20% | 83.19% | 83.24% | 83.20% |
| Asyz3.1 | 63.10% | 63.14% | 63.14% | 63.14% | 63.10% | 60.72% | 60.80% | 60.68% | 60.72% |
| Asyz3.2 | 63.08% | 63.13% | 63.13% | 63.13% | 63.08% | 60.71% | 60.78% | 60.66% | 60.71% |
| Asyz3.3 | 63.08% | 63.13% | 63.13% | 63.13% | 63.08% | 60.71% | 60.78% | 60.66% | 60.71% |
| Asyz3.4 | 63.08% | 63.13% | 63.13% | 63.13% | 63.08% | 60.62% | 60.69% | 60.57% | 60.62% |
| Asyz3.5 | 63.10% | 63.14% | 63.14% | 63.14% | 63.10% | 60.68% | 60.75% | 60.63% | 60.68% |
| Asyz5.1 | 73.02% | 73.06% | 73.06% | 73.06% | 73.02% | 70.03% | 70.11% | 70.08% | 70.03% |
| Asyz5.2 | 73.02% | 73.06% | 73.06% | 73.06% | 73.02% | 70.03% | 70.11% | 70.08% | 70.03% |
| Asyz5.3 | 73.02% | 73.06% | 73.06% | 73.06% | 73.02% | 70.03% | 70.11% | 70.08% | 70.03% |
| Asyz5.4 | 73.02% | 73.06% | 73.06% | 73.06% | 73.02% | 70.03% | 70.11% | 70.08% | 70.03% |
| Asyz5.5 | 72.97% | 73.02% | 73.02% | 73.02% | 72.97% | 69.94% | 70.02% | 69.99% | 69.94% |
| Asyz6.1 | 74.18% | 74.22% | 74.22% | 74.22% | 74.18% | 76.28% | 76.36% | 76.24% | 76.28% |
| Asyz6.2 | 72.88% | 72.93% | 72.93% | 72.93% | 72.88% | 77.76% | 77.84% | 77.71% | 77.76% |
| Asyz6.3 | 74.22% | 74.26% | 74.26% | 74.26% | 74.22% | 76.28% | 76.36% | 76.24% | 76.28% |
| Asyz6.4 | 72.93% | 72.97% | 72.97% | 72.97% | 72.93% | 77.76% | 77.84% | 77.71% | 77.76% |
| Asyz7.1 | 70.29% | 70.34% | 70.34% | 70.34% | 70.29% | 76.55% | 76.63% | 76.60% | 76.55% |
| Asyz7.2 | 70.21% | 70.25% | 70.25% | 70.25% | 70.21% | 76.51% | 76.59% | 76.55% | 76.51% |
| Asyz7.3 | 70.33% | 70.37% | 70.37% | 70.37% | 70.33% | 76.59% | 76.67% | 76.63% | 76.59% |
| Asyz7.5 | 70.25% | 70.29% | 70.29% | 70.29% | 70.25% | 76.55% | 76.63% | 76.60% | 76.55% |
| Asyz8.1 | 78.90% | 78.95% | 78.95% | 78.95% | 78.90% | 80.84% | 80.92% | 80.80% | 80.84% |
| Asyz8.2 | 78.40% | 78.45% | 78.45% | 78.45% | 78.40% | 80.61% | 80.69% | 80.65% | 80.61% |
| Asyz8.3 | 79.63% | 79.68% | 79.68% | 79.68% | 79.63% | 81.40% | 81.48% | 81.44% | 81.40% |
| Asyz8.4 | 78.36% | 78.40% | 78.40% | 78.40% | 78.36% | 80.56% | 80.64% | 80.61% | 80.56% |
| Asyz8.5 | 79.62% | 79.66% | 79.66% | 79.66% | 79.62% | 81.42% | 81.50% | 81.47% | 81.42% |
| Asyz9.1 | 77.43% | 77.43% | 77.43% | 77.43% | 77.43% | 78.43% | 78.51% | 78.38% | 78.43% |
| Asyz9.2 | 77.33% | 77.33% | 77.33% | 77.33% | 77.33% | 78.24% | 78.32% | 78.20% | 78.24% |
| Asyz9.4 | 77.39% | 77.39% | 77.39% | 77.39% | 77.39% | 78.25% | 78.33% | 78.21% | 78.25% |
| Asyz9.5 | 77.47% | 77.47% | 77.47% | 77.47% | 77.47% | 78.20% | 78.27% | 78.15% | 78.20% |

TABLE 4
Similarity calculation results among 49 parts of Avena sativa samples, Xiaoyoumai and
Baylor

| Material | | | | | | | | |
|---|---|---|---|---|---|---|---|---|
| No. | Asyz2.2 | Asyz2.3 | Asyz2.4 | Asyz20.1 | Asyz20.2 | Asyz20.3 | Asyz20.4 | Asyz20.5 |
| Asyz11.1 | 55.11% | 55.09% | 55.05% | 78.05% | 78.10% | 79.80% | 79.48% | 79.92% |
| Asyz11.2 | 55.16% | 55.14% | 55.09% | 78.09% | 78.15% | 79.84% | 79.53% | 79.88% |
| Asyz11.3 | 55.16% | 55.14% | 55.09% | 78.09% | 78.15% | 79.84% | 79.53% | 79.88% |
| Asyz11.4 | 55.16% | 55.14% | 55.09% | 78.09% | 78.15% | 79.84% | 79.53% | 79.88% |
| Asyz11.5 | 55.11% | 55.09% | 55.05% | 78.05% | 78.10% | 79.80% | 79.48% | 79.92% |
| Asyz12.2 | 55.05% | 55.03% | 54.98% | 88.61% | 88.66% | 86.69% | 87.32% | 86.77% |
| Asyz12.3 | 55.03% | 55.01% | 54.96% | 88.69% | 88.74% | 86.77% | 87.40% | 86.86% |
| Asyz12.4 | 55.01% | 54.99% | 54.94% | 88.65% | 88.70% | 86.65% | 87.36% | 86.82% |
| Asyz12.5 | 55.05% | 55.03% | 54.98% | 88.61% | 88.66% | 86.69% | 87.32% | 86.77% |
| Asyz2.2 | 100.00% | 100.00% | 100.00% | 55.23% | 55.20% | 55.07% | 54.44% | 54.96% |
| Asyz2.3 | 100.00% | 100.00% | 100.00% | 55.21% | 55.18% | 55.05% | 54.42% | 54.94% |
| Asyz2.4 | 100.00% | 100.00% | 100.00% | 55.16% | 55.13% | 55.00% | 54.38% | 54.89% |
| Asyz20.1 | 55.23% | 55.21% | 55.16% | 100.00% | 99.96% | 97.81% | 98.44% | 97.86% |
| Asyz20.2 | 55.20% | 55.18% | 55.13% | 99.96% | 100.00% | 97.77% | 98.48% | 97.90% |
| Asyz20.3 | 55.07% | 55.05% | 55.00% | 97.81% | 97.77% | 100.00% | 99.15% | 99.73% |
| Asyz20.4 | 54.44% | 54.42% | 54.38% | 98.44% | 98.48% | 99.15% | 100.00% | 99.33% |
| Asyz20.5 | 54.96% | 54.94% | 54.89% | 97.86% | 97.90% | 99.73% | 99.33% | 100.00% |
| Asyz21.1 | 56.88% | 56.86% | 56.81% | 86.61% | 86.57% | 85.99% | 85.36% | 85.89% |
| Asyz21.2 | 56.97% | 56.95% | 56.90% | 86.61% | 86.57% | 85.99% | 85.36% | 85.89% |
| Asyz21.3 | 56.93% | 56.91% | 56.86% | 86.74% | 86.70% | 86.17% | 85.54% | 86.07% |
| Asyz21.4 | 56.84% | 56.82% | 56.77% | 86.25% | 86.21% | 85.59% | 84.96% | 85.49% |
| Asyz21.5 | 56.66% | 56.64% | 56.59% | 86.30% | 86.26% | 85.68% | 85.05% | 85.58% |
| Asyz3.1 | 62.96% | 62.94% | 62.89% | 59.11% | 59.04% | 59.97% | 60.42% | 59.91% |
| Asyz3.2 | 62.94% | 62.93% | 62.87% | 59.09% | 59.02% | 59.96% | 60.40% | 59.89% |
| Asyz3.3 | 62.94% | 62.93% | 62.87% | 59.09% | 59.02% | 59.96% | 60.40% | 59.89% |
| Asyz3.4 | 63.03% | 63.01% | 62.96% | 59.00% | 58.93% | 59.87% | 60.31% | 59.80% |
| Asyz3.5 | 62.91% | 62.90% | 62.84% | 59.06% | 58.99% | 59.93% | 60.38% | 59.87% |
| Asyz5.1 | 51.85% | 51.83% | 51.79% | 68.32% | 68.24% | 69.18% | 69.72% | 69.21% |
| Asyz5.2 | 51.85% | 51.83% | 51.79% | 68.32% | 68.24% | 69.18% | 69.72% | 69.21% |
| Asyz5.3 | 51.85% | 51.83% | 51.79% | 68.32% | 68.24% | 69.18% | 69.72% | 69.21% |
| Asyz5.4 | 51.85% | 51.83% | 51.79% | 68.32% | 68.24% | 69.18% | 69.72% | 69.21% |
| Asyz5.5 | 51.94% | 51.92% | 51.88% | 68.36% | 68.29% | 69.14% | 69.67% | 69.17% |
| Asyz6.1 | 56.68% | 56.66% | 56.61% | 78.89% | 78.86% | 78.37% | 77.65% | 78.18% |
| Asyz6.2 | 55.47% | 55.45% | 55.40% | 80.37% | 80.33% | 79.93% | 79.13% | 79.65% |
| Asyz6.3 | 56.72% | 56.70% | 56.65% | 78.94% | 78.90% | 78.41% | 77.70% | 78.22% |
| Asyz6.4 | 55.52% | 55.50% | 55.45% | 80.41% | 80.38% | 79.97% | 79.17% | 79.70% |
| Asyz7.1 | 55.65% | 55.63% | 55.58% | 75.81% | 75.83% | 74.00% | 74.71% | 74.12% |
| Asyz7.2 | 55.61% | 55.59% | 55.54% | 75.73% | 75.74% | 73.91% | 74.62% | 74.03% |
| Asyz7.3 | 55.68% | 55.66% | 55.61% | 75.85% | 75.86% | 74.03% | 74.74% | 74.15% |
| Asyz7.5 | 55.65% | 55.63% | 55.58% | 75.77% | 75.78% | 73.95% | 74.67% | 74.07% |
| Asyz8.1 | 55.34% | 55.32% | 55.27% | 80.10% | 80.15% | 79.66% | 79.04% | 79.56% |
| Asyz8.2 | 53.40% | 53.38% | 53.33% | 81.83% | 81.88% | 81.21% | 80.72% | 81.25% |
| Asyz8.3 | 54.03% | 54.00% | 53.96% | 80.65% | 80.71% | 80.08% | 79.54% | 80.07% |
| Asyz8.4 | 53.44% | 53.42% | 53.37% | 81.79% | 81.84% | 81.26% | 80.72% | 81.25% |
| Asyz8.5 | 54.04% | 54.02% | 53.97% | 80.63% | 80.69% | 80.11% | 79.57% | 80.10% |
| Asyz9.1 | 55.11% | 55.09% | 55.05% | 77.60% | 77.57% | 79.22% | 78.64% | 79.12% |
| Asyz9.2 | 55.23% | 55.21% | 55.16% | 77.28% | 77.24% | 78.85% | 78.27% | 78.75% |
| Asyz9.4 | 55.34% | 55.32% | 55.27% | 77.82% | 77.79% | 79.35% | 78.77% | 79.25% |
| Asyz9.5 | 55.32% | 55.30% | 55.25% | 77.46% | 77.42% | 79.16% | 78.54% | 79.02% |

TABLE 5
Similarity calculation results among 49 parts of Avena sativa samples and Modern and
578

| Material | | | | | | | | | | |
|---|---|---|---|---|---|---|---|---|---|---|
| No | Asyz21.1 | Asyz21.2 | Asyz21.3 | Asyz21.4 | Asyz21.5 | Asyz3.1 | Asyz3.2 | Asyz3.3 | Asyz3.4 | Asyz3.5 |
| Asyz11.1 | 81.48% | 81.53% | 81.62% | 81.17% | 81.21% | 63.10% | 63.08% | 63.08% | 63.08% | 63.10% |
| Asyz11.2 | 81.53% | 81.57% | 81.66% | 81.21% | 81.26% | 63.14% | 63.13% | 63.13% | 63.13% | 63.14% |
| Asyz11.3 | 81.53% | 81.57% | 81.66% | 81.21% | 81.26% | 63.14% | 63.13% | 63.13% | 63.13% | 63.14% |
| Asyz11.4 | 81.53% | 81.57% | 81.66% | 81.21% | 81.26% | 63.14% | 63.13% | 63.13% | 63.13% | 63.14% |
| Asyz11.5 | 81.48% | 81.53% | 81.62% | 81.17% | 81.21% | 63.10% | 63.08% | 63.08% | 63.08% | 63.10% |
| Asyz12.2 | 83.24% | 83.33% | 83.33% | 83.20% | 83.20% | 60.72% | 60.71% | 60.71% | 60.62% | 60.68% |
| Asyz12.3 | 83.24% | 83.33% | 83.33% | 83.19% | 83.19% | 60.80% | 60.78% | 60.78% | 60.69% | 60.75% |
| Asyz12.4 | 83.29% | 83.38% | 83.38% | 83.24% | 83.24% | 60.68% | 60.66% | 60.66% | 60.57% | 60.63% |
| Asyz12.5 | 83.24% | 83.33% | 83.33% | 83.20% | 83.20% | 60.72% | 60.71% | 60.71% | 60.62% | 60.68% |
| Asyz2.2 | 56.88% | 56.97% | 56.93% | 56.84% | 56.66% | 62.96% | 62.94% | 62.94% | 63.03% | 62.91% |
| Asyz2.3 | 56.86% | 56.95% | 56.91% | 56.82% | 56.64% | 62.94% | 62.93% | 62.93% | 63.01% | 62.90% |
| Asyz2.4 | 56.81% | 56.90% | 56.86% | 56.77% | 56.59% | 62.89% | 62.87% | 62.87% | 62.96% | 62.84% |
| Asyz20.1 | 86.61% | 86.61% | 86.74% | 86.25% | 86.30% | 59.11% | 59.09% | 59.09% | 59.00% | 59.06% |
| Asyz20.2 | 86.57% | 86.57% | 86.70% | 86.21% | 86.26% | 59.04% | 59.02% | 59.02% | 58.93% | 58.99% |
| Asyz20.3 | 85.99% | 85.99% | 86.17% | 85.59% | 85.68% | 59.97% | 59.96% | 59.96% | 59.87% | 59.93% |
| Asyz20.4 | 85.36% | 85.36% | 85.54% | 84.96% | 85.05% | 60.42% | 60.40% | 60.40% | 60.31% | 60.38% |
| Asyz20.5 | 85.89% | 85.89% | 86.07% | 85.49% | 85.58% | 59.91% | 59.89% | 59.89% | 59.80% | 59.87% |
| Asyz21.1 | 100.00% | 99.82% | 99.69% | 99.33% | 99.42% | 62.86% | 62.84% | 62.84% | 62.75% | 62.81% |
| Asyz21.2 | 99.82% | 100.00% | 99.64% | 99.42% | 99.51% | 62.86% | 62.84% | 62.84% | 62.75% | 62.81% |
| Asyz21.3 | 99.69% | 99.64% | 100.00% | 99.29% | 99.33% | 62.90% | 62.89% | 62.89% | 62.80% | 62.86% |
| Asyz21.4 | 99.33% | 99.42% | 99.29% | 100.00% | 99.73% | 62.63% | 62.62% | 62.62% | 62.53% | 62.59% |
| Asyz21.5 | 99.42% | 99.51% | 99.33% | 99.73% | 100.00% | 62.72% | 62.71% | 62.71% | 62.62% | 62.68% |
| Asyz3.1 | 62.86% | 62.86% | 62.90% | 62.63% | 62.72% | 100.00% | 100.00% | 100.00% | 99.91% | 100.00% |
| Asyz3.2 | 62.84% | 62.84% | 62.89% | 62.62% | 62.71% | 100.00% | 100.00% | 100.00% | 99.91% | 100.00% |
| Asyz3.3 | 62.84% | 62.84% | 62.89% | 62.62% | 62.71% | 100.00% | 100.00% | 100.00% | 99.91% | 100.00% |
| Asyz3.4 | 62.75% | 62.75% | 62.80% | 62.53% | 62.62% | 99.91% | 99.91% | 99.91% | 100.00% | 99.91% |
| Asyz3.5 | 62.81% | 62.81% | 62.86% | 62.59% | 62.68% | 100.00% | 100.00% | 100.00% | 99.91% | 100.00% |
| Asyz5.1 | 72.87% | 72.87% | 72.87% | 72.56% | 72.56% | 78.72% | 78.71% | 78.71% | 78.62% | 78.67% |
| Asyz5.2 | 72.87% | 72.87% | 72.87% | 72.56% | 72.56% | 78.72% | 78.71% | 78.71% | 78.62% | 78.67% |
| Asyz5.3 | 72.87% | 72.87% | 72.87% | 72.56% | 72.56% | 78.72% | 78.71% | 78.71% | 78.62% | 78.67% |
| Asyz5.4 | 72.87% | 72.87% | 72.87% | 72.56% | 72.56% | 78.72% | 78.71% | 78.71% | 78.62% | 78.67% |
| Asyz5.5 | 72.87% | 72.87% | 72.83% | 72.56% | 72.56% | 78.85% | 78.84% | 78.84% | 78.75% | 78.80% |
| Asyz6.1 | 77.82% | 77.82% | 78.05% | 77.87% | 77.78% | 58.64% | 58.62% | 58.62% | 58.53% | 58.59% |
| Asyz6.2 | 79.21% | 79.21% | 79.43% | 79.25% | 79.16% | 59.84% | 59.82% | 59.82% | 59.73% | 59.80% |
| Asyz6.3 | 77.87% | 77.87% | 78.09% | 77.91% | 77.82% | 58.68% | 58.66% | 58.66% | 58.57% | 58.64% |
| Asyz6.4 | 79.25% | 79.25% | 79.47% | 79.30% | 79.21% | 59.88% | 59.87% | 59.87% | 59.78% | 59.84% |
| Asyz7.1 | 76.84% | 76.89% | 76.80% | 77.15% | 77.29% | 71.31% | 71.30% | 71.30% | 71.21% | 71.26% |
| Asyz7.2 | 76.75% | 76.80% | 76.71% | 77.11% | 77.20% | 71.22% | 71.21% | 71.21% | 71.12% | 71.17% |
| Asyz7.3 | 76.88% | 76.92% | 76.83% | 77.19% | 77.32% | 71.34% | 71.33% | 71.33% | 71.24% | 71.30% |
| Asyz7.5 | 76.80% | 76.84% | 76.75% | 77.15% | 77.24% | 71.26% | 71.25% | 71.25% | 71.16% | 71.22% |
| Asyz8.1 | 81.30% | 81.30% | 81.48% | 81.21% | 81.26% | 61.89% | 61.88% | 61.88% | 61.79% | 61.85% |
| Asyz8.2 | 83.08% | 83.08% | 83.26% | 83.04% | 83.04% | 60.71% | 60.70% | 60.70% | 60.61% | 60.67% |
| Asyz8.3 | 82.17% | 82.17% | 82.26% | 82.05% | 82.13% | 61.08% | 61.06% | 61.06% | 60.98% | 61.04% |
| Asyz8.4 | 83.13% | 83.13% | 83.30% | 83.08% | 83.08% | 60.76% | 60.74% | 60.74% | 60.65% | 60.71% |
| Asyz8.5 | 82.11% | 82.11% | 82.29% | 82.02% | 82.06% | 61.09% | 61.07% | 61.07% | 60.98% | 61.04% |
| Asyz9.1 | 81.79% | 81.79% | 81.97% | 81.75% | 81.84% | 64.35% | 64.33% | 64.33% | 64.33% | 64.30% |
| Asyz9.2 | 81.38% | 81.38% | 81.61% | 81.43% | 81.43% | 64.38% | 64.36% | 64.36% | 64.36% | 64.33% |
| Asyz9.4 | 81.66% | 81.66% | 81.88% | 81.71% | 81.71% | 64.44% | 64.42% | 64.42% | 64.42% | 64.39% |
| Asyz9.5 | 81.65% | 81.65% | 81.83% | 81.61% | 81.70% | 64.42% | 64.40% | 64.40% | 64.40% | 64.38% |

TABLE 6
Similarity calculation results among 49 parts of Avena sativa samples and Yanke No. 2
and Mengsiyan No. 7

| Material | | | | | | | | | |
|---|---|---|---|---|---|---|---|---|---|
| No. | Asyz5.1 | Asyz5.2 | Asyz5.3 | Asyz5.4 | Asyz5.5 | Asyz6.1 | Asyz6.2 | Asyz6.3 | Asyz6.4 |
| Asyz11.1 | 73.02% | 73.02% | 73.02% | 73.02% | 72.97% | 74.18% | 72.88% | 74.22% | 72.93% |
| Asyz11.2 | 73.06% | 73.06% | 73.06% | 73.06% | 73.02% | 74.22% | 72.93% | 74.26% | 72.97% |
| Asyz11.3 | 73.06% | 73.06% | 73.06% | 73.06% | 73.02% | 74.22% | 72.93% | 74.26% | 72.97% |
| Asyz11.4 | 73.06% | 73.06% | 73.06% | 73.06% | 73.02% | 74.22% | 72.93% | 74.26% | 72.97% |
| Asyz11.5 | 73.02% | 73.02% | 73.02% | 73.02% | 72.97% | 74.18% | 72.88% | 74.22% | 72.93% |
| Asyz12.2 | 70.03% | 70.03% | 70.03% | 70.03% | 69.94% | 76.28% | 77.76% | 76.28% | 77.76% |
| Asyz12.3 | 70.11% | 70.11% | 70.11% | 70.11% | 70.02% | 76.36% | 77.84% | 76.36% | 77.84% |
| Asyz12.4 | 70.08% | 70.08% | 70.08% | 70.08% | 69.99% | 76.24% | 77.71% | 76.24% | 77.71% |
| Asyz12.5 | 70.03% | 70.03% | 70.03% | 70.03% | 69.94% | 76.28% | 77.76% | 76.28% | 77.76% |
| Asyz2.2 | 51.85% | 51.85% | 51.85% | 51.85% | 51.94% | 56.68% | 55.47% | 56.72% | 55.52% |
| Asyz2.3 | 51.83% | 51.83% | 51.83% | 51.83% | 51.92% | 56.66% | 55.45% | 56.70% | 55.50% |
| Asyz2.4 | 51.79% | 51.79% | 51.79% | 51.79% | 51.88% | 56.61% | 55.40% | 56.65% | 55.45% |
| Asyz20.1 | 68.32% | 68.32% | 68.32% | 68.32% | 68.36% | 78.89% | 80.37% | 78.94% | 80.41% |
| Asyz20.2 | 68.24% | 68.24% | 68.24% | 68.24% | 68.29% | 78.86% | 80.33% | 78.90% | 80.38% |
| Asyz20.3 | 69.18% | 69.18% | 69.18% | 69.18% | 69.14% | 78.37% | 79.93% | 78.41% | 79.97% |
| Asyz20.4 | 69.72% | 69.72% | 69.72% | 69.72% | 69.67% | 77.65% | 79.13% | 77.70% | 79.17% |
| Asyz20.5 | 69.21% | 69.21% | 69.21% | 69.21% | 69.17% | 78.18% | 79.65% | 78.22% | 79.70% |
| Asyz21.1 | 72.87% | 72.87% | 72.87% | 72.87% | 72.87% | 77.82% | 79.21% | 77.87% | 79.25% |
| Asyz21.2 | 72.87% | 72.87% | 72.87% | 72.87% | 72.87% | 77.82% | 79.21% | 77.87% | 79.25% |
| Asyz21.3 | 72.87% | 72.87% | 72.87% | 72.87% | 72.83% | 78.05% | 79.43% | 78.09% | 79.47% |
| Asyz21.4 | 72.56% | 72.56% | 72.56% | 72.56% | 72.56% | 77.87% | 79.25% | 77.91% | 79.30% |
| Asyz21.5 | 72.56% | 72.56% | 72.56% | 72.56% | 72.56% | 77.78% | 79.16% | 77.82% | 79.21% |
| Asyz3.1 | 78.72% | 78.72% | 78.72% | 78.72% | 78.85% | 58.64% | 59.84% | 58.68% | 59.88% |
| Asyz3.2 | 78.71% | 78.71% | 78.71% | 78.71% | 78.84% | 58.62% | 59.82% | 58.66% | 59.87% |
| Asyz3.3 | 78.71% | 78.71% | 78.71% | 78.71% | 78.84% | 58.62% | 59.82% | 58.66% | 59.87% |
| Asyz3.4 | 78.62% | 78.62% | 78.62% | 78.62% | 78.75% | 58.53% | 59.73% | 58.57% | 59.78% |
| Asyz3.5 | 78.67% | 78.67% | 78.67% | 78.67% | 78.80% | 58.59% | 59.80% | 58.64% | 59.84% |
| Asyz5.1 | 100.00% | 100.00% | 100.00% | 100.00% | 99.87% | 65.70% | 66.91% | 65.75% | 66.95% |
| Asyz5.2 | 100.00% | 100.00% | 100.00% | 100.00% | 99.87% | 65.70% | 66.91% | 65.75% | 66.95% |
| Asyz5.3 | 100.00% | 100.00% | 100.00% | 100.00% | 99.87% | 65.70% | 66.91% | 65.75% | 66.95% |
| Asyz5.4 | 100.00% | 100.00% | 100.00% | 100.00% | 99.87% | 65.70% | 66.91% | 65.75% | 66.95% |
| Asyz5.5 | 99.87% | 99.87% | 99.87% | 99.87% | 100.00% | 65.70% | 66.91% | 65.75% | 66.95% |
| Asyz6.1 | 65.70% | 65.70% | 65.70% | 65.70% | 65.70% | 100.00% | 98.26% | 99.96% | 98.22% |
| Asyz6.2 | 66.91% | 66.91% | 66.91% | 66.91% | 66.91% | 98.26% | 100.00% | 98.22% | 99.96% |
| Asyz6.3 | 65.75% | 65.75% | 65.75% | 65.75% | 65.75% | 99.96% | 98.22% | 100.00% | 98.26% |
| Asyz6.4 | 66.95% | 66.95% | 66.95% | 66.95% | 66.95% | 98.22% | 99.96% | 98.26% | 100.00% |
| Asyz7.1 | 80.24% | 80.24% | 80.24% | 80.24% | 80.20% | 70.03% | 71.50% | 70.07% | 71.54% |
| Asyz7.2 | 80.29% | 80.29% | 80.29% | 80.29% | 80.15% | 70.03% | 71.50% | 70.07% | 71.54% |
| Asyz7.3 | 80.28% | 80.28% | 80.28% | 80.28% | 80.23% | 70.06% | 71.53% | 70.10% | 71.58% |
| Asyz7.5 | 80.29% | 80.29% | 80.29% | 80.29% | 80.15% | 70.03% | 71.50% | 70.07% | 71.54% |
| Asyz8.1 | 73.64% | 73.64% | 73.64% | 73.64% | 73.51% | 83.23% | 84.79% | 83.23% | 84.79% |
| Asyz8.2 | 73.23% | 73.23% | 73.23% | 73.23% | 73.09% | 85.01% | 86.57% | 85.05% | 86.61% |
| Asyz8.3 | 73.34% | 73.34% | 73.34% | 73.34% | 73.29% | 84.23% | 85.80% | 84.23% | 85.80% |
| Asyz8.4 | 73.23% | 73.23% | 73.23% | 73.23% | 73.09% | 85.05% | 86.61% | 85.10% | 86.66% |
| Asyz8.5 | 73.42% | 73.42% | 73.42% | 73.42% | 73.28% | 84.21% | 85.77% | 84.21% | 85.77% |
| Asyz9.1 | 70.38% | 70.38% | 70.38% | 70.38% | 70.29% | 77.70% | 79.26% | 77.74% | 79.30% |
| Asyz9.2 | 70.37% | 70.37% | 70.37% | 70.37% | 70.33% | 77.47% | 79.03% | 77.51% | 79.07% |
| Asyz9.4 | 70.16% | 70.16% | 70.16% | 70.16% | 70.12% | 77.92% | 79.48% | 77.97% | 79.53% |
| Asyz9.5 | 70.55% | 70.55% | 70.55% | 70.55% | 70.46% | 77.38% | 78.94% | 77.42% | 78.98% |

TABLE 7
Similarity calculation results among 49 parts of Avena sativa samples and Mengsiyan
No. 1 and Huazao No. 2

| Material | | | | | | | | | |
|---|---|---|---|---|---|---|---|---|---|
| No. | Asyz7.1 | Asyz7.2 | Asyz7.3 | Asyz7.5 | Asyz8.1 | Asyz8.2 | Asyz8.3 | Asyz8.4 | Asyz8.5 |
| Asyz11.1 | 70.29% | 70.21% | 70.33% | 70.25% | 78.90% | 78.40% | 79.63% | 78.36% | 79.62% |
| Asyz11.2 | 70.34% | 70.25% | 70.37% | 70.29% | 78.95% | 78.45% | 79.68% | 78.40% | 79.66% |
| Asyz11.3 | 70.34% | 70.25% | 70.37% | 70.29% | 78.95% | 78.45% | 79.68% | 78.40% | 79.66% |
| Asyz11.4 | 70.34% | 70.25% | 70.37% | 70.29% | 78.95% | 78.45% | 79.68% | 78.40% | 79.66% |
| Asyz11.5 | 70.29% | 70.21% | 70.33% | 70.25% | 78.90% | 78.40% | 79.63% | 78.36% | 79.62% |
| Asyz12.2 | 76.55% | 76.51% | 76.59% | 76.55% | 80.84% | 80.61% | 81.40% | 80.56% | 81.42% |
| Asyz12.3 | 76.63% | 76.59% | 76.67% | 76.63% | 80.92% | 80.69% | 81.48% | 80.64% | 81.50% |
| Asyz12.4 | 76.60% | 76.55% | 76.63% | 76.60% | 80.80% | 80.65% | 81.44% | 80.61% | 81.47% |
| Asyz12.5 | 76.55% | 76.51% | 76.59% | 76.55% | 80.84% | 80.61% | 81.40% | 80.56% | 81.42% |
| Asyz2.2 | 55.65% | 55.61% | 55.68% | 55.65% | 55.34% | 53.40% | 54.03% | 53.44% | 54.04% |
| Asyz2.3 | 55.63% | 55.59% | 55.66% | 55.63% | 55.32% | 53.38% | 54.00% | 53.42% | 54.02% |
| Asyz2.4 | 55.58% | 55.54% | 55.61% | 55.58% | 55.27% | 53.33% | 53.96% | 53.37% | 53.97% |
| Asyz20.1 | 75.81% | 75.73% | 75.85% | 75.77% | 80.10% | 81.83% | 80.65% | 81.79% | 80.63% |
| Asyz20.2 | 75.83% | 75.74% | 75.86% | 75.78% | 80.15% | 81.88% | 80.71% | 81.84% | 80.69% |
| Asyz20.3 | 74.00% | 73.91% | 74.03% | 73.95% | 79.66% | 81.21% | 80.08% | 81.26% | 80.11% |
| Asyz20.4 | 74.71% | 74.62% | 74.74% | 74.67% | 79.04% | 80.72% | 79.54% | 80.72% | 79.57% |
| Asyz20.5 | 74.12% | 74.03% | 74.15% | 74.07% | 79.56% | 81.25% | 80.07% | 81.25% | 80.10% |
| Asyz21.1 | 76.84% | 76.75% | 76.88% | 76.80% | 81.30% | 83.08% | 82.17% | 83.13% | 82.11% |
| Asyz21.2 | 76.89% | 76.80% | 76.92% | 76.84% | 81.30% | 83.08% | 82.17% | 83.13% | 82.11% |
| Asyz21.3 | 76.80% | 76.71% | 76.83% | 76.75% | 81.48% | 83.26% | 82.26% | 83.30% | 82.29% |
| Asyz21.4 | 77.15% | 77.11% | 77.19% | 77.15% | 81.21% | 83.04% | 82.05% | 83.08% | 82.02% |
| Asyz21.5 | 77.29% | 77.20% | 77.32% | 77.24% | 81.26% | 83.04% | 82.13% | 83.08% | 82.06% |
| Asyz3.1 | 71.31% | 71.22% | 71.34% | 71.26% | 61.89% | 60.71% | 61.08% | 60.76% | 61.09% |
| Asyz3.2 | 71.30% | 71.21% | 71.33% | 71.25% | 61.88% | 60.70% | 61.06% | 60.74% | 61.07% |
| Asyz3.3 | 71.30% | 71.21% | 71.33% | 71.25% | 61.88% | 60.70% | 61.06% | 60.74% | 61.07% |
| Asyz3.4 | 71.21% | 71.12% | 71.24% | 71.16% | 61.79% | 60.61% | 60.98% | 60.65% | 60.98% |
| Asyz3.5 | 71.26% | 71.17% | 71.30% | 71.22% | 61.85% | 60.67% | 61.04% | 60.71% | 61.04% |
| Asyz5.1 | 80.24% | 80.29% | 80.28% | 80.29% | 73.64% | 73.23% | 73.34% | 73.23% | 73.42% |
| Asyz5.2 | 80.24% | 80.29% | 80.28% | 80.29% | 73.64% | 73.23% | 73.34% | 73.23% | 73.42% |
| Asyz5.3 | 80.24% | 80.29% | 80.28% | 80.29% | 73.64% | 73.23% | 73.34% | 73.23% | 73.42% |
| Asyz5.4 | 80.24% | 80.29% | 80.28% | 80.29% | 73.64% | 73.23% | 73.34% | 73.23% | 73.42% |
| Asyz5.5 | 80.20% | 80.15% | 80.23% | 80.15% | 73.51% | 73.09% | 73.29% | 73.09% | 73.28% |
| Asyz6.1 | 70.03% | 70.03% | 70.06% | 70.03% | 83.23% | 85.01% | 84.23% | 85.05% | 84.21% |
| Asyz6.2 | 71.50% | 71.50% | 71.53% | 71.50% | 84.79% | 86.57% | 85.80% | 86.61% | 85.77% |
| Asyz6.3 | 70.07% | 70.07% | 70.10% | 70.07% | 83.23% | 85.05% | 84.23% | 85.10% | 84.21% |
| Asyz6.4 | 71.54% | 71.54% | 71.58% | 71.54% | 84.79% | 86.61% | 85.80% | 86.66% | 85.77% |
| Asyz7.1 | 100.00% | 99.91% | 100.00% | 99.96% | 74.98% | 75.41% | 75.21% | 75.41% | 75.25% |
| Asyz7.2 | 99.91% | 100.00% | 99.91% | 99.96% | 75.02% | 75.46% | 75.21% | 75.46% | 75.29% |
| Asyz7.3 | 100.00% | 99.91% | 100.00% | 99.96% | 75.01% | 75.45% | 75.25% | 75.45% | 75.28% |
| Asyz7.5 | 99.96% | 99.96% | 99.96% | 100.00% | 75.02% | 75.46% | 75.21% | 75.46% | 75.29% |
| Asyz8.1 | 74.98% | 75.02% | 75.01% | 75.02% | 100.00% | 96.74% | 97.90% | 96.79% | 97.95% |
| Asyz8.2 | 75.41% | 75.46% | 75.45% | 75.46% | 96.74% | 100.00% | 97.68% | 99.96% | 97.72% |
| Asyz8.3 | 75.21% | 75.21% | 75.25% | 75.21% | 97.90% | 97.68% | 100.00% | 97.72% | 99.96% |
| Asyz8.4 | 75.41% | 75.46% | 75.45% | 75.46% | 96.79% | 99.96% | 97.72% | 100.00% | 97.77% |
| Asyz8.5 | 75.25% | 75.29% | 75.28% | 75.29% | 97.95% | 97.72% | 99.96% | 97.77% | 100.00% |
| Asyz9.1 | 73.37% | 73.37% | 73.41% | 73.42% | 82.61% | 83.71% | 82.63% | 83.67% | 82.61% |
| Asyz9.2 | 73.09% | 73.09% | 73.13% | 73.14% | 82.33% | 83.53% | 82.31% | 83.48% | 82.33% |
| Asyz9.4 | 73.37% | 73.37% | 73.41% | 73.42% | 82.69% | 83.85% | 82.67% | 83.76% | 82.69% |
| Asyz9.5 | 73.27% | 73.27% | 73.30% | 73.32% | 82.64% | 83.75% | 82.66% | 83.80% | 82.64% |

TABLE 8
Similarity calculation results among 49 parts
of Avena sativa samples and Bai Yan No.

| Material No. | Asyz9.1 | Asyz9.2 | Asyz9.4 | Asyz9.5 |
|---|---|---|---|---|
| Asyz11.1 | 77.43% | 77.33% | 77.39% | 77.47% |
| Asyz11.2 | 77.43% | 77.33% | 77.39% | 77.47% |
| Asyz11.3 | 77.43% | 77.33% | 77.39% | 77.47% |
| Asyz11.4 | 77.43% | 77.33% | 77.39% | 77.47% |
| Asyz11.5 | 77.43% | 77.33% | 77.39% | 77.47% |
| Asyz12.2 | 78.43% | 78.24% | 78.25% | 78.20% |
| Asyz12.3 | 78.51% | 78.32% | 78.33% | 78.27% |
| Asyz12.4 | 78.38% | 78.20% | 78.21% | 78.15% |
| Asyz12.5 | 78.43% | 78.24% | 78.25% | 78.20% |
| Asyz2.2 | 55.11% | 55.23% | 55.34% | 55.32% |
| Asyz2.3 | 55.09% | 55.21% | 55.32% | 55.30% |
| Asyz2.4 | 55.05% | 55.16% | 55.27% | 55.25% |
| Asyz20.1 | 77.60% | 77.28% | 77.82% | 77.46% |
| Asyz20.2 | 77.57% | 77.24% | 77.79% | 77.42% |
| Asyz20.3 | 79.22% | 78.85% | 79.35% | 79.16% |
| Asyz20.4 | 78.64% | 78.27% | 78.77% | 78.54% |
| Asyz20.5 | 79.12% | 78.75% | 79.25% | 79.02% |
| Asyz21.1 | 81.79% | 81.38% | 81.66% | 81.65% |
| Asyz21.2 | 81.79% | 81.38% | 81.66% | 81.65% |
| Asyz21.3 | 81.97% | 81.61% | 81.88% | 81.83% |
| Asyz21.4 | 81.75% | 81.43% | 81.71% | 81.61% |
| Asyz21.5 | 81.84% | 81.43% | 81.71% | 81.70% |
| Asyz3.1 | 64.35% | 64.38% | 64.44% | 64.42% |
| Asyz3.2 | 64.33% | 64.36% | 64.42% | 64.40% |
| Asyz3.3 | 64.33% | 64.36% | 64.42% | 64.40% |
| Asyz3.4 | 64.33% | 64.36% | 64.42% | 64.40% |
| Asyz3.5 | 64.30% | 64.33% | 64.39% | 64.38% |
| Asyz5.1 | 70.38% | 70.37% | 70.16% | 70.55% |
| Asyz5.2 | 70.38% | 70.37% | 70.16% | 70.55% |
| Asyz5.3 | 70.38% | 70.37% | 70.16% | 70.55% |
| Asyz5.4 | 70.38% | 70.37% | 70.16% | 70.55% |
| Asyz5.5 | 70.29% | 70.33% | 70.12% | 70.46% |
| Asyz6.1 | 77.70% | 77.47% | 77.92% | 77.38% |
| Asyz6.2 | 79.26% | 79.03% | 79.48% | 78.94% |
| Asyz6.3 | 77.74% | 77.51% | 77.97% | 77.42% |
| Asyz6.4 | 79.30% | 79.07% | 79.53% | 78.98% |
| Asyz7.1 | 73.37% | 73.09% | 73.37% | 73.27% |
| Asyz7.2 | 73.37% | 73.09% | 73.37% | 73.27% |
| Asyz7.3 | 73.41% | 73.13% | 73.41% | 73.30% |
| Asyz7.5 | 73.42% | 73.14% | 73.42% | 73.32% |
| Asyz8.1 | 82.61% | 82.33% | 82.69% | 82.64% |
| Asyz8.2 | 83.71% | 83.53% | 83.85% | 83.75% |
| Asyz8.3 | 82.63% | 82.31% | 82.67% | 82.66% |
| Asyz8.4 | 83.67% | 83.48% | 83.76% | 83.80% |
| Asyz8.5 | 82.61% | 82.33% | 82.69% | 82.64% |
| Asyz9.1 | 100.00% | 99.51% | 99.38% | 99.42% |
| Asyz9.2 | 99.51% | 100.00% | 99.06% | 99.64% |
| Asyz9.4 | 99.38% | 99.06% | 100.00% | 98.88% |
| Asyz9.5 | 99.42% | 99.64% | 98.88% | 100.00% |

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. An application of an SNP loci combination in variety identifying, seed purity analysis, hybrid breeding and fingerprint construction of Avena sativa, wherein the SNP loci combination comprises SNP loci located in reference genomes sanfensan and OT3098 as shown in a following table:

| Chrom- | Physical | Allelic | Reference | Chrom- | Physical | Allelic | Reference | Chrom- | Physical | Allelic | Reference |
|---|---|---|---|---|---|---|---|---|---|---|---|
| osome | location | variation | genome | osome | location | variation | genome | osome | location | variation | genome |
| 1A | 4473517 | C, A | sanfensan | 3C | 488844690 | A, C | sanfensan | 6A | 108982250 | C, T | sanfensan |
| 1A | 9349344 | G, A | sanfensan | 3C | 494056611 | C, T | sanfensan | 6A | 109794291 | G, A | sanfensan |
| 1A | 14898617 | C, T | sanfensan | 3C | 499140906 | G, A | sanfensan | 6A | 113440903 | T, G | sanfensan |
| 1A | 19788850 | T, C | sanfensan | 3C | 504946938 | C, A | sanfensan | 6A | 118377169 | C, G | sanfensan |
| 1A | 24134605 | A, C | sanfensan | 3C | 505015277 | T, G | sanfensan | 6A | 124172048 | A, G | sanfensan |
| 1A | 25204308 | G, A | sanfensan | 3C | 513611369 | G, A | sanfensan | 6A | 125350773 | A, C | sanfensan |
| 1A | 34911838 | T, A | sanfensan | 3C | 515368248 | C, T | sanfensan | 6A | 130843545 | T, C | sanfensan |
| 1A | 35091182 | A, G | sanfensan | 3C | 520707894 | G, A | sanfensan | 6A | 135683529 | T, C | sanfensan |
| 1A | 39926834 | G, A | sanfensan | 3C | 529897647 | C, T | sanfensan | 6A | 143547400 | A, T | sanfensan |
| 1A | 40243841 | C, A | sanfensan | 3C | 534373018 | A, G | sanfensan | 6A | 147670247 | A, G | sanfensan |
| 1A | 46676991 | T, C | sanfensan | 3C | 536903652 | C, T | sanfensan | 6A | 151304761 | T, C | sanfensan |
| 1A | 50477703 | C, T | sanfensan | 3C | 540735781 | A, C | sanfensan | 6A | 157973297 | G, C | sanfensan |
| 1A | 55467045 | T, C | sanfensan | 3C | 545800871 | T, C | sanfensan | 6A | 158294119 | A, C | sanfensan |
| 1A | 61641698 | C, A | sanfensan | 3C | 551522255 | A, C | sanfensan | 6A | 163354468 | G, C | sanfensan |
| 1A | 65935052 | C, A | sanfensan | 3C | 556937242 | T, C | sanfensan | 6A | 165693464 | T, C | sanfensan |
| 1A | 71753358 | C, T | sanfensan | 3C | 561945091 | C, A | sanfensan | 6A | 173487971 | G, A | sanfensan |
| 1A | 76746486 | T, C | sanfensan | 3C | 565260776 | C, T | sanfensan | 6A | 176286584 | G, A | sanfensan |
| 1A | 82946413 | T, C | sanfensan | 3C | 572949143 | C, G | sanfensan | 6A | 183958426 | C, T | sanfensan |
| 1A | 85796042 | G, A | sanfensan | 3C | 577651667 | G, A | sanfensan | 6A | 186274335 | G, A | sanfensan |
| 1A | 92427340 | G, A | sanfensan | 3C | 580350958 | C, T | sanfensan | 6A | 194312081 | C, T | sanfensan |
| 1A | 98303200 | A, C | sanfensan | 3C | 589499247 | A, G | sanfensan | 6A | 198052953 | G, A | sanfensan |
| 1A | 100886423 | T, C | sanfensan | 3C | 593401324 | A, G | sanfensan | 6A | 203626460 | T, G | sanfensan |
| 1A | 105665342 | A, G | sanfensan | 3C | 596059950 | G, A | sanfensan | 6A | 208551969 | G, A | sanfensan |
| 1A | 106585584 | T, C | sanfensan | 3C | 602749897 | C, G | sanfensan | 6A | 212325078 | A, G | sanfensan |
| 1A | 110857330 | A, C | sanfensan | 3C | 606686603 | A, G | sanfensan | 6A | 216393559 | G, A | sanfensan |
| 1A | 119266034 | T, C | sanfensan | 3C | 612645249 | A, G | sanfensan | 6A | 222443511 | C, G | sanfensan |
| 1A | 120319599 | T, G | sanfensan | 3C | 615205024 | T, A | sanfensan | 6A | 227146163 | G, A | sanfensan |
| 1A | 128479703 | G, A | sanfensan | 3D | 3451014 | A, G | sanfensan | 6A | 232355131 | C, A | sanfensan |
| 1A | 131129360 | A, G | sanfensan | 3D | 6304975 | C, G | sanfensan | 6A | 236306595 | G, A | sanfensan |
| 1A | 135686787 | G, A | sanfensan | 3D | 8431856 | G, A | sanfensan | 6A | 242551165 | C, T | sanfensan |
| 1A | 140893664 | A, G | sanfensan | 3D | 12739908 | G, A | sanfensan | 6A | 248683146 | T, A | sanfensan |
| 1A | 149729422 | A, G | sanfensan | 3D | 17626366 | T, C | sanfensan | 6A | 254218064 | C, T | sanfensan |
| 1A | 154018712 | A, G | sanfensan | 3D | 22243586 | T, C | sanfensan | 6A | 256311166 | G, A | sanfensan |
| 1A | 156986726 | T, C | sanfensan | 3D | 29901822 | T, C | sanfensan | 6A | 263945630 | T, C | sanfensan |
| 1A | 160810612 | G, C | sanfensan | 3D | 34199181 | T, A | sanfensan | 6A | 265291617 | G, A | sanfensan |
| 1A | 168045464 | T, G | sanfensan | 3D | 38215402 | C, T | sanfensan | 6A | 272551424 | T, C | sanfensan |
| 1A | 174968050 | A, G | sanfensan | 3D | 40736696 | C, T | sanfensan | 6A | 276146901 | A, C | sanfensan |
| 1A | 177054360 | C, T | sanfensan | 3D | 45922953 | C, T | sanfensan | 6A | 283068354 | T, C | sanfensan |
| 1A | 184641750 | G, A | sanfensan | 3D | 54465933 | T, C | sanfensan | 6A | 287654071 | G, A | sanfensan |
| 1A | 186347220 | A, G | sanfensan | 3D | 59996227 | A, G | sanfensan | 6A | 291439784 | A, C | sanfensan |
| 1A | 190161955 | T, C | sanfensan | 3D | 63704529 | G, A | sanfensan | 6A | 296813413 | T, A | sanfensan |
| 1A | 197891296 | A, G | sanfensan | 3D | 66789700 | C, G | sanfensan | 6A | 304331839 | T, C | sanfensan |
| 1A | 202332235 | G, C | sanfensan | 3D | 70216974 | C, T | sanfensan | 6A | 309571759 | G, C | sanfensan |
| 1A | 206929081 | A, G | sanfensan | 3D | 78492763 | A, G | sanfensan | 6A | 311843254 | A, C | sanfensan |
| 1A | 213307997 | A, C | sanfensan | 3D | 82560649 | G, A | sanfensan | 6A | 318306732 | G, A | sanfensan |
| 1A | 215902606 | A, C | sanfensan | 3D | 89278480 | C, T | sanfensan | 6A | 324491687 | A, C | sanfensan |
| 1A | 223276058 | G, T | sanfensan | 3D | 94976535 | T, A | sanfensan | 6A | 326644360 | G, A | sanfensan |
| 1A | 225939090 | G, A | sanfensan | 3D | 99870793 | C, A | sanfensan | 6A | 330034066 | C, T | sanfensan |
| 1A | 229684564 | T, C | sanfensan | 3D | 102284469 | G, A | sanfensan | 6A | 335513362 | C, T | sanfensan |
| 1A | 230432457 | A, G | sanfensan | 3D | 105085292 | G, T | sanfensan | 6A | 339809705 | C, G | sanfensan |
| 1A | 239890900 | C, T | sanfensan | 3D | 112272049 | A, G | sanfensan | 6A | 343191906 | T, C | sanfensan |
| 1A | 243567990 | G, A | sanfensan | 3D | 117725865 | G, A | sanfensan | 6A | 349219222 | G, A | sanfensan |
| 1A | 245948969 | G, A | sanfensan | 3D | 124920499 | T, C | sanfensan | 6A | 351759840 | A, G | sanfensan |
| 1A | 250969537 | G, C | sanfensan | 3D | 125085251 | G, A | sanfensan | 6A | 358242236 | G, T | sanfensan |
| 1A | 257803247 | T, G | sanfensan | 3D | 130241097 | G, T | sanfensan | 6A | 362754991 | T, C | sanfensan |
| 1A | 263812100 | A, G | sanfensan | 3D | 136299812 | G, A | sanfensan | 6A | 367473345 | G, A | sanfensan |
| 1A | 268553550 | T, C | sanfensan | 3D | 140453087 | G, T | sanfensan | 6A | 371831728 | G, A | sanfensan |
| 1A | 271645348 | T, G | sanfensan | 3D | 149490075 | G, A | sanfensan | 6A | 377260484 | T, C | sanfensan |
| 1A | 283816950 | G, A | sanfensan | 3D | 154494846 | C, T | sanfensan | 6A | 382654181 | C, T | sanfensan |
| 1A | 286799892 | T, C | sanfensan | 3D | 155736382 | A, G | sanfensan | 6A | 389448736 | A, G | sanfensan |
| 1A | 292498165 | C, A | sanfensan | 3D | 160258419 | T, G | sanfensan | 6A | 394354567 | T, C | sanfensan |
| 1A | 298383411 | A, G | sanfensan | 3D | 165382934 | G, C | sanfensan | 6A | 395689953 | A, G | sanfensan |
| 1A | 300811687 | A, G | sanfensan | 3D | 174233842 | A, G | sanfensan | 6A | 402143190 | A, G | sanfensan |
| 1A | 307060043 | T, G | sanfensan | 3D | 178210807 | A, G | sanfensan | 6A | 409084859 | A, C | sanfensan |
| 1A | 314835969 | G, A | sanfensan | 3D | 183045672 | C, T | sanfensan | 6A | 414438357 | A, G | sanfensan |
| 1A | 315910953 | A, G | sanfensan | 3D | 185605470 | A, G | sanfensan | 6A | 415355947 | T, C | sanfensan |
| 1A | 321206313 | T, G | sanfensan | 3D | 191181967 | T, C | sanfensan | 6A | 421445718 | A, C | sanfensan |
| 1A | 329104188 | A, C | sanfensan | 3D | 199127188 | C, A | sanfensan | 6A | 422458226 | G, A | sanfensan |
| 1A | 333516925 | G, T | sanfensan | 3D | 201408393 | T, C | sanfensan | 6A | 425035666 | C, A | sanfensan |
| 1A | 335522334 | G, A | sanfensan | 3D | 208325818 | C, A | sanfensan | 6A | 433725430 | A, G | sanfensan |
| 1A | 342234031 | G, A | sanfensan | 3D | 213296950 | G, T | sanfensan | 6A | 437585163 | T, C | sanfensan |
| 1A | 348750824 | C, A | sanfensan | 3D | 217423350 | T, C | sanfensan | 6A | 444959115 | G, C | sanfensan |
| 1A | 354606484 | C, T | sanfensan | 3D | 223809923 | G, C | sanfensan | 6A | 445547963 | C, T | sanfensan |
| 1A | 355044825 | G, A | sanfensan | 3D | 226483167 | G, A | sanfensan | 6A | 454994988 | A, G | sanfensan |
| 1A | 361899430 | G, A | sanfensan | 3D | 231132075 | C, T | sanfensan | 6C | 4740123 | A, C | sanfensan |
| 1A | 365228574 | T, G | sanfensan | 3D | 237287427 | T, A | sanfensan | 6C | 7818111 | T, G | sanfensan |
| 1A | 370165053 | A, G | sanfensan | 3D | 241417679 | G, A | sanfensan | 6C | 10608939 | A, T | sanfensan |
| 1A | 371882871 | T, C | sanfensan | 3D | 248327064 | G, A | sanfensan | 6C | 17502954 | A, C | sanfensan |
| 1A | 379396033 | A, C | sanfensan | 3D | 250955048 | C, T | sanfensan | 6C | 21919209 | C, T | sanfensan |
| 1A | 380814167 | C, T | sanfensan | 3D | 257222395 | C, T | sanfensan | 6C | 27489398 | T, C | sanfensan |
| 1A | 385881054 | T, G | sanfensan | 3D | 263680149 | G, A | sanfensan | 6C | 32883642 | T, G | sanfensan |
| 1A | 394799443 | C, G | sanfensan | 3D | 268114489 | C, A | sanfensan | 6C | 35200287 | C, A | sanfensan |
| 1A | 396233056 | A, T | sanfensan | 3D | 273619731 | A, C | sanfensan | 6C | 38472337 | T, C | sanfensan |
| 1A | 400131929 | A, G | sanfensan | 3D | 276710058 | C, T | sanfensan | 6C | 43196331 | A, G | sanfensan |
| 1A | 408599208 | C, T | sanfensan | 3D | 284403130 | G, C | sanfensan | 6C | 47589408 | A, G | sanfensan |
| 1A | 410583439 | T, C | sanfensan | 3D | 288065151 | G, A | sanfensan | 6C | 51088171 | A, G | sanfensan |
| 1A | 417767787 | G, A | sanfensan | 3D | 294630726 | C, T | sanfensan | 6C | 59096246 | G, A | sanfensan |
| 1A | 423934932 | G, T | sanfensan | 3D | 297227408 | C, T | sanfensan | 6C | 60407986 | C, T | sanfensan |
| 1A | 427534714 | A, G | sanfensan | 3D | 300199691 | T, C | sanfensan | 6C | 65757372 | C, T | sanfensan |
| 1A | 430802770 | T, C | sanfensan | 3D | 306425893 | C, A | sanfensan | 6C | 66609935 | G, A | sanfensan |
| 1A | 439615284 | A, G | sanfensan | 3D | 314722897 | A, G | sanfensan | 6C | 72292296 | A, G | sanfensan |
| 1A | 443388417 | C, T | sanfensan | 3D | 315890257 | A, T | sanfensan | 6C | 75973462 | A, T | sanfensan |
| 1A | 447639906 | G, A | sanfensan | 3D | 323402379 | G, A | sanfensan | 6C | 81974276 | C, T | sanfensan |
| 1A | 453204191 | T, C | sanfensan | 3D | 325861485 | A, G | sanfensan | 6C | 88129794 | G, C | sanfensan |
| 1A | 457075413 | G, A | sanfensan | 3D | 330563924 | A, G | sanfensan | 6C | 94193475 | T, C | sanfensan |
| 1A | 463117262 | G, A | sanfensan | 3D | 335625837 | G, A | sanfensan | 6C | 95536874 | T, C | sanfensan |
| 1A | 465118330 | G, T | sanfensan | 3D | 340772061 | G, A | sanfensan | 6C | 101717822 | C, T | sanfensan |
| 1A | 472500626 | T, C | sanfensan | 3D | 348100481 | T, G | sanfensan | 6C | 107891612 | C, T | sanfensan |
| 1A | 475569735 | T, G | sanfensan | 3D | 352819049 | C, T | sanfensan | 6C | 112851715 | A, T | sanfensan |
| 1A | 481563062 | G, A | sanfensan | 3D | 358682575 | T, C | sanfensan | 6C | 116496712 | C, T | sanfensan |
| 1A | 486246321 | T, C | sanfensan | 3D | 361158177 | G, A | sanfensan | 6C | 121111029 | T, C | sanfensan |
| 1A | 490249212 | G, A | sanfensan | 3D | 369050731 | T, C | sanfensan | 6C | 125320836 | G, A | sanfensan |
| 1A | 495026085 | C, T | sanfensan | 3D | 373598604 | C, T | sanfensan | 6C | 132900758 | T, A | sanfensan |
| 1A | 500157268 | G, A | sanfensan | 3D | 378481165 | T, C | sanfensan | 6C | 135785484 | G, C | sanfensan |
| 1A | 505420346 | T, C | sanfensan | 3D | 381350490 | G, A | sanfensan | 6C | 143030880 | C, A | sanfensan |
| 1A | 513237629 | C, A | sanfensan | 3D | 388861217 | T, C | sanfensan | 6C | 148851945 | G, A | sanfensan |
| 1A | 516943173 | A, G | sanfensan | 3D | 389878476 | T, G | sanfensan | 6C | 154103608 | A, C | sanfensan |
| 1A | 520725745 | C, G | sanfensan | 3D | 391163814 | T, C | sanfensan | 6C | 156034908 | T, G | sanfensan |
| 1A | 529117339 | C, T | sanfensan | 3D | 397016298 | C, T | sanfensan | 6C | 162091462 | A, C | sanfensan |
| 1A | 532148235 | T, C | sanfensan | 3D | 403496100 | A, G | sanfensan | 6C | 165806162 | A, T | sanfensan |
| 1A | 538845292 | C, T | sanfensan | 3D | 406924921 | A, G | sanfensan | 6C | 172155825 | G, A | sanfensan |
| 1A | 540384422 | G, C | sanfensan | 3D | 410226093 | G, A | sanfensan | 6C | 175590346 | C, T | sanfensan |
| 1A | 546023901 | C, G | sanfensan | 3D | 417099879 | A, G | sanfensan | 6C | 180731171 | G, A | sanfensan |
| 1A | 551040971 | C, T | sanfensan | 3D | 419164521 | C, G | sanfensan | 6C | 185475790 | A, G | sanfensan |
| 1C | 4866196 | A, C | sanfensan | 3D | 423904272 | T, C | sanfensan | 6C | 194315523 | G, A | sanfensan |
| 1C | 8009186 | G, A | sanfensan | 3D | 426092994 | A, G | sanfensan | 6C | 196088194 | G, A | sanfensan |
| 1C | 12114531 | T, C | sanfensan | 3D | 433821318 | G, A | sanfensan | 6C | 196851358 | C, T | sanfensan |
| 1C | 17916692 | G, A | sanfensan | 3D | 436973559 | T, G | sanfensan | 6C | 200122712 | A, C | sanfensan |
| 1C | 22093157 | T, C | sanfensan | 3D | 439655087 | A, G | sanfensan | 6C | 207092401 | A, G | sanfensan |
| 1C | 25463149 | C, T | sanfensan | 3D | 441290663 | T, C | sanfensan | 6C | 213333184 | T, G | sanfensan |
| 1C | 32954922 | G, T | sanfensan | 3D | 448791478 | T, C | sanfensan | 6C | 217526046 | G, A | sanfensan |
| 1C | 38255313 | G, A | sanfensan | 3D | 451012282 | C, G | sanfensan | 6C | 222420550 | C, T | sanfensan |
| 1C | 40225359 | C, T | sanfensan | 3D | 456635851 | G, A | sanfensan | 6C | 227353579 | G, A | sanfensan |
| 1C | 47044975 | C, T | sanfensan | 3D | 460647997 | A, T | sanfensan | 6C | 234060684 | A, G | sanfensan |
| 1C | 54713053 | C, T | sanfensan | 4A | 167665 | A, G | sanfensan | 6C | 235857775 | G, A | sanfensan |
| 1C | 57342838 | G, T | sanfensan | 4A | 5170696 | A, G | sanfensan | 6C | 244931366 | T, C | sanfensan |
| 1C | 64447286 | C, T | sanfensan | 4A | 12321972 | G, A | sanfensan | 6C | 246070168 | G, A | sanfensan |
| 1C | 68342251 | C, G | sanfensan | 4A | 19482740 | T, C | sanfensan | 6C | 253789587 | C, T | sanfensan |
| 1C | 73736931 | T, C | sanfensan | 4A | 22870941 | T, C | sanfensan | 6C | 257684248 | G, C | sanfensan |
| 1C | 75426247 | G, A | sanfensan | 4A | 27380296 | T, C | sanfensan | 6C | 260070968 | C, T | sanfensan |
| 1C | 81261715 | C, T | sanfensan | 4A | 30862932 | G, T | sanfensan | 6C | 266510278 | C, T | sanfensan |
| 1C | 89052462 | A, C | sanfensan | 4A | 36420099 | T, G | sanfensan | 6C | 272777330 | C, T | sanfensan |
| 1C | 93949401 | C, T | sanfensan | 4A | 38087452 | T, G | sanfensan | 6C | 275890891 | G, A | sanfensan |
| 1C | 97665480 | T, A | sanfensan | 4A | 44284137 | A, C | sanfensan | 6C | 284003530 | C, T | sanfensan |
| 1C | 104414265 | C, T | sanfensan | 4A | 45337009 | T, C | sanfensan | 6C | 289057124 | C, T | sanfensan |
| 1C | 107889124 | C, T | sanfensan | 4A | 50385302 | T, C | sanfensan | 6C | 292273925 | G, A | sanfensan |
| 1C | 112840945 | A, G | sanfensan | 4A | 58613149 | T, C | sanfensan | 6C | 295852267 | A, C | sanfensan |
| 1C | 117607567 | C, A | sanfensan | 4A | 62632618 | T, C | sanfensan | 6C | 301581063 | G, A | sanfensan |
| 1C | 123766670 | G, C | sanfensan | 4A | 69360237 | A, C | sanfensan | 6C | 308002832 | T, C | sanfensan |
| 1C | 124698034 | C, T | sanfensan | 4A | 72548515 | T, C | sanfensan | 6C | 310533434 | G, A | sanfensan |
| 1C | 125624389 | G, A | sanfensan | 4A | 78860501 | A, G | sanfensan | 6C | 316651599 | G, C | sanfensan |
| 1C | 132272583 | G, T | sanfensan | 4A | 83477002 | A, G | sanfensan | 6C | 320349680 | G, A | sanfensan |
| 1C | 137221231 | G, A | sanfensan | 4A | 86718114 | A, C | sanfensan | 6C | 325082414 | T, C | sanfensan |
| 1C | 140336177 | G, A | sanfensan | 4A | 90186259 | T, C | sanfensan | 6C | 331834200 | C, T | sanfensan |
| 1C | 148863724 | C, T | sanfensan | 4A | 99717884 | C, G | sanfensan | 6C | 335435627 | T, C | sanfensan |
| 1C | 152576285 | T, G | sanfensan | 4A | 103226101 | C, T | sanfensan | 6C | 341601920 | C, A | sanfensan |
| 1C | 158557656 | G, C | sanfensan | 4A | 108841089 | C, A | sanfensan | 6C | 348384420 | T, C | sanfensan |
| 1C | 161866271 | C, A | sanfensan | 4A | 109097229 | G, A | sanfensan | 6C | 354770875 | T, C | sanfensan |
| 1C | 166850618 | G, A | sanfensan | 4A | 110362403 | T, C | sanfensan | 6C | 355787593 | T, C | sanfensan |
| 1C | 171246538 | C, A | sanfensan | 4A | 119524477 | G, C | sanfensan | 6C | 363981785 | C, T | sanfensan |
| 1C | 177121539 | G, A | sanfensan | 4A | 124335960 | C, T | sanfensan | 6C | 367279098 | C, G | sanfensan |
| 1C | 184861598 | C, T | sanfensan | 4A | 129987141 | C, T | sanfensan | 6C | 372097294 | G, T | sanfensan |
| 1C | 187896025 | C, A | sanfensan | 4A | 132188846 | A, G | sanfensan | 6C | 376086340 | G, A | sanfensan |
| 1C | 192704844 | C, T | sanfensan | 4A | 138010184 | A, G | sanfensan | 6C | 382987219 | C, T | sanfensan |
| 1C | 196342850 | G, A | sanfensan | 4A | 141248665 | T, C | sanfensan | 6C | 385017932 | A, G | sanfensan |
| 1C | 203152613 | A, G | sanfensan | 4A | 141974369 | A, G | sanfensan | 6C | 393722890 | C, T | sanfensan |
| 1C | 206698681 | G, A | sanfensan | 4A | 146462646 | G, A | sanfensan | 6C | 396489334 | T, C | sanfensan |
| 1C | 212808498 | G, A | sanfensan | 4A | 151979768 | T, C | sanfensan | 6C | 402999061 | G, A | sanfensan |
| 1C | 219911347 | A, G | sanfensan | 4A | 158451505 | G, A | sanfensan | 6C | 404034553 | G, A | sanfensan |
| 1C | 223588927 | G, A | sanfensan | 4A | 163350428 | A, G | sanfensan | 6C | 407439238 | G, C | sanfensan |
| 1C | 228818861 | G, T | sanfensan | 4A | 167565220 | A, G | sanfensan | 6C | 412317336 | C, T | sanfensan |
| 1C | 231062523 | C, T | sanfensan | 4A | 173953101 | T, G | sanfensan | 6C | 416156258 | T, C | sanfensan |
| 1C | 244308309 | G, A | sanfensan | 4A | 178883204 | G, C | sanfensan | 6C | 422586122 | G, C | sanfensan |
| 1C | 244949293 | G, C | sanfensan | 4A | 184596923 | C, T | sanfensan | 6C | 429700897 | C, T | sanfensan |
| 1C | 247382592 | G, A | sanfensan | 4A | 189201669 | A, C | sanfensan | 6C | 430532023 | A, G | sanfensan |
| 1C | 251563472 | C, G | sanfensan | 4A | 193547898 | G, A | sanfensan | 6C | 436366975 | G, A | sanfensan |
| 1C | 258583411 | G, C | sanfensan | 4A | 199508298 | A, G | sanfensan | 6C | 440291570 | T, G | sanfensan |
| 1C | 264870633 | C, T | sanfensan | 4A | 201195563 | C, T | sanfensan | 6C | 447368142 | A, G | sanfensan |
| 1C | 269866051 | G, A | sanfensan | 4A | 207185396 | C, A | sanfensan | 6C | 454158229 | C, T | sanfensan |
| 1C | 270041152 | T, C | sanfensan | 4A | 213373498 | T, C | sanfensan | 6C | 457431878 | G, A | sanfensan |
| 1C | 279714245 | T, C | sanfensan | 4A | 216222538 | A, G | sanfensan | 6C | 464408570 | A, G | sanfensan |
| 1C | 281426354 | G, A | sanfensan | 4A | 221795376 | C, T | sanfensan | 6C | 466196657 | G, A | sanfensan |
| 1C | 289269367 | C, G | sanfensan | 4A | 228345642 | A, T | sanfensan | 6C | 470467140 | G, T | sanfensan |
| 1C | 290192142 | C, A | sanfensan | 4A | 231330723 | A, C | sanfensan | 6C | 475226773 | G, A | sanfensan |
| 1C | 295651800 | G, T | sanfensan | 4A | 235514032 | G, A | sanfensan | 6C | 484622983 | G, A | sanfensan |
| 1C | 301166258 | G, C | sanfensan | 4A | 242193423 | C, T | sanfensan | 6C | 489733584 | G, T | sanfensan |
| 1C | 305159269 | G, T | sanfensan | 4A | 247945600 | C, T | sanfensan | 6C | 491124804 | T, C | sanfensan |
| 1C | 311692892 | C, T | sanfensan | 4A | 252369025 | T, C | sanfensan | 6C | 496637243 | G, A | sanfensan |
| 1C | 318201468 | T, G | sanfensan | 4A | 259286784 | G, C | sanfensan | 6C | 502828531 | C, T | sanfensan |
| 1C | 322797850 | G, A | sanfensan | 4A | 261531070 | A, G | sanfensan | 6C | 505002174 | G, A | sanfensan |
| 1C | 329636395 | C, T | sanfensan | 4A | 268127413 | G, T | sanfensan | 6C | 507736024 | C, T | sanfensan |
| 1C | 331069160 | G, A | sanfensan | 4A | 273296067 | A, G | sanfensan | 6C | 512237599 | C, T | sanfensan |
| 1C | 338302628 | G, A | sanfensan | 4A | 276596131 | T, C | sanfensan | 6C | 516058707 | C, T | sanfensan |
| 1C | 343573672 | A, G | sanfensan | 4A | 280307793 | C, T | sanfensan | 6C | 522649701 | C, G | sanfensan |
| 1C | 346562089 | G, A | sanfensan | 4A | 289509128 | C, T | sanfensan | 6C | 529463088 | G, T | sanfensan |
| 1C | 350268981 | C, T | sanfensan | 4A | 293451571 | G, A | sanfensan | 6C | 532838127 | C, T | sanfensan |
| 1C | 359261478 | T, C | sanfensan | 4A | 299169983 | T, G | sanfensan | 6C | 538744714 | C, T | sanfensan |
| 1C | 361793363 | G, A | sanfensan | 4A | 300515091 | T, C | sanfensan | 6C | 541894652 | T, C | sanfensan |
| 1C | 366795285 | C, G | sanfensan | 4A | 305599904 | G, A | sanfensan | 6C | 548890274 | A, G | sanfensan |
| 1C | 373361346 | G, A | sanfensan | 4A | 311085314 | A, C | sanfensan | 6C | 550597198 | C, T | sanfensan |
| 1C | 377285872 | C, T | sanfensan | 4A | 316042196 | C, T | sanfensan | 6C | 555553384 | T, C | sanfensan |
| 1C | 384514418 | T, G | sanfensan | 4A | 322946253 | T, C | sanfensan | 6C | 563333574 | G, T | sanfensan |
| 1C | 388701553 | G, A | sanfensan | 4A | 325478648 | G, C | sanfensan | 6C | 566938058 | T, C | sanfensan |
| 1C | 391109953 | C, T | sanfensan | 4A | 330347981 | A, G | sanfensan | 6C | 574826495 | A, C | sanfensan |
| 1C | 398781194 | A, C | sanfensan | 4A | 337443775 | C, T | sanfensan | 6C | 576805876 | G, A | sanfensan |
| 1C | 404904380 | A, C | sanfensan | 4A | 340462080 | A, G | sanfensan | 6C | 581584319 | G, C | sanfensan |
| 1C | 409167422 | A, G | sanfensan | 4A | 347382275 | A, G | sanfensan | 6C | 585343852 | C, T | sanfensan |
| 1C | 410239696 | G, A | sanfensan | 4A | 354497490 | T, G | sanfensan | 6C | 590741273 | T, C | sanfensan |
| 1C | 418466563 | A, G | sanfensan | 4A | 359050714 | C, A | sanfensan | 6C | 599908901 | G, A | sanfensan |
| 1C | 424408198 | C, T | sanfensan | 4A | 362340167 | C, T | sanfensan | 6C | 601571029 | C, T | sanfensan |
| 1C | 426142267 | T, C | sanfensan | 4A | 367751846 | G, A | sanfensan | 6C | 605547418 | C, T | sanfensan |
| 1C | 434346817 | G, A | sanfensan | 4A | 370622530 | G, T | sanfensan | 6D | 2777295 | C, T | sanfensan |
| 1C | 435126767 | A, G | sanfensan | 4A | 379177676 | T, C | sanfensan | 6D | 6267655 | C, A | sanfensan |
| 1C | 444951853 | C, T | sanfensan | 4A | 381166365 | T, C | sanfensan | 6D | 13107420 | C, T | sanfensan |
| 1C | 446632589 | T, C | sanfensan | 4A | 385406079 | C, G | sanfensan | 6D | 16132609 | C, A | sanfensan |
| 1C | 451346048 | G, A | sanfensan | 4A | 398015147 | A, C | sanfensan | 6D | 23982220 | A, C | sanfensan |
| 1C | 458628576 | C, T | sanfensan | 4A | 401416507 | G, A | sanfensan | 6D | 26754468 | T, G | sanfensan |
| 1D | 4748051 | C, T | sanfensan | 4A | 406634471 | A, G | sanfensan | 6D | 32172782 | C, T | sanfensan |
| 1D | 9972757 | C, T | sanfensan | 4A | 413012139 | A, G | sanfensan | 6D | 35628869 | T, C | sanfensan |
| 1D | 14428103 | T, C | sanfensan | 4A | 418384264 | C, T | sanfensan | 6D | 44217561 | A, G | sanfensan |
| 1D | 17910637 | T, G | sanfensan | 4A | 424141863 | C, T | sanfensan | 6D | 45240610 | T, G | sanfensan |
| 1D | 22886105 | A, C | sanfensan | 4A | 426462945 | A, T | sanfensan | 6D | 50128337 | G, C | sanfensan |
| 1D | 26189931 | T, C | sanfensan | 4A | 434286106 | G, T | sanfensan | 6D | 55030113 | A, G | sanfensan |
| 1D | 34096819 | T, G | sanfensan | 4A | 435134503 | C, T | sanfensan | 6D | 64553482 | T, C | sanfensan |
| 1D | 36812285 | T, C | sanfensan | 4A | 440807368 | C, G | sanfensan | 6D | 66247035 | T, C | sanfensan |
| 1D | 44167411 | T, C | sanfensan | 4A | 445324027 | C, T | sanfensan | 6D | 73309196 | A, G | sanfensan |
| 1D | 48753097 | C, T | sanfensan | 4A | 450745492 | T, C | sanfensan | 6D | 77363272 | A, G | sanfensan |
| 1D | 52943769 | G, A | sanfensan | 4A | 458822974 | A, G | sanfensan | 6D | 84655953 | T, C | sanfensan |
| 1D | 59943693 | C, T | sanfensan | 4A | 462418830 | A, C | sanfensan | 6D | 89015326 | T, C | sanfensan |
| 1D | 61299591 | T, A | sanfensan | 4A | 466738641 | T, C | sanfensan | 6D | 91516090 | A, G | sanfensan |
| 1D | 67828154 | C, T | sanfensan | 4C | 3595818 | G, A | sanfensan | 6D | 98584233 | A, G | sanfensan |
| 1D | 74812678 | T, C | sanfensan | 4C | 6028133 | A, G | sanfensan | 6D | 104170504 | T, G | sanfensan |
| 1D | 76558850 | A, G | sanfensan | 4C | 13049345 | T, A | sanfensan | 6D | 108654428 | T, C | sanfensan |
| 1D | 83489940 | C, T | sanfensan | 4C | 19972099 | A, G | sanfensan | 6D | 114351697 | C, G | sanfensan |
| 1D | 85424309 | G, A | sanfensan | 4C | 20771307 | T, C | sanfensan | 6D | 118475182 | G, A | sanfensan |
| 1D | 90502825 | G, T | sanfensan | 4C | 25886686 | G, C | sanfensan | 6D | 120657246 | A, G | sanfensan |
| 1D | 99173014 | C, A | sanfensan | 4C | 32211660 | C, A | sanfensan | 6D | 128950046 | A, G | sanfensan |
| 1D | 101866462 | A, G | sanfensan | 4C | 36873418 | G, C | sanfensan | 6D | 132670154 | T, C | sanfensan |
| 1D | 105176621 | G, T | sanfensan | 4C | 40449202 | C, G | sanfensan | 6D | 139598945 | A, G | sanfensan |
| 1D | 112466846 | A, G | sanfensan | 4C | 47158151 | C, A | sanfensan | 6D | 142833751 | A, C | sanfensan |
| 1D | 116735117 | T, C | sanfensan | 4C | 51926597 | T, C | sanfensan | 6D | 146878955 | C, T | sanfensan |
| 1D | 120724766 | T, G | sanfensan | 4C | 57722231 | G, A | sanfensan | 6D | 150804995 | G, A | sanfensan |
| 1D | 125083241 | A, G | sanfensan | 4C | 61365967 | G, A | sanfensan | 6D | 159442646 | T, C | sanfensan |
| 1D | 134949445 | T, C | sanfensan | 4C | 66290570 | C, T | sanfensan | 6D | 161979848 | G, A | sanfensan |
| 1D | 136722735 | T, C | sanfensan | 4C | 69406750 | G, A | sanfensan | 6D | 166007399 | C, T | sanfensan |
| 1D | 140036387 | C, T | sanfensan | 4C | 71656189 | G, A | sanfensan | 6D | 171110144 | T, C | sanfensan |
| 1D | 149197821 | G, A | sanfensan | 4C | 75202185 | A, G | sanfensan | 6D | 178157758 | C, T | sanfensan |
| 1D | 154586415 | GT | sanfensan | 4C | 80202847 | G, T | sanfensan | 6D | 181144200 | A, C | sanfensan |
| 1D | 156993466 | T, G | sanfensan | 4C | 86069619 | A, G | sanfensan | 6D | 181398145 | T, G | sanfensan |
| 1D | 160591807 | T, G | sanfensan | 4C | 92181840 | C, T | sanfensan | 6D | 188963826 | T, C | sanfensan |
| 1D | 169373396 | G, A | sanfensan | 4C | 95153438 | G, A | sanfensan | 6D | 190320583 | G, A | sanfensan |
| 1D | 172161852 | G, T | sanfensan | 4C | 101779057 | T, C | sanfensan | 6D | 198738426 | G, A | sanfensan |
| 1D | 178558164 | T, C | sanfensan | 4C | 106362411 | C, T | sanfensan | 6D | 204695478 | C, T | sanfensan |
| 1D | 182138470 | A, G | sanfensan | 4C | 113373499 | T, G | sanfensan | 6D | 209111889 | A, C | sanfensan |
| 1D | 188332946 | T, C | sanfensan | 4C | 116319458 | C, T | sanfensan | 6D | 213388210 | T, C | sanfensan |
| 1D | 191338972 | T, C | sanfensan | 4C | 122530727 | A, G | sanfensan | 6D | 217365606 | G, A | sanfensan |
| 1D | 197805002 | G, A | sanfensan | 4C | 128925968 | T, G | sanfensan | 6D | 220497638 | C, T | sanfensan |
| 1D | 201653641 | A, C | sanfensan | 4C | 130658845 | T, C | sanfensan | 6D | 226248655 | G, T | sanfensan |
| 1D | 205231760 | T, C | sanfensan | 4C | 139094233 | A, G | sanfensan | 6D | 230357776 | T, C | sanfensan |
| 1D | 213849974 | A, G | sanfensan | 4C | 141439434 | G, T | sanfensan | 6D | 239771210 | A, G | sanfensan |
| 1D | 218342762 | G, C | sanfensan | 4C | 149617745 | A, C | sanfensan | 6D | 240712867 | T, A | sanfensan |
| 1D | 221295907 | A, C | sanfensan | 4C | 151871212 | T, C | sanfensan | 6D | 245616215 | G, A | sanfensan |
| 1D | 225627642 | A, G | sanfensan | 4C | 159944864 | C, T | sanfensan | 6D | 254910351 | T, C | sanfensan |
| 1D | 233309591 | C, T | sanfensan | 4C | 164691088 | T, C | sanfensan | 6D | 257401569 | T, C | sanfensan |
| 1D | 234707599 | G, A | sanfensan | 4C | 169970304 | C, A | sanfensan | 6D | 263701170 | T, G | sanfensan |
| 1D | 239408154 | C, T | sanfensan | 4C | 170331200 | A, C | sanfensan | 6D | 268768437 | A, G | sanfensan |
| 1D | 241284677 | T, G | sanfensan | 4C | 178692748 | C, T | sanfensan | 6D | 270928208 | C, A | sanfensan |
| 1D | 248921833 | T, G | sanfensan | 4C | 184841316 | C, T | sanfensan | 6D | 278371196 | C, T | sanfensan |
| 1D | 250623142 | A, T | sanfensan | 4C | 185887772 | T, C | sanfensan | 6D | 284570630 | A, G | sanfensan |
| 1D | 258844136 | C, T | sanfensan | 4C | 193734961 | C, T | sanfensan | 6D | 285173852 | A, G | sanfensan |
| 1D | 262320123 | A, C | sanfensan | 4C | 197024216 | G, A | sanfensan | 6D | 290592914 | T, A | sanfensan |
| 1D | 265785336 | T, C | sanfensan | 4C | 202625381 | G, A | sanfensan | 7A | 556988 | G, A | sanfensan |
| 1D | 270470362 | C, T | sanfensan | 4C | 209016901 | A, G | sanfensan | 7A | 9387452 | T, C | sanfensan |
| 1D | 275515735 | A, G | sanfensan | 4C | 213200350 | C, T | sanfensan | 7A | 10624667 | T, C | sanfensan |
| 1D | 284647168 | G, A | sanfensan | 4C | 215000144 | C, T | sanfensan | 7A | 16247028 | G, A | sanfensan |
| 1D | 288750391 | A, G | sanfensan | 4C | 224645115 | C, T | sanfensan | 7A | 24258432 | T, A | sanfensan |
| 1D | 293777596 | A, G | sanfensan | 4C | 229625246 | A, G | sanfensan | 7A | 29212189 | T, C | sanfensan |
| 1D | 298973779 | C, A | sanfensan | 4C | 234547399 | T, C | sanfensan | 7A | 32277248 | T, G | sanfensan |
| 1D | 304978046 | T, C | sanfensan | 4C | 236119582 | A, C | sanfensan | 7A | 38743380 | T, C | sanfensan |
| 1D | 306430734 | T, G | sanfensan | 4C | 241687009 | G, A | sanfensan | 7A | 42755867 | A, G | sanfensan |
| 1D | 313322569 | T, C | sanfensan | 4C | 245369143 | G, T | sanfensan | 7A | 46387061 | C, T | sanfensan |
| 1D | 317482376 | T, C | sanfensan | 4C | 252253086 | T, A | sanfensan | 7A | 51855350 | A, G | sanfensan |
| 1D | 321285642 | T, C | sanfensan | 4C | 255164714 | C, T | sanfensan | 7A | 57885922 | C, A | sanfensan |
| 1D | 328096417 | A, G | sanfensan | 4C | 260860063 | G, A | sanfensan | 7A | 63281807 | T, C | sanfensan |
| 1D | 334194238 | G, A | sanfensan | 4C | 267013553 | G, A | sanfensan | 7A | 65480794 | G, A | sanfensan |
| 1D | 336604011 | G, A | sanfensan | 4C | 273797546 | G, A | sanfensan | 7A | 71137163 | T, C | sanfensan |
| 1D | 342605207 | A, C | sanfensan | 4C | 277862984 | C, T | sanfensan | 7A | 75733143 | T, A | sanfensan |
| 1D | 348951141 | A, C | sanfensan | 4C | 282087726 | G, A | sanfensan | 7A | 82158130 | G, C | sanfensan |
| 1D | 352790704 | A, G | sanfensan | 4C | 287337582 | G, A | sanfensan | 7A | 85385227 | C, T | sanfensan |
| 1D | 355455291 | G, C | sanfensan | 4C | 292238812 | C, T | sanfensan | 7A | 91146020 | C, T | sanfensan |
| 1D | 364550878 | A, G | sanfensan | 4C | 296562693 | C, T | sanfensan | 7A | 99518702 | T, C | sanfensan |
| 1D | 369719851 | A, G | sanfensan | 4C | 301499481 | T, C | sanfensan | 7A | 100100809 | C, A | sanfensan |
| 1D | 372379876 | A, C | sanfensan | 4C | 308695483 | G, A | sanfensan | 7A | 108559177 | C, T | sanfensan |
| 1D | 379801584 | A, C | sanfensan | 4C | 311224923 | G, A | sanfensan | 7A | 110159568 | G, T | sanfensan |
| 1D | 380728291 | A, G | sanfensan | 4C | 316095744 | G, A | sanfensan | 7A | 119859092 | T, C | sanfensan |
| 1D | 388559569 | T, C | sanfensan | 4C | 324951331 | C, T | sanfensan | 7A | 120329198 | C, A | sanfensan |
| 1D | 394958911 | T, C | sanfensan | 4C | 325766510 | T, G | sanfensan | 7A | 125074054 | T, C | sanfensan |
| 1D | 395282752 | A, G | sanfensan | 4C | 331060999 | G, A | sanfensan | 7A | 131168556 | C, T | sanfensan |
| 1D | 403815045 | T, C | sanfensan | 4C | 339044922 | C, T | sanfensan | 7A | 139918960 | A, G | sanfensan |
| 1D | 409759489 | T, C | sanfensan | 4C | 342159259 | G, A | sanfensan | 7A | 143266476 | T, C | sanfensan |
| 1D | 412133901 | T, C | sanfensan | 4C | 345139072 | G, A | sanfensan | 7A | 149300335 | G, A | sanfensan |
| 1D | 418421166 | C, G | sanfensan | 4C | 350531907 | G, T | sanfensan | 7A | 154734171 | T, C | sanfensan |
| 1D | 423994932 | T, C | sanfensan | 4C | 357426076 | G, A | sanfensan | 7A | 159313052 | C, T | sanfensan |
| 1D | 429511063 | A, G | sanfensan | 4C | 362889043 | G, A | sanfensan | 7A | 160075815 | C, G | sanfensan |
| 1D | 433441263 | A, G | sanfensan | 4C | 369798229 | G, A | sanfensan | 7A | 167272883 | C, T | sanfensan |
| 1D | 435135220 | T, A | sanfensan | 4C | 372472310 | C, T | sanfensan | 7A | 171591003 | T, G | sanfensan |
| 1D | 442087046 | G, A | sanfensan | 4C | 378265862 | T, C | sanfensan | 7A | 177018083 | G, T | sanfensan |
| 1D | 442124461 | G, A | sanfensan | 4C | 381319655 | G, A | sanfensan | 7A | 184807238 | C, A | sanfensan |
| 1D | 449745797 | G, C | sanfensan | 4C | 385095261 | T, G | sanfensan | 7A | 186333580 | A, G | sanfensan |
| 1D | 450598659 | A, G | sanfensan | 4C | 387723697 | C, T | sanfensan | 7A | 190961467 | T, C | sanfensan |
| 1D | 459581711 | T, C | sanfensan | 4C | 392326058 | T, C | sanfensan | 7A | 197600313 | G, A | sanfensan |
| 1D | 460387603 | C, G | sanfensan | 4C | 395186922 | T, C | sanfensan | 7A | 201074764 | C, A | sanfensan |
| 1D | 468646779 | G, A | sanfensan | 4C | 404320541 | A, C | sanfensan | 7A | 208272139 | T, G | sanfensan |
| 1D | 470605204 | C, T | sanfensan | 4C | 407798339 | C, G | sanfensan | 7A | 212498087 | C, T | sanfensan |
| 2A | 1294852 | A, G | sanfensan | 4C | 414198946 | A, G | sanfensan | 7A | 219185506 | G, T | sanfensan |
| 2A | 8354341 | G, A | sanfensan | 4C | 418786637 | C, T | sanfensan | 7A | 222607909 | A, G | sanfensan |
| 2A | 10205647 | T, G | sanfensan | 4C | 420987303 | G, C | sanfensan | 7A | 226206685 | T, C | sanfensan |
| 2A | 17133037 | A, G | sanfensan | 4C | 425274444 | A, T | sanfensan | 7A | 234382860 | A, T | sanfensan |
| 2A | 22147985 | C, G | sanfensan | 4C | 437039125 | T, C | sanfensan | 7A | 235670912 | C, T | sanfensan |
| 2A | 25577776 | C, A | sanfensan | 4C | 442594181 | C, T | sanfensan | 7A | 242252774 | A, G | sanfensan |
| 2A | 33113184 | G, T | sanfensan | 4C | 446643052 | G, T | sanfensan | 7A | 246670561 | A, C | sanfensan |
| 2A | 38311226 | G, A | sanfensan | 4C | 451884828 | C, A | sanfensan | 7A | 250206642 | T, C | sanfensan |
| 2A | 43688833 | A, C | sanfensan | 4C | 455850824 | G, A | sanfensan | 7A | 259731844 | C, T | sanfensan |
| 2A | 45363179 | C, T | sanfensan | 4C | 463494104 | C, T | sanfensan | 7A | 261123685 | T, C | sanfensan |
| 2A | 54582062 | A, G | sanfensan | 4C | 467165660 | C, T | sanfensan | 7A | 267061512 | G, C | sanfensan |
| 2A | 58712383 | A, G | sanfensan | 4C | 472240988 | T, G | sanfensan | 7A | 273875056 | A, T | sanfensan |
| 2A | 60354747 | A, G | sanfensan | 4C | 475026617 | T, C | sanfensan | 7A | 279451537 | C, G | sanfensan |
| 2A | 69920614 | A, G | sanfensan | 4C | 481585885 | G, A | sanfensan | 7A | 283880271 | G, A | sanfensan |
| 2A | 74714222 | T, C | sanfensan | 4C | 487240271 | G, T | sanfensan | 7A | 289470204 | T, C | sanfensan |
| 2A | 78592686 | A, C | sanfensan | 4C | 493243160 | C, T | sanfensan | 7A | 294405482 | T, C | sanfensan |
| 2A | 79665300 | C, A | sanfensan | 4C | 499531487 | C, A | sanfensan | 7A | 296729065 | G, T | sanfensan |
| 2A | 83491335 | A, G | sanfensan | 4C | 504518607 | G, A | sanfensan | 7A | 304733749 | T, C | sanfensan |
| 2A | 88992475 | A, C | sanfensan | 4C | 509955623 | G, A | sanfensan | 7A | 306515921 | T, C | sanfensan |
| 2A | 91196574 | C, T | sanfensan | 4C | 511514837 | A, G | sanfensan | 7A | 310678817 | G, A | sanfensan |
| 2A | 98762331 | C, G | sanfensan | 4C | 514138096 | G, A | sanfensan | 7A | 317998480 | C, T | sanfensan |
| 2A | 102839151 | T, C | sanfensan | 4C | 519111243 | A, T | sanfensan | 7A | 324083519 | A, G | sanfensan |
| 2A | 105115673 | C, T | sanfensan | 4C | 520487689 | C, T | sanfensan | 7A | 326015574 | A, G | sanfensan |
| 2A | 113143954 | G, A | sanfensan | 4C | 526516206 | A, G | sanfensan | 7A | 334096551 | G, A | sanfensan |
| 2A | 115778719 | C, T | sanfensan | 4C | 530500162 | C, T | sanfensan | 7A | 337503568 | G, A | sanfensan |
| 2A | 123726669 | G, C | sanfensan | 4C | 537644622 | T, C | sanfensan | 7A | 342389640 | T, C | sanfensan |
| 2A | 125030830 | G, A | sanfensan | 4C | 540730312 | G, A | sanfensan | 7A | 349080210 | A, G | sanfensan |
| 2A | 133605969 | C, A | sanfensan | 4C | 545836115 | G, T | sanfensan | 7A | 352615237 | T, C | sanfensan |
| 2A | 136067584 | A, C | sanfensan | 4C | 549932188 | A, G | sanfensan | 7A | 355460008 | G, A | sanfensan |
| 2A | 142891390 | C, T | sanfensan | 4C | 550915489 | A, T | sanfensan | 7A | 361858462 | T, C | sanfensan |
| 2A | 148925271 | C, T | sanfensan | 4C | 558054888 | C, T | sanfensan | 7A | 363873233 | C, T | sanfensan |
| 2A | 150242064 | A, G | sanfensan | 4C | 562963997 | A, G | sanfensan | 7A | 367416741 | A, G | sanfensan |
| 2A | 159722254 | T, C | sanfensan | 4C | 569467277 | A, G | sanfensan | 7A | 370900705 | C, A | sanfensan |
| 2A | 161633504 | G, A | sanfensan | 4C | 572657475 | T, C | sanfensan | 7A | 377494585 | C, T | sanfensan |
| 2A | 166801852 | G, A | sanfensan | 4C | 575327801 | A, G | sanfensan | 7A | 380620357 | T, C | sanfensan |
| 2A | 172884455 | G, C | sanfensan | 4C | 584368593 | T, G | sanfensan | 7A | 387542205 | A, G | sanfensan |
| 2A | 177892881 | A, G | sanfensan | 4C | 588543504 | A, G | sanfensan | 7A | 393193897 | T, C | sanfensan |
| 2A | 180161463 | A, C | sanfensan | 4C | 589445451 | A, G | sanfensan | 7A | 397890567 | A, G | sanfensan |
| 2A | 187191668 | G, A | sanfensan | 4C | 593669777 | A, C | sanfensan | 7A | 402356276 | A, T | sanfensan |
| 2A | 194352120 | C, A | sanfensan | 4C | 599151537 | A, G | sanfensan | 7A | 408034177 | T, G | sanfensan |
| 2A | 195938867 | T, C | sanfensan | 4C | 600053663 | T, C | sanfensan | 7A | 411938390 | C, T | sanfensan |
| 2A | 202623332 | G, T | sanfensan | 4C | 607995668 | A, G | sanfensan | 7A | 415820845 | C, T | sanfensan |
| 2A | 209957751 | G, T | sanfensan | 4C | 610178903 | T, C | sanfensan | 7A | 421650338 | A, G | sanfensan |
| 2A | 210875267 | T, G | sanfensan | 4C | 614566248 | G, C | sanfensan | 7A | 429369869 | A, G | sanfensan |
| 2A | 215784372 | C, T | sanfensan | 4C | 616610794 | C, T | sanfensan | 7A | 433335698 | G, A | sanfensan |
| 2A | 224379638 | G, C | sanfensan | 4C | 624701501 | T, C | sanfensan | 7A | 438899194 | T, C | sanfensan |
| 2A | 227479068 | C, T | sanfensan | 4C | 626629588 | G, A | sanfensan | 7A | 442020982 | T, C | sanfensan |
| 2A | 231405711 | A, C | sanfensan | 4C | 631097435 | A, C | sanfensan | 7A | 449206870 | A, G | sanfensan |
| 2A | 237629147 | C, T | sanfensan | 4C | 635505874 | C, A | sanfensan | 7A | 452644138 | A, C | sanfensan |
| 2A | 240260139 | T, C | sanfensan | 4C | 642622496 | T, C | sanfensan | 7A | 455480446 | T, G | sanfensan |
| 2A | 248913514 | G, C | sanfensan | 4C | 645881564 | A, C | sanfensan | 7A | 463118331 | A, G | sanfensan |
| 2A | 251101436 | C, T | sanfensan | 4C | 653135330 | G, T | sanfensan | 7A | 467735097 | C, T | sanfensan |
| 2A | 258858124 | C, T | sanfensan | 4C | 655135466 | A, G | sanfensan | 7A | 471902717 | A, G | sanfensan |
| 2A | 262168685 | G, A | sanfensan | 4C | 660684760 | G, A | sanfensan | 7A | 476107323 | A, G | sanfensan |
| 2A | 269432173 | A, C | sanfensan | 4C | 665124776 | G, C | sanfensan | 7A | 482021904 | C, T | sanfensan |
| 2A | 272066634 | T, C | sanfensan | 4C | 674949846 | A, G | sanfensan | 7A | 485131125 | A, G | sanfensan |
| 2A | 275191428 | C, T | sanfensan | 4C | 675185316 | A, G | sanfensan | 7A | 493482291 | G, A | sanfensan |
| 2A | 282263456 | T, G | sanfensan | 4C | 683318163 | T, C | sanfensan | 7A | 495121727 | T, C | sanfensan |
| 2A | 288156444 | C, G | sanfensan | 4C | 689664776 | T, C | sanfensan | 7C | 4614858 | T, G | sanfensan |
| 2A | 291947063 | A, C | sanfensan | 4C | 694624379 | C, T | sanfensan | 7C | 5317219 | C, T | sanfensan |
| 2A | 298698676 | T, C | sanfensan | 4C | 695265876 | A, G | sanfensan | 7C | 14340861 | A, G | sanfensan |
| 2A | 302165782 | G, A | sanfensan | 4C | 701273830 | A, G | sanfensan | 7C | 19478034 | G, A | sanfensan |
| 2A | 307413298 | T, C | sanfensan | 4D | 3022923 | A, G | sanfensan | 7C | 24565676 | G, A | sanfensan |
| 2A | 314041736 | C, G | sanfensan | 4D | 7849878 | C, T | sanfensan | 7C | 29930105 | T, C | sanfensan |
| 2A | 319115852 | C, T | sanfensan | 4D | 10672344 | A, G | sanfensan | 7C | 32494696 | G, A | sanfensan |
| 2A | 324309925 | C, T | sanfensan | 4D | 16331251 | T, G | sanfensan | 7C | 39005353 | C, T | sanfensan |
| 2A | 325423488 | A, C | sanfensan | 4D | 23073905 | A, G | sanfensan | 7C | 41317427 | A, G | sanfensan |
| 2A | 332013465 | T, C | sanfensan | 4D | 28279824 | T, G | sanfensan | 7C | 47961168 | A, C | sanfensan |
| 2A | 336329704 | T, G | sanfensan | 4D | 32649688 | A, G | sanfensan | 7C | 50182911 | G, C | sanfensan |
| 2A | 337567230 | G, T | sanfensan | 4D | 37038379 | A, C | sanfensan | 7C | 55925952 | A, G | sanfensan |
| 2A | 340385583 | C, T | sanfensan | 4D | 43180451 | T, C | sanfensan | 7C | 61239403 | C, G | sanfensan |
| 2A | 347331178 | A, G | sanfensan | 4D | 45472604 | A, G | sanfensan | 7C | 67047337 | T, G | sanfensan |
| 2A | 354110393 | C, T | sanfensan | 4D | 50482410 | A, G | sanfensan | 7C | 71975946 | G, T | sanfensan |
| 2A | 355224874 | C, T | sanfensan | 4D | 58490879 | G, A | sanfensan | 7C | 72522696 | C, T | sanfensan |
| 2A | 361450337 | A, G | sanfensan | 4D | 62917919 | G, A | sanfensan | 7C | 79287234 | A, C | sanfensan |
| 2A | 365973983 | T, C | sanfensan | 4D | 65612418 | G, A | sanfensan | 7C | 84848962 | G, A | sanfensan |
| 2A | 374152559 | T, C | sanfensan | 4D | 71637400 | C, T | sanfensan | 70 | 88219749 | C, T | sanfensan |
| 2A | 378774926 | A, G | sanfensan | 4D | 76187635 | C, T | sanfensan | 7C | 94995046 | A, G | sanfensan |
| 2A | 380652237 | C, T | sanfensan | 4D | 82058789 | T, C | sanfensan | 7C | 97771126 | T, C | sanfensan |
| 2A | 386433427 | G, A | sanfensan | 4D | 88887772 | G, A | sanfensan | 7C | 104891291 | G, A | sanfensan |
| 2A | 391759319 | C, T | sanfensan | 4D | 92323177 | A, G | sanfensan | 7C | 108552236 | A, G | sanfensan |
| 2A | 397676516 | C, G | sanfensan | 4D | 97430755 | A, G | sanfensan | 7C | 113384152 | G, T | sanfensan |
| 2A | 404431299 | T, C | sanfensan | 4D | 102821671 | C, G | sanfensan | 7C | 118141656 | A, G | sanfensan |
| 2A | 406323117 | T, C | sanfensan | 4D | 106018930 | T, G | sanfensan | 7C | 123007101 | G, C | sanfensan |
| 2A | 414368443 | A, G | sanfensan | 4D | 110865436 | A, G | sanfensan | 7C | 129029468 | T, C | sanfensan |
| 2A | 418701849 | C, A | sanfensan | 4D | 116039043 | C, T | sanfensan | 7C | 134291990 | T, C | sanfensan |
| 2A | 421611597 | T, C | sanfensan | 4D | 118774001 | C, G | sanfensan | 7C | 137256580 | T.C | sanfensan |
| 2A | 429392443 | C, A | sanfensan | 4D | 121609230 | A, G | sanfensan | 7C | 140877690 | G, A | sanfensan |
| 2A | 432088076 | C, A | sanfensan | 4D | 128046905 | T, C | sanfensan | 7C | 145949568 | G, C | sanfensan |
| 2A | 432433952 | T, G | sanfensan | 4D | 134025258 | T, C | sanfensan | 7C | 151007290 | G, A | sanfensan |
| 2A | 437058043 | T, C | sanfensan | 4D | 137854184 | C, G | sanfensan | 7C | 157054447 | C, T | sanfensan |
| 2A | 442675069 | G, C | sanfensan | 4D | 141417395 | A, G | sanfensan | 7C | 163909424 | G, A | sanfensan |
| 2A | 447978464 | A, G | sanfensan | 4D | 148636319 | A, G | sanfensan | 7C | 166612341 | A, C | sanfensan |
| 2A | 452816108 | C, A | sanfensan | 4D | 151219343 | C, T | sanfensan | 7C | 171906105 | A, G | sanfensan |
| 2A | 456332224 | T, C | sanfensan | 4D | 153032013 | T, C | sanfensan | 7C | 177293559 | T, C | sanfensan |
| 2A | 460704136 | T, C | sanfensan | 4D | 159949170 | T, C | sanfensan | 7C | 179283192 | T, C | sanfensan |
| 2C | 3755642 | C, G | sanfensan | 4D | 163913258 | A, G | sanfensan | 7C | 180628837 | C, T | sanfensan |
| 2C | 6523209 | T, C | sanfensan | 4D | 165337682 | T, A | sanfensan | 7C | 185998111 | A, G | sanfensan |
| 2C | 14666414 | G, A | sanfensan | 4D | 173510202 | T, G | sanfensan | 7C | 191666515 | A, G | sanfensan |
| 2C | 18219538 | G, A | sanfensan | 4D | 179301600 | C, G | sanfensan | 7C | 197044982 | A, G | sanfensan |
| 2C | 20598121 | C, T | sanfensan | 4D | 184426060 | G, A | sanfensan | 7C | 204791800 | A, C | sanfensan |
| 2C | 25528699 | G, A | sanfensan | 4D | 187340496 | G, T | sanfensan | 7C | 206297454 | T, C | sanfensan |
| 2C | 34351879 | G, A | sanfensan | 4D | 193603755 | A, G | sanfensan | 7C | 214665049 | T, C | sanfensan |
| 2C | 37480085 | G, A | sanfensan | 4D | 196382892 | T, C | sanfensan | 7C | 216739159 | A, G | sanfensan |
| 2C | 40282520 | C, T | sanfensan | 4D | 202074844 | C, T | sanfensan | 7C | 220606602 | T, C | sanfensan |
| 2C | 47255505 | G, A | sanfensan | 4D | 206447702 | G, A | sanfensan | 7C | 225604579 | C, A | sanfensan |
| 2C | 52177917 | C, T | sanfensan | 4D | 212380635 | C, T | sanfensan | 7C | 232971786 | G, C | sanfensan |
| 2C | 55006635 | C, A | sanfensan | 4D | 215885690 | T, C | sanfensan | 7C | 236226644 | C, T | sanfensan |
| 2C | 62911833 | A, C | sanfensan | 4D | 220033123 | T, G | sanfensan | 7C | 242089348 | C, G | sanfensan |
| 2C | 69182434 | C, T | sanfensan | 4D | 227048389 | A, G | sanfensan | 7C | 247003405 | C, T | sanfensan |
| 2C | 73235690 | G, A | sanfensan | 4D | 232039750 | C, T | sanfensan | 7C | 249178004 | T, G | sanfensan |
| 2C | 75663383 | C, T | sanfensan | 4D | 236815937 | T, A | sanfensan | 7C | 250146710 | C, T | sanfensan |
| 2C | 80698781 | C, T | sanfensan | 4D | 242091618 | C, T | sanfensan | 7C | 257100796 | G, A | sanfensan |
| 2C | 88439094 | G, C | sanfensan | 4D | 245101303 | C, A | sanfensan | 7C | 261648943 | C, A | sanfensan |
| 2C | 93854384 | C, T | sanfensan | 4D | 250407222 | A, G | sanfensan | 7C | 268903706 | T, C | sanfensan |
| 2C | 97362350 | G, A | sanfensan | 4D | 256019130 | T, C | sanfensan | 7C | 273820692 | C, G | sanfensan |
| 2C | 104745256 | C, T | sanfensan | 4D | 260909900 | T, C | sanfensan | 7C | 278686128 | T, C | sanfensan |
| 2C | 108205218 | C, T | sanfensan | 4D | 267887871 | A, G | sanfensan | 7C | 280947265 | G, A | sanfensan |
| 2C | 110557690 | G, A | sanfensan | 4D | 271126060 | C, A | sanfensan | 7C | 286809973 | T, C | sanfensan |
| 2C | 119588975 | C, T | sanfensan | 4D | 277162174 | T, G | sanfensan | 7C | 291012394 | G, C | sanfensan |
| 2C | 122248235 | C, T | sanfensan | 4D | 283660950 | G, A | sanfensan | 7C | 296841339 | A, C | sanfensan |
| 2C | 129849765 | C, T | sanfensan | 4D | 285824135 | G, T | sanfensan | 7C | 301552328 | T, C | sanfensan |
| 2C | 134962790 | G, A | sanfensan | 4D | 294845699 | G, A | sanfensan | 7C | 304378578 | A, G | sanfensan |
| 2C | 135013067 | T, G | sanfensan | 4D | 299632398 | G, A | sanfensan | 7C | 306040659 | G, A | sanfensan |
| 2C | 140602512 | C, T | sanfensan | 4D | 303350038 | T, C | sanfensan | 7C | 314412301 | G, T | sanfensan |
| 2C | 145478940 | T, C | sanfensan | 4D | 307597358 | T, A | sanfensan | 7C | 318527191 | C, T | sanfensan |
| 2C | 150692118 | G, A | sanfensan | 4D | 314228425 | C, T | sanfensan | 7C | 320205502 | A, G | sanfensan |
| 2C | 156314693 | G, A | sanfensan | 4D | 314253165 | G, A | sanfensan | 7C | 320293039 | G, T | sanfensan |
| 2C | 160485851 | C, T | sanfensan | 4D | 315607825 | A, C | sanfensan | 7C | 327857134 | C, A | sanfensan |
| 2C | 167462260 | G, A | sanfensan | 4D | 321533893 | G, T | sanfensan | 7C | 331712162 | T, C | sanfensan |
| 2C | 173642372 | C, T | sanfensan | 4D | 325136857 | A, T | sanfensan | 7C | 339499335 | T, C | sanfensan |
| 2C | 179174429 | C, A | sanfensan | 4D | 326786150 | A, T | sanfensan | 7C | 340372302 | C, T | sanfensan |
| 2C | 182456426 | G, A | sanfensan | 4D | 332125743 | A, G | sanfensan | 7C | 349292850 | C, T | sanfensan |
| 2C | 188223122 | C, T | sanfensan | 4D | 335136720 | A, G | sanfensan | 7C | 350022239 | G, A | sanfensan |
| 2C | 190626883 | C, T | sanfensan | 4D | 340382269 | C, T | sanfensan | 7C | 350993553 | A, G | sanfensan |
| 2C | 196959827 | G, A | sanfensan | 4D | 348383382 | A, G | sanfensan | 7C | 358188563 | T, C | sanfensan |
| 2C | 201765496 | G, T | sanfensan | 4D | 352477479 | T, C | sanfensan | 7C | 363492217 | C, T | sanfensan |
| 2C | 207043151 | C, T | sanfensan | 4D | 356651761 | A, G | sanfensan | 7C | 369432993 | G, A | sanfensan |
| 2C | 208215965 | C, T | sanfensan | 4D | 363724413 | C, G | sanfensan | 7C | 371369521 | T, G | sanfensan |
| 2C | 213490479 | G, A | sanfensan | 4D | 366480345 | A, G | sanfensan | 7C | 377634589 | A, G | sanfensan |
| 2C | 217643681 | G, A | sanfensan | 4D | 374238192 | T, C | sanfensan | 7C | 384171632 | C, T | sanfensan |
| 2C | 222136297 | G, A | sanfensan | 4D | 378149215 | T, C | sanfensan | 7C | 386369415 | G, A | sanfensan |
| 2C | 226477864 | C, T | sanfensan | 4D | 384366440 | G, A | sanfensan | 7C | 392363537 | C, T | sanfensan |
| 2C | 231505039 | C, T | sanfensan | 4D | 389105351 | A, C | sanfensan | 7C | 396114953 | A, G | sanfensan |
| 2C | 237675775 | C, T | sanfensan | 4D | 390762487 | A, G | sanfensan | 7C | 400812310 | C, T | sanfensan |
| 2C | 240667277 | G, A | sanfensan | 4D | 398109909 | C, G | sanfensan | 7C | 405139125 | A, G | sanfensan |
| 2C | 246770167 | A, C | sanfensan | 4D | 402374421 | T, C | sanfensan | 7C | 413833654 | A, C | sanfensan |
| 2C | 252643615 | G, A | sanfensan | 4D | 407356318 | G, A | sanfensan | 7C | 414551856 | G, T | sanfensan |
| 2C | 256169319 | G, A | sanfensan | 4D | 413268611 | T, C | sanfensan | 7C | 416262975 | C, T | sanfensan |
| 2C | 261833087 | G, A | sanfensan | 4D | 418031647 | G, A | sanfensan | 7C | 420923703 | T, C | sanfensan |
| 2C | 262787175 | A, T | sanfensan | 4D | 420702529 | G, A | sanfensan | 7C | 426911439 | C, A | sanfensan |
| 2C | 267745937 | G, A | sanfensan | 4D | 426669740 | A, G | sanfensan | 7C | 430613225 | G, A | sanfensan |
| 2C | 271735685 | G, A | sanfensan | 4D | 432669116 | G, A | sanfensan | 7C | 432461759 | G, A | sanfensan |
| 2C | 277919032 | G, A | sanfensan | 4D | 437532533 | G, T | sanfensan | 7C | 435550002 | C, T | sanfensan |
| 20 | 281899341 | C, T | sanfensan | 4D | 443034471 | C, T | sanfensan | 7C | 444070312 | G, T | sanfensan |
| 2C | 287576313 | C, T | sanfensan | 4D | 445235008 | A, C | sanfensan | 7C | 445489155 | G, A | sanfensan |
| 2C | 294884196 | G, A | sanfensan | 4D | 451513413 | A, G | sanfensan | 7C | 452005889 | A, T | sanfensan |
| 2C | 298708412 | T, A | sanfensan | 5A | 2255953 | A, C | sanfensan | 7C | 457711724 | T, C | sanfensan |
| 2C | 304070232 | A, G | sanfensan | 5A | 6260213 | C, A | sanfensan | 7C | 462688367 | G, A | sanfensan |
| 2C | 305356521 | G, T | sanfensan | 5A | 13803621 | C, T | sanfensan | 7C | 464956118 | A, G | sanfensan |
| 2C | 312681417 | G, A | sanfensan | 5A | 15561310 | G, C | sanfensan | 7C | 465254207 | G, T | sanfensan |
| 2C | 317317802 | T, C | sanfensan | 5A | 24452734 | T, C | sanfensan | 7C | 471154727 | G, A | sanfensan |
| 2C | 324693110 | G, T | sanfensan | 5A | 27224463 | G, A | sanfensan | 7C | 476266406 | C, A | sanfensan |
| 2C | 329639427 | C, G | sanfensan | 5A | 34502392 | A, G | sanfensan | 7C | 480020947 | C, T | sanfensan |
| 2C | 334559533 | C, A | sanfensan | 5A | 36710142 | G, A | sanfensan | 7C | 485814933 | A, G | sanfensan |
| 2C | 335512805 | C, T | sanfensan | 5A | 44344522 | G, A | sanfensan | 7C | 491712274 | T, C | sanfensan |
| 2C | 342249614 | G, A | sanfensan | 5A | 49562544 | T, C | sanfensan | 7C | 496402779 | G, C | sanfensan |
| 2C | 349065985 | C, T | sanfensan | 5A | 53433795 | T, C | sanfensan | 7C | 500349440 | G, A | sanfensan |
| 2C | 354184855 | C, T | sanfensan | 5A | 56109353 | G, A | sanfensan | 7C | 505836662 | C, T | sanfensan |
| 2C | 358792181 | C, T | sanfensan | 5A | 64589225 | C, T | sanfensan | 7C | 514393934 | A, G | sanfensan |
| 2C | 361602927 | A, C | sanfensan | 5A | 68491563 | T, C | sanfensan | 7C | 518087154 | C, G | sanfensan |
| 2C | 365574905 | T, C | sanfensan | 5A | 70531911 | C, T | sanfensan | 7C | 520313196 | A, G | sanfensan |
| 2C | 372578823 | A, T | sanfensan | 5A | 79896986 | T, C | sanfensan | 7C | 528181463 | G, C | sanfensan |
| 2C | 377682611 | C, T | sanfensan | 5A | 83113930 | T, C | sanfensan | 7C | 530421340 | G, C | sanfensan |
| 2C | 383773493 | G, A | sanfensan | 5A | 86401636 | T, C | sanfensan | 7D | 2445778 | G, A | sanfensan |
| 2C | 388264545 | G, T | sanfensan | 5A | 94264887 | T, C | sanfensan | 7D | 9742047 | A, G | sanfensan |
| 2C | 392647080 | T, A | sanfensan | 5A | 95056890 | T, C | sanfensan | 7D | 9847518 | A, G | sanfensan |
| 2C | 399910470 | G, A | sanfensan | 5A | 102064390 | A, G | sanfensan | 7D | 13815389 | A, T | sanfensan |
| 2C | 402251417 | T, G | sanfensan | 5A | 106428331 | G, A | sanfensan | 7D | 18736551 | G, T | sanfensan |
| 2C | 405239508 | A, G | sanfensan | 5A | 110419074 | G, A | sanfensan | 7D | 20505929 | C, T | sanfensan |
| 2C | 412396338 | A, G | sanfensan | 5A | 116006562 | G, A | sanfensan | 7D | 25511353 | G, A | sanfensan |
| 2C | 416260936 | G, A | sanfensan | 5A | 121002541 | A, T | sanfensan | 7D | 30499092 | G, A | sanfensan |
| 2C | 423413408 | G, A | sanfensan | 5A | 127363246 | T, C | sanfensan | 7D | 36614804 | T, G | sanfensan |
| 2C | 429152113 | C, T | sanfensan | 5A | 132453075 | T, C | sanfensan | 7D | 40700604 | G, C | sanfensan |
| 2C | 433743437 | G, A | sanfensan | 5A | 137069112 | A, C | sanfensan | 7D | 46604719 | C, T | sanfensan |
| 2C | 438694947 | G, A | sanfensan | 5A | 142368295 | C, T | sanfensan | 7D | 50727705 | C, G | sanfensan |
| 2C | 441372926 | G, A | sanfensan | 5A | 147275003 | T, G | sanfensan | 7D | 57700439 | C, T | sanfensan |
| 2C | 446094456 | T, G | sanfensan | 5A | 152777713 | C, T | sanfensan | 7D | 60169262 | C, A | sanfensan |
| 2C | 451391070 | A, G | sanfensan | 5A | 156493283 | C, T | sanfensan | 7D | 69063876 | A, C | sanfensan |
| 2C | 456022723 | A, C | sanfensan | 5A | 160244662 | A, G | sanfensan | 7D | 71993254 | T, C | sanfensan |
| 2C | 462451470 | G, A | sanfensan | 5A | 166708997 | C, G | sanfensan | 7D | 79211559 | A, G | sanfensan |
| 2C | 469973109 | G, A | sanfensan | 5A | 174872733 | T, C | sanfensan | 7D | 82904994 | C, T | sanfensan |
| 2C | 473490736 | G, A | sanfensan | 5A | 176197051 | A, G | sanfensan | 7D | 85615821 | A, G | sanfensan |
| 2C | 476541629 | C, T | sanfensan | 5A | 181213909 | T, C | sanfensan | 7D | 91224657 | T, C | sanfensan |
| 2C | 482708999 | T, G | sanfensan | 5A | 188859714 | T, C | sanfensan | 7D | 98952743 | G, A | sanfensan |
| 2C | 488486085 | C, G | sanfensan | 5A | 190215196 | C, T | sanfensan | 7D | 100841898 | C, T | sanfensan |
| 2C | 492299240 | T, G | sanfensan | 5A | 195549254 | T, C | sanfensan | 7D | 108477777 | G, A | sanfensan |
| 2C | 497502793 | C, T | sanfensan | 5A | 198785103 | A, G | sanfensan | 7D | 110334702 | C, T | sanfensan |
| 2C | 504463768 | G, A | sanfensan | 5A | 202377152 | T, C | sanfensan | 7D | 116643732 | C, T | sanfensan |
| 2C | 506288307 | G, A | sanfensan | 5A | 207723478 | G, A | sanfensan | 7D | 123307950 | T, C | sanfensan |
| 2C | 511067660 | T, C | sanfensan | 5A | 212325208 | A, G | sanfensan | 7D | 127161912 | A, G | sanfensan |
| 2C | 515199747 | G, T | sanfensan | 5A | 216205965 | A, G | sanfensan | 7D | 133410614 | T, G | sanfensan |
| 2C | 520028290 | C, G | sanfensan | 5A | 221258102 | C, T | sanfensan | 7D | 138641756 | C, T | sanfensan |
| 2C | 526639651 | C, T | sanfensan | 5A | 225234818 | G, T | sanfensan | 7D | 141652553 | T, G | sanfensan |
| 2C | 530779807 | G, A | sanfensan | 5A | 230456063 | A, T | sanfensan | 7D | 146207944 | C, T | sanfensan |
| 2C | 537336195 | A, G | sanfensan | 5A | 238470416 | T, C | sanfensan | 7D | 151573471 | G, A | sanfensan |
| 2C | 540940586 | T, C | sanfensan | 5A | 240660477 | G, C | sanfensan | 7D | 156040797 | C, A | sanfensan |
| 2C | 547202871 | G, A | sanfensan | 5A | 245072572 | A, G | sanfensan | 7D | 161660385 | A, G | sanfensan |
| 2C | 552060518 | G, C | sanfensan | 5A | 250830642 | G, C | sanfensan | 7D | 167061192 | A, G | sanfensan |
| 2D | 4090001 | G, A | sanfensan | 5A | 259737745 | A, G | sanfensan | 7D | 170724248 | A, G | sanfensan |
| 2D | 8884387 | G, A | sanfensan | 5A | 261185047 | G, A | sanfensan | 7D | 178681077 | A, C | sanfensan |
| 2D | 13578443 | C, G | sanfensan | 5A | 269804753 | A, G | sanfensan | 7D | 183800740 | A, C | sanfensan |
| 2D | 17675396 | A, G | sanfensan | 5A | 273021787 | T, C | sanfensan | 7D | 186691354 | A, G | sanfensan |
| 2D | 23696567 | G, C | sanfensan | 5A | 276143054 | T, C | sanfensan | 7D | 192032150 | G, A | sanfensan |
| 2D | 29926609 | C, A | sanfensan | 5A | 282716721 | A, C | sanfensan | 7D | 197240877 | G, A | sanfensan |
| 2D | 31795322 | C, G | sanfensan | 5A | 289601566 | A, C | sanfensan | 7D | 203331302 | A, G | sanfensan |
| 2D | 38891629 | C, T | sanfensan | 5A | 290520143 | A, T | sanfensan | 7D | 206895614 | A, G | sanfensan |
| 2D | 43755471 | A, C | sanfensan | 5A | 297192869 | T, G | sanfensan | 7D | 214500259 | A, T | sanfensan |
| 2D | 49839859 | T, C | sanfensan | 5A | 301832004 | T, C | sanfensan | 7D | 216095702 | C, A | sanfensan |
| 2D | 52853593 | T, G | sanfensan | 5A | 308399750 | G, T | sanfensan | 7D | 223545306 | G, A | sanfensan |
| 2D | 59369523 | A, G | sanfensan | 5A | 311247256 | G, A | sanfensan | 7D | 226229762 | G, A | sanfensan |
| 2D | 64844533 | C, T | sanfensan | 5A | 317923031 | C, A | sanfensan | 7D | 232450357 | G, C | sanfensan |
| 2D | 67973342 | G, C | sanfensan | 5A | 321801333 | T, C | sanfensan | 7D | 237413151 | T, C | sanfensan |
| 2D | 72762945 | G, T | sanfensan | 5A | 327241850 | C, T | sanfensan | 7D | 243355850 | T, C | sanfensan |
| 2D | 77924703 | G, A | sanfensan | 5A | 333717258 | A, G | sanfensan | 7D | 247929240 | G, T | sanfensan |
| 2D | 82246788 | C, G | sanfensan | 5A | 335041321 | G, A | sanfensan | 7D | 254107993 | C, G | sanfensan |
| 2D | 85545627 | A, G | sanfensan | 5A | 341600328 | C, T | sanfensan | 7D | 255422386 | T, C | sanfensan |
| 2D | 93391163 | T, C | sanfensan | 5A | 347629159 | C, T | sanfensan | 7D | 264369755 | A, C | sanfensan |
| 2D | 97511713 | T, C | sanfensan | 5A | 350824983 | T, C | sanfensan | 7D | 267080055 | G, C | sanfensan |
| 2D | 103508833 | A, G | sanfensan | 5A | 356271802 | A, G | sanfensan | 7D | 272102782 | T, C | sanfensan |
| 2D | 105773491 | C, T | sanfensan | 5A | 363526707 | C, T | sanfensan | 7D | 276724318 | T, C | sanfensan |
| 2D | 112039326 | T, C | sanfensan | 5A | 369477353 | T, C | sanfensan | 7D | 278615324 | G, C | sanfensan |
| 2D | 118313919 | A, G | sanfensan | 5A | 370529828 | C, G | sanfensan | 7D | 283701259 | T, G | sanfensan |
| 2D | 124707738 | G, A | sanfensan | 5A | 375579309 | T, C | sanfensan | 7D | 285490224 | C, T | sanfensan |
| 2D | 127418722 | T, C | sanfensan | 5A | 383503810 | C, T | sanfensan | 7D | 290974761 | C, T | sanfensan |
| 2D | 134051671 | A, G | sanfensan | 5A | 387182267 | G, C | sanfensan | 7D | 299205902 | T, C | sanfensan |
| 2D | 139923233 | T, C | sanfensan | 5A | 390102471 | A, G | sanfensan | 7D | 302081398 | G, A | sanfensan |
| 2D | 141313127 | G, T | sanfensan | 5A | 398649985 | C, A | sanfensan | 7D | 305799487 | A, G | sanfensan |
| 2D | 147473787 | C, A | sanfensan | 5A | 404206934 | A, G | sanfensan | 7D | 310083219 | A, G | sanfensan |
| 2D | 153131701 | A, G | sanfensan | 5A | 409217956 | G, A | sanfensan | 7D | 318265612 | C, T | sanfensan |
| 2D | 155782705 | A, C | sanfensan | 5A | 411203550 | C, T | sanfensan | 7D | 324554035 | C, A | sanfensan |
| 2D | 161543833 | T, G | sanfensan | 5A | 415301859 | A, G | sanfensan | 7D | 328162737 | A, G | sanfensan |
| 2D | 168209065 | T, C | sanfensan | 5A | 416065809 | T, G | sanfensan | 7D | 332476238 | C, T | sanfensan |
| 2D | 171707336 | G, A | sanfensan | 5A | 421891077 | A, G | sanfensan | 7D | 336554421 | T, C | sanfensan |
| 2D | 175227041 | C, G | sanfensan | 5A | 428858871 | C, T | sanfensan | 7D | 342546157 | T, C | sanfensan |
| 2D | 183727141 | C, A | sanfensan | 5A | 434243983 | A, G | sanfensan | 7D | 348921802 | A, G | sanfensan |
| 2D | 186245952 | T, C | sanfensan | 5A | 436126819 | A, G | sanfensan | 7D | 352024567 | A, C | sanfensan |
| 2D | 191571350 | T, C | sanfensan | 5A | 441185565 | C, T | sanfensan | 7D | 358950077 | C, T | sanfensan |
| 2D | 197886074 | C, A | sanfensan | 5A | 447327633 | G, T | sanfensan | 7D | 362820207 | G, A | sanfensan |
| 2D | 201218813 | A, G | sanfensan | 5A | 452417280 | C, G | sanfensan | 7D | 368623531 | T, C | sanfensan |
| 2D | 206194560 | G, T | sanfensan | 5A | 458701937 | C, G | sanfensan | 7D | 371406914 | G, T | sanfensan |
| 2D | 209315382 | T, C | sanfensan | 5A | 460085569 | A, G | sanfensan | 7D | 379681421 | T, C | sanfensan |
| 2D | 214621383 | T, G | sanfensan | 5A | 468161069 | T, G | sanfensan | 7D | 382815770 | C, T | sanfensan |
| 2D | 217094666 | T, C | sanfensan | 5A | 474226937 | C, G | sanfensan | 7D | 389570780 | G, A | sanfensan |
| 2D | 224088866 | C, T | sanfensan | 5A | 478302848 | A, G | sanfensan | 7D | 390101972 | G, A | sanfensan |
| 2D | 228951287 | A, G | sanfensan | 5A | 483995877 | C, A | sanfensan | 7D | 399143078 | C, T | sanfensan |
| 2D | 233694297 | A, C | sanfensan | 5A | 488177553 | G, T | sanfensan | 7D | 400807921 | C, T | sanfensan |
| 2D | 237373003 | T, C | sanfensan | 5A | 488401160 | A, T | sanfensan | 7D | 406112763 | T, G | sanfensan |
| 2D | 240374430 | G, T | sanfensan | 5C | 3489579 | C, T | sanfensan | 7D | 411104681 | A, G | sanfensan |
| 2D | 247507939 | G, A | sanfensan | 5C | 6268971 | C, T | sanfensan | 7D | 415490064 | G, A | sanfensan |
| 2D | 253371162 | C, T | sanfensan | 5C | 12203702 | G, A | sanfensan | 7D | 423220521 | A, G | sanfensan |
| 2D | 259426340 | T, C | sanfensan | 5C | 16396954 | A, G | sanfensan | 7D | 426049443 | T, C | sanfensan |
| 2D | 262144878 | T, C | sanfensan | 5C | 22928378 | C, A | sanfensan | 7D | 432545626 | A, G | sanfensan |
| 2D | 268152175 | C, T | sanfensan | 5C | 26060685 | T, C | sanfensan | 7D | 438677802 | A, T | sanfensan |
| 2D | 271878835 | A, C | sanfensan | 5C | 31503941 | C, T | sanfensan | 7D | 442595402 | T, C | sanfensan |
| 2D | 278388739 | G, A | sanfensan | 5C | 39150034 | T, C | sanfensan | 7D | 447536505 | C, T | sanfensan |
| 2D | 281916083 | G, A | sanfensan | 5C | 44163319 | T, G | sanfensan | 7D | 453173144 | T, A | sanfensan |
| 2D | 289003292 | T, C | sanfensan | 5C | 47044843 | C, T | sanfensan | 7D | 456025113 | C, T | sanfensan |
| 2D | 293861011 | T, C | sanfensan | 5C | 50558717 | C, A | sanfensan | 7D | 463196054 | A, T | sanfensan |
| 2D | 297746679 | G, A | sanfensan | 5C | 53047612 | G, A | sanfensan | 7D | 466703278 | A, G | sanfensan |
| 2D | 301635985 | T, C | sanfensan | 5C | 56146722 | T, G | sanfensan | 7D | 474383321 | G, T | sanfensan |
| 2D | 307441210 | A, G | sanfensan | 5C | 63376645 | G, A | sanfensan | 7D | 478751163 | T, C | sanfensan |
| 2D | 313997317 | C, T | sanfensan | 5C | 69905284 | T, C | sanfensan | 7D | 481249084 | C, G | sanfensan |
| 2D | 317780757 | T, C | sanfensan | 5C | 74387265 | T, C | sanfensan | 7D | 486113092 | A, T | sanfensan |
| 2D | 324471320 | G, C | sanfensan | 5C | 77896048 | C, T | sanfensan | 7D | 491181988 | G, T | sanfensan |
| 2D | 325394234 | C, T | sanfensan | 5C | 81012623 | C, T | sanfensan | 7D | 497047184 | A, G | sanfensan |
| 2D | 332243555 | G, A | sanfensan | 5C | 86566325 | A, G | sanfensan | 1A | 106635350 | G, C | OT3098 |
| 2D | 336723088 | C, T | sanfensan | 5C | 94254331 | G, C | sanfensan | 1A | 304418445 | G, A | OT3098 |
| 2D | 344502614 | T, C | sanfensan | 5C | 95909941 | T, C | sanfensan | 1A | 338092374 | C, T | OT3098 |
| 2D | 348558097 | G, A | sanfensan | 5C | 101827763 | A, G | sanfensan | 1A | 348499239 | C, T | OT3098 |
| 2D | 354230586 | G, A | sanfensan | 5C | 109797422 | C, A | sanfensan | 1A | 369374878 | A, G | OT3098 |
| 2D | 356506435 | C, T | sanfensan | 5C | 114362886 | G, T | sanfensan | 1A | 383912262 | C, T | OT3098 |
| 2D | 361372636 | C, A | sanfensan | 5C | 119134297 | A, C | sanfensan | 1A | 397750385 | A, G | OT3098 |
| 2D | 367508960 | T, C | sanfensan | 5C | 121784178 | G, A | sanfensan | 1A | 512105703 | G, A | OT3098 |
| 2D | 374623643 | C, T | sanfensan | 5C | 125633452 | C, A | sanfensan | 1C | 40349872 | G, T | OT3098 |
| 2D | 376574690 | G, A | sanfensan | 5C | 132720505 | G, A | sanfensan | 1C | 80742591 | G, T | OT3098 |
| 2D | 381350972 | A, G | sanfensan | 5C | 138374966 | A, G | sanfensan | 1C | 104538708 | A, G | OT3098 |
| 2D | 385713492 | C, T | sanfensan | 5C | 143890607 | C, T | sanfensan | 1C | 119650459 | G, T | OT3098 |
| 2D | 389191706 | C, A | sanfensan | 5C | 145866226 | G, A | sanfensan | 1C | 120468329 | T, G | OT3098 |
| 2D | 393186784 | T, C | sanfensan | 5C | 150225588 | G, A | sanfensan | 1C | 181195253 | G, A | OT3098 |
| 2D | 398053878 | C, T | sanfensan | 5C | 156966699 | A, G | sanfensan | 1C | 242119623 | C, T | OT3098 |
| 2D | 402113963 | G, A | sanfensan | 5C | 160214876 | G, C | sanfensan | 1C | 265897428 | C, T | OT3098 |
| 2D | 407714293 | C, T | sanfensan | 5C | 168811131 | T, C | sanfensan | 1C | 280066160 | G, A | OT3098 |
| 2D | 413300157 | C, G | sanfensan | 5C | 174809389 | G, C | sanfensan | 1C | 291282749 | A, T | OT3098 |
| 2D | 416994083 | C, T | sanfensan | 5C | 177125115 | A, C | sanfensan | 1C | 319005689 | C, A | OT3098 |
| 2D | 422486122 | A, G | sanfensan | 5C | 182633756 | G, A | sanfensan | 1C | 342223334 | G, A | OT3098 |
| 2D | 429086006 | C, T | sanfensan | 5C | 189642953 | T, C | sanfensan | 1C | 362511327 | C, T | OT3098 |
| 2D | 430932257 | T, G | sanfensan | 5C | 193221965 | A, G | sanfensan | 1C | 403231603 | G, T | OT3098 |
| 2D | 435517110 | T, C | sanfensan | 5C | 199654787 | C, T | sanfensan | 1D | 28155193 | G, A | OT3098 |
| 2D | 440681525 | G, T | sanfensan | 5C | 201058413 | C, T | sanfensan | 1D | 105612570 | C, T | OT3098 |
| 2D | 446278114 | T, C | sanfensan | 5C | 208411390 | T, C | sanfensan | ID | 114754382 | T, A | OT3098 |
| 2D | 452744585 | A, G | sanfensan | 5C | 214681134 | T, C | sanfensan | 1D | 153648523 | C, T | OT3098 |
| 2D | 458588987 | G, A | sanfensan | 5C | 216349665 | C, T | sanfensan | 1D | 168181978 | G, A | OT3098 |
| 2D | 460269576 | T, G | sanfensan | 5C | 220308120 | C, T | sanfensan | 1D | 178507460 | T, C | OT3098 |
| 2D | 466552364 | C, T | sanfensan | 5C | 223909826 | T, C | sanfensan | 1D | 186530691 | C, G | OT3098 |
| 2D | 473536427 | A, C | sanfensan | 5C | 227213415 | T, C | sanfensan | 1D | 199381843 | C, T | OT3098 |
| 2D | 478638656 | T, G | sanfensan | 5C | 234663041 | C, T | sanfensan | 1D | 213294278 | C, A | OT3098 |
| 2D | 484074145 | G, A | sanfensan | 5C | 236099260 | A, C | sanfensan | 1D | 234770750 | A, G | OT3098 |
| 2D | 486599496 | C, T | sanfensan | 5C | 244246397 | C, A | sanfensan | 1D | 249534977 | G, A | OT3098 |
| 2D | 492020467 | C, T | sanfensan | 5C | 246386706 | C, A | sanfensan | 1D | 336367249 | T, C | OT3098 |
| 2D | 498876308 | C, T | sanfensan | 5C | 252990985 | A, T | sanfensan | 1D | 458302223 | A, G | OT3098 |
| 2D | 499971233 | T, C | sanfensan | 5C | 255616974 | C, A | sanfensan | 2A | 288229046 | G, T | OT3098 |
| 2D | 501118019 | C, T | sanfensan | 5C | 261629346 | G, A | sanfensan | 2A | 298965546 | A, G | OT3098 |
| 2D | 509719761 | G, T | sanfensan | 5C | 266182002 | C, T | sanfensan | 2A | 312180949 | G, A | OT3098 |
| 2D | 511708692 | G, A | sanfensan | 5C | 271236736 | C, A | sanfensan | 2A | 348182068 | C, T | OT3098 |
| 2D | 515022757 | T, A | sanfensan | 5C | 275627914 | A, G | sanfensan | 2A | 374638766 | A, G | OT3098 |
| 2D | 523356028 | C, A | sanfensan | 5C | 283728720 | C, T | sanfensan | 2A | 399111341 | G, A | OT3098 |
| 2D | 525941172 | A, G | sanfensan | 5C | 285163996 | T, C | sanfensan | 2C | 28799948 | T, C | OT3098 |
| 2D | 529724172 | A, C | sanfensan | 5C | 292163090 | G, A | sanfensan | 2C | 42159271 | T, G | OT3098 |
| 2D | 530568058 | G, A | sanfensan | 5C | 297500311 | T, C | sanfensan | 2C | 61574030 | G, T | OT3098 |
| 3A | 349403 | C, T | sanfensan | 5C | 304040309 | T, C | sanfensan | 2C | 87111306 | T, C | OT3098 |
| 3A | 5686447 | A, G | sanfensan | 5C | 306535191 | C, T | sanfensan | 2C | 163497553 | G, A | OT3098 |
| 3A | 14820154 | G, C | sanfensan | 5C | 313032349 | T, G | sanfensan | 2C | 384573213 | A, G | OT3098 |
| 3A | 19103892 | A, G | sanfensan | 5C | 318243088 | T, G | sanfensan | 2C | 526110948 | A, G | OT3098 |
| 3A | 24307017 | G, A | sanfensan | 5C | 320862543 | G, A | sanfensan | 2C | 530336382 | G, A | OT3098 |
| 3A | 28596998 | G, A | sanfensan | 5C | 325755346 | C, T | sanfensan | 2D | 25489315 | A, G | OT3098 |
| 3A | 34850179 | A, G | sanfensan | 5C | 333233541 | T, C | sanfensan | 2D | 48295932 | C, T | OT3098 |
| 3A | 39937020 | C, T | sanfensan | 5C | 338139569 | T, C | sanfensan | 2D | 136287278 | G, A | OT3098 |
| 3A | 43520691 | A, G | sanfensan | 5C | 342626437 | A, G | sanfensan | 2D | 257949893 | T, C | OT3098 |
| 3A | 49770087 | T, C | sanfensan | 5C | 347361434 | G, A | sanfensan | 2D | 374246423 | T, G | OT3098 |
| 3A | 54743914 | A, G | sanfensan | 5C | 354441762 | C, T | sanfensan | 2D | 439808678 | A, G | OT3098 |
| 3A | 56540660 | A, C | sanfensan | 5C | 358341050 | C, G | sanfensan | 2D | 464345033 | G, A | OT3098 |
| 3A | 60015652 | C, T | sanfensan | 5C | 363432176 | T, C | sanfensan | 3A | 13251626 | G, A | OT3098 |
| 3A | 68312753 | T, G | sanfensan | 5C | 369584563 | T, C | sanfensan | 3A | 65224422 | C, T | OT3098 |
| 3A | 70210131 | A, G | sanfensan | 5C | 373117738 | G, A | sanfensan | 3A | 80331258 | G, A | OT3098 |
| 3A | 75548656 | C, T | sanfensan | 5C | 377549147 | G, A | sanfensan | 3A | 102356888 | G, A | OT3098 |
| 3A | 81960668 | G, A | sanfensan | 5C | 384699195 | G, A | sanfensan | 3A | 235151032 | C, T | OT3098 |
| 3A | 87385909 | G, A | sanfensan | 5C | 385128549 | C, T | sanfensan | 3A | 290609726 | A, G | OT3098 |
| 3A | 91466974 | T, C | sanfensan | 5C | 391966210 | C, T | sanfensan | 3A | 342060468 | A, G | OT3098 |
| 3A | 99324808 | G, A | sanfensan | 5C | 399070404 | C, T | sanfensan | 3A | 375362853 | G, T | OT3098 |
| 3A | 103702261 | A, T | sanfensan | 5C | 402179080 | A, G | sanfensan | 3C | 83743224 | C, T | OT3098 |
| 3A | 106902222 | A, G | sanfensan | 5C | 405507477 | T, C | sanfensan | 3C | 105875209 | T, A | OT3098 |
| 3A | 114734802 | C, T | sanfensan | 5C | 411038650 | A, G | sanfensan | 3C | 153371948 | G, A | OT3098 |
| 3A | 117018962 | T, G | sanfensan | 5C | 416614236 | G, C | sanfensan | 3C | 160415743 | A, G | OT3098 |
| 3A | 123014541 | G, T | sanfensan | 5C | 421435620 | T, G | sanfensan | 3C | 188382442 | C, T | OT3098 |
| 3A | 125235584 | G, A | sanfensan | 5C | 427497128 | A, G | sanfensan | 3C | 199311115 | T, C | OT3098 |
| 3A | 132736923 | G, T | sanfensan | 5C | 432532568 | A, C | sanfensan | 3C | 215249361 | C, T | OT3098 |
| 3A | 133400727 | T, C | sanfensan | 5C | 435770963 | C, T | sanfensan | 3C | 242293326 | A, C | OT3098 |
| 3A | 135881496 | A, G | sanfensan | 5C | 440258824 | A, G | sanfensan | 3C | 253359904 | T, C | OT3098 |
| 3A | 140132188 | A, C | sanfensan | 5C | 447827826 | T, C | sanfensan | 3C | 272308234 | G, T | OT3098 |
| 3A | 149216557 | G, T | sanfensan | 5C | 451307256 | C, A | sanfensan | 3C | 302216223 | G, A | OT3098 |
| 3A | 153688728 | T, C | sanfensan | 5C | 458701577 | A, C | sanfensan | 3C | 312338539 | T, C | OT3098 |
| 3A | 157365828 | T, G | sanfensan | 5C | 462771476 | G, T | sanfensan | 3C | 327416798 | T, C | OT3098 |
| 3A | 162629353 | G, A | sanfensan | 5C | 468438848 | G, A | sanfensan | 3C | 349649824 | A, C | OT3098 |
| 3A | 163249925 | C, T | sanfensan | 5C | 473285230 | T, C | sanfensan | 3C | 351983098 | G, A | OT3098 |
| 3A | 165620860 | A, G | sanfensan | 5C | 477766211 | T, C | sanfensan | 3C | 416217578 | T, A | OT3098 |
| 3A | 170114512 | T, C | sanfensan | 5C | 480911717 | C, G | sanfensan | 3C | 533296338 | T, C | OT3098 |
| 3A | 175321928 | G, A | sanfensan | 5C | 489509201 | C, G | sanfensan | 3C | 543025252 | A, G | OT3098 |
| 3A | 183395685 | C, T | sanfensan | 5C | 490176188 | G, C | sanfensan | 3C | 551825075 | C, T | OT3098 |
| 3A | 188224937 | A, C | sanfensan | 5C | 498023277 | A, G | sanfensan | 3C | 571627195 | T, C | OT3098 |
| 3A | 194044731 | G, A | sanfensan | 5C | 503467351 | T, C | sanfensan | 3C | 606797452 | C, A | OT3098 |
| 3A | 197334481 | G, A | sanfensan | 5C | 509322035 | T, C | sanfensan | 3C | 620635579 | G, A | OT3098 |
| 3A | 204024961 | T, C | sanfensan | 5C | 512872294 | A, G | sanfensan | 3D | 471246706 | C, T | OT3098 |
| 3A | 209320496 | A, G | sanfensan | 5C | 515412167 | A, G | sanfensan | 4A | 4383966 | T, C | OT3098 |
| 3A | 214269791 | G, A | sanfensan | 5C | 520108101 | T, C | sanfensan | 4A | 91672697 | A, G | OT3098 |
| 3A | 215870959 | A, G | sanfensan | 5C | 526886988 | G, A | sanfensan | 4A | 176172514 | T, C | OT3098 |
| 3A | 220058696 | A, C | sanfensan | 5C | 533665172 | G, A | sanfensan | 4A | 262281263 | T, G | OT3098 |
| 3A | 228807812 | G, A | sanfensan | 5C | 536150519 | G, A | sanfensan | 4A | 281599989 | C, T | OT3098 |
| 3A | 234527749 | A, G | sanfensan | 5C | 542459731 | T, C | sanfensan | 4A | 315467166 | T, C | OT3098 |
| 3A | 237989422 | G, A | sanfensan | 5C | 548440305 | T, C | sanfensan | 4A | 330624554 | C, T | OT3098 |
| 3A | 241256197 | C, T | sanfensan | 5C | 551331730 | A, G | sanfensan | 4A | 344502223 | G, A | OT3098 |
| 3A | 248608009 | C, T | sanfensan | 5C | 555077838 | C, G | sanfensan | 4A | 370035053 | A, G | OT3098 |
| 3A | 254861238 | T, C | sanfensan | 5C | 560097889 | T, C | sanfensan | 4A | 409954087 | G, A | OT3098 |
| 3A | 255490400 | A, G | sanfensan | 5C | 569206294 | C, T | sanfensan | 4A | 438257237 | C, G | OT3098 |
| 3A | 261499403 | T, C | sanfensan | 5C | 572903980 | A, G | sanfensan | 4A | 452257049 | G, A | OT3098 |
| 3A | 268848139 | T, G | sanfensan | 5C | 579001353 | A, G | sanfensan | 4C | 228404747 | C, T | OT3098 |
| 3A | 273512863 | T, C | sanfensan | 5C | 582680547 | C, T | sanfensan | 4C | 326801689 | T, C | OT3098 |
| 3A | 279998619 | A, G | sanfensan | 5C | 585411392 | C, T | sanfensan | 4C | 372872071 | G, A | OT3098 |
| 3A | 280635220 | A, C | sanfensan | 5C | 593970148 | C, T | sanfensan | 4C | 405897221 | T, C | OT3098 |
| 3A | 289497088 | C, T | sanfensan | 5C | 598395218 | T, C | sanfensan | 4C | 415253727 | C, G | OT3098 |
| 3A | 291617575 | G, A | sanfensan | 5C | 601401624 | C, A | sanfensan | 4C | 501843966 | T, G | OT3098 |
| 3A | 296690651 | T, C | sanfensan | 5D | 4994611 | T, G | sanfensan | 4C | 589184920 | A, T | OT3098 |
| 3A | 303858433 | G, A | sanfensan | 5D | 6622542 | G, C | sanfensan | 4C | 629827712 | C, A | OT3098 |
| 3A | 308390029 | A, G | sanfensan | 5D | 10884878 | C, G | sanfensan | 4C | 646042521 | A, T | OT3098 |
| 3A | 313163176 | C, T | sanfensan | 5D | 16839171 | G, T | sanfensan | 4C | 660379855 | G, A | OT3098 |
| 3A | 315321758 | G, T | sanfensan | 5D | 21309845 | G, A | sanfensan | 4C | 680363280 | G, A | OT3098 |
| 3A | 323449820 | C, T | sanfensan | 5D | 27535507 | T, A | sanfensan | 4C | 690482618 | G, A | OT3098 |
| 3A | 326487847 | G, T | sanfensan | 5D | 34803208 | G, A | sanfensan | 4D | 323386091 | T, C | OT3098 |
| 3A | 334646062 | C, G | sanfensan | 5D | 36778603 | T, G | sanfensan | 4D | 348557099 | G, A | OT3098 |
| 3A | 339385657 | T, G | sanfensan | 5D | 41959313 | T, C | sanfensan | 4D | 377370815 | A, C | OT3098 |
| 3A | 344905994 | C, T | sanfensan | 5D | 47178435 | T, C | sanfensan | 4D | 386553314 | C, T | OT3098 |
| 3A | 346539460 | G, A | sanfensan | 5D | 52127682 | C, T | sanfensan | 4D | 434004607 | A, G | OT3098 |
| 3A | 354806237 | C, T | sanfensan | 5D | 54096343 | T, G | sanfensan | 4D | 455273707 | C, T | OT3098 |
| 3A | 358217253 | C, T | sanfensan | 5D | 56457835 | G, A | sanfensan | 5A | 12250117 | C, T | OT3098 |
| 3A | 363057750 | C, T | sanfensan | 5D | 63243306 | A, C | sanfensan | 5A | 66713969 | G, T | OT3098 |
| 3A | 368774140 | C, T | sanfensan | 5D | 64721781 | C, T | sanfensan | 5A | 163771340 | C, T | OT3098 |
| 3A | 370439246 | C, T | sanfensan | 5D | 65173806 | A, C | sanfensan | 5A | 229347037 | A, G | OT3098 |
| 3A | 379158179 | A, G | sanfensan | 5D | 66436115 | C, T | sanfensan | 5A | 247731935 | C, T | OT3098 |
| 3A | 380572844 | T, C | sanfensan | 5D | 74642065 | T, C | sanfensan | 5A | 259020640 | C, T | OT3098 |
| 3A | 385843902 | C, T | sanfensan | 5D | 79567830 | G, A | sanfensan | 5A | 275554651 | C, T | OT3098 |
| 3A | 393548401 | C, T | sanfensan | 5D | 83382549 | G, A | sanfensan | 5A | 450812604 | G, A | OT3098 |
| 3A | 396511248 | A, G | sanfensan | 5D | 87296427 | T, C | sanfensan | 5A | 464203725 | C, T | OT3098 |
| 3A | 400231496 | G, A | sanfensan | 5D | 93053910 | T, C | sanfensan | 5C | 19684941 | G, A | OT3098 |
| 3A | 407861434 | T, C | sanfensan | 5D | 99261576 | C, T | sanfensan | 5C | 34175263 | G, T | OT3098 |
| 3A | 410745937 | A, G | sanfensan | 5D | 101623688 | C, T | sanfensan | 5C | 56717315 | T, G | OT3098 |
| 3A | 417480860 | A, C | sanfensan | 5D | 109259133 | T, C | sanfensan | 5C | 103289073 | G, T | OT3098 |
| 3C | 4934705 | T, C | sanfensan | 5D | 110187772 | C, T | sanfensan | 5C | 162396640 | T, C | OT3098 |
| 3C | 9789448 | T, C | sanfensan | 5D | 116420976 | T, C | sanfensan | 5C | 276244815 | G, A | OT3098 |
| 3C | 14114292 | A, G | sanfensan | 5D | 120360110 | G, A | sanfensan | 5C | 313968741 | G, A | OT3098 |
| 3C | 16111717 | T, C | sanfensan | 5D | 128589324 | T, C | sanfensan | 5C | 330901610 | A, G | OT3098 |
| 3C | 20060240 | T, C | sanfensan | 5D | 134480766 | G, A | sanfensan | 5C | 491868915 | T, C | OT3098 |
| 3C | 29738599 | G, T | sanfensan | 5D | 136403599 | C, T | sanfensan | 5C | 501340024 | G, A | OT3098 |
| 3C | 34251466 | G, A | sanfensan | 5D | 142232990 | C, T | sanfensan | 5C | 537051000 | T, C | OT3098 |
| 3C | 39605336 | A, C | sanfensan | 5D | 145960007 | A, G | sanfensan | 5C | 567644495 | G, A | OT3098 |
| 3C | 41571752 | T, C | sanfensan | 5D | 153395376 | G, A | sanfensan | 5C | 570184840 | A, C | OT3098 |
| 3C | 48463788 | G, T | sanfensan | 5D | 155066422 | T, C | sanfensan | 5D | 94959246 | G, A | OT3098 |
| 3C | 50619185 | G, A | sanfensan | 5D | 162991627 | T, C | sanfensan | 5D | 100600265 | G, A | OT3098 |
| 3C | 59812660 | G, A | sanfensan | 5D | 167232512 | C, T | sanfensan | 5D | 124321696 | T, C | OT3098 |
| 3C | 63059450 | T, C | sanfensan | 5D | 172000670 | T, C | sanfensan | 5D | 149532876 | G, A | OT3098 |
| 3C | 69756699 | C, T | sanfensan | 5D | 177160582 | G, A | sanfensan | 5D | 193714037 | A, G | OT3098 |
| 3C | 72007306 | T, C | sanfensan | 5D | 182553299 | C, T | sanfensan | 5D | 220790821 | A, G | OT3098 |
| 3C | 75582282 | G, C | sanfensan | 5D | 187357553 | C, T | sanfensan | 5D | 247323372 | T, C | OT3098 |
| 3C | 81453337 | T, C | sanfensan | 5D | 191107356 | A, G | sanfensan | 5D | 341143271 | A, G | OT3098 |
| 3C | 89524634 | T, C | sanfensan | 5D | 199208574 | C, T | sanfensan | 5D | 392150519 | A, C | OT3098 |
| 3C | 94729319 | C, A | sanfensan | 5D | 203029590 | A, G | sanfensan | 5D | 487408616 | A, G | OT3098 |
| 3C | 96052025 | C, T | sanfensan | 5D | 209809131 | T, G | sanfensan | 5D | 496068151 | G, T | OT3098 |
| 3C | 100397477 | A, G | sanfensan | 5D | 211555734 | A, T | sanfensan | 6A | 47261549 | G, C | OT3098 |
| 3C | 108020814 | A, G | sanfensan | 5D | 216140070 | T, G | sanfensan | 6A | 79634441 | C, A | OT3098 |
| 3C | 113507812 | A, C | sanfensan | 5D | 221499002 | A, G | sanfensan | 6A | 90176068 | T, C | OT3098 |
| 3C | 119056248 | C, T | sanfensan | 5D | 228576676 | A, G | sanfensan | 6A | 103858240 | T, C | OT3098 |
| 3C | 121539032 | G, T | sanfensan | 5D | 230582553 | G, C | sanfensan | 6A | 143659802 | T, C | OT3098 |
| 3C | 127564610 | C, T | sanfensan | 5D | 238425107 | C, T | sanfensan | 6A | 159101934 | A, T | OT3098 |
| 3C | 130874626 | G, A | sanfensan | 5D | 242649305 | G, A | sanfensan | 6A | 171391185 | C, A | OT3098 |
| 3C | 137072359 | G, A | sanfensan | 5D | 245087633 | T, C | sanfensan | 6A | 184973728 | A, G | OT3098 |
| 3C | 142064744 | G, A | sanfensan | 5D | 250750865 | G, T | sanfensan | 6A | 192005904 | G, A | OT3098 |
| 3C | 147139993 | T, C | sanfensan | 5D | 258642655 | G, A | sanfensan | 6A | 263939310 | C, T | OT3098 |
| 3C | 152287508 | G, A | sanfensan | 5D | 262006477 | A, G | sanfensan | 6A | 300620942 | A, G | OT3098 |
| 3C | 158231130 | C, T | sanfensan | 5D | 266745740 | G, A | sanfensan | 6A | 419205117 | T, A | OT3098 |
| 3C | 161849544 | C, T | sanfensan | 5D | 271633260 | G, A | sanfensan | 6C | 59229976 | C, T | OT3098 |
| 3C | 167803980 | A, G | sanfensan | 5D | 278837964 | C, T | sanfensan | 6C | 84591374 | C, A | OT3098 |
| 3C | 171441935 | T, C | sanfensan | 5D | 280282084 | T, C | sanfensan | 6C | 87587254 | G, C | OT3098 |
| 3C | 178608408 | T, G | sanfensan | 5D | 289610610 | A, T | sanfensan | 6C | 144536268 | C, A | OT3098 |
| 3C | 180422132 | T, C | sanfensan | 5D | 293820840 | C, T | sanfensan | 6C | 238362770 | C, A | OT3098 |
| 3C | 186181001 | G, A | sanfensan | 5D | 298575083 | A, T | sanfensan | 6C | 246928523 | C, T | OT3098 |
| 3C | 192589566 | T, C | sanfensan | 5D | 302837192 | T, C | sanfensan | 6C | 292396236 | T, C | OT3098 |
| 3C | 195188213 | G, A | sanfensan | 5D | 305502791 | T, C | sanfensan | 6C | 342467150 | A, G | OT3098 |
| 3C | 204778810 | C, T | sanfensan | 5D | 314628778 | A, G | sanfensan | 6C | 366301685 | G, A | OT3098 |
| 3C | 207904801 | T, C | sanfensan | 5D | 319756146 | G, A | sanfensan | 6C | 467269710 | C, T | OT3098 |
| 3C | 214065619 | T, G | sanfensan | 5D | 322226424 | C, A | sanfensan | 6C | 485083612 | C, T | OT3098 |
| 3C | 217226719 | T, C | sanfensan | 5D | 328035262 | C, T | sanfensan | 6C | 574716448 | C, T | OT3098 |
| 3C | 221780018 | G, C | sanfensan | 5D | 332326959 | C, T | sanfensan | 6C | 602967220 | T, C | OT3098 |
| 3C | 228014218 | G, A | sanfensan | 5D | 339720781 | T, A | sanfensan | 6C | 611213471 | T, C | OT3098 |
| 3C | 233526022 | G, A | sanfensan | 5D | 344363610 | A, C | sanfensan | 6D | 67717241 | G, A | OT3098 |
| 3C | 235353093 | A, C | sanfensan | 5D | 345547743 | T, C | sanfensan | 6D | 87582596 | A, G | OT3098 |
| 3C | 244162351 | G, A | sanfensan | 5D | 353592498 | G, A | sanfensan | 6D | 130251011 | A, G | OT3098 |
| 3C | 248122047 | C, T | sanfensan | 5D | 357027031 | C, T | sanfensan | 6D | 143238944 | C, T | OT3098 |
| 3C | 253823594 | C, T | sanfensan | 5D | 363095927 | T, G | sanfensan | 6D | 161653963 | A, G | OT3098 |
| 3C | 259462643 | A, G | sanfensan | 5D | 365082132 | T, C | sanfensan | 6D | 193391379 | C, T | OT3098 |
| 3C | 260199510 | T, C | sanfensan | 5D | 372087578 | A, C | sanfensan | 6D | 202580655 | G, A | OT3098 |
| 3C | 269465277 | G, T | sanfensan | 5D | 375075448 | A, G | sanfensan | 6D | 240523148 | G, C | OT3098 |
| 3C | 274931211 | G, A | sanfensan | 5D | 382109948 | T, C | sanfensan | 6D | 256237708 | G, T | OT3098 |
| 3C | 277523171 | C, A | sanfensan | 5D | 385044778 | A, G | sanfensan | 6D | 265237433 | T, C | OT3098 |
| 3C | 284109639 | G, C | sanfensan | 5D | 394858589 | C, T | sanfensan | 6D | 283670697 | G, A | OT3098 |
| 3C | 285346116 | A, G | sanfensan | 5D | 399910298 | C, T | sanfensan | 7A | 6894726 | A, G | OT3098 |
| 3C | 294939142 | A, C | sanfensan | 5D | 402227007 | G, A | sanfensan | 7A | 93976095 | A, C | OT3098 |
| 3C | 296525393 | A, G | sanfensan | 5D | 407502206 | T, C | sanfensan | 7A | 212399895 | C, G | OT3098 |
| 3C | 304406734 | T, C | sanfensan | 5D | 410635861 | A, G | sanfensan | 7A | 226861026 | A, C | OT3098 |
| 3C | 309329884 | C, T | sanfensan | 5D | 419664992 | T, C | sanfensan | 7A | 295965773 | A, G | OT3098 |
| 3C | 310447021 | T, G | sanfensan | 5D | 421256053 | A, G | sanfensan | 7A | 423933210 | T, C | OT3098 |
| 3C | 319961145 | T, C | sanfensan | 5D | 427332097 | C, G | sanfensan | 7A | 440302050 | T, C | OT3098 |
| 3C | 321458383 | T, C | sanfensan | 5D | 433626893 | G, T | sanfensan | 7A | 457458508 | A, G | OT3098 |
| 3C | 325121366 | A, G | sanfensan | 5D | 437055110 | C, G | sanfensan | 7A | 488323283 | A, G | OT3098 |
| 3C | 330206369 | A, G | sanfensan | 5D | 440529761 | C, T | sanfensan | 7C | 2133869 | C, T | OT3098 |
| 3C | 332130807 | C, T | sanfensan | 5D | 446386566 | C, T | sanfensan | 7C | 6687935 | A, G | OT3098 |
| 3C | 338543178 | T, C | sanfensan | 5D | 454896966 | T, C | sanfensan | 7C | 40731489 | G, A | OT3098 |
| 3C | 343287383 | T, C | sanfensan | 5D | 458673318 | G, A | sanfensan | 7C | 135716079 | C, A | OT3098 |
| 3C | 349175645 | T, C | sanfensan | 5D | 462992703 | G, A | sanfensan | 7C | 192742152 | C, T | OT3098 |
| 3C | 353910191 | C, T | sanfensan | 5D | 466900163 | C, T | sanfensan | 7C | 213883268 | A, G | OT3098 |
| 3C | 358917955 | T, C | sanfensan | 5D | 474924404 | C, T | sanfensan | 7C | 225224321 | T, C | OT3098 |
| 3C | 363942089 | G, A | sanfensan | 5D | 478407877 | T, C | sanfensan | 7C | 271932318 | G, A | OT3098 |
| 3C | 366028325 | A, C | sanfensan | 5D | 482793426 | T, C | sanfensan | 7C | 303692756 | A, G | OT3098 |
| 3C | 372945120 | C, T | sanfensan | 5D | 488236543 | A, C | sanfensan | 7C | 314575954 | T, C | OT3098 |
| 3C | 375489817 | C, G | sanfensan | 5D | 492418351 | A, G | sanfensan | 7C | 433548395 | T, C | OT3098 |
| 3C | 384457117 | A, G | sanfensan | 5D | 497166345 | G, T | sanfensan | 7C | 461822950 | C, T | OT3098 |
| 3C | 388958566 | A, G | sanfensan | 5D | 500430897 | T, G | sanfensan | 7C | 478807736 | A, G | OT3098 |
| 3C | 394935493 | A, T | sanfensan | 6A | 4492157 | T, C | sanfensan | 7D | 43006316 | G, A | OT3098 |
| 3C | 395752355 | G, A | sanfensan | 6A | 8762221 | G, T | sanfensan | 7D | 53808953 | G, A | OT3098 |
| 3C | 403204424 | G, A | sanfensan | 6A | 11190798 | T, C | sanfensan | 7D | 72525182 | T, A | OT3098 |
| 3C | 408346907 | A, C | sanfensan | 6A | 17691406 | C, T | sanfensan | 7D | 100996318 | C, T | OT3098 |
| 3C | 412555939 | T, C | sanfensan | 6A | 20852081 | T, C | sanfensan | 7D | 119049740 | A, G | OT3098 |
| 3C | 418959428 | A, G | sanfensan | 6A | 26524896 | C, T | sanfensan | 7D | 123197785 | C, T | OT3098 |
| 3C | 424637971 | G, A | sanfensan | 6A | 33584644 | C, T | sanfensan | 7D | 131225276 | A, G | OT3098 |
| 3C | 425426160 | G, A | sanfensan | 6A | 36710498 | A, G | sanfensan | 7D | 196173654 | T, G | OT3098 |
| 3C | 431869178 | A, C | sanfensan | 6A | 42587073 | A, G | sanfensan | 7D | 227132197 | T, C | OT3098 |
| 3C | 437116797 | T, G | sanfensan | 6A | 48196250 | G, C | sanfensan | 7D | 237053803 | T, G | OT3098 |
| 3C | 442997546 | A, G | sanfensan | 6A | 54285408 | C, G | sanfensan | 7D | 248735393 | T, C | OT3098 |
| 3C | 445451564 | C, A | sanfensan | 6A | 57328665 | T, A | sanfensan | 7D | 256172681 | C, T | OT3098 |
| 3C | 449848039 | C, T | sanfensan | 6A | 64386015 | C, A | sanfensan | 7D | 291312060 | C, T | OT3098 |
| 3C | 450156513 | C, T | sanfensan | 6A | 67285702 | C, T | sanfensan | 7D | 363340392 | T, C | OT3098 |
| 3C | 458930099 | T, C | sanfensan | 6A | 71815074 | C, A | sanfensan | 7D | 370273697 | A, G | OT3098 |
| 3C | 460938762 | C, T | sanfensan | 6A | 77438434 | C, A | sanfensan | 7D | 382112487 | G, A | OT3098 |
| 3C | 465773933 | A, G | sanfensan | 6A | 83659654 | G, A | sanfensan | 7D | 391806334 | A, G | OT3098 |
| 3C | 466127904 | C, A | sanfensan | 6A | 85235001 | C, A | sanfensan | 7D | 416042498 | C, T | OT3098 |
| 3C | 472480324 | G, T | sanfensan | 6A | 94561841 | T, G | sanfensan | 7D | 430774655 | T, C | OT3098 |
| 3C | 476852155 | T, C | sanfensan | 6A | 95323662 | A, C | sanfensan | 7D | 466485714 | C, T | OT3098 |
| 3C | 482946601 | C, T | sanfensan | 6A | 103614490 | T, C | sanfensan | | | | |

2. An application of an SNP loci combination in preparing 2K SNP liquid chip for Avena sativa variety identifying, wherein the SNP loci combination comprises SNP loci located in reference genomes sanfensan and OT3098 as shown in a following table:

| Chromosome | Physical location | Allelic variation | Reference genome |
|---|---|---|---|
| 1A | 4473517 | C, A | sanfensan |
| 1A | 9349344 | G, A | sanfensan |
| 1A | 14898617 | C, T | sanfensan |
| 1A | 19788850 | T, C | sanfensan |
| 1A | 24134605 | A, C | sanfensan |
| 1A | 25204308 | G, A | sanfensan |
| 1A | 34911838 | T, A | sanfensan |
| 1A | 35091182 | A, G | sanfensan |
| 1A | 39926834 | G, A | sanfensan |
| 1A | 40243841 | C, A | sanfensan |
| 1A | 46676991 | T, C | sanfensan |
| 1A | 50477703 | C, T | sanfensan |
| 1A | 55467045 | T, C | sanfensan |
| 1A | 61641698 | C, A | sanfensan |
| 1A | 65935052 | C, A | sanfensan |
| 1A | 71753358 | C, T | sanfensan |
| 1A | 76746486 | T, C | sanfensan |
| 1A | 82946413 | T, C | sanfensan |
| 1A | 85796042 | G, A | sanfensan |
| 1A | 92427340 | G, A | sanfensan |
| 1A | 98303200 | A, C | sanfensan |
| 1A | 100886423 | T, C | sanfensan |
| 1A | 105665342 | A, G | sanfensan |
| 1A | 106585584 | T, C | sanfensan |
| 1A | 110857330 | A, C | sanfensan |
| 1A | 119266034 | T, C | sanfensan |
| 1A | 120319599 | T, G | sanfensan |
| 1A | 128479703 | G, A | sanfensan |
| 1A | 131129360 | A, G | sanfensan |
| 1A | 135686787 | G, A | sanfensan |
| 1A | 140893664 | A, G | sanfensan |
| 1A | 149729422 | A, G | sanfensan |
| 1A | 154018712 | A, G | sanfensan |
| 1A | 156986726 | T, C | sanfensan |
| 1A | 160810612 | G, C | sanfensan |
| 1A | 168045464 | T, G | sanfensan |
| 1A | 174968050 | A, G | sanfensan |
| 1A | 177054360 | C, T | sanfensan |
| 1A | 184641750 | G, A | sanfensan |
| 1A | 186347220 | A, G | sanfensan |
| 1A | 190161955 | T, C | sanfensan |
| 1A | 197891296 | A, G | sanfensan |
| 1A | 202332235 | G, C | sanfensan |
| 1A | 206929081 | A, G | sanfensan |
| 1A | 213307997 | A, C | sanfensan |
| 1A | 215902606 | A, C | sanfensan |
| 1A | 223276058 | G, T | sanfensan |
| 1A | 225939090 | G, A | sanfensan |
| 1A | 229684564 | T, C | sanfensan |
| 1A | 230432457 | A, G | sanfensan |
| 1A | 239890900 | C, T | sanfensan |
| 1A | 243567990 | G, A | sanfensan |
| 1A | 245948969 | G, A | sanfensan |
| 1A | 250969537 | G, C | sanfensan |
| 1A | 257803247 | T, G | sanfensan |
| 1A | 263812100 | A, G | sanfensan |
| 1A | 268553550 | T, C | sanfensan |
| 1A | 271645348 | T, G | sanfensan |
| 1A | 283816950 | G, A | sanfensan |
| 1A | 286799892 | T, C | sanfensan |
| 1A | 292498165 | C, A | sanfensan |
| 1A | 298383411 | A, G | sanfensan |
| 1A | 300811687 | A, G | sanfensan |
| 1A | 307060043 | T, G | sanfensan |
| 1A | 314835969 | G, A | sanfensan |
| 1A | 315910953 | A, G | sanfensan |
| 1A | 321206313 | T, G | sanfensan |
| 1A | 329104188 | A, C | sanfensan |
| 1A | 333516925 | G, T | sanfensan |
| 1A | 335522334 | G, A | sanfensan |
| 1A | 342234031 | G, A | sanfensan |
| 1A | 348750824 | C, A | sanfensan |
| 1A | 354606484 | C, T | sanfensan |
| 1A | 355044825 | G, A | sanfensan |
| 1A | 361899430 | G, A | sanfensan |
| 1A | 365228574 | T, G | sanfensan |
| 1A | 370165053 | A, G | sanfensan |
| 1A | 371882871 | T, C | sanfensan |
| 1A | 379396033 | A, C | sanfensan |
| 1A | 380814167 | C, T | sanfensan |
| 1A | 385881054 | T, G | sanfensan |
| 1A | 394799443 | C, G | sanfensan |
| 1A | 396233056 | A, T | sanfensan |
| 1A | 400131929 | A, G | sanfensan |
| 1A | 408599208 | C, T | sanfensan |
| 1A | 410583439 | T, C | sanfensan |
| 1A | 417767787 | G, A | sanfensan |
| 1A | 423934932 | G, T | sanfensan |
| 1A | 427534714 | A, G | sanfensan |
| 1A | 430802770 | T, C | sanfensan |
| 1A | 439615284 | A, G | sanfensan |
| 1A | 443388417 | C, T | sanfensan |
| 1A | 447639906 | G, A | sanfensan |
| 1A | 453204191 | T, C | sanfensan |
| 1A | 457075413 | G, A | sanfensan |
| 1A | 463117262 | G, A | sanfensan |
| 1A | 465118330 | G, T | sanfensan |
| 1A | 472500626 | T, C | sanfensan |
| 1A | 475569735 | T, G | sanfensan |
| 1A | 481563062 | G, A | sanfensan |
| 1A | 486246321 | T, C | sanfensan |
| 1A | 490249212 | G, A | sanfensan |
| 1A | 495026085 | C, T | sanfensan |
| 1A | 500157268 | G, A | sanfensan |
| 1A | 505420346 | T, C | sanfensan |
| 1A | 513237629 | C, A | sanfensan |
| 1A | 516943173 | A, G | sanfensan |
| 1A | 520725745 | C, G | sanfensan |
| 1A | 529117339 | C, T | sanfensan |
| 1A | 532148235 | T, C | sanfensan |
| 1A | 538845292 | C, T | sanfensan |
| 1A | 540384422 | G, C | sanfensan |
| 1A | 546023901 | C, G | sanfensan |
| 1A | 551040971 | C, T | sanfensan |
| 1C | 4866196 | A, C | sanfensan |
| 1C | 8009186 | G, A | sanfensan |
| 1C | 12114531 | T, C | sanfensan |
| 1C | 17916692 | G, A | sanfensan |
| 1C | 22093157 | T, C | sanfensan |
| 1C | 25463149 | C, T | sanfensan |
| 1C | 32954922 | G, T | sanfensan |
| 1C | 38255313 | G, A | sanfensan |
| 1C | 40225359 | C, T | sanfensan |
| 1C | 47044975 | C, T | sanfensan |
| 1C | 54713053 | C, T | sanfensan |
| 1C | 57342838 | G, T | sanfensan |
| 1C | 64447286 | C, T | sanfensan |
| 1C | 68342251 | C, G | sanfensan |
| 1C | 73736931 | T, C | sanfensan |
| 1C | 75426247 | G, A | sanfensan |
| 1C | 81261715 | C, T | sanfensan |
| 1C | 89052462 | A, C | sanfensan |
| 1C | 93949401 | C, T | sanfensan |
| 1C | 97665480 | T, A | sanfensan |
| 1C | 104414265 | C, T | sanfensan |
| 1C | 107889124 | C, T | sanfensan |
| 1C | 112840945 | A, G | sanfensan |
| 1C | 117607567 | C, A | sanfensan |
| 1C | 123766670 | G, C | sanfensan |
| 1C | 124698034 | C, T | sanfensan |
| 1C | 125624389 | G, A | sanfensan |
| 1C | 132272583 | G, T | sanfensan |
| 1C | 137221231 | G, A | sanfensan |
| 1C | 140336177 | G, A | sanfensan |
| 1C | 148863724 | C, T | sanfensan |
| 1C | 152576285 | T, G | sanfensan |
| 1C | 158557656 | G, C | sanfensan |
| 1C | 161866271 | C, A | sanfensan |
| 1C | 166850618 | G, A | sanfensan |
| 1C | 171246538 | C, A | sanfensan |
| 1C | 177121539 | G, A | sanfensan |
| 1C | 184861598 | C, T | sanfensan |
| 1C | 187896025 | C, A | sanfensan |
| 1C | 192704844 | C, T | sanfensan |
| 1C | 196342850 | G, A | sanfensan |
| 1C | 203152613 | A, G | sanfensan |
| 1C | 206698681 | G, A | sanfensan |
| 1C | 212808498 | G, A | sanfensan |
| 1C | 219911347 | A, G | sanfensan |
| 1C | 223588927 | G, A | sanfensan |
| 1C | 228818861 | G, T | sanfensan |
| 1C | 231062523 | C, T | sanfensan |
| 1C | 244308309 | G, A | sanfensan |
| 1C | 244949293 | G, C | sanfensan |
| 1C | 247382592 | G, A | sanfensan |
| 1C | 251563472 | C, G | sanfensan |
| 1C | 258583411 | G, C | sanfensan |
| 1C | 264870633 | C, T | sanfensan |
| 1C | 269866051 | G, A | sanfensan |
| 1C | 270041152 | T, C | sanfensan |
| 1C | 279714245 | T, C | sanfensan |
| 1C | 281426354 | G, A | sanfensan |
| 1C | 289269367 | C, G | sanfensan |
| 1C | 290192142 | C, A | sanfensan |
| 1C | 295651800 | G, T | sanfensan |
| 1C | 301166258 | G, C | sanfensan |
| 1C | 305159269 | G, T | sanfensan |
| 1C | 311692892 | C, T | sanfensan |
| 1C | 318201468 | T, G | sanfensan |
| 1C | 322797850 | G, A | sanfensan |
| 1C | 329636395 | C, T | sanfensan |
| 1C | 331069160 | G, A | sanfensan |
| 1C | 338302628 | G, A | sanfensan |
| 1C | 343573672 | A, G | sanfensan |
| 1C | 346562089 | G, A | sanfensan |
| 1C | 350268981 | C, T | sanfensan |
| 1C | 359261478 | T, C | sanfensan |
| 1C | 361793363 | G, A | sanfensan |
| 1C | 366795285 | C, G | sanfensan |
| 1C | 373361346 | G, A | sanfensan |
| 1C | 377285872 | C, T | sanfensan |
| 1C | 384514418 | T, G | sanfensan |
| 1C | 388701553 | G, A | sanfensan |
| 1C | 391109953 | C, T | sanfensan |
| 1C | 398781194 | A, C | sanfensan |
| 1C | 404904380 | A, C | sanfensan |
| 1C | 409167422 | A, G | sanfensan |
| 1C | 410239696 | G, A | sanfensan |
| 1C | 418466563 | A, G | sanfensan |
| 1C | 424408198 | C, T | sanfensan |
| 1C | 426142267 | T, C | sanfensan |
| 1C | 434346817 | G, A | sanfensan |
| 1C | 435126767 | A, G | sanfensan |
| 1C | 444951853 | C, T | sanfensan |
| 1C | 446632589 | T, C | sanfensan |
| 1C | 451346048 | G, A | sanfensan |
| 1C | 458628576 | C, T | sanfensan |
| 1D | 4748051 | C, T | sanfensan |
| 1D | 9972757 | C, T | sanfensan |
| 1D | 14428103 | T, C | sanfensan |
| 1D | 17910637 | T, G | sanfensan |
| 1D | 22886105 | A, C | sanfensan |
| 1D | 26189931 | T, C | sanfensan |
| 1D | 34096819 | T, G | sanfensan |
| 1D | 36812285 | T, C | sanfensan |
| 1D | 44167411 | T, C | sanfensan |
| 1D | 48753097 | C, T | sanfensan |
| 1D | 52943769 | G, A | sanfensan |
| 1D | 59943693 | C, T | sanfensan |
| 1D | 61299591 | T, A | sanfensan |
| 1D | 67828154 | C, T | sanfensan |
| 1D | 74812678 | T, C | sanfensan |
| 1D | 76558850 | A, G | sanfensan |
| 1D | 83489940 | C, T | sanfensan |
| 1D | 85424309 | G, A | sanfensan |
| 1D | 90502825 | G, T | sanfensan |
| 1D | 99173014 | C, A | sanfensan |
| 1D | 101866462 | A, G | sanfensan |
| 1D | 105176621 | G, T | sanfensan |
| 1D | 112466846 | A, G | sanfensan |
| 1D | 116735117 | T, C | sanfensan |
| 1D | 120724766 | T, G | sanfensan |
| 1D | 125083241 | A, G | sanfensan |
| 1D | 134949445 | T, C | sanfensan |
| 1D | 136722735 | T, C | sanfensan |
| 1D | 140036387 | C, T | sanfensan |
| 1D | 149197821 | G, A | sanfensan |
| 1D | 154586415 | G, T | sanfensan |
| 1D | 156993466 | T, G | sanfensan |
| 1D | 160591807 | T, G | sanfensan |
| 1D | 169373396 | G, A | sanfensan |
| 1D | 172161852 | G, T | sanfensan |
| 1D | 178558164 | T, C | sanfensan |
| 1D | 182138470 | A, G | sanfensan |
| 1D | 188332946 | T, C | sanfensan |
| 1D | 191338972 | T, C | sanfensan |
| 1D | 197805002 | G, A | sanfensan |
| 1D | 201653641 | A, C | sanfensan |
| 1D | 205231760 | T, C | sanfensan |
| 1D | 213849974 | A, G | sanfensan |
| 1D | 218342762 | G, C | sanfensan |
| 1D | 221295907 | A, C | sanfensan |
| 1D | 225627642 | A, G | sanfensan |
| 1D | 233309591 | C, T | sanfensan |
| 1D | 234707599 | G, A | sanfensan |
| 1D | 239408154 | C, T | sanfensan |
| 1D | 241284677 | T, G | sanfensan |
| 1D | 248921833 | T, G | sanfensan |
| 1D | 250623142 | A, T | sanfensan |
| 1D | 258844136 | C, T | sanfensan |
| 1D | 262320123 | A, C | sanfensan |
| 1D | 265785336 | T, C | sanfensan |
| 1D | 270470362 | C, T | sanfensan |
| 1D | 275515735 | A, G | sanfensan |
| 1D | 284647168 | G, A | sanfensan |
| 1D | 288750391 | A, G | sanfensan |
| 1D | 293777596 | A, G | sanfensan |
| 1D | 298973779 | C, A | sanfensan |
| 1D | 304978046 | T, C | sanfensan |
| 1D | 306430734 | T, G | sanfensan |
| 1D | 313322569 | T, C | sanfensan |
| 1D | 317482376 | T, C | sanfensan |
| 1D | 321285642 | T, C | sanfensan |
| 1D | 328096417 | A, G | sanfensan |
| 1D | 334194238 | G, A | sanfensan |
| 1D | 336604011 | G, A | sanfensan |
| 1D | 342605207 | A, C | sanfensan |
| 1D | 348951141 | A, C | sanfensan |
| 1D | 352790704 | A, G | sanfensan |
| 1D | 355455291 | G, C | sanfensan |
| 1D | 364550878 | A, G | sanfensan |
| 1D | 369719851 | A, G | sanfensan |
| 1D | 372379876 | A, C | sanfensan |
| 1D | 379801584 | A, C | sanfensan |
| 1D | 380728291 | A, G | sanfensan |
| 1D | 388559569 | T, C | sanfensan |
| 1D | 394958911 | T, C | sanfensan |
| 1D | 395282752 | A, G | sanfensan |
| 1D | 403815045 | T, C | sanfensan |
| 1D | 409759489 | T, C | sanfensan |
| 1D | 412133901 | T, C | sanfensan |
| 1D | 418421166 | C, G | sanfensan |
| 1D | 423994932 | T, C | sanfensan |
| 1D | 429511063 | A, G | sanfensan |
| 1D | 433441263 | A, G | sanfensan |
| 1D | 435135220 | T, A | sanfensan |
| 1D | 442087046 | G, A | sanfensan |
| 1D | 442124461 | G, A | sanfensan |
| 1D | 449745797 | G, C | sanfensan |
| 1D | 450598659 | A, G | sanfensan |
| 1D | 459581711 | T, C | sanfensan |
| 1D | 460387603 | C, G | sanfensan |
| 1D | 468646779 | G, A | sanfensan |
| 1D | 470605204 | C, T | sanfensan |
| 2A | 1294852 | A, G | sanfensan |
| 2A | 8354341 | G, A | sanfensan |
| 2A | 10205647 | T, G | sanfensan |
| 2A | 17133037 | A, G | sanfensan |
| 2A | 22147985 | C, G | sanfensan |
| 2A | 25577776 | C, A | sanfensan |
| 2A | 33113184 | G, T | sanfensan |
| 2A | 38311226 | G, A | sanfensan |
| 2A | 43688833 | A, C | sanfensan |
| 2A | 45363179 | C, T | sanfensan |
| 2A | 54582062 | A, G | sanfensan |
| 2A | 58712383 | A, G | sanfensan |
| 2A | 60354747 | A, G | sanfensan |
| 2A | 69920614 | A, G | sanfensan |
| 2A | 74714222 | T, C | sanfensan |
| 2A | 78592686 | A, C | sanfensan |
| 2A | 79665300 | C, A | sanfensan |
| 2A | 83491335 | A, G | sanfensan |
| 2A | 88992475 | A, C | sanfensan |
| 2A | 91196574 | C, T | sanfensan |
| 2A | 98762331 | C, G | sanfensan |
| 2A | 102839151 | T, C | sanfensan |
| 2A | 105115673 | C, T | sanfensan |
| 2A | 113143954 | G, A | sanfensan |
| 2A | 115778719 | C, T | sanfensan |
| 2A | 123726669 | G, C | sanfensan |
| 2A | 125030830 | G, A | sanfensan |
| 2A | 133605969 | C, A | sanfensan |
| 2A | 136067584 | A, C | sanfensan |
| 2A | 142891390 | C, T | sanfensan |
| 2A | 148925271 | C, T | sanfensan |
| 2A | 150242064 | A, G | sanfensan |
| 2A | 159722254 | T, C | sanfensan |
| 2A | 161633504 | G, A | sanfensan |
| 2A | 166801852 | G, A | sanfensan |
| 2A | 172884455 | G, C | sanfensan |
| 2A | 177892881 | A, G | sanfensan |
| 2A | 180161463 | A, C | sanfensan |
| 2A | 187191668 | G, A | sanfensan |
| 2A | 194352120 | C, A | sanfensan |
| 2A | 195938867 | T, C | sanfensan |
| 2A | 202623332 | G, T | sanfensan |
| 2A | 209957751 | G, T | sanfensan |
| 2A | 210875267 | T, G | sanfensan |
| 2A | 215784372 | C, T | sanfensan |
| 2A | 224379638 | G, C | sanfensan |
| 2A | 227479068 | C, T | sanfensan |
| 2A | 231405711 | A, C | sanfensan |
| 2A | 237629147 | C, T | sanfensan |
| 2A | 240260139 | T, C | sanfensan |
| 2A | 248913514 | G, C | sanfensan |
| 2A | 251101436 | C, T | sanfensan |
| 2A | 258858124 | C, T | sanfensan |
| 2A | 262168685 | G, A | sanfensan |
| 2A | 269432173 | A, C | sanfensan |
| 2A | 272066634 | T, C | sanfensan |
| 2A | 275191428 | C, T | sanfensan |
| 2A | 282263456 | T, G | sanfensan |
| 2A | 288156444 | C, G | sanfensan |
| 2A | 291947063 | A, C | sanfensan |
| 2A | 298698676 | T, C | sanfensan |
| 2A | 302165782 | G, A | sanfensan |
| 2A | 307413298 | T, C | sanfensan |
| 2A | 314041736 | C, G | sanfensan |
| 2A | 319115852 | C, T | sanfensan |
| 2A | 324309925 | C, T | sanfensan |
| 2A | 325423488 | A, C | sanfensan |
| 2A | 332013465 | T, C | sanfensan |
| 2A | 336329704 | T, G | sanfensan |
| 2A | 337567230 | G, T | sanfensan |
| 2A | 340385583 | C, T | sanfensan |
| 2A | 347331178 | A, G | sanfensan |
| 2A | 354110393 | C, T | sanfensan |
| 2A | 355224874 | C, T | sanfensan |
| 2A | 361450337 | A, G | sanfensan |
| 2A | 365973983 | T, C | sanfensan |
| 2A | 374152559 | T, C | sanfensan |
| 2A | 378774926 | A, G | sanfensan |
| 2A | 380652237 | C, T | sanfensan |
| 2A | 386433427 | G, A | sanfensan |
| 2A | 391759319 | C, T | sanfensan |
| 2A | 397676516 | C, G | sanfensan |
| 2A | 404431299 | T, C | sanfensan |
| 2A | 406323117 | T, C | sanfensan |
| 2A | 414368443 | A, G | sanfensan |
| 2A | 418701849 | C, A | sanfensan |
| 2A | 421611597 | T, C | sanfensan |
| 2A | 429392443 | C, A | sanfensan |
| 2A | 432088076 | C, A | sanfensan |
| 2A | 432433952 | T, G | sanfensan |
| 2A | 437058043 | T, C | sanfensan |
| 2A | 442675069 | G, C | sanfensan |
| 2A | 447978464 | A, G | sanfensan |
| 2A | 452816108 | C, A | sanfensan |
| 2A | 456332224 | T, C | sanfensan |
| 2A | 460704136 | T, C | sanfensan |
| 2C | 3755642 | C, G | sanfensan |
| 2C | 6523209 | T, C | sanfensan |
| 2C | 14666414 | G, A | sanfensan |
| 2C | 18219538 | G, A | sanfensan |
| 2C | 20598121 | C, T | sanfensan |
| 2C | 25528699 | G, A | sanfensan |
| 2C | 34351879 | G, A | sanfensan |
| 2C | 37480085 | G, A | sanfensan |
| 2C | 40282520 | C, T | sanfensan |
| 2C | 47255505 | G, A | sanfensan |
| 2C | 52177917 | C, T | sanfensan |
| 2C | 55006635 | C, A | sanfensan |
| 2C | 62911833 | A, C | sanfensan |
| 2C | 69182434 | C, T | sanfensan |
| 2C | 73235690 | G, A | sanfensan |
| 2C | 75663383 | C, T | sanfensan |
| 2C | 80698781 | C, T | sanfensan |
| 2C | 88439094 | G, C | sanfensan |
| 2C | 93854384 | C, T | sanfensan |
| 2C | 97362350 | G, A | sanfensan |
| 2C | 104745256 | C, T | sanfensan |
| 2C | 108205218 | C, T | sanfensan |
| 2C | 110557690 | G, A | sanfensan |
| 2C | 119588975 | C, T | sanfensan |
| 2C | 122248235 | C, T | sanfensan |
| 2C | 129849765 | C, T | sanfensan |
| 2C | 134962790 | G, A | sanfensan |
| 2C | 135013067 | T, G | sanfensan |
| 2C | 140602512 | C, T | sanfensan |
| 2C | 145478940 | T, C | sanfensan |
| 2C | 150692118 | G, A | sanfensan |
| 2C | 156314693 | G, A | sanfensan |
| 2C | 160485851 | C, T | sanfensan |
| 2C | 167462260 | G, A | sanfensan |
| 2C | 173642372 | C, T | sanfensan |
| 2C | 179174429 | C, A | sanfensan |
| 2C | 182456426 | G, A | sanfensan |
| 2C | 188223122 | C, T | sanfensan |
| 2C | 190626883 | C, T | sanfensan |
| 2C | 196959827 | G, A | sanfensan |
| 2C | 201765496 | G, T | sanfensan |
| 2C | 207043151 | C, T | sanfensan |
| 2C | 208215965 | C, T | sanfensan |
| 2C | 213490479 | G, A | sanfensan |
| 2C | 217643681 | G, A | sanfensan |
| 2C | 222136297 | G, A | sanfensan |
| 2C | 226477864 | C, T | sanfensan |
| 2C | 231505039 | C, T | sanfensan |
| 2C | 237675775 | C, T | sanfensan |
| 2C | 240667277 | G, A | sanfensan |
| 2C | 246770167 | A, C | sanfensan |
| 2C | 252643615 | G, A | sanfensan |
| 2C | 256169319 | G, A | sanfensan |
| 2C | 261833087 | G, A | sanfensan |
| 2C | 262787175 | A, T | sanfensan |
| 2C | 267745937 | G, A | sanfensan |
| 2C | 271735685 | G, A | sanfensan |
| 2C | 277919032 | G, A | sanfensan |
| 2C | 281899341 | C, T | sanfensan |
| 2C | 287576313 | C, T | sanfensan |
| 2C | 294884196 | G, A | sanfensan |
| 2C | 298708412 | T, A | sanfensan |
| 2C | 304070232 | A, G | sanfensan |
| 2C | 305356521 | G, T | sanfensan |
| 2C | 312681417 | G, A | sanfensan |
| 2C | 317317802 | T, C | sanfensan |
| 2C | 324693110 | G, T | sanfensan |
| 2C | 329639427 | C, G | sanfensan |
| 2C | 334559533 | C, A | sanfensan |
| 2C | 335512805 | C, T | sanfensan |
| 2C | 342249614 | G, A | sanfensan |
| 2C | 349065985 | C, T | sanfensan |
| 2C | 354184855 | C, T | sanfensan |
| 2C | 358792181 | C, T | sanfensan |
| 2C | 361602927 | A, C | sanfensan |
| 2C | 365574905 | T, C | sanfensan |
| 2C | 372578823 | A, T | sanfensan |
| 2C | 377682611 | C, T | sanfensan |
| 2C | 383773493 | G, A | sanfensan |
| 2C | 388264545 | G, T | sanfensan |
| 2C | 392647080 | T, A | sanfensan |
| 2C | 399910470 | G, A | sanfensan |
| 2C | 402251417 | T, G | sanfensan |
| 2C | 405239508 | A, G | sanfensan |
| 2C | 412396338 | A, G | sanfensan |
| 2C | 416260936 | G, A | sanfensan |
| 2C | 423413408 | G, A | sanfensan |
| 2C | 429152113 | C, T | sanfensan |
| 2C | 433743437 | G, A | sanfensan |
| 2C | 438694947 | G, A | sanfensan |
| 2C | 441372926 | G, A | sanfensan |
| 2C | 446094456 | T, G | sanfensan |
| 2C | 451391070 | A, G | sanfensan |
| 2C | 456022723 | A, C | sanfensan |
| 2C | 462451470 | G, A | sanfensan |
| 2C | 469973109 | G, A | sanfensan |
| 2C | 473490736 | G, A | sanfensan |
| 2C | 476541629 | C, T | sanfensan |
| 2C | 482708999 | T, G | sanfensan |
| 2C | 488486085 | C, G | sanfensan |
| 2C | 492299240 | T, G | sanfensan |
| 2C | 497502793 | C, T | sanfensan |
| 2C | 504463768 | G, A | sanfensan |
| 2C | 506288307 | G, A | sanfensan |
| 2C | 511067660 | T, C | sanfensan |
| 2C | 515199747 | G, T | sanfensan |
| 2C | 520028290 | C, G | sanfensan |
| 2C | 526639651 | C, T | sanfensan |
| 2C | 530779807 | G, A | sanfensan |
| 2C | 537336195 | A, G | sanfensan |
| 2C | 540940586 | T, C | sanfensan |
| 2C | 547202871 | G, A | sanfensan |
| 2C | 552060518 | G, C | sanfensan |
| 2D | 4090001 | G, A | sanfensan |
| 2D | 8884387 | G, A | sanfensan |
| 2D | 13578443 | C, G | sanfensan |
| 2D | 17675396 | A, G | sanfensan |
| 2D | 23696567 | G, C | sanfensan |
| 2D | 29926609 | C, A | sanfensan |
| 2D | 31795322 | C, G | sanfensan |
| 2D | 38891629 | C, T | sanfensan |
| 2D | 43755471 | A, C | sanfensan |
| 2D | 49839859 | T, C | sanfensan |
| 2D | 52853593 | T, G | sanfensan |
| 2D | 59369523 | A, G | sanfensan |
| 2D | 64844533 | C, T | sanfensan |
| 2D | 67973342 | G, C | sanfensan |
| 2D | 72762945 | G, T | sanfensan |
| 2D | 77924703 | G, A | sanfensan |
| 2D | 82246788 | C, G | sanfensan |
| 2D | 85545627 | A, G | sanfensan |
| 2D | 93391163 | T, C | sanfensan |
| 2D | 97511713 | T, C | sanfensan |
| 2D | 103508833 | A, G | sanfensan |
| 2D | 105773491 | C, T | sanfensan |
| 2D | 112039326 | T, C | sanfensan |
| 2D | 118313919 | A, G | sanfensan |
| 2D | 124707738 | G, A | sanfensan |
| 2D | 127418722 | T, C | sanfensan |
| 2D | 134051671 | A, G | sanfensan |
| 2D | 139923233 | T, C | sanfensan |
| 2D | 141313127 | G, T | sanfensan |
| 2D | 147473787 | C, A | sanfensan |
| 2D | 153131701 | A, G | sanfensan |
| 2D | 155782705 | A, C | sanfensan |
| 2D | 161543833 | T, G | sanfensan |
| 2D | 168209065 | T, C | sanfensan |
| 2D | 171707336 | G, A | sanfensan |
| 2D | 175227041 | C, G | sanfensan |
| 2D | 183727141 | C, A | sanfensan |
| 2D | 186245952 | T, C | sanfensan |
| 2D | 191571350 | T, C | sanfensan |
| 2D | 197886074 | C, A | sanfensan |
| 2D | 201218813 | A, G | sanfensan |
| 2D | 206194560 | G, T | sanfensan |
| 2D | 209315382 | T, C | sanfensan |
| 2D | 214621383 | T, G | sanfensan |
| 2D | 217094666 | T, C | sanfensan |
| 2D | 224088866 | C, T | sanfensan |
| 2D | 228951287 | A, G | sanfensan |
| 2D | 233694297 | A, C | sanfensan |
| 2D | 237373003 | T, C | sanfensan |
| 2D | 240374430 | G, T | sanfensan |
| 2D | 247507939 | G, A | sanfensan |
| 2D | 253371162 | C, T | sanfensan |
| 2D | 259426340 | T, C | sanfensan |
| 2D | 262144878 | T, C | sanfensan |
| 2D | 268152175 | C, T | sanfensan |
| 2D | 271878835 | A, C | sanfensan |
| 2D | 278388739 | G, A | sanfensan |
| 2D | 281916083 | G, A | sanfensan |
| 2D | 289003292 | T, C | sanfensan |
| 2D | 293861011 | T, C | sanfensan |
| 2D | 297746679 | G, A | sanfensan |
| 2D | 301635985 | T, C | sanfensan |
| 2D | 307441210 | A, G | sanfensan |
| 2D | 313997317 | C, T | sanfensan |
| 2D | 317780757 | T, C | sanfensan |
| 2D | 324471320 | G, C | sanfensan |
| 2D | 325394234 | C, T | sanfensan |
| 2D | 332243555 | G, A | sanfensan |
| 2D | 336723088 | C, T | sanfensan |
| 2D | 344502614 | T, C | sanfensan |
| 2D | 348558097 | G, A | sanfensan |
| 2D | 354230586 | G, A | sanfensan |
| 2D | 356506435 | C, T | sanfensan |
| 2D | 361372636 | C, A | sanfensan |
| 2D | 367508960 | T, C | sanfensan |
| 2D | 374623643 | C, T | sanfensan |
| 2D | 376574690 | G, A | sanfensan |
| 2D | 381350972 | A, G | sanfensan |
| 2D | 385713492 | C, T | sanfensan |
| 2D | 389191706 | C, A | sanfensan |
| 2D | 393186784 | T, C | sanfensan |
| 2D | 398053878 | C, T | sanfensan |
| 2D | 402113963 | G, A | sanfensan |
| 2D | 407714293 | C, T | sanfensan |
| 2D | 413300157 | C, G | sanfensan |
| 2D | 416994083 | C, T | sanfensan |
| 2D | 422486122 | A, G | sanfensan |
| 2D | 429086006 | C, T | sanfensan |
| 2D | 430932257 | T, G | sanfensan |
| 2D | 435517110 | T, C | sanfensan |
| 2D | 440681525 | G, T | sanfensan |
| 2D | 446278114 | T, C | sanfensan |
| 2D | 452744585 | A, G | sanfensan |
| 2D | 458588987 | G, A | sanfensan |
| 2D | 460269576 | T, G | sanfensan |
| 2D | 466552364 | C, T | sanfensan |
| 2D | 473536427 | A, C | sanfensan |
| 2D | 478638656 | T, G | sanfensan |
| 2D | 484074145 | G, A | sanfensan |
| 2D | 486599496 | C, T | sanfensan |
| 2D | 492020467 | C, T | sanfensan |
| 2D | 498876308 | C, T | sanfensan |
| 2D | 499971233 | T, C | sanfensan |
| 2D | 501118019 | C, T | sanfensan |
| 2D | 509719761 | G, T | sanfensan |
| 2D | 511708692 | G, A | sanfensan |
| 2D | 515022757 | T, A | sanfensan |
| 2D | 523356028 | C, A | sanfensan |
| 2D | 525941172 | A, G | sanfensan |
| 2D | 529724172 | A, C | sanfensan |
| 2D | 530568058 | G, A | sanfensan |
| 3A | 349403 | C, T | sanfensan |
| 3A | 5686447 | A, G | sanfensan |
| 3A | 14820154 | G, C | sanfensan |
| 3A | 19103892 | A, G | sanfensan |
| 3A | 24307017 | G, A | sanfensan |
| 3A | 28596998 | G, A | sanfensan |
| 3A | 34850179 | A, G | sanfensan |
| 3A | 39937020 | C, T | sanfensan |
| 3A | 43520691 | A, G | sanfensan |
| 3A | 49770087 | T, C | sanfensan |
| 3A | 54743914 | A, G | sanfensan |
| 3A | 56540660 | A, C | sanfensan |
| 3A | 60015652 | C, T | sanfensan |
| 3A | 68312753 | T, G | sanfensan |
| 3A | 70210131 | A, G | sanfensan |
| 3A | 75548656 | C, T | sanfensan |
| 3A | 81960668 | G, A | sanfensan |
| 3A | 87385909 | G, A | sanfensan |
| 3A | 91466974 | T, C | sanfensan |
| 3A | 99324808 | G, A | sanfensan |
| 3A | 103702261 | A, T | sanfensan |
| 3A | 106902222 | A, G | sanfensan |
| 3A | 114734802 | C, T | sanfensan |
| 3A | 117018962 | T, G | sanfensan |
| 3A | 123014541 | G, T | sanfensan |
| 3A | 125235584 | G, A | sanfensan |
| 3A | 132736923 | G, T | sanfensan |
| 3A | 133400727 | T, C | sanfensan |
| 3A | 135881496 | A, G | sanfensan |
| 3A | 140132188 | A, C | sanfensan |
| 3A | 149216557 | G, T | sanfensan |
| 3A | 153688728 | T, C | sanfensan |
| 3A | 157365828 | T, G | sanfensan |
| 3A | 162629353 | G, A | sanfensan |
| 3A | 163249925 | C, T | sanfensan |
| 3A | 165620860 | A, G | sanfensan |
| 3A | 170114512 | T, C | sanfensan |
| 3A | 175321928 | G, A | sanfensan |
| 3A | 183395685 | C, T | sanfensan |
| 3A | 188224937 | A, C | sanfensan |
| 3A | 194044731 | G, A | sanfensan |
| 3A | 197334481 | G, A | sanfensan |
| 3A | 204024961 | T, C | sanfensan |
| 3A | 209320496 | A, G | sanfensan |
| 3A | 214269791 | G, A | sanfensan |
| 3A | 215870959 | A, G | sanfensan |
| 3A | 220058696 | A, C | sanfensan |
| 3A | 228807812 | G, A | sanfensan |
| 3A | 234527749 | A, G | sanfensan |
| 3A | 237989422 | G, A | sanfensan |
| 3A | 241256197 | C, T | sanfensan |
| 3A | 248608009 | C, T | sanfensan |
| 3A | 254861238 | T, C | sanfensan |
| 3A | 255490400 | A, G | sanfensan |
| 3A | 261499403 | T, C | sanfensan |
| 3A | 268848139 | T, G | sanfensan |
| 3A | 273512863 | T, C | sanfensan |
| 3A | 279998619 | A, G | sanfensan |
| 3A | 280635220 | A, C | sanfensan |
| 3A | 289497088 | C, T | sanfensan |
| 3A | 291617575 | G, A | sanfensan |
| 3A | 296690651 | T, C | sanfensan |
| 3A | 303858433 | G, A | sanfensan |
| 3A | 308390029 | A, G | sanfensan |
| 3A | 313163176 | C, T | sanfensan |
| 3A | 315321758 | G, T | sanfensan |
| 3A | 323449820 | C, T | sanfensan |
| 3A | 326487847 | G, T | sanfensan |
| 3A | 334646062 | C, G | sanfensan |
| 3A | 339385657 | T, G | sanfensan |
| 3A | 344905994 | C, T | sanfensan |
| 3A | 346539460 | G, A | sanfensan |
| 3A | 354806237 | C, T | sanfensan |
| 3A | 358217253 | C, T | sanfensan |
| 3A | 363057750 | C, T | sanfensan |
| 3A | 368774140 | C, T | sanfensan |
| 3A | 370439246 | C, T | sanfensan |
| 3A | 379158179 | A, G | sanfensan |
| 3A | 380572844 | T, C | sanfensan |
| 3A | 385843902 | C, T | sanfensan |
| 3A | 393548401 | C, T | sanfensan |
| 3A | 396511248 | A, G | sanfensan |
| 3A | 400231496 | G, A | sanfensan |
| 3A | 407861434 | T, C | sanfensan |
| 3A | 410745937 | A, G | sanfensan |
| 3A | 417480860 | A, C | sanfensan |
| 3C | 4934705 | T, C | sanfensan |
| 3C | 9789448 | T, C | sanfensan |
| 3C | 14114292 | A, G | sanfensan |
| 3C | 16111717 | T, C | sanfensan |
| 3C | 20060240 | T, C | sanfensan |
| 3C | 29738599 | G, T | sanfensan |
| 3C | 34251466 | G, A | sanfensan |
| 3C | 39605336 | A, C | sanfensan |
| 3C | 41571752 | T, C | sanfensan |
| 3C | 48463788 | G, T | sanfensan |
| 3C | 50619185 | G, A | sanfensan |
| 3C | 59812660 | G, A | sanfensan |
| 3C | 63059450 | T, C | sanfensan |
| 3C | 69756699 | C, T | sanfensan |
| 3C | 72007306 | T, C | sanfensan |
| 3C | 75582282 | G, C | sanfensan |
| 3C | 81453337 | T, C | sanfensan |
| 3C | 89524634 | T, C | sanfensan |
| 3C | 94729319 | C, A | sanfensan |
| 3C | 96052025 | C, T | sanfensan |
| 3C | 100397477 | A, G | sanfensan |
| 3C | 108020814 | A, G | sanfensan |
| 3C | 113507812 | A, C | sanfensan |
| 3C | 119056248 | C, T | sanfensan |
| 3C | 121539032 | G, T | sanfensan |
| 3C | 127564610 | C, T | sanfensan |
| 3C | 130874626 | G, A | sanfensan |
| 3C | 137072359 | G, A | sanfensan |
| 3C | 142064744 | G, A | sanfensan |
| 3C | 147139993 | T, C | sanfensan |
| 3C | 152287508 | G, A | sanfensan |
| 3C | 158231130 | C, T | sanfensan |
| 3C | 161849544 | C, T | sanfensan |
| 3C | 167803980 | A, G | sanfensan |
| 3C | 171441935 | T, C | sanfensan |
| 3C | 178608408 | T, G | sanfensan |
| 3C | 180422132 | T, C | sanfensan |
| 3C | 186181001 | G, A | sanfensan |
| 3C | 192589566 | T, C | sanfensan |
| 3C | 195188213 | G, A | sanfensan |
| 3C | 204778810 | C, T | sanfensan |
| 3C | 207904801 | T, C | sanfensan |
| 3C | 214065619 | T, G | sanfensan |
| 3C | 217226719 | T, C | sanfensan |
| 3C | 221780018 | G, C | sanfensan |
| 3C | 228014218 | G, A | sanfensan |
| 3C | 233526022 | G, A | sanfensan |
| 3C | 235353093 | A, C | sanfensan |
| 3C | 244162351 | G, A | sanfensan |
| 3C | 248122047 | C, T | sanfensan |
| 3C | 253823594 | C, T | sanfensan |
| 3C | 259462643 | A, G | sanfensan |
| 3C | 260199510 | T, C | sanfensan |
| 3C | 269465277 | G, T | sanfensan |
| 3C | 274931211 | G, A | sanfensan |
| 3C | 277523171 | C, A | sanfensan |
| 3C | 284109639 | G, C | sanfensan |
| 3C | 285346116 | A, G | sanfensan |
| 3C | 294939142 | A, C | sanfensan |
| 3C | 296525393 | A, G | sanfensan |
| 3C | 304406734 | T, C | sanfensan |
| 3C | 309329884 | C, T | sanfensan |
| 3C | 310447021 | T, G | sanfensan |
| 3C | 319961145 | T, C | sanfensan |
| 3C | 321458383 | T, C | sanfensan |
| 3C | 325121366 | A, G | sanfensan |
| 3C | 330206369 | A, G | sanfensan |
| 3C | 332130807 | C, T | sanfensan |
| 3C | 338543178 | T, C | sanfensan |
| 3C | 343287383 | T, C | sanfensan |
| 3C | 349175645 | T, C | sanfensan |
| 3C | 353910191 | C, T | sanfensan |
| 3C | 358917955 | T, C | sanfensan |
| 3C | 363942089 | G, A | sanfensan |
| 3C | 366028325 | A, C | sanfensan |
| 3C | 372945120 | C, T | sanfensan |
| 3C | 375489817 | C, G | sanfensan |
| 3C | 384457117 | A, G | sanfensan |
| 3C | 388958566 | A, G | sanfensan |
| 3C | 394935493 | A, T | sanfensan |
| 3C | 395752355 | G, A | sanfensan |
| 3C | 403204424 | G, A | sanfensan |
| 3C | 408346907 | A, C | sanfensan |
| 3C | 412555939 | T, C | sanfensan |
| 3C | 418959428 | A, G | sanfensan |
| 3C | 424637971 | G, A | sanfensan |
| 3C | 425426160 | G, A | sanfensan |
| 3C | 431869178 | A, C | sanfensan |
| 3C | 437116797 | T, G | sanfensan |
| 3C | 442997546 | A, G | sanfensan |
| 3C | 445451564 | C, A | sanfensan |
| 3C | 449848039 | C, T | sanfensan |
| 3C | 450156513 | C, T | sanfensan |
| 3C | 458930099 | T, C | sanfensan |
| 3C | 460938762 | C, T | sanfensan |
| 3C | 465773933 | A, G | sanfensan |
| 3C | 466127904 | C, A | sanfensan |
| 3C | 472480324 | G, T | sanfensan |
| 3C | 476852155 | T, C | sanfensan |
| 3C | 482946601 | C, T | sanfensan |
| 3C | 488844690 | A, C | sanfensan |
| 3C | 494056611 | C, T | sanfensan |
| 3C | 499140906 | G, A | sanfensan |
| 3C | 504946938 | C, A | sanfensan |
| 3C | 505015277 | T, G | sanfensan |
| 3C | 513611369 | G, A | sanfensan |
| 3C | 515368248 | C, T | sanfensan |
| 3C | 520707894 | G, A | sanfensan |
| 3C | 529897647 | C, T | sanfensan |
| 3C | 534373018 | A, G | sanfensan |
| 3C | 536903652 | C, T | sanfensan |
| 3C | 540735781 | A, C | sanfensan |
| 3C | 545800871 | T, C | sanfensan |
| 3C | 551522255 | A, C | sanfensan |
| 3C | 556937242 | T, C | sanfensan |
| 3C | 561945091 | C, A | sanfensan |
| 3C | 565260776 | C, T | sanfensan |
| 3C | 572949143 | C, G | sanfensan |
| 3C | 577651667 | G, A | sanfensan |
| 3C | 580350958 | C, T | sanfensan |
| 3C | 589499247 | A, G | sanfensan |
| 3C | 593401324 | A, G | sanfensan |
| 3C | 596059950 | G, A | sanfensan |
| 3C | 602749897 | C, G | sanfensan |
| 3C | 606686603 | A, G | sanfensan |
| 3C | 612645249 | A, G | sanfensan |
| 3C | 615205024 | T, A | sanfensan |
| 3D | 3451014 | A, G | sanfensan |
| 3D | 6304975 | C, G | sanfensan |
| 3D | 8431856 | G, A | sanfensan |
| 3D | 12739908 | G, A | sanfensan |
| 3D | 17626366 | T, C | sanfensan |
| 3D | 22243586 | T, C | sanfensan |
| 3D | 29901822 | T, C | sanfensan |
| 3D | 34199181 | T, A | sanfensan |
| 3D | 38215402 | C, T | sanfensan |
| 3D | 40736696 | C, T | sanfensan |
| 3D | 45922953 | C, T | sanfensan |
| 3D | 54465933 | T, C | sanfensan |
| 3D | 59996227 | A, G | sanfensan |
| 3D | 63704529 | G, A | sanfensan |
| 3D | 66789700 | C, G | sanfensan |
| 3D | 70216974 | C, T | sanfensan |
| 3D | 78492763 | A, G | sanfensan |
| 3D | 82560649 | G, A | sanfensan |
| 3D | 89278480 | C, T | sanfensan |
| 3D | 94976535 | T, A | sanfensan |
| 3D | 99870793 | C, A | sanfensan |
| 3D | 102284469 | G, A | sanfensan |
| 3D | 105085292 | G, T | sanfensan |
| 3D | 112272049 | A, G | sanfensan |
| 3D | 117725865 | G, A | sanfensan |
| 3D | 124920499 | T, C | sanfensan |
| 3D | 125085251 | G, A | sanfensan |
| 3D | 130241097 | G, T | sanfensan |
| 3D | 136299812 | G, A | sanfensan |
| 3D | 140453087 | G, T | sanfensan |
| 3D | 149490075 | G, A | sanfensan |
| 3D | 154494846 | C, T | sanfensan |
| 3D | 155736382 | A, G | sanfensan |
| 3D | 160258419 | T, G | sanfensan |
| 3D | 165382934 | G, C | sanfensan |
| 3D | 174233842 | A, G | sanfensan |
| 3D | 178210807 | A, G | sanfensan |
| 3D | 183045672 | C, T | sanfensan |
| 3D | 185605470 | A, G | sanfensan |
| 3D | 191181967 | T, C | sanfensan |
| 3D | 199127188 | C, A | sanfensan |
| 3D | 201408393 | T, C | sanfensan |
| 3D | 208325818 | C, A | sanfensan |
| 3D | 213296950 | G, T | sanfensan |
| 3D | 217423350 | T, C | sanfensan |
| 3D | 223809923 | G, C | sanfensan |
| 3D | 226483167 | G, A | sanfensan |
| 3D | 231132075 | C, T | sanfensan |
| 3D | 237287427 | T, A | sanfensan |
| 3D | 241417679 | G, A | sanfensan |
| 3D | 248327064 | G, A | sanfensan |
| 3D | 250955048 | C, T | sanfensan |
| 3D | 257222395 | C, T | sanfensan |
| 3D | 263680149 | G, A | sanfensan |
| 3D | 268114489 | C, A | sanfensan |
| 3D | 273619731 | A, C | sanfensan |
| 3D | 276710058 | C, T | sanfensan |
| 3D | 284403130 | G, C | sanfensan |
| 3D | 288065151 | G, A | sanfensan |
| 3D | 294630726 | C, T | sanfensan |
| 3D | 297227408 | C, T | sanfensan |
| 3D | 300199691 | T, C | sanfensan |
| 3D | 306425893 | C, A | sanfensan |
| 3D | 314722897 | A, G | sanfensan |
| 3D | 315890257 | A, T | sanfensan |
| 3D | 323402379 | G, A | sanfensan |
| 3D | 325861485 | A, G | sanfensan |
| 3D | 330563924 | A, G | sanfensan |
| 3D | 335625837 | G, A | sanfensan |
| 3D | 340772061 | G, A | sanfensan |
| 3D | 348100481 | T, G | sanfensan |
| 3D | 352819049 | C, T | sanfensan |
| 3D | 358682575 | T, C | sanfensan |
| 3D | 361158177 | G, A | sanfensan |
| 3D | 369050731 | T, C | sanfensan |
| 3D | 373598604 | C, T | sanfensan |
| 3D | 378481165 | T, C | sanfensan |
| 3D | 381350490 | G, A | sanfensan |
| 3D | 388861217 | T, C | sanfensan |
| 3D | 389878476 | T, G | sanfensan |
| 3D | 391163814 | T, C | sanfensan |
| 3D | 397016298 | C, T | sanfensan |
| 3D | 403496100 | A, G | sanfensan |
| 3D | 406924921 | A, G | sanfensan |
| 3D | 410226093 | G, A | sanfensan |
| 3D | 417099879 | A, G | sanfensan |
| 3D | 419164521 | C, G | sanfensan |
| 3D | 423904272 | T, C | sanfensan |
| 3D | 426092994 | A, G | sanfensan |
| 3D | 433821318 | G, A | sanfensan |
| 3D | 436973559 | T, G | sanfensan |
| 3D | 439655087 | A, G | sanfensan |
| 3D | 441290663 | T, C | sanfensan |
| 3D | 448791478 | T, C | sanfensan |
| 3D | 451012282 | C, G | sanfensan |
| 3D | 456635851 | G, A | sanfensan |
| 3D | 460647997 | A, T | sanfensan |
| 4A | 167665 | A, G | sanfensan |
| 4A | 5170696 | A, G | sanfensan |
| 4A | 12321972 | G, A | sanfensan |
| 4A | 19482740 | T, C | sanfensan |
| 4A | 22870941 | T, C | sanfensan |
| 4A | 27380296 | T, C | sanfensan |
| 4A | 30862932 | G, T | sanfensan |
| 4A | 36420099 | T, G | sanfensan |
| 4A | 38087452 | T, G | sanfensan |
| 4A | 44284137 | A, C | sanfensan |
| 4A | 45337009 | T, C | sanfensan |
| 4A | 50385302 | T, C | sanfensan |
| 4A | 58613149 | T, C | sanfensan |
| 4A | 62632618 | T, C | sanfensan |
| 4A | 69360237 | A, C | sanfensan |
| 4A | 72548515 | T, C | sanfensan |
| 4A | 78860501 | A, G | sanfensan |
| 4A | 83477002 | A, G | sanfensan |
| 4A | 86718114 | A, C | sanfensan |
| 4A | 90186259 | T, C | sanfensan |
| 4A | 99717884 | C, G | sanfensan |
| 4A | 103226101 | C, T | sanfensan |
| 4A | 108841089 | C, A | sanfensan |
| 4A | 109097229 | G, A | sanfensan |
| 4A | 110362403 | T, C | sanfensan |
| 4A | 119524477 | G, C | sanfensan |
| 4A | 124335960 | C, T | sanfensan |
| 4A | 129987141 | C, T | sanfensan |
| 4A | 132188846 | A, G | sanfensan |
| 4A | 138010184 | A, G | sanfensan |
| 4A | 141248665 | T, C | sanfensan |
| 4A | 141974369 | A, G | sanfensan |
| 4A | 146462646 | G, A | sanfensan |
| 4A | 151979768 | T, C | sanfensan |
| 4A | 158451505 | G, A | sanfensan |
| 4A | 163350428 | A, G | sanfensan |
| 4A | 167565220 | A, G | sanfensan |
| 4A | 173953101 | T, G | sanfensan |
| 4A | 178883204 | G, C | sanfensan |
| 4A | 184596923 | C, T | sanfensan |
| 4A | 189201669 | A, C | sanfensan |
| 4A | 193547898 | G, A | sanfensan |
| 4A | 199508298 | A, G | sanfensan |
| 4A | 201195563 | C, T | sanfensan |
| 4A | 207185396 | C, A | sanfensan |
| 4A | 213373498 | T, C | sanfensan |
| 4A | 216222538 | A, G | sanfensan |
| 4A | 221795376 | C, T | sanfensan |
| 4A | 228345642 | A, T | sanfensan |
| 4A | 231330723 | A, C | sanfensan |
| 4A | 235514032 | G, A | sanfensan |
| 4A | 242193423 | C, T | sanfensan |
| 4A | 247945600 | C, T | sanfensan |
| 4A | 252369025 | T, C | sanfensan |
| 4A | 259286784 | G, C | sanfensan |
| 4A | 261531070 | A, G | sanfensan |
| 4A | 268127413 | G, T | sanfensan |
| 4A | 273296067 | A, G | sanfensan |
| 4A | 276596131 | T, C | sanfensan |
| 4A | 280307793 | C, T | sanfensan |
| 4A | 289509128 | C, T | sanfensan |
| 4A | 293451571 | G, A | sanfensan |
| 4A | 299169983 | T, G | sanfensan |
| 4A | 300515091 | T, C | sanfensan |
| 4A | 305599904 | G, A | sanfensan |
| 4A | 311085314 | A, C | sanfensan |
| 4A | 316042196 | C, T | sanfensan |
| 4A | 322946253 | T, C | sanfensan |
| 4A | 325478648 | G, C | sanfensan |
| 4A | 330347981 | A, G | sanfensan |
| 4A | 337443775 | C, T | sanfensan |
| 4A | 340462080 | A, G | sanfensan |
| 4A | 347382275 | A, G | sanfensan |
| 4A | 354497490 | T, G | sanfensan |
| 4A | 359050714 | C, A | sanfensan |
| 4A | 362340167 | C, T | sanfensan |
| 4A | 367751846 | G, A | sanfensan |
| 4A | 370622530 | G, T | sanfensan |
| 4A | 379177676 | T, C | sanfensan |
| 4A | 381166365 | T, C | sanfensan |
| 4A | 385406079 | C, G | sanfensan |
| 4A | 398015147 | A, C | sanfensan |
| 4A | 401416507 | G, A | sanfensan |
| 4A | 406634471 | A, G | sanfensan |
| 4A | 413012139 | A, G | sanfensan |
| 4A | 418384264 | C, T | sanfensan |
| 4A | 424141863 | C, T | sanfensan |
| 4A | 426462945 | A, T | sanfensan |
| 4A | 434286106 | G, T | sanfensan |
| 4A | 435134503 | C, T | sanfensan |
| 4A | 440807368 | C, G | sanfensan |
| 4A | 445324027 | C, T | sanfensan |
| 4A | 450745492 | T, C | sanfensan |
| 4A | 458822974 | A, G | sanfensan |
| 4A | 462418830 | A, C | sanfensan |
| 4A | 466738641 | T, C | sanfensan |
| 4C | 3595818 | G, A | sanfensan |
| 4C | 6028133 | A, G | sanfensan |
| 4C | 13049345 | T, A | sanfensan |
| 4C | 19972099 | A, G | sanfensan |
| 4C | 20771307 | T, C | sanfensan |
| 4C | 25886686 | G, C | sanfensan |
| 4C | 32211660 | C, A | sanfensan |
| 4C | 36873418 | G, C | sanfensan |
| 4C | 40449202 | C, G | sanfensan |
| 4C | 47158151 | C, A | sanfensan |
| 4C | 51926597 | T, C | sanfensan |
| 4C | 57722231 | G, A | sanfensan |
| 4C | 61365967 | G, A | sanfensan |
| 4C | 66290570 | C, T | sanfensan |
| 4C | 69406750 | G, A | sanfensan |
| 4C | 71656189 | G, A | sanfensan |
| 4C | 75202185 | A, G | sanfensan |
| 4C | 80202847 | G, T | sanfensan |
| 4C | 86069619 | A, G | sanfensan |
| 4C | 92181840 | C, T | sanfensan |
| 4C | 95153438 | G, A | sanfensan |
| 4C | 101779057 | T, C | sanfensan |
| 4C | 106362411 | C, T | sanfensan |
| 4C | 113373499 | T, G | sanfensan |
| 4C | 116319458 | C, T | sanfensan |
| 4C | 122530727 | A, G | sanfensan |
| 4C | 128925968 | T, G | sanfensan |
| 4C | 130658845 | T, C | sanfensan |
| 4C | 139094233 | A, G | sanfensan |
| 4C | 141439434 | G, T | sanfensan |
| 4C | 149617745 | A, C | sanfensan |
| 4C | 151871212 | T, C | sanfensan |
| 4C | 159944864 | C, T | sanfensan |
| 4C | 164691088 | T, C | sanfensan |
| 4C | 169970304 | C, A | sanfensan |
| 4C | 170331200 | A, C | sanfensan |
| 4C | 178692748 | C, T | sanfensan |
| 4C | 184841316 | C, T | sanfensan |
| 4C | 185887772 | T, C | sanfensan |
| 4C | 193734961 | C, T | sanfensan |
| 4C | 197024216 | G, A | sanfensan |
| 4C | 202625381 | G, A | sanfensan |
| 4C | 209016901 | A, G | sanfensan |
| 4C | 213200350 | C, T | sanfensan |
| 4C | 215000144 | C, T | sanfensan |
| 4C | 224645115 | C, T | sanfensan |
| 4C | 229625246 | A, G | sanfensan |
| 4C | 234547399 | T, C | sanfensan |
| 4C | 236119582 | A, C | sanfensan |
| 4C | 241687009 | G, A | sanfensan |
| 4C | 245369143 | G, T | sanfensan |
| 4C | 252253086 | T, A | sanfensan |
| 4C | 255164714 | C, T | sanfensan |
| 4C | 260860063 | G, A | sanfensan |
| 4C | 267013553 | G, A | sanfensan |
| 4C | 273797546 | G, A | sanfensan |
| 4C | 277862984 | C, T | sanfensan |
| 4C | 282087726 | G, A | sanfensan |
| 4C | 287337582 | G, A | sanfensan |
| 4C | 292238812 | C, T | sanfensan |
| 4C | 296562693 | C, T | sanfensan |
| 4C | 301499481 | T, C | sanfensan |
| 4C | 308695483 | G, A | sanfensan |
| 4C | 311224923 | G, A | sanfensan |
| 4C | 316095744 | G, A | sanfensan |
| 4C | 324951331 | C, T | sanfensan |
| 4C | 325766510 | T, G | sanfensan |
| 4C | 331060999 | G, A | sanfensan |
| 4C | 339044922 | C, T | sanfensan |
| 4C | 342159259 | G, A | sanfensan |
| 4C | 345139072 | G, A | sanfensan |
| 4C | 350531907 | G, T | sanfensan |
| 4C | 357426076 | G, A | sanfensan |
| 4C | 362889043 | G, A | sanfensan |
| 4C | 369798229 | G, A | sanfensan |
| 4C | 372472310 | C, T | sanfensan |
| 4C | 378265862 | T, C | sanfensan |
| 4C | 381319655 | G, A | sanfensan |
| 4C | 385095261 | T, G | sanfensan |
| 4C | 387723697 | C, T | sanfensan |
| 4C | 392326058 | T, C | sanfensan |
| 4C | 395186922 | T, C | sanfensan |
| 4C | 404320541 | A, C | sanfensan |
| 4C | 407798339 | C, G | sanfensan |
| 4C | 414198946 | A, G | sanfensan |
| 4C | 418786637 | C, T | sanfensan |
| 4C | 420987303 | G, C | sanfensan |
| 4C | 425274444 | A, T | sanfensan |
| 4C | 437039125 | T, C | sanfensan |
| 4C | 442594181 | C, T | sanfensan |
| 4C | 446643052 | G, T | sanfensan |
| 4C | 451884828 | C, A | sanfensan |
| 4C | 455850824 | G, A | sanfensan |
| 4C | 463494104 | C, T | sanfensan |
| 4C | 467165660 | C, T | sanfensan |
| 4C | 472240988 | T, G | sanfensan |
| 4C | 475026617 | T, C | sanfensan |
| 4C | 481585885 | G, A | sanfensan |
| 4C | 487240271 | G, T | sanfensan |
| 4C | 493243160 | C, T | sanfensan |
| 4C | 499531487 | C, A | sanfensan |
| 4C | 504518607 | G, A | sanfensan |
| 4C | 509955623 | G, A | sanfensan |
| 4C | 511514837 | A, G | sanfensan |
| 4C | 514138096 | G, A | sanfensan |
| 4C | 519111243 | A, T | sanfensan |
| 4C | 520487689 | C, T | sanfensan |
| 4C | 526516206 | A, G | sanfensan |
| 4C | 530500162 | C, T | sanfensan |
| 4C | 537644622 | T, C | sanfensan |
| 4C | 540730312 | G, A | sanfensan |
| 4C | 545836115 | G, T | sanfensan |
| 4C | 549932188 | A, G | sanfensan |
| 4C | 550915489 | A, T | sanfensan |
| 4C | 558054888 | C, T | sanfensan |
| 4C | 562963997 | A, G | sanfensan |
| 4C | 569467277 | A, G | sanfensan |
| 4C | 572657475 | T, C | sanfensan |
| 4C | 575327801 | A, G | sanfensan |
| 4C | 584368593 | T, G | sanfensan |
| 4C | 588543504 | A, G | sanfensan |
| 4C | 589445451 | A, G | sanfensan |
| 4C | 593669777 | A, C | sanfensan |
| 4C | 599151537 | A, G | sanfensan |
| 4C | 600053663 | T, C | sanfensan |
| 4C | 607995668 | A, G | sanfensan |
| 4C | 610178903 | T, C | sanfensan |
| 4C | 614566248 | G, C | sanfensan |
| 4C | 616610794 | C, T | sanfensan |
| 4C | 624701501 | T, C | sanfensan |
| 4C | 626629588 | G, A | sanfensan |
| 4C | 631097435 | A, C | sanfensan |
| 4C | 635505874 | C, A | sanfensan |
| 4C | 642622496 | T, C | sanfensan |
| 4C | 645881564 | A, C | sanfensan |
| 4C | 653135330 | G, T | sanfensan |
| 4C | 655135466 | A, G | sanfensan |
| 4C | 660684760 | G, A | sanfensan |
| 4C | 665124776 | G, C | sanfensan |
| 4C | 674949846 | A, G | sanfensan |
| 4C | 675185316 | A, G | sanfensan |
| 4C | 683318163 | T, C | sanfensan |
| 4C | 689664776 | T, C | sanfensan |
| 4C | 694624379 | C, T | sanfensan |
| 4C | 695265876 | A, G | sanfensan |
| 4C | 701273830 | A, G | sanfensan |
| 4D | 3022923 | A, G | sanfensan |
| 4D | 7849878 | C, T | sanfensan |
| 4D | 10672344 | A, G | sanfensan |
| 4D | 16331251 | T, G | sanfensan |
| 4D | 23073905 | A, G | sanfensan |
| 4D | 28279824 | T, G | sanfensan |
| 4D | 32649688 | A, G | sanfensan |
| 4D | 37038379 | A, C | sanfensan |
| 4D | 43180451 | T, C | sanfensan |
| 4D | 45472604 | A, G | sanfensan |
| 4D | 50482410 | A, G | sanfensan |
| 4D | 58490879 | G, A | sanfensan |
| 4D | 62917919 | G, A | sanfensan |
| 4D | 65612418 | G, A | sanfensan |
| 4D | 71637400 | C, T | sanfensan |
| 4D | 76187635 | C, T | sanfensan |
| 4D | 82058789 | T, C | sanfensan |
| 4D | 88887772 | G, A | sanfensan |
| 4D | 92323177 | A, G | sanfensan |
| 4D | 97430755 | A, G | sanfensan |
| 4D | 102821671 | C, G | sanfensan |
| 4D | 106018930 | T, G | sanfensan |
| 4D | 110865436 | A, G | sanfensan |
| 4D | 116039043 | C, T | sanfensan |
| 4D | 118774001 | C, G | sanfensan |
| 4D | 121609230 | A, G | sanfensan |
| 4D | 128046905 | T, C | sanfensan |
| 4D | 134025258 | T, C | sanfensan |
| 4D | 137854184 | C, G | sanfensan |
| 4D | 141417395 | A, G | sanfensan |
| 4D | 148636319 | A, G | sanfensan |
| 4D | 151219343 | C, T | sanfensan |
| 4D | 153032013 | T, C | sanfensan |
| 4D | 159949170 | T, C | sanfensan |
| 4D | 163913258 | A, G | sanfensan |
| 4D | 165337682 | T, A | sanfensan |
| 4D | 173510202 | T, G | sanfensan |
| 4D | 179301600 | C, G | sanfensan |
| 4D | 184426060 | G, A | sanfensan |
| 4D | 187340496 | G, T | sanfensan |
| 4D | 193603755 | A, G | sanfensan |
| 4D | 196382892 | T, C | sanfensan |
| 4D | 202074844 | C, T | sanfensan |
| 4D | 206447702 | G, A | sanfensan |
| 4D | 212380635 | C, T | sanfensan |
| 4D | 215885690 | T, C | sanfensan |
| 4D | 220033123 | T, G | sanfensan |
| 4D | 227048389 | A, G | sanfensan |
| 4D | 232039750 | C, T | sanfensan |
| 4D | 236815937 | T, A | sanfensan |
| 4D | 242091618 | C, T | sanfensan |
| 4D | 245101303 | C, A | sanfensan |
| 4D | 250407222 | A, G | sanfensan |
| 4D | 256019130 | T, C | sanfensan |
| 4D | 260909900 | T, C | sanfensan |
| 4D | 267887871 | A, G | sanfensan |
| 4D | 271126060 | C, A | sanfensan |
| 4D | 277162174 | T, G | sanfensan |
| 4D | 283660950 | G, A | sanfensan |
| 4D | 285824135 | G, T | sanfensan |
| 4D | 294845699 | G, A | sanfensan |
| 4D | 299632398 | G, A | sanfensan |
| 4D | 303350038 | T, C | sanfensan |
| 4D | 307597358 | T, A | sanfensan |
| 4D | 314228425 | C, T | sanfensan |
| 4D | 314253165 | G, A | sanfensan |
| 4D | 315607825 | A, C | sanfensan |
| 4D | 321533893 | G, T | sanfensan |
| 4D | 325136857 | A, T | sanfensan |
| 4D | 326786150 | A, T | sanfensan |
| 4D | 332125743 | A, G | sanfensan |
| 4D | 335136720 | A, G | sanfensan |
| 4D | 340382269 | C, T | sanfensan |
| 4D | 348383382 | A, G | sanfensan |
| 4D | 352477479 | T, C | sanfensan |
| 4D | 356651761 | A, G | sanfensan |
| 4D | 363724413 | C, G | sanfensan |
| 4D | 366480345 | A, G | sanfensan |
| 4D | 374238192 | T, C | sanfensan |
| 4D | 378149215 | T, C | sanfensan |
| 4D | 384366440 | G, A | sanfensan |
| 4D | 389105351 | A, C | sanfensan |
| 4D | 390762487 | A, G | sanfensan |
| 4D | 398109909 | C, G | sanfensan |
| 4D | 402374421 | T, C | sanfensan |
| 4D | 407356318 | G, A | sanfensan |
| 4D | 413268611 | T, C | sanfensan |
| 4D | 418031647 | G, A | sanfensan |
| 4D | 420702529 | G, A | sanfensan |
| 4D | 426669740 | A, G | sanfensan |
| 4D | 432669116 | G, A | sanfensan |
| 4D | 437532533 | G, T | sanfensan |
| 4D | 443034471 | C, T | sanfensan |
| 4D | 445235008 | A, C | sanfensan |
| 4D | 451513413 | A, G | sanfensan |
| 5A | 2255953 | A, C | sanfensan |
| 5A | 6260213 | C, A | sanfensan |
| 5A | 13803621 | C, T | sanfensan |
| 5A | 15561310 | G, C | sanfensan |
| 5A | 24452734 | T, C | sanfensan |
| 5A | 27224463 | G, A | sanfensan |
| 5A | 34502392 | A, G | sanfensan |
| 5A | 36710142 | G, A | sanfensan |
| 5A | 44344522 | G, A | sanfensan |
| 5A | 49562544 | T, C | sanfensan |
| 5A | 53433795 | T, C | sanfensan |
| 5A | 56109353 | G, A | sanfensan |
| 5A | 64589225 | C, T | sanfensan |
| 5A | 68491563 | T, C | sanfensan |
| 5A | 70531911 | C, T | sanfensan |
| 5A | 79896986 | T, C | sanfensan |
| 5A | 83113930 | T, C | sanfensan |
| 5A | 86401636 | T, C | sanfensan |
| 5A | 94264887 | T, C | sanfensan |
| 5A | 95056890 | T, C | sanfensan |
| 5A | 102064390 | A, G | sanfensan |
| 5A | 106428331 | G, A | sanfensan |
| 5A | 110419074 | G, A | sanfensan |
| 5A | 116006562 | G, A | sanfensan |
| 5A | 121002541 | A, T | sanfensan |
| 5A | 127363246 | T, C | sanfensan |
| 5A | 132453075 | T, C | sanfensan |
| 5A | 137069112 | A, C | sanfensan |
| 5A | 142368295 | C, T | sanfensan |
| 5A | 147275003 | T, G | sanfensan |
| 5A | 152777713 | C, T | sanfensan |
| 5A | 156493283 | C, T | sanfensan |
| 5A | 160244662 | A, G | sanfensan |
| 5A | 166708997 | C, G | sanfensan |
| 5A | 174872733 | T, C | sanfensan |
| 5A | 176197051 | A, G | sanfensan |
| 5A | 181213909 | T, C | sanfensan |
| 5A | 188859714 | T, C | sanfensan |
| 5A | 190215196 | C, T | sanfensan |
| 5A | 195549254 | T, C | sanfensan |
| 5A | 198785103 | A, G | sanfensan |
| 5A | 202377152 | T, C | sanfensan |
| 5A | 207723478 | G, A | sanfensan |
| 5A | 212325208 | A, G | sanfensan |
| 5A | 216205965 | A, G | sanfensan |
| 5A | 221258102 | C, T | sanfensan |
| 5A | 225234818 | G, T | sanfensan |
| 5A | 230456063 | A, T | sanfensan |
| 5A | 238470416 | T, C | sanfensan |
| 5A | 240660477 | G, C | sanfensan |
| 5A | 245072572 | A, G | sanfensan |
| 5A | 250830642 | G, C | sanfensan |
| 5A | 259737745 | A, G | sanfensan |
| 5A | 261185047 | G, A | sanfensan |
| 5A | 269804753 | A, G | sanfensan |
| 5A | 273021787 | T, C | sanfensan |
| 5A | 276143054 | T, C | sanfensan |
| 5A | 282716721 | A, C | sanfensan |
| 5A | 289601566 | A, C | sanfensan |
| 5A | 290520143 | A, T | sanfensan |
| 5A | 297192869 | T, G | sanfensan |
| 5A | 301832004 | T, C | sanfensan |
| 5A | 308399750 | G, T | sanfensan |
| 5A | 311247256 | G, A | sanfensan |
| 5A | 317923031 | C, A | sanfensan |
| 5A | 321801333 | T, C | sanfensan |
| 5A | 327241850 | C, T | sanfensan |
| 5A | 333717258 | A, G | sanfensan |
| 5A | 335041321 | G, A | sanfensan |
| 5A | 341600328 | C, T | sanfensan |
| 5A | 347629159 | C, T | sanfensan |
| 5A | 350824983 | T, C | sanfensan |
| 5A | 356271802 | A, G | sanfensan |
| 5A | 363526707 | C, T | sanfensan |
| 5A | 369477353 | T, C | sanfensan |
| 5A | 370529828 | C, G | sanfensan |
| 5A | 375579309 | T, C | sanfensan |
| 5A | 383503810 | C, T | sanfensan |
| 5A | 387182267 | G, C | sanfensan |
| 5A | 390102471 | A, G | sanfensan |
| 5A | 398649985 | C, A | sanfensan |
| 5A | 404206934 | A, G | sanfensan |
| 5A | 409217956 | G, A | sanfensan |
| 5A | 411203550 | C, T | sanfensan |
| 5A | 415301859 | A, G | sanfensan |
| 5A | 416065809 | T, G | sanfensan |
| 5A | 421891077 | A, G | sanfensan |
| 5A | 428858871 | C, T | sanfensan |
| 5A | 434243983 | A, G | sanfensan |
| 5A | 436126819 | A, G | sanfensan |
| 5A | 441185565 | C, T | sanfensan |
| 5A | 447327633 | G, T | sanfensan |
| 5A | 452417280 | C, G | sanfensan |
| 5A | 458701937 | C, G | sanfensan |
| 5A | 460085569 | A, G | sanfensan |
| 5A | 468161069 | T, G | sanfensan |
| 5A | 474226937 | C, G | sanfensan |
| 5A | 478302848 | A, G | sanfensan |
| 5A | 483995877 | C, A | sanfensan |
| 5A | 488177553 | G, T | sanfensan |
| 5A | 488401160 | A, T | sanfensan |
| 5C | 3489579 | C, T | sanfensan |
| 5C | 6268971 | C, T | sanfensan |
| 5C | 12203702 | G, A | sanfensan |
| 5C | 16396954 | A, G | sanfensan |
| 5C | 22928378 | C, A | sanfensan |
| 5C | 26060685 | T, C | sanfensan |
| 5C | 31503941 | C, T | sanfensan |
| 5C | 39150034 | T, C | sanfensan |
| 5C | 44163319 | T, G | sanfensan |
| 5C | 47044843 | C, T | sanfensan |
| 5C | 50558717 | C, A | sanfensan |
| 5C | 53047612 | G, A | sanfensan |
| 5C | 56146722 | T, G | sanfensan |
| 5C | 63376645 | G, A | sanfensan |
| 5C | 69905284 | T, C | sanfensan |
| 5C | 74387265 | T, C | sanfensan |
| 5C | 77896048 | C, T | sanfensan |
| 5C | 81012623 | C, T | sanfensan |
| 5C | 86566325 | A, G | sanfensan |
| 5C | 94254331 | G, C | sanfensan |
| 5C | 95909941 | T, C | sanfensan |
| 5C | 101827763 | A, G | sanfensan |
| 5C | 109797422 | C, A | sanfensan |
| 5C | 114362886 | G, T | sanfensan |
| 5C | 119134297 | A, C | sanfensan |
| 5C | 121784178 | G, A | sanfensan |
| 5C | 125633452 | C, A | sanfensan |
| 5C | 132720505 | G, A | sanfensan |
| 5C | 138374966 | A, G | sanfensan |
| 5C | 143890607 | C, T | sanfensan |
| 5C | 145866226 | G, A | sanfensan |
| 5C | 150225588 | G, A | sanfensan |
| 5C | 156966699 | A, G | sanfensan |
| 5C | 160214876 | G, C | sanfensan |
| 5C | 168811131 | T, C | sanfensan |
| 5C | 174809389 | G, C | sanfensan |
| 5C | 177125115 | A, C | sanfensan |
| 5C | 182633756 | G, A | sanfensan |
| 5C | 189642953 | T, C | sanfensan |
| 5C | 193221965 | A, G | sanfensan |
| 5C | 199654787 | C, T | sanfensan |
| 5C | 201058413 | C, T | sanfensan |
| 5C | 208411390 | T, C | sanfensan |
| 5C | 214681134 | T, C | sanfensan |
| 5C | 216349665 | C, T | sanfensan |
| 5C | 220308120 | C, T | sanfensan |
| 5C | 223909826 | T, C | sanfensan |
| 5C | 227213415 | T, C | sanfensan |
| 5C | 234663041 | C, T | sanfensan |
| 5C | 236099260 | A, C | sanfensan |
| 5C | 244246397 | C, A | sanfensan |
| 5C | 246386706 | C, A | sanfensan |
| 5C | 252990985 | A, T | sanfensan |
| 5C | 255616974 | C, A | sanfensan |
| 5C | 261629346 | G, A | sanfensan |
| 5C | 266182002 | C, T | sanfensan |
| 5C | 271236736 | C, A | sanfensan |
| 5C | 275627914 | A, G | sanfensan |
| 5C | 283728720 | C, T | sanfensan |
| 5C | 285163996 | T, C | sanfensan |
| 5C | 292163090 | G, A | sanfensan |
| 5C | 297500311 | T, C | sanfensan |
| 5C | 304040309 | T, C | sanfensan |
| 5C | 306535191 | C, T | sanfensan |
| 5C | 313032349 | T, G | sanfensan |
| 5C | 318243088 | T, G | sanfensan |
| 5C | 320862543 | G, A | sanfensan |
| 5C | 325755346 | C, T | sanfensan |
| 5C | 333233541 | T, C | sanfensan |
| 5C | 338139569 | T, C | sanfensan |
| 5C | 342626437 | A, G | sanfensan |
| 5C | 347361434 | G, A | sanfensan |
| 5C | 354441762 | C, T | sanfensan |
| 5C | 358341050 | C, G | sanfensan |
| 5C | 363432176 | T, C | sanfensan |
| 5C | 369584563 | T, C | sanfensan |
| 5C | 373117738 | G, A | sanfensan |
| 5C | 377549147 | G, A | sanfensan |
| 5C | 384699195 | G, A | sanfensan |
| 5C | 385128549 | C, T | sanfensan |
| 5C | 391966210 | C, T | sanfensan |
| 5C | 399070404 | C, T | sanfensan |
| 5C | 402179080 | A, G | sanfensan |
| 5C | 405507477 | T, C | sanfensan |
| 5C | 411038650 | A, G | sanfensan |
| 5C | 416614236 | G, C | sanfensan |
| 5C | 421435620 | T, G | sanfensan |
| 5C | 427497128 | A, G | sanfensan |
| 5C | 432532568 | A, C | sanfensan |
| 5C | 435770963 | C, T | sanfensan |
| 5C | 440258824 | A, G | sanfensan |
| 5C | 447827826 | T, C | sanfensan |
| 5C | 451307256 | C, A | sanfensan |
| 5C | 458701577 | A, C | sanfensan |
| 5C | 462771476 | G, T | sanfensan |
| 5C | 468438848 | G, A | sanfensan |
| 5C | 473285230 | T, C | sanfensan |
| 5C | 477766211 | T, C | sanfensan |
| 5C | 480911717 | C, G | sanfensan |
| 5C | 489509201 | C, G | sanfensan |
| 5C | 490176188 | G, C | sanfensan |
| 5C | 498023277 | A, G | sanfensan |
| 5C | 503467351 | T, C | sanfensan |
| 5C | 509322035 | T, C | sanfensan |
| 5C | 512872294 | A, G | sanfensan |
| 5C | 515412167 | A, G | sanfensan |
| 5C | 520108101 | T, C | sanfensan |
| 5C | 526886988 | G, A | sanfensan |
| 5C | 533665172 | G, A | sanfensan |
| 5C | 536150519 | G, A | sanfensan |
| 5C | 542459731 | T, C | sanfensan |
| 5C | 548440305 | T, C | sanfensan |
| 5C | 551331730 | A, G | sanfensan |
| 5C | 555077838 | C, G | sanfensan |
| 5C | 560097889 | T, C | sanfensan |
| 5C | 569206294 | C, T | sanfensan |
| 5C | 572903980 | A, G | sanfensan |
| 5C | 579001353 | A, G | sanfensan |
| 5C | 582680547 | C, T | sanfensan |
| 5C | 585411392 | C, T | sanfensan |
| 5C | 593970148 | C, T | sanfensan |
| 5C | 598395218 | T, C | sanfensan |
| 5C | 601401624 | C, A | sanfensan |
| 5D | 4994611 | T, G | sanfensan |
| 5D | 6622542 | G, C | sanfensan |
| 5D | 10884878 | C, G | sanfensan |
| 5D | 16839171 | G, T | sanfensan |
| 5D | 21309845 | G, A | sanfensan |
| 5D | 27535507 | T, A | sanfensan |
| 5D | 34803208 | G, A | sanfensan |
| 5D | 36778603 | T, G | sanfensan |
| 5D | 41959313 | T, C | sanfensan |
| 5D | 47178435 | T, C | sanfensan |
| 5D | 52127682 | C, T | sanfensan |
| 5D | 54096343 | T, G | sanfensan |
| 5D | 56457835 | G, A | sanfensan |
| 5D | 63243306 | A, C | sanfensan |
| 5D | 64721781 | C, T | sanfensan |
| 5D | 65173806 | A, C | sanfensan |
| 5D | 66436115 | C, T | sanfensan |
| 5D | 74642065 | T, C | sanfensan |
| 5D | 79567830 | G, A | sanfensan |
| 5D | 83382549 | G, A | sanfensan |
| 5D | 87296427 | T, C | sanfensan |
| 5D | 93053910 | T, C | sanfensan |
| 5D | 99261576 | C, T | sanfensan |
| 5D | 101623688 | C, T | sanfensan |
| 5D | 109259133 | T, C | sanfensan |
| 5D | 110187772 | C, T | sanfensan |
| 5D | 116420976 | T, C | sanfensan |
| 5D | 120360110 | G, A | sanfensan |
| 5D | 128589324 | T, C | sanfensan |
| 5D | 134480766 | G, A | sanfensan |
| 5D | 136403599 | C, T | sanfensan |
| 5D | 142232990 | C, T | sanfensan |
| 5D | 145960007 | A, G | sanfensan |
| 5D | 153395376 | G, A | sanfensan |
| 5D | 155066422 | T, C | sanfensan |
| 5D | 162991627 | T, C | sanfensan |
| 5D | 167232512 | C, T | sanfensan |
| 5D | 172000670 | T, C | sanfensan |
| 5D | 177160582 | G, A | sanfensan |
| 5D | 182553299 | C, T | sanfensan |
| 5D | 187357553 | C, T | sanfensan |
| 5D | 191107356 | A, G | sanfensan |
| 5D | 199208574 | C, T | sanfensan |
| 5D | 203029590 | A, G | sanfensan |
| 5D | 209809131 | T, G | sanfensan |
| 5D | 211555734 | A, T | sanfensan |
| 5D | 216140070 | T, G | sanfensan |
| 5D | 221499002 | A, G | sanfensan |
| 5D | 228576676 | A, G | sanfensan |
| 5D | 230582553 | G, C | sanfensan |
| 5D | 238425107 | C, T | sanfensan |
| 5D | 242649305 | G, A | sanfensan |
| 5D | 245087633 | T, C | sanfensan |
| 5D | 250750865 | G, T | sanfensan |
| 5D | 258642655 | G, A | sanfensan |
| 5D | 262006477 | A, G | sanfensan |
| 5D | 266745740 | G, A | sanfensan |
| 5D | 271633260 | G, A | sanfensan |
| 5D | 278837964 | C, T | sanfensan |
| 5D | 280282084 | T, C | sanfensan |
| 5D | 289610610 | A, T | sanfensan |
| 5D | 293820840 | C, T | sanfensan |
| 5D | 298575083 | A, T | sanfensan |
| 5D | 302837192 | T, C | sanfensan |
| 5D | 305502791 | T, C | sanfensan |
| 5D | 314628778 | A, G | sanfensan |
| 5D | 319756146 | G, A | sanfensan |
| 5D | 322226424 | C, A | sanfensan |
| 5D | 328035262 | C, T | sanfensan |
| 5D | 332326959 | C, T | sanfensan |
| 5D | 339720781 | T, A | sanfensan |
| 5D | 344363610 | A, C | sanfensan |
| 5D | 345547743 | T, C | sanfensan |
| 5D | 353592498 | G, A | sanfensan |
| 5D | 357027031 | C, T | sanfensan |
| 5D | 363095927 | T, G | sanfensan |
| 5D | 365082132 | T, C | sanfensan |
| 5D | 372087578 | A, C | sanfensan |
| 5D | 375075448 | A, G | sanfensan |
| 5D | 382109948 | T, C | sanfensan |
| 5D | 385044778 | A, G | sanfensan |
| 5D | 394858589 | C, T | sanfensan |
| 5D | 399910298 | C, T | sanfensan |
| 5D | 402227007 | G, A | sanfensan |
| 5D | 407502206 | T, C | sanfensan |
| 5D | 410635861 | A, G | sanfensan |
| 5D | 419664992 | T, C | sanfensan |
| 5D | 421256053 | A, G | sanfensan |
| 5D | 427332097 | C, G | sanfensan |
| 5D | 433626893 | G, T | sanfensan |
| 5D | 437055110 | C, G | sanfensan |
| 5D | 440529761 | C, T | sanfensan |
| 5D | 446386566 | C, T | sanfensan |
| 5D | 454896966 | T, C | sanfensan |
| 5D | 458673318 | G, A | sanfensan |
| 5D | 462992703 | G, A | sanfensan |
| 5D | 466900163 | C, T | sanfensan |
| 5D | 474924404 | C, T | sanfensan |
| 5D | 478407877 | T, C | sanfensan |
| 5D | 482793426 | T, C | sanfensan |
| 5D | 488236543 | A, C | sanfensan |
| 5D | 492418351 | A, G | sanfensan |
| 5D | 497166345 | G, T | sanfensan |
| 5D | 500430897 | T, G | sanfensan |
| 6A | 4492157 | T, C | sanfensan |
| 6A | 8762221 | G, T | sanfensan |
| 6A | 11190798 | T, C | sanfensan |
| 6A | 17691406 | C, T | sanfensan |
| 6A | 20852081 | T, C | sanfensan |
| 6A | 26524896 | C, T | sanfensan |
| 6A | 33584644 | C, T | sanfensan |
| 6A | 36710498 | A, G | sanfensan |
| 6A | 42587073 | A, G | sanfensan |
| 6A | 48196250 | G, C | sanfensan |
| 6A | 54285408 | C, G | sanfensan |
| 6A | 57328665 | T, A | sanfensan |
| 6A | 64386015 | C, A | sanfensan |
| 6A | 67285702 | C, T | sanfensan |
| 6A | 71815074 | C, A | sanfensan |
| 6A | 77438434 | C, A | sanfensan |
| 6A | 83659654 | G, A | sanfensan |
| 6A | 85235001 | C, A | sanfensan |
| 6A | 94561841 | T, G | sanfensan |
| 6A | 95323662 | A, C | sanfensan |
| 6A | 103614490 | T, C | sanfensan |
| 6A | 108982250 | C, T | sanfensan |
| 6A | 109794291 | G, A | sanfensan |
| 6A | 113440903 | T, G | sanfensan |
| 6A | 118377169 | C, G | sanfensan |
| 6A | 124172048 | A, G | sanfensan |
| 6A | 125350773 | A, C | sanfensan |
| 6A | 130843545 | T, C | sanfensan |
| 6A | 135683529 | T, C | sanfensan |
| 6A | 143547400 | A, T | sanfensan |
| 6A | 147670247 | A, G | sanfensan |
| 6A | 151304761 | T, C | sanfensan |
| 6A | 157973297 | G, C | sanfensan |
| 6A | 158294119 | A, C | sanfensan |
| 6A | 163354468 | G, C | sanfensan |
| 6A | 165693464 | T, C | sanfensan |
| 6A | 173487971 | G, A | sanfensan |
| 6A | 176286584 | G, A | sanfensan |
| 6A | 183958426 | C, T | sanfensan |
| 6A | 186274335 | G, A | sanfensan |
| 6A | 194312081 | C, T | sanfensan |
| 6A | 198052953 | G, A | sanfensan |
| 6A | 203626460 | T, G | sanfensan |
| 6A | 208551969 | G, A | sanfensan |
| 6A | 212325078 | A, G | sanfensan |
| 6A | 216393559 | G, A | sanfensan |
| 6A | 222443511 | C, G | sanfensan |
| 6A | 227146163 | G, A | sanfensan |
| 6A | 232355131 | C, A | sanfensan |
| 6A | 236306595 | G, A | sanfensan |
| 6A | 242551165 | C, T | sanfensan |
| 6A | 248683146 | T, A | sanfensan |
| 6A | 254218064 | C, T | sanfensan |
| 6A | 256311166 | G, A | sanfensan |
| 6A | 263945630 | T, C | sanfensan |
| 6A | 265291617 | G, A | sanfensan |
| 6A | 272551424 | T, C | sanfensan |
| 6A | 276146901 | A, C | sanfensan |
| 6A | 283068354 | T, C | sanfensan |
| 6A | 287654071 | G, A | sanfensan |
| 6A | 291439784 | A, C | sanfensan |
| 6A | 296813413 | T, A | sanfensan |
| 6A | 304331839 | T, C | sanfensan |
| 6A | 309571759 | G, C | sanfensan |
| 6A | 311843254 | A, C | sanfensan |
| 6A | 318306732 | G, A | sanfensan |
| 6A | 324491687 | A, C | sanfensan |
| 6A | 326644360 | G, A | sanfensan |
| 6A | 330034066 | C, T | sanfensan |
| 6A | 335513362 | C, T | sanfensan |
| 6A | 339809705 | C, G | sanfensan |
| 6A | 343191906 | T, C | sanfensan |
| 6A | 349219222 | G, A | sanfensan |
| 6A | 351759840 | A, G | sanfensan |
| 6A | 358242236 | G, T | sanfensan |
| 6A | 362754991 | T, C | sanfensan |
| 6A | 367473345 | G, A | sanfensan |
| 6A | 371831728 | G, A | sanfensan |
| 6A | 377260484 | T, C | sanfensan |
| 6A | 382654181 | C, T | sanfensan |
| 6A | 389448736 | A, G | sanfensan |
| 6A | 394354567 | T, C | sanfensan |
| 6A | 395689953 | A, G | sanfensan |
| 6A | 402143190 | A, G | sanfensan |
| 6A | 409084859 | A, C | sanfensan |
| 6A | 414438357 | A, G | sanfensan |
| 6A | 415355947 | T, C | sanfensan |
| 6A | 421445718 | A, C | sanfensan |
| 6A | 422458226 | G, A | sanfensan |
| 6A | 425035666 | C, A | sanfensan |
| 6A | 433725430 | A, G | sanfensan |
| 6A | 437585163 | T, C | sanfensan |
| 6A | 444959115 | G, C | sanfensan |
| 6A | 445547963 | C, T | sanfensan |
| 6A | 454994988 | A, G | sanfensan |
| 6C | 4740123 | A, C | sanfensan |
| 6C | 7818111 | T, G | sanfensan |
| 6C | 10608939 | A, T | sanfensan |
| 6C | 17502954 | A, C | sanfensan |
| 6C | 21919209 | C, T | sanfensan |
| 6C | 27489398 | T, C | sanfensan |
| 6C | 32883642 | T, G | sanfensan |
| 6C | 35200287 | C, A | sanfensan |
| 6C | 38472337 | T, C | sanfensan |
| 6C | 43196331 | A, G | sanfensan |
| 6C | 47589408 | A, G | sanfensan |
| 6C | 51088171 | A, G | sanfensan |
| 6C | 59096246 | G, A | sanfensan |
| 6C | 60407986 | C, T | sanfensan |
| 6C | 65757372 | C, T | sanfensan |
| 6C | 66609935 | G, A | sanfensan |
| 6C | 72292296 | A, G | sanfensan |
| 6C | 75973462 | A, T | sanfensan |
| 6C | 81974276 | C, T | sanfensan |
| 6C | 88129794 | G, C | sanfensan |
| 6C | 94193475 | T, C | sanfensan |
| 6C | 95536874 | T, C | sanfensan |
| 6C | 101717822 | C, T | sanfensan |
| 6C | 107891612 | C, T | sanfensan |
| 6C | 112851715 | A, T | sanfensan |
| 6C | 116496712 | C, T | sanfensan |
| 6C | 121111029 | T, C | sanfensan |
| 6C | 125320836 | G, A | sanfensan |
| 6C | 132900758 | T, A | sanfensan |
| 6C | 135785484 | G, C | sanfensan |
| 6C | 143030880 | C, A | sanfensan |
| 6C | 148851945 | G, A | sanfensan |
| 6C | 154103608 | A, C | sanfensan |
| 6C | 156034908 | T, G | sanfensan |
| 6C | 162091462 | A, C | sanfensan |
| 6C | 165806162 | A, T | sanfensan |
| 6C | 172155825 | G, A | sanfensan |
| 6C | 175590346 | C, T | sanfensan |
| 6C | 180731171 | G, A | sanfensan |
| 6C | 185475790 | A, G | sanfensan |
| 6C | 194315523 | G, A | sanfensan |
| 6C | 196088194 | G, A | sanfensan |
| 6C | 196851358 | C, T | sanfensan |
| 6C | 200122712 | A, C | sanfensan |
| 6C | 207092401 | A, G | sanfensan |
| 6C | 213333184 | T, G | sanfensan |
| 6C | 217526046 | G, A | sanfensan |
| 6C | 222420550 | C, T | sanfensan |
| 6C | 227353579 | G, A | sanfensan |
| 6C | 234060684 | A, G | sanfensan |
| 6C | 235857775 | G, A | sanfensan |
| 6C | 244931366 | T, C | sanfensan |
| 6C | 246070168 | G, A | sanfensan |
| 6C | 253789587 | C, T | sanfensan |
| 6C | 257684248 | G, C | sanfensan |
| 6C | 260070968 | C, T | sanfensan |
| 6C | 266510278 | C, T | sanfensan |
| 6C | 272777330 | C, T | sanfensan |
| 6C | 275890891 | G, A | sanfensan |
| 6C | 284003530 | C, T | sanfensan |
| 6C | 289057124 | C, T | sanfensan |
| 6C | 292273925 | G, A | sanfensan |
| 6C | 295852267 | A, C | sanfensan |
| 6C | 301581063 | G, A | sanfensan |
| 6C | 308002832 | T, C | sanfensan |
| 6C | 310533434 | G, A | sanfensan |
| 6C | 316651599 | G, C | sanfensan |
| 6C | 320349680 | G, A | sanfensan |
| 6C | 325082414 | T, C | sanfensan |
| 6C | 331834200 | C, T | sanfensan |
| 6C | 335435627 | T, C | sanfensan |
| 6C | 341601920 | C, A | sanfensan |
| 6C | 348384420 | T, C | sanfensan |
| 6C | 354770875 | T, C | sanfensan |
| 6C | 355787593 | T, C | sanfensan |
| 6C | 363981785 | C, T | sanfensan |
| 6C | 367279098 | C, G | sanfensan |
| 6C | 372097294 | G, T | sanfensan |
| 6C | 376086340 | G, A | sanfensan |
| 6C | 382987219 | C, T | sanfensan |
| 6C | 385017932 | A, G | sanfensan |
| 6C | 393722890 | C, T | sanfensan |
| 6C | 396489334 | T, C | sanfensan |
| 6C | 402999061 | G, A | sanfensan |
| 6C | 404034553 | G, A | sanfensan |
| 6C | 407439238 | G, C | sanfensan |
| 6C | 412317336 | C, T | sanfensan |
| 6C | 416156258 | T, C | sanfensan |
| 6C | 422586122 | G, C | sanfensan |
| 6C | 429700897 | C, T | sanfensan |
| 6C | 430532023 | A, G | sanfensan |
| 6C | 436366975 | G, A | sanfensan |
| 6C | 440291570 | T, G | sanfensan |
| 6C | 447368142 | A, G | sanfensan |
| 6C | 454158229 | C, T | sanfensan |
| 6C | 457431878 | G, A | sanfensan |
| 6C | 464408570 | A, G | sanfensan |
| 6C | 466196657 | G, A | sanfensan |
| 6C | 470467140 | G, T | sanfensan |
| 6C | 475226773 | G, A | sanfensan |
| 6C | 484622983 | G, A | sanfensan |
| 6C | 489733584 | G, T | sanfensan |
| 6C | 491124804 | T, C | sanfensan |
| 6C | 496637243 | G, A | sanfensan |
| 6C | 502828531 | C, T | sanfensan |
| 6C | 505002174 | G, A | sanfensan |
| 6C | 507736024 | C, T | sanfensan |
| 6C | 512237599 | C, T | sanfensan |
| 6C | 516058707 | C, T | sanfensan |
| 6C | 522649701 | C, G | sanfensan |
| 6C | 529463088 | G, T | sanfensan |
| 6C | 532838127 | C, T | sanfensan |
| 6C | 538744714 | C, T | sanfensan |
| 6C | 541894652 | T, C | sanfensan |
| 6C | 548890274 | A, G | sanfensan |
| 6C | 550597198 | C, T | sanfensan |
| 6C | 555553384 | T, C | sanfensan |
| 6C | 563333574 | G, T | sanfensan |
| 6C | 566938058 | T, C | sanfensan |
| 6C | 574826495 | A, C | sanfensan |
| 6C | 576805876 | G, A | sanfensan |
| 6C | 581584319 | G, C | sanfensan |
| 6C | 585343852 | C, T | sanfensan |
| 6C | 590741273 | T, C | sanfensan |
| 6C | 599908901 | G, A | sanfensan |
| 6C | 601571029 | C, T | sanfensan |
| 6C | 605547418 | C, T | sanfensan |
| 6D | 2777295 | C, T | sanfensan |
| 6D | 6267655 | C, A | sanfensan |
| 6D | 13107420 | C, T | sanfensan |
| 6D | 16132609 | C, A | sanfensan |
| 6D | 23982220 | A, C | sanfensan |
| 6D | 26754468 | T, G | sanfensan |
| 6D | 32172782 | C, T | sanfensan |
| 6D | 35628869 | T, C | sanfensan |
| 6D | 44217561 | A, G | sanfensan |
| 6D | 45240610 | T, G | sanfensan |
| 6D | 50128337 | G, C | sanfensan |
| 6D | 55030113 | A, G | sanfensan |
| 6D | 64553482 | T, C | sanfensan |
| 6D | 66247035 | T, C | sanfensan |
| 6D | 73309196 | A, G | sanfensan |
| 6D | 77363272 | A, G | sanfensan |
| 6D | 84655953 | T, C | sanfensan |
| 6D | 89015326 | T, C | sanfensan |
| 6D | 91516090 | A, G | sanfensan |
| 6D | 98584233 | A, G | sanfensan |
| 6D | 104170504 | T, G | sanfensan |
| 6D | 108654428 | T, C | sanfensan |
| 6D | 114351697 | C, G | sanfensan |
| 6D | 118475182 | G, A | sanfensan |
| 6D | 120657246 | A, G | sanfensan |
| 6D | 128950046 | A, G | sanfensan |
| 6D | 132670154 | T, C | sanfensan |
| 6D | 139598945 | A, G | sanfensan |
| 6D | 142833751 | A, C | sanfensan |
| 6D | 146878955 | C, T | sanfensan |
| 6D | 150804995 | G, A | sanfensan |
| 6D | 159442646 | T, C | sanfensan |
| 6D | 161979848 | G, A | sanfensan |
| 6D | 166007399 | C, T | sanfensan |
| 6D | 171110144 | T, C | sanfensan |
| 6D | 178157758 | C, T | sanfensan |
| 6D | 181144200 | A, C | sanfensan |
| 6D | 181398145 | T, G | sanfensan |
| 6D | 188963826 | T, C | sanfensan |
| 6D | 190320583 | G, A | sanfensan |
| 6D | 198738426 | G, A | sanfensan |
| 6D | 204695478 | C, T | sanfensan |
| 6D | 209111889 | A, C | sanfensan |
| 6D | 213388210 | T, C | sanfensan |
| 6D | 217365606 | G, A | sanfensan |
| 6D | 220497638 | C, T | sanfensan |
| 6D | 226248655 | G, T | sanfensan |
| 6D | 230357776 | T, C | sanfensan |
| 6D | 239771210 | A, G | sanfensan |
| 6D | 240712867 | T, A | sanfensan |
| 6D | 245616215 | G, A | sanfensan |
| 6D | 254910351 | T, C | sanfensan |
| 6D | 257401569 | T, C | sanfensan |
| 6D | 263701170 | T, G | sanfensan |
| 6D | 268768437 | A, G | sanfensan |
| 6D | 270928208 | C, A | sanfensan |
| 6D | 278371196 | C, T | sanfensan |
| 6D | 284570630 | A, G | sanfensan |
| 6D | 285173852 | A, G | sanfensan |
| 6D | 290592914 | T, A | sanfensan |
| 7A | 556988 | G, A | sanfensan |
| 7A | 9387452 | T, C | sanfensan |
| 7A | 10624667 | T, C | sanfensan |
| 7A | 16247028 | G, A | sanfensan |
| 7A | 24258432 | T, A | sanfensan |
| 7A | 29212189 | T, C | sanfensan |
| 7A | 32277248 | T, G | sanfensan |
| 7A | 38743380 | T, C | sanfensan |
| 7A | 42755867 | A, G | sanfensan |
| 7A | 46387061 | C, T | sanfensan |
| 7A | 51855350 | A, G | sanfensan |
| 7A | 57885922 | C, A | sanfensan |
| 7A | 63281807 | T, C | sanfensan |
| 7A | 65480794 | G, A | sanfensan |
| 7A | 71137163 | T, C | sanfensan |
| 7A | 75733143 | T, A | sanfensan |
| 7A | 82158130 | G, C | sanfensan |
| 7A | 85385227 | C, T | sanfensan |
| 7A | 91146020 | C, T | sanfensan |
| 7A | 99518702 | T, C | sanfensan |
| 7A | 100100809 | C, A | sanfensan |
| 7A | 108559177 | C, T | sanfensan |
| 7A | 110159568 | G, T | sanfensan |
| 7A | 119859092 | T, C | sanfensan |
| 7A | 120329198 | C, A | sanfensan |
| 7A | 125074054 | T, C | sanfensan |
| 7A | 131168556 | C, T | sanfensan |
| 7A | 139918960 | A, G | sanfensan |
| 7A | 143266476 | T, C | sanfensan |
| 7A | 149300335 | G, A | sanfensan |
| 7A | 154734171 | T, C | sanfensan |
| 7A | 159313052 | C, T | sanfensan |
| 7A | 160075815 | C, G | sanfensan |
| 7A | 167272883 | C, T | sanfensan |
| 7A | 171591003 | T, G | sanfensan |
| 7A | 177018083 | G, T | sanfensan |
| 7A | 184807238 | C, A | sanfensan |
| 7A | 186333580 | A, G | sanfensan |
| 7A | 190961467 | T, C | sanfensan |
| 7A | 197600313 | G, A | sanfensan |
| 7A | 201074764 | C, A | sanfensan |
| 7A | 208272139 | T, G | sanfensan |
| 7A | 212498087 | C, T | sanfensan |
| 7A | 219185506 | G, T | sanfensan |
| 7A | 222607909 | A, G | sanfensan |
| 7A | 226206685 | T, C | sanfensan |
| 7A | 234382860 | A, T | sanfensan |
| 7A | 235670912 | C, T | sanfensan |
| 7A | 242252774 | A, G | sanfensan |
| 7A | 246670561 | A, C | sanfensan |
| 7A | 250206642 | T, C | sanfensan |
| 7A | 259731844 | C, T | sanfensan |
| 7A | 261123685 | T, C | sanfensan |
| 7A | 267061512 | G, C | sanfensan |
| 7A | 273875056 | A, T | sanfensan |
| 7A | 279451537 | C, G | sanfensan |
| 7A | 283880271 | G, A | sanfensan |
| 7A | 289470204 | T, C | sanfensan |
| 7A | 294405482 | T, C | sanfensan |
| 7A | 296729065 | G, T | sanfensan |
| 7A | 304733749 | T, C | sanfensan |
| 7A | 306515921 | T, C | sanfensan |
| 7A | 310678817 | G, A | sanfensan |
| 7A | 317998480 | C, T | sanfensan |
| 7A | 324083519 | A, G | sanfensan |
| 7A | 326015574 | A, G | sanfensan |
| 7A | 334096551 | G, A | sanfensan |
| 7A | 337503568 | G, A | sanfensan |
| 7A | 342389640 | T, C | sanfensan |
| 7A | 349080210 | A, G | sanfensan |
| 7A | 352615237 | T, C | sanfensan |
| 7A | 355460008 | G, A | sanfensan |
| 7A | 361858462 | T, C | sanfensan |
| 7A | 363873233 | C, T | sanfensan |
| 7A | 367416741 | A, G | sanfensan |
| 7A | 370900705 | C, A | sanfensan |
| 7A | 377494585 | C, T | sanfensan |
| 7A | 380620357 | T, C | sanfensan |
| 7A | 387542205 | A, G | sanfensan |
| 7A | 393193897 | T, C | sanfensan |
| 7A | 397890567 | A, G | sanfensan |
| 7A | 402356276 | A, T | sanfensan |
| 7A | 408034177 | T, G | sanfensan |
| 7A | 411938390 | C, T | sanfensan |
| 7A | 415820845 | C, T | sanfensan |
| 7A | 421650338 | A, G | sanfensan |
| 7A | 429369869 | A, G | sanfensan |
| 7A | 433335698 | G, A | sanfensan |
| 7A | 438899194 | T, C | sanfensan |
| 7A | 442020982 | T, C | sanfensan |
| 7A | 449206870 | A, G | sanfensan |
| 7A | 452644138 | A, C | sanfensan |
| 7A | 455480446 | T, G | sanfensan |
| 7A | 463118331 | A, G | sanfensan |
| 7A | 467735097 | C, T | sanfensan |
| 7A | 471902717 | A, G | sanfensan |
| 7A | 476107323 | A, G | sanfensan |
| 7A | 482021904 | C, T | sanfensan |
| 7A | 485131125 | A, G | sanfensan |
| 7A | 493482291 | G, A | sanfensan |
| 7A | 495121727 | T, C | sanfensan |
| 7C | 4614858 | T, G | sanfensan |
| 7C | 5317219 | C, T | sanfensan |
| 7C | 14340861 | A, G | sanfensan |
| 7C | 19478034 | G, A | sanfensan |
| 7C | 24565676 | G, A | sanfensan |
| 7C | 29930105 | T, C | sanfensan |
| 7C | 32494696 | G, A | sanfensan |
| 7C | 39005353 | C, T | sanfensan |
| 7C | 41317427 | A, G | sanfensan |
| 7C | 47961168 | A, C | sanfensan |
| 7C | 50182911 | G, C | sanfensan |
| 7C | 55925952 | A, G | sanfensan |
| 7C | 61239403 | C, G | sanfensan |
| 7C | 67047337 | T, G | sanfensan |
| 7C | 71975946 | G, T | sanfensan |
| 7C | 72522696 | C, T | sanfensan |
| 7C | 79287234 | A, C | sanfensan |
| 7C | 84848962 | G, A | sanfensan |
| 7C | 88219749 | C, T | sanfensan |
| 7C | 94995046 | A, G | sanfensan |
| 7C | 97771126 | T, C | sanfensan |
| 7C | 104891291 | G, A | sanfensan |
| 7C | 108552236 | A, G | sanfensan |
| 7C | 113384152 | G, T | sanfensan |
| 7C | 118141656 | A, G | sanfensan |
| 7C | 123007101 | G, C | sanfensan |
| 7C | 129029468 | T, C | sanfensan |
| 7C | 134291990 | T, C | sanfensan |
| 7C | 137256580 | T, C | sanfensan |
| 7C | 140877690 | G, A | sanfensan |
| 7C | 145949568 | G, C | sanfensan |
| 7C | 151007290 | G, A | sanfensan |
| 7C | 157054447 | C, T | sanfensan |
| 7C | 163909424 | G, A | sanfensan |
| 7C | 166612341 | A, C | sanfensan |
| 7C | 171906105 | A, G | sanfensan |
| 7C | 177293559 | T, C | sanfensan |
| 7C | 179283192 | T, C | sanfensan |
| 7C | 180628837 | C, T | sanfensan |
| 7C | 185998111 | A, G | sanfensan |
| 7C | 191666515 | A, G | sanfensan |
| 7C | 197044982 | A, G | sanfensan |
| 7C | 204791800 | A, C | sanfensan |
| 7C | 206297454 | T, C | sanfensan |
| 7C | 214665049 | T, C | sanfensan |
| 7C | 216739159 | A, G | sanfensan |
| 7C | 220606602 | T, C | sanfensan |
| 7C | 225604579 | C, A | sanfensan |
| 7C | 232971786 | G, C | sanfensan |
| 7C | 236226644 | C, T | sanfensan |
| 7C | 242089348 | C, G | sanfensan |
| 7C | 247003405 | C, T | sanfensan |
| 7C | 249178004 | T, G | sanfensan |
| 7C | 250146710 | C, T | sanfensan |
| 7C | 257100796 | G, A | sanfensan |
| 7C | 261648943 | C, A | sanfensan |
| 7C | 268903706 | T, C | sanfensan |
| 7C | 273820692 | C, G | sanfensan |
| 7C | 278686128 | T, C | sanfensan |
| 7C | 280947265 | G, A | sanfensan |
| 7C | 286809973 | T, C | sanfensan |
| 7C | 291012394 | G, C | sanfensan |
| 7C | 296841339 | A, C | sanfensan |
| 7C | 301552328 | T, C | sanfensan |
| 7C | 304378578 | A, G | sanfensan |
| 7C | 306040659 | G, A | sanfensan |
| 7C | 314412301 | G, T | sanfensan |
| 7C | 318527191 | C, T | sanfensan |
| 7C | 320205502 | A, G | sanfensan |
| 7C | 320293039 | G, T | sanfensan |
| 7C | 327857134 | C, A | sanfensan |
| 7C | 331712162 | T, C | sanfensan |
| 7C | 339499335 | T, C | sanfensan |
| 7C | 340372302 | C, T | sanfensan |
| 7C | 349292850 | C, T | sanfensan |
| 7C | 350022239 | G, A | sanfensan |
| 7C | 350993553 | A, G | sanfensan |
| 7C | 358188563 | T, C | sanfensan |
| 7C | 363492217 | C, T | sanfensan |
| 7C | 369432993 | G, A | sanfensan |
| 7C | 371369521 | T, G | sanfensan |
| 7C | 377634589 | A, G | sanfensan |
| 7C | 384171632 | C, T | sanfensan |
| 7C | 386369415 | G, A | sanfensan |
| 7C | 392363537 | C, T | sanfensan |
| 7C | 396114953 | A, G | sanfensan |
| 7C | 400812310 | C, T | sanfensan |
| 7C | 405139125 | A, G | sanfensan |
| 7C | 413833654 | A, C | sanfensan |
| 7C | 414551856 | G, T | sanfensan |
| 7C | 416262975 | C, T | sanfensan |
| 7C | 420923703 | T, C | sanfensan |
| 7C | 426911439 | C, A | sanfensan |
| 7C | 430613225 | G, A | sanfensan |
| 7C | 432461759 | G, A | sanfensan |
| 7C | 435550002 | C, T | sanfensan |
| 7C | 444070312 | G, T | sanfensan |
| 7C | 445489155 | G, A | sanfensan |
| 7C | 452005889 | A, T | sanfensan |
| 7C | 457711724 | T, C | sanfensan |
| 7C | 462688367 | G, A | sanfensan |
| 7C | 464956118 | A, G | sanfensan |
| 7C | 465254207 | G, T | sanfensan |
| 7C | 471154727 | G, A | sanfensan |
| 7C | 476266406 | C, A | sanfensan |
| 7C | 480020947 | C, T | sanfensan |
| 7C | 485814933 | A, G | sanfensan |
| 7C | 491712274 | T, C | sanfensan |
| 7C | 496402779 | G, C | sanfensan |
| 7C | 500349440 | G, A | sanfensan |
| 7C | 505836662 | C, T | sanfensan |
| 7C | 514393934 | A, G | sanfensan |
| 7C | 518087154 | C, G | sanfensan |
| 7C | 520313196 | A, G | sanfensan |
| 7C | 528181463 | G, C | sanfensan |
| 7C | 530421340 | G, C | sanfensan |
| 7D | 2445778 | G, A | sanfensan |
| 7D | 9742047 | A, G | sanfensan |
| 7D | 9847518 | A, G | sanfensan |
| 7D | 13815389 | A, T | sanfensan |
| 7D | 18736551 | G, T | sanfensan |
| 7D | 20505929 | C, T | sanfensan |
| 7D | 25511353 | G, A | sanfensan |
| 7D | 30499092 | G, A | sanfensan |
| 7D | 36614804 | T, G | sanfensan |
| 7D | 40700604 | G, C | sanfensan |
| 7D | 46604719 | C, T | sanfensan |
| 7D | 50727705 | C, G | sanfensan |
| 7D | 57700439 | C, T | sanfensan |
| 7D | 60169262 | C, A | sanfensan |
| 7D | 69063876 | A, C | sanfensan |
| 7D | 71993254 | T, C | sanfensan |
| 7D | 79211559 | A, G | sanfensan |
| 7D | 82904994 | C, T | sanfensan |
| 7D | 85615821 | A, G | sanfensan |
| 7D | 91224657 | T, C | sanfensan |
| 7D | 98952743 | G, A | sanfensan |
| 7D | 100841898 | C, T | sanfensan |
| 7D | 108477777 | G, A | sanfensan |
| 7D | 110334702 | C, T | sanfensan |
| 7D | 116643732 | C, T | sanfensan |
| 7D | 123307950 | T, C | sanfensan |
| 7D | 127161912 | A, G | sanfensan |
| 7D | 133410614 | T, G | sanfensan |
| 7D | 138641756 | C, T | sanfensan |
| 7D | 141652553 | T, G | sanfensan |
| 7D | 146207944 | C, T | sanfensan |
| 7D | 151573471 | G, A | sanfensan |
| 7D | 156040797 | C, A | sanfensan |
| 7D | 161660385 | A, G | sanfensan |
| 7D | 167061192 | A, G | sanfensan |
| 7D | 170724248 | A, G | sanfensan |
| 7D | 178681077 | A, C | sanfensan |
| 7D | 183800740 | A, C | sanfensan |
| 7D | 186691354 | A, G | sanfensan |
| 7D | 192032150 | G, A | sanfensan |
| 7D | 197240877 | G, A | sanfensan |
| 7D | 203331302 | A, G | sanfensan |
| 7D | 206895614 | A, G | sanfensan |
| 7D | 214500259 | A, T | sanfensan |
| 7D | 216095702 | C, A | sanfensan |
| 7D | 223545306 | G, A | sanfensan |
| 7D | 226229762 | G, A | sanfensan |
| 7D | 232450357 | G, C | sanfensan |
| 7D | 237413151 | T, C | sanfensan |
| 7D | 243355850 | T, C | sanfensan |
| 7D | 247929240 | G, T | sanfensan |
| 7D | 254107993 | C, G | sanfensan |
| 7D | 255422386 | T, C | sanfensan |
| 7D | 264369755 | A, C | sanfensan |
| 7D | 267080055 | G, C | sanfensan |
| 7D | 272102782 | T, C | sanfensan |
| 7D | 276724318 | T, C | sanfensan |
| 7D | 278615324 | G, C | sanfensan |
| 7D | 283701259 | T, G | sanfensan |
| 7D | 285490224 | C, T | sanfensan |
| 7D | 290974761 | C, T | sanfensan |
| 7D | 299205902 | T, C | sanfensan |
| 7D | 302081398 | G, A | sanfensan |
| 7D | 305799487 | A, G | sanfensan |
| 7D | 310083219 | A, G | sanfensan |
| 7D | 318265612 | C, T | sanfensan |
| 7D | 324554035 | C, A | sanfensan |
| 7D | 328162737 | A, G | sanfensan |
| 7D | 332476238 | C, T | sanfensan |
| 7D | 336554421 | T, C | sanfensan |
| 7D | 342546157 | T, C | sanfensan |
| 7D | 348921802 | A, G | sanfensan |
| 7D | 352024567 | A, C | sanfensan |
| 7D | 358950077 | C, T | sanfensan |
| 7D | 362820207 | G, A | sanfensan |
| 7D | 368623531 | T, C | sanfensan |
| 7D | 371406914 | G, T | sanfensan |
| 7D | 379681421 | T, C | sanfensan |
| 7D | 382815770 | C, T | sanfensan |
| 7D | 389570780 | G, A | sanfensan |
| 7D | 390101972 | G, A | sanfensan |
| 7D | 399143078 | C, T | sanfensan |
| 7D | 400807921 | C, T | sanfensan |
| 7D | 406112763 | T, G | sanfensan |
| 7D | 411104681 | A, G | sanfensan |
| 7D | 415490064 | G, A | sanfensan |
| 7D | 423220521 | A, G | sanfensan |
| 7D | 426049443 | T, C | sanfensan |
| 7D | 432545626 | A, G | sanfensan |
| 7D | 438677802 | A, T | sanfensan |
| 7D | 442595402 | T, C | sanfensan |
| 7D | 447536505 | C, T | sanfensan |
| 7D | 453173144 | T, A | sanfensan |
| 7D | 456025113 | C, T | sanfensan |
| 7D | 463196054 | A, T | sanfensan |
| 7D | 466703278 | A, G | sanfensan |
| 7D | 474383321 | G, T | sanfensan |
| 7D | 478751163 | T, C | sanfensan |
| 7D | 481249084 | C, G | sanfensan |
| 7D | 486113092 | A, T | sanfensan |
| 7D | 491181988 | G, T | sanfensan |
| 7D | 497047184 | A, G | sanfensan |
| 1A | 106635350 | G, C | OT3098 |
| 1A | 304418445 | G, A | OT3098 |
| 1A | 338092374 | C, T | OT3098 |
| 1A | 348499239 | C, T | OT3098 |
| 1A | 369374878 | A, G | OT3098 |
| 1A | 383912262 | C, T | OT3098 |
| 1A | 397750385 | A, G | OT3098 |
| 1A | 512105703 | G, A | OT3098 |
| 1C | 40349872 | G, T | OT3098 |
| 1C | 80742591 | G, T | OT3098 |
| 1C | 104538708 | A, G | OT3098 |
| 1C | 119650459 | G, T | OT3098 |
| 1C | 120468329 | T, G | OT3098 |
| 1C | 181195253 | G, A | OT3098 |
| 1C | 242119623 | C, T | OT3098 |
| 1C | 265897428 | C, T | OT3098 |
| 1C | 280066160 | G, A | OT3098 |
| 1C | 291282749 | A, T | OT3098 |
| 1C | 319005689 | C, A | OT3098 |
| 1C | 342223334 | G, A | OT3098 |
| 1C | 362511327 | C, T | OT3098 |
| 1C | 403231603 | G, T | OT3098 |
| 1D | 28155193 | G, A | OT3098 |
| 1D | 105612570 | C, T | OT3098 |
| 1D | 114754382 | T, A | OT3098 |
| 1D | 153648523 | C, T | OT3098 |
| 1D | 168181978 | G, A | OT3098 |
| 1D | 178507460 | T, C | OT3098 |
| 1D | 186530691 | C, G | OT3098 |
| 1D | 199381843 | C, T | OT3098 |
| 1D | 213294278 | C, A | OT3098 |
| 1D | 234770750 | A, G | OT3098 |
| 1D | 249534977 | G, A | OT3098 |
| 1D | 336367249 | T, C | OT3098 |
| 1D | 458302223 | A, G | OT3098 |
| 2A | 288229046 | G, T | OT3098 |
| 2A | 298965546 | A, G | OT3098 |
| 2A | 312180949 | G, A | OT3098 |
| 2A | 348182068 | C, T | OT3098 |
| 2A | 374638766 | A, G | OT3098 |
| 2A | 399111341 | G, A | OT3098 |
| 2C | 28799948 | T, C | OT3098 |
| 2C | 42159271 | T, G | OT3098 |
| 2C | 61574030 | G, T | OT3098 |
| 2C | 87111306 | T, C | OT3098 |
| 2C | 163497553 | G, A | OT3098 |
| 2C | 384573213 | A, G | OT3098 |
| 2C | 526110948 | A, G | OT3098 |
| 2C | 530336382 | G, A | OT3098 |
| 2D | 25489315 | A, G | OT3098 |
| 2D | 48295932 | C, T | OT3098 |
| 2D | 136287278 | G, A | OT3098 |
| 2D | 257949893 | T, C | OT3098 |
| 2D | 374246423 | T, G | OT3098 |
| 2D | 439808678 | A, G | OT3098 |
| 2D | 464345033 | G, A | OT3098 |
| 3A | 13251626 | G, A | OT3098 |
| 3A | 65224422 | C, T | OT3098 |
| 3A | 80331258 | G, A | OT3098 |
| 3A | 102356888 | G, A | OT3098 |
| 3A | 235151032 | C, T | OT3098 |
| 3A | 290609726 | A, G | OT3098 |
| 3A | 342060468 | A, G | OT3098 |
| 3A | 375362853 | G, T | OT3098 |
| 3C | 83743224 | C, T | OT3098 |
| 3C | 105875209 | T, A | OT3098 |
| 3C | 153371948 | G, A | OT3098 |
| 3C | 160415743 | A, G | OT3098 |
| 3C | 188382442 | C, T | OT3098 |
| 3C | 199311115 | T, C | OT3098 |
| 3C | 215249361 | C, T | OT3098 |
| 3C | 242293326 | A, C | OT3098 |
| 3C | 253359904 | T, C | OT3098 |
| 3C | 272308234 | G, T | OT3098 |
| 3C | 302216223 | G, A | OT3098 |
| 3C | 312338539 | T, C | OT3098 |
| 3C | 327416798 | T, C | OT3098 |
| 3C | 349649824 | A, C | OT3098 |
| 3C | 351983098 | G, A | OT3098 |
| 3C | 416217578 | T, A | OT3098 |
| 3C | 533296338 | T, C | OT3098 |
| 3C | 543025252 | A, G | OT3098 |
| 3C | 551825075 | C, T | OT3098 |
| 3C | 571627195 | T, C | OT3098 |
| 3C | 606797452 | C, A | OT3098 |
| 3C | 620635579 | G, A | OT3098 |
| 3D | 471246706 | C, T | OT3098 |
| 4A | 4383966 | T, C | OT3098 |
| 4A | 91672697 | A, G | OT3098 |
| 4A | 176172514 | T, C | OT3098 |
| 4A | 262281263 | T, G | OT3098 |
| 4A | 281599989 | C, T | OT3098 |
| 4A | 315467166 | T, C | OT3098 |
| 4A | 330624554 | C, T | OT3098 |
| 4A | 344502223 | G, A | OT3098 |
| 4A | 370035053 | A, G | OT3098 |
| 4A | 409954087 | G, A | OT3098 |
| 4A | 438257237 | C, G | OT3098 |
| 4A | 452257049 | G, A | OT3098 |
| 4C | 228404747 | C, T | OT3098 |
| 4C | 326801689 | T, C | OT3098 |
| 4C | 372872071 | G, A | OT3098 |
| 4C | 405897221 | T, C | OT3098 |
| 4C | 415253727 | C, G | OT3098 |
| 4C | 501843966 | T, G | OT3098 |
| 4C | 589184920 | A, T | OT3098 |
| 4C | 629827712 | C, A | OT3098 |
| 4C | 646042521 | A, T | OT3098 |
| 4C | 660379855 | G, A | OT3098 |
| 4C | 680363280 | G, A | OT3098 |
| 4C | 690482618 | G, A | OT3098 |
| 4D | 323386091 | T, C | OT3098 |
| 4D | 348557099 | G, A | OT3098 |
| 4D | 377370815 | A, C | OT3098 |
| 4D | 386553314 | C, T | OT3098 |
| 4D | 434004607 | A, G | OT3098 |
| 4D | 455273707 | C, T | OT3098 |
| 5A | 12250117 | C, T | OT3098 |
| 5A | 66713969 | G, T | OT3098 |
| 5A | 163771340 | C, T | OT3098 |
| 5A | 229347037 | A, G | OT3098 |
| 5A | 247731935 | C, T | OT3098 |
| 5A | 259020640 | C, T | OT3098 |
| 5A | 275554651 | C, T | OT3098 |
| 5A | 450812604 | G, A | OT3098 |
| 5A | 464203725 | C, T | OT3098 |
| 5C | 19684941 | G, A | OT3098 |
| 5C | 34175263 | G, T | OT3098 |
| 5C | 56717315 | T, G | OT3098 |
| 5C | 103289073 | G, T | OT3098 |
| 5C | 162396640 | T, C | OT3098 |
| 5C | 276244815 | G, A | OT3098 |
| 5C | 313968741 | G, A | OT3098 |
| 5C | 330901610 | A, G | OT3098 |
| 5C | 491868915 | T, C | OT3098 |
| 5C | 501340024 | G, A | OT3098 |
| 5C | 537051000 | T, C | OT3098 |
| 5C | 567644495 | G, A | OT3098 |
| 5C | 570184840 | A, C | OT3098 |
| 5D | 94959246 | G, A | OT3098 |
| 5D | 100600265 | G, A | OT3098 |
| 5D | 124321696 | T, C | OT3098 |
| 5D | 149532876 | G, A | OT3098 |
| 5D | 193714037 | A, G | OT3098 |
| 5D | 220790821 | A, G | OT3098 |
| 5D | 247323372 | T, C | OT3098 |
| 5D | 341143271 | A, G | OT3098 |
| 5D | 392150519 | A, C | OT3098 |
| 5D | 487408616 | A, G | OT3098 |
| 5D | 496068151 | G, T | OT3098 |
| 6A | 47261549 | G, C | OT3098 |
| 6A | 79634441 | C, A | OT3098 |
| 6A | 90176068 | T, C | OT3098 |
| 6A | 103858240 | T, C | OT3098 |
| 6A | 143659802 | T, C | OT3098 |
| 6A | 159101934 | A, T | OT3098 |
| 6A | 171391185 | C, A | OT3098 |
| 6A | 184973728 | A, G | OT3098 |
| 6A | 192005904 | G, A | OT3098 |
| 6A | 263939310 | C, T | OT3098 |
| 6A | 300620942 | A, G | OT3098 |
| 6A | 419205117 | T, A | OT3098 |
| 6C | 59229976 | C, T | OT3098 |
| 6C | 84591374 | C, A | OT3098 |
| 6C | 87587254 | G, C | OT3098 |
| 6C | 144536268 | C, A | OT3098 |
| 6C | 238362770 | C, A | OT3098 |
| 6C | 246928523 | C, T | OT3098 |
| 6C | 292396236 | T, C | OT3098 |
| 6C | 342467150 | A, G | OT3098 |
| 6C | 366301685 | G, A | OT3098 |
| 6C | 467269710 | C, T | OT3098 |
| 6C | 485083612 | C, T | OT3098 |
| 6C | 574716448 | C, T | OT3098 |
| 6C | 602967220 | T, C | OT3098 |
| 6C | 611213471 | T, C | OT3098 |
| 6D | 67717241 | G, A | OT3098 |
| 6D | 87582596 | A, G | OT3098 |
| 6D | 130251011 | A, G | OT3098 |
| 6D | 143238944 | C, T | OT3098 |
| 6D | 161653963 | A, G | OT3098 |
| 6D | 193391379 | C, T | OT3098 |
| 6D | 202580655 | G, A | OT3098 |
| 6D | 240523148 | G, C | OT3098 |
| 6D | 256237708 | G, T | OT3098 |
| 6D | 265237433 | T, C | OT3098 |
| 6D | 283670697 | G, A | OT3098 |
| 7A | 6894726 | A, G | OT3098 |
| 7A | 93976095 | A, C | OT3098 |
| 7A | 212399895 | C, G | OT3098 |
| 7A | 226861026 | A, C | OT3098 |
| 7A | 295965773 | A, G | OT3098 |
| 7A | 423933210 | T, C | OT3098 |
| 7A | 440302050 | T, C | OT3098 |
| 7A | 457458508 | A, G | OT3098 |
| 7A | 488323283 | A, G | OT3098 |
| 7C | 2133869 | C, T | OT3098 |
| 7C | 6687935 | A, G | OT3098 |
| 7C | 40731489 | G, A | OT3098 |
| 7C | 135716079 | C, A | OT3098 |
| 7C | 192742152 | C, T | OT3098 |
| 7C | 213883268 | A, G | OT3098 |
| 7C | 225224321 | T, C | OT3098 |
| 7C | 271932318 | G, A | OT3098 |
| 7C | 303692756 | A, G | OT3098 |
| 7C | 314575954 | T, C | OT3098 |
| 7C | 433548395 | T, C | OT3098 |
| 7C | 461822950 | C, T | OT3098 |
| 7C | 478807736 | A, G | OT3098 |
| 7D | 43006316 | G, A | OT3098 |
| 7D | 53808953 | G, A | OT3098 |
| 7D | 72525182 | T, A | OT3098 |
| 7D | 100996318 | C, T | OT3098 |
| 7D | 119049740 | A, G | OT3098 |
| 7D | 123197785 | C, T | OT3098 |
| 7D | 131225276 | A, G | OT3098 |
| 7D | 196173654 | T, G | OT3098 |
| 7D | 227132197 | T, C | OT3098 |
| 7D | 237053803 | T, G | OT3098 |
| 7D | 248735393 | T, C | OT3098 |
| 7D | 256172681 | C, T | OT3098 |
| 7D | 291312060 | C, T | OT3098 |
| 7D | 363340392 | T, C | OT3098 |
| 7D | 370273697 | A, G | OT3098 |
| 7D | 382112487 | G, A | OT3098 |
| 7D | 391806334 | A, G | OT3098 |
| 7D | 416042498 | C, T | OT3098 |
| 7D | 430774655 | T, C | OT3098 |
| 7D | 466485714 | C, T | OT3098. |

3. A 2K SNP liquid chip of Avena sativa, comprising a probe combination, wherein the probe combination is used for identifying a genotype of each SNP locus in an SNP loci combination; and the SNP loci combination comprises SNP loci located in reference genomes sanfensan and OT3098 as shown in a following table:

| | Physical | Allelic | Reference |
|---|---|---|---|
| Chromosome | location | variation | genome |
| 1A | 4473517 | C, A | sanfensan |
| 1A | 9349344 | G, A | sanfensan |
| 1A | 14898617 | C, T | sanfensan |
| 1A | 19788850 | T, C | sanfensan |
| 1A | 24134605 | A, C | sanfensan |
| 1A | 25204308 | G, A | sanfensan |
| 1A | 34911838 | T, A | sanfensan |
| 1A | 35091182 | A, G | sanfensan |
| 1A | 39926834 | G, A | sanfensan |
| 1A | 40243841 | C, A | sanfensan |
| 1A | 46676991 | T, C | sanfensan |
| 1A | 50477703 | C, T | sanfensan |
| 1A | 55467045 | T, C | sanfensan |
| 1A | 61641698 | C, A | sanfensan |
| 1A | 65935052 | C, A | sanfensan |
| 1A | 71753358 | C, T | sanfensan |
| 1A | 76746486 | T, C | sanfensan |
| 1A | 82946413 | T, C | sanfensan |
| 1A | 85796042 | G, A | sanfensan |
| 1A | 92427340 | G, A | sanfensan |
| 1A | 98303200 | A, C | sanfensan |
| 1A | 100886423 | T, C | sanfensan |
| 1A | 105665342 | A, G | sanfensan |
| 1A | 106585584 | T, C | sanfensan |
| 1A | 110857330 | A, C | sanfensan |
| 1A | 119266034 | T, C | sanfensan |
| 1A | 120319599 | T, G | sanfensan |
| 1A | 128479703 | G, A | sanfensan |
| 1A | 131129360 | A, G | sanfensan |
| 1A | 135686787 | G, A | sanfensan |
| 1A | 140893664 | A, G | sanfensan |
| 1A | 149729422 | A, G | sanfensan |
| 1A | 154018712 | A, G | sanfensan |
| 1A | 156986726 | T, C | sanfensan |
| 1A | 160810612 | G, C | sanfensan |
| 1A | 168045464 | T, G | sanfensan |
| 1A | 174968050 | A, G | sanfensan |
| 1A | 177054360 | C, T | sanfensan |
| 1A | 184641750 | G, A | sanfensan |
| 1A | 186347220 | A, G | sanfensan |
| 1A | 190161955 | T, C | sanfensan |
| 1A | 197891296 | A, G | sanfensan |
| 1A | 202332235 | G, C | sanfensan |
| 1A | 206929081 | A, G | sanfensan |
| 1A | 213307997 | A, C | sanfensan |
| 1A | 215902606 | A, C | sanfensan |
| 1A | 223276058 | G, T | sanfensan |
| 1A | 225939090 | G, A | sanfensan |
| 1A | 229684564 | T, C | sanfensan |
| 1A | 230432457 | A, G | sanfensan |
| 1A | 239890900 | C, T | sanfensan |
| 1A | 243567990 | G, A | sanfensan |
| 1A | 245948969 | G, A | sanfensan |
| 1A | 250969537 | G, C | sanfensan |
| 1A | 257803247 | T, G | sanfensan |
| 1A | 263812100 | A, G | sanfensan |
| 1A | 268553550 | T, C | sanfensan |
| 1A | 271645348 | T, G | sanfensan |
| 1A | 283816950 | G, A | sanfensan |
| 1A | 286799892 | T, C | sanfensan |
| 1A | 292498165 | C, A | sanfensan |
| 1A | 298383411 | A, G | sanfensan |
| 1A | 300811687 | A, G | sanfensan |
| 1A | 307060043 | T, G | sanfensan |
| 1A | 314835969 | G, A | sanfensan |
| 1A | 315910953 | A, G | sanfensan |
| 1A | 321206313 | T, G | sanfensan |
| 1A | 329104188 | A, C | sanfensan |
| 1A | 333516925 | G, T | sanfensan |
| 1A | 335522334 | G, A | sanfensan |
| 1A | 342234031 | G, A | sanfensan |
| 1A | 348750824 | C, A | sanfensan |
| 1A | 354606484 | C, T | sanfensan |
| 1A | 355044825 | G, A | sanfensan |
| 1A | 361899430 | G, A | sanfensan |
| 1A | 365228574 | T, G | sanfensan |
| 1A | 370165053 | A, G | sanfensan |
| 1A | 371882871 | T, C | sanfensan |
| 1A | 379396033 | A, C | sanfensan |
| 1A | 380814167 | C, T | sanfensan |
| 1A | 385881054 | T, G | sanfensan |
| 1A | 394799443 | C, G | sanfensan |
| 1A | 396233056 | A, T | sanfensan |
| 1A | 400131929 | A, G | sanfensan |
| 1A | 408599208 | C, T | sanfensan |
| 1A | 410583439 | T, C | sanfensan |
| 1A | 417767787 | G, A | sanfensan |
| 1A | 423934932 | G, T | sanfensan |
| 1A | 427534714 | A, G | sanfensan |
| 1A | 430802770 | T, C | sanfensan |
| 1A | 439615284 | A, G | sanfensan |
| 1A | 443388417 | C, T | sanfensan |
| 1A | 447639906 | G, A | sanfensan |
| 1A | 453204191 | T, C | sanfensan |
| 1A | 457075413 | G, A | sanfensan |
| 1A | 463117262 | G, A | sanfensan |
| 1A | 465118330 | G, T | sanfensan |
| 1A | 472500626 | T, C | sanfensan |
| 1A | 475569735 | T, G | sanfensan |
| 1A | 481563062 | G, A | sanfensan |
| 1A | 486246321 | T, C | sanfensan |
| 1A | 490249212 | G, A | sanfensan |
| 1A | 495026085 | C, T | sanfensan |
| 1A | 500157268 | G, A | sanfensan |
| 1A | 505420346 | T, C | sanfensan |
| 1A | 513237629 | C, A | sanfensan |
| 1A | 516943173 | A, G | sanfensan |
| 1A | 520725745 | C, G | sanfensan |
| 1A | 529117339 | C, T | sanfensan |
| 1A | 532148235 | T, C | sanfensan |
| 1A | 538845292 | C, T | sanfensan |
| 1A | 540384422 | G, C | sanfensan |
| 1A | 546023901 | C, G | sanfensan |
| 1A | 551040971 | C, T | sanfensan |
| 1C | 4866196 | A, C | sanfensan |
| 1C | 8009186 | G, A | sanfensan |
| 1C | 12114531 | T, C | sanfensan |
| 1C | 17916692 | G, A | sanfensan |
| 1C | 22093157 | T, C | sanfensan |
| 1C | 25463149 | C, T | sanfensan |
| 1C | 32954922 | G, T | sanfensan |
| 1C | 38255313 | G, A | sanfensan |
| 1C | 40225359 | C, T | sanfensan |
| 1C | 47044975 | C, T | sanfensan |
| 1C | 54713053 | C, T | sanfensan |
| 1C | 57342838 | G, T | sanfensan |
| 1C | 64447286 | C, T | sanfensan |
| 1C | 68342251 | C, G | sanfensan |
| 1C | 73736931 | T, C | sanfensan |
| 1C | 75426247 | G, A | sanfensan |
| 1C | 81261715 | C, T | sanfensan |
| 1C | 89052462 | A, C | sanfensan |
| 1C | 93949401 | C, T | sanfensan |
| 1C | 97665480 | T, A | sanfensan |
| 1C | 104414265 | C, T | sanfensan |
| 1C | 107889124 | C, T | sanfensan |
| 1C | 112840945 | A, G | sanfensan |
| 1C | 117607567 | C, A | sanfensan |
| 1C | 123766670 | G, C | sanfensan |
| 1C | 124698034 | C, T | sanfensan |
| 1C | 125624389 | G, A | sanfensan |
| 1C | 132272583 | G, T | sanfensan |
| 1C | 137221231 | G, A | sanfensan |
| 1C | 140336177 | G, A | sanfensan |
| 1C | 148863724 | C, T | sanfensan |
| 1C | 152576285 | T, G | sanfensan |
| 1C | 158557656 | G, C | sanfensan |
| 1C | 161866271 | C, A | sanfensan |
| 1C | 166850618 | G, A | sanfensan |
| 1C | 171246538 | C, A | sanfensan |
| 1C | 177121539 | G, A | sanfensan |
| 1C | 184861598 | C, T | sanfensan |
| 1C | 187896025 | C, A | sanfensan |
| 1C | 192704844 | C, T | sanfensan |
| 1C | 196342850 | G, A | sanfensan |
| 1C | 203152613 | A, G | sanfensan |
| 1C | 206698681 | G, A | sanfensan |
| 1C | 212808498 | G, A | sanfensan |
| 1C | 219911347 | A, G | sanfensan |
| 1C | 223588927 | G, A | sanfensan |
| 1C | 228818861 | G, T | sanfensan |
| 1C | 231062523 | C, T | sanfensan |
| 1C | 244308309 | G, A | sanfensan |
| 1C | 244949293 | G, C | sanfensan |
| 1C | 247382592 | G, A | sanfensan |
| 1C | 251563472 | C, G | sanfensan |
| 1C | 258583411 | G, C | sanfensan |
| 1C | 264870633 | C, T | sanfensan |
| 1C | 269866051 | G, A | sanfensan |
| 1C | 270041152 | T, C | sanfensan |
| 1C | 279714245 | T, C | sanfensan |
| 1C | 281426354 | G, A | sanfensan |
| 1C | 289269367 | C, G | sanfensan |
| 1C | 290192142 | C, A | sanfensan |
| 1C | 295651800 | G, T | sanfensan |
| 1C | 301166258 | G, C | sanfensan |
| 1C | 305159269 | G, T | sanfensan |
| 1C | 311692892 | C, T | sanfensan |
| 1C | 318201468 | T, G | sanfensan |
| 1C | 322797850 | G, A | sanfensan |
| 1C | 329636395 | C, T | sanfensan |
| 1C | 331069160 | G, A | sanfensan |
| 1C | 338302628 | G, A | sanfensan |
| 1C | 343573672 | A, G | sanfensan |
| 1C | 346562089 | G, A | sanfensan |
| 1C | 350268981 | C, T | sanfensan |
| 1C | 359261478 | T, C | sanfensan |
| 1C | 361793363 | G, A | sanfensan |
| 1C | 366795285 | C, G | sanfensan |
| 1C | 373361346 | G, A | sanfensan |
| 1C | 377285872 | C, T | sanfensan |
| 1C | 384514418 | T, G | sanfensan |
| 1C | 388701553 | G, A | sanfensan |
| 1C | 391109953 | C, T | sanfensan |
| 1C | 398781194 | A, C | sanfensan |
| 1C | 404904380 | A, C | sanfensan |
| 1C | 409167422 | A, G | sanfensan |
| 1C | 410239696 | G, A | sanfensan |
| 1C | 418466563 | A, G | sanfensan |
| 1C | 424408198 | C, T | sanfensan |
| 1C | 426142267 | T, C | sanfensan |
| 1C | 434346817 | G, A | sanfensan |
| 1C | 435126767 | A, G | sanfensan |
| 1C | 444951853 | C, T | sanfensan |
| 1C | 446632589 | T, C | sanfensan |
| 1C | 451346048 | G, A | sanfensan |
| 1C | 458628576 | C, T | sanfensan |
| 1D | 4748051 | C, T | sanfensan |
| 1D | 9972757 | C, T | sanfensan |
| 1D | 14428103 | T, C | sanfensan |
| 1D | 17910637 | T, G | sanfensan |
| 1D | 22886105 | A, C | sanfensan |
| 1D | 26189931 | T, C | sanfensan |
| 1D | 34096819 | T, G | sanfensan |
| 1D | 36812285 | T, C | sanfensan |
| 1D | 44167411 | T, C | sanfensan |
| 1D | 48753097 | C, T | sanfensan |
| 1D | 52943769 | G, A | sanfensan |
| 1D | 59943693 | C, T | sanfensan |
| 1D | 61299591 | T, A | sanfensan |
| 1D | 67828154 | C, T | sanfensan |
| 1D | 74812678 | T, C | sanfensan |
| 1D | 76558850 | A, G | sanfensan |
| 1D | 83489940 | C, T | sanfensan |
| 1D | 85424309 | G, A | sanfensan |
| 1D | 90502825 | G, T | sanfensan |
| 1D | 99173014 | C, A | sanfensan |
| 1D | 101866462 | A, G | sanfensan |
| 1D | 105176621 | G, T | sanfensan |
| 1D | 112466846 | A, G | sanfensan |
| 1D | 116735117 | T, C | sanfensan |
| 1D | 120724766 | T, G | sanfensan |
| 1D | 125083241 | A, G | sanfensan |
| 1D | 134949445 | T, C | sanfensan |
| 1D | 136722735 | T, C | sanfensan |
| 1D | 140036387 | C, T | sanfensan |
| 1D | 149197821 | G, A | sanfensan |
| 1D | 154586415 | G, T | sanfensan |
| 1D | 156993466 | T, G | sanfensan |
| 1D | 160591807 | T, G | sanfensan |
| 1D | 169373396 | G, A | sanfensan |
| 1D | 172161852 | G, T | sanfensan |
| 1D | 178558164 | T, C | sanfensan |
| 1D | 182138470 | A, G | sanfensan |
| 1D | 188332946 | T, C | sanfensan |
| 1D | 191338972 | T, C | sanfensan |
| 1D | 197805002 | G, A | sanfensan |
| 1D | 201653641 | A, C | sanfensan |
| 1D | 205231760 | T, C | sanfensan |
| 1D | 213849974 | A, G | sanfensan |
| 1D | 218342762 | G, C | sanfensan |
| 1D | 221295907 | A, C | sanfensan |
| 1D | 225627642 | A, G | sanfensan |
| 1D | 233309591 | C, T | sanfensan |
| 1D | 234707599 | G, A | sanfensan |
| 1D | 239408154 | C, T | sanfensan |
| 1D | 241284677 | T, G | sanfensan |
| 1D | 248921833 | T, G | sanfensan |
| 1D | 250623142 | A, T | sanfensan |
| 1D | 258844136 | C, T | sanfensan |
| 1D | 262320123 | A, C | sanfensan |
| 1D | 265785336 | T, C | sanfensan |
| 1D | 270470362 | C, T | sanfensan |
| 1D | 275515735 | A, G | sanfensan |
| 1D | 284647168 | G, A | sanfensan |
| 1D | 288750391 | A, G | sanfensan |
| 1D | 293777596 | A, G | sanfensan |
| 1D | 298973779 | C, A | sanfensan |
| 1D | 304978046 | T, C | sanfensan |
| 1D | 306430734 | T, G | sanfensan |
| 1D | 313322569 | T, C | sanfensan |
| 1D | 317482376 | T, C | sanfensan |
| 1D | 321285642 | T, C | sanfensan |
| 1D | 328096417 | A, G | sanfensan |
| 1D | 334194238 | G, A | sanfensan |
| 1D | 336604011 | G, A | sanfensan |
| 1D | 342605207 | A, C | sanfensan |
| 1D | 348951141 | A, C | sanfensan |
| 1D | 352790704 | A, G | sanfensan |
| 1D | 355455291 | G, C | sanfensan |
| 1D | 364550878 | A, G | sanfensan |
| 1D | 369719851 | A, G | sanfensan |
| 1D | 372379876 | A, C | sanfensan |
| 1D | 379801584 | A, C | sanfensan |
| 1D | 380728291 | A, G | sanfensan |
| 1D | 388559569 | T, C | sanfensan |
| 1D | 394958911 | T, C | sanfensan |
| 1D | 395282752 | A, G | sanfensan |
| 1D | 403815045 | T, C | sanfensan |
| 1D | 409759489 | T, C | sanfensan |
| 1D | 412133901 | T, C | sanfensan |
| 1D | 418421166 | C, G | sanfensan |
| 1D | 423994932 | T, C | sanfensan |
| 1D | 429511063 | A, G | sanfensan |
| 1D | 433441263 | A, G | sanfensan |
| 1D | 435135220 | T, A | sanfensan |
| 1D | 442087046 | G, A | sanfensan |
| 1D | 442124461 | G, A | sanfensan |
| 1D | 449745797 | G, C | sanfensan |
| 1D | 450598659 | A, G | sanfensan |
| 1D | 459581711 | T, C | sanfensan |
| 1D | 460387603 | C, G | sanfensan |
| 1D | 468646779 | G, A | sanfensan |
| 1D | 470605204 | C, T | sanfensan |
| 2A | 1294852 | A, G | sanfensan |
| 2A | 8354341 | G, A | sanfensan |
| 2A | 10205647 | T, G | sanfensan |
| 2A | 17133037 | A, G | sanfensan |
| 2A | 22147985 | C, G | sanfensan |
| 2A | 25577776 | C, A | sanfensan |
| 2A | 33113184 | G, T | sanfensan |
| 2A | 38311226 | G, A | sanfensan |
| 2A | 43688833 | A, C | sanfensan |
| 2A | 45363179 | C, T | sanfensan |
| 2A | 54582062 | A, G | sanfensan |
| 2A | 58712383 | A, G | sanfensan |
| 2A | 60354747 | A, G | sanfensan |
| 2A | 69920614 | A, G | sanfensan |
| 2A | 74714222 | T, C | sanfensan |
| 2A | 78592686 | A, C | sanfensan |
| 2A | 79665300 | C, A | sanfensan |
| 2A | 83491335 | A, G | sanfensan |
| 2A | 88992475 | A, C | sanfensan |
| 2A | 91196574 | C, T | sanfensan |
| 2A | 98762331 | C, G | sanfensan |
| 2A | 102839151 | T, C | sanfensan |
| 2A | 105115673 | C, T | sanfensan |
| 2A | 113143954 | G, A | sanfensan |
| 2A | 115778719 | C, T | sanfensan |
| 2A | 123726669 | G, C | sanfensan |
| 2A | 125030830 | G, A | sanfensan |
| 2A | 133605969 | C, A | sanfensan |
| 2A | 136067584 | A, C | sanfensan |
| 2A | 142891390 | C, T | sanfensan |
| 2A | 148925271 | C, T | sanfensan |
| 2A | 150242064 | A, G | sanfensan |
| 2A | 159722254 | T, C | sanfensan |
| 2A | 161633504 | G, A | sanfensan |
| 2A | 166801852 | G, A | sanfensan |
| 2A | 172884455 | G, C | sanfensan |
| 2A | 177892881 | A, G | sanfensan |
| 2A | 180161463 | A, C | sanfensan |
| 2A | 187191668 | G, A | sanfensan |
| 2A | 194352120 | C, A | sanfensan |
| 2A | 195938867 | T, C | sanfensan |
| 2A | 202623332 | G, T | sanfensan |
| 2A | 209957751 | G, T | sanfensan |
| 2A | 210875267 | T, G | sanfensan |
| 2A | 215784372 | C, T | sanfensan |
| 2A | 224379638 | G, C | sanfensan |
| 2A | 227479068 | C, T | sanfensan |
| 2A | 231405711 | A, C | sanfensan |
| 2A | 237629147 | C, T | sanfensan |
| 2A | 240260139 | T, C | sanfensan |
| 2A | 248913514 | G, C | sanfensan |
| 2A | 251101436 | C, T | sanfensan |
| 2A | 258858124 | C, T | sanfensan |
| 2A | 262168685 | G, A | sanfensan |
| 2A | 269432173 | A, C | sanfensan |
| 2A | 272066634 | T, C | sanfensan |
| 2A | 275191428 | C, T | sanfensan |
| 2A | 282263456 | T, G | sanfensan |
| 2A | 288156444 | C, G | sanfensan |
| 2A | 291947063 | A, C | sanfensan |
| 2A | 298698676 | T, C | sanfensan |
| 2A | 302165782 | G, A | sanfensan |
| 2A | 307413298 | T, C | sanfensan |
| 2A | 314041736 | C, G | sanfensan |
| 2A | 319115852 | C, T | sanfensan |
| 2A | 324309925 | C, T | sanfensan |
| 2A | 325423488 | A, C | sanfensan |
| 2A | 332013465 | T, C | sanfensan |
| 2A | 336329704 | T, G | sanfensan |
| 2A | 337567230 | G, T | sanfensan |
| 2A | 340385583 | C, T | sanfensan |
| 2A | 347331178 | A, G | sanfensan |
| 2A | 354110393 | C, T | sanfensan |
| 2A | 355224874 | C, T | sanfensan |
| 2A | 361450337 | A, G | sanfensan |
| 2A | 365973983 | T, C | sanfensan |
| 2A | 374152559 | T, C | sanfensan |
| 2A | 378774926 | A, G | sanfensan |
| 2A | 380652237 | C, T | sanfensan |
| 2A | 386433427 | G, A | sanfensan |
| 2A | 391759319 | C, T | sanfensan |
| 2A | 397676516 | C, G | sanfensan |
| 2A | 404431299 | T, C | sanfensan |
| 2A | 406323117 | T, C | sanfensan |
| 2A | 414368443 | A, G | sanfensan |
| 2A | 418701849 | C, A | sanfensan |
| 2A | 421611597 | T, C | sanfensan |
| 2A | 429392443 | C, A | sanfensan |
| 2A | 432088076 | C, A | sanfensan |
| 2A | 432433952 | T, G | sanfensan |
| 2A | 437058043 | T, C | sanfensan |
| 2A | 442675069 | G, C | sanfensan |
| 2A | 447978464 | A, G | sanfensan |
| 2A | 452816108 | C, A | sanfensan |
| 2A | 456332224 | T, C | sanfensan |
| 2A | 460704136 | T, C | sanfensan |
| 2C | 3755642 | C, G | sanfensan |
| 2C | 6523209 | T, C | sanfensan |
| 2C | 14666414 | G, A | sanfensan |
| 2C | 18219538 | G, A | sanfensan |
| 2C | 20598121 | C, T | sanfensan |
| 2C | 25528699 | G, A | sanfensan |
| 2C | 34351879 | G, A | sanfensan |
| 2C | 37480085 | G, A | sanfensan |
| 2C | 40282520 | C, T | sanfensan |
| 2C | 47255505 | G, A | sanfensan |
| 2C | 52177917 | C, T | sanfensan |
| 2C | 55006635 | C, A | sanfensan |
| 2C | 62911833 | A, C | sanfensan |
| 2C | 69182434 | C, T | sanfensan |
| 2C | 73235690 | G, A | sanfensan |
| 2C | 75663383 | C, T | sanfensan |
| 2C | 80698781 | C, T | sanfensan |
| 2C | 88439094 | G, C | sanfensan |
| 2C | 93854384 | C, T | sanfensan |
| 2C | 97362350 | G, A | sanfensan |
| 2C | 104745256 | C, T | sanfensan |
| 2C | 108205218 | C, T | sanfensan |
| 2C | 110557690 | G, A | sanfensan |
| 2C | 119588975 | C, T | sanfensan |
| 2C | 122248235 | C, T | sanfensan |
| 2C | 129849765 | C, T | sanfensan |
| 2C | 134962790 | G, A | sanfensan |
| 2C | 135013067 | T, G | sanfensan |
| 2C | 140602512 | C, T | sanfensan |
| 2C | 145478940 | T, C | sanfensan |
| 2C | 150692118 | G, A | sanfensan |
| 2C | 156314693 | G, A | sanfensan |
| 2C | 160485851 | C, T | sanfensan |
| 2C | 167462260 | G, A | sanfensan |
| 2C | 173642372 | C, T | sanfensan |
| 2C | 179174429 | C, A | sanfensan |
| 2C | 182456426 | G, A | sanfensan |
| 2C | 188223122 | C, T | sanfensan |
| 2C | 190626883 | C, T | sanfensan |
| 2C | 196959827 | G, A | sanfensan |
| 2C | 201765496 | G, T | sanfensan |
| 2C | 207043151 | C, T | sanfensan |
| 2C | 208215965 | C, T | sanfensan |
| 2C | 213490479 | G, A | sanfensan |
| 2C | 217643681 | G, A | sanfensan |
| 2C | 222136297 | G, A | sanfensan |
| 2C | 226477864 | C, T | sanfensan |
| 2C | 231505039 | C, T | sanfensan |
| 2C | 237675775 | C, T | sanfensan |
| 2C | 240667277 | G, A | sanfensan |
| 2C | 246770167 | A, C | sanfensan |
| 2C | 252643615 | G, A | sanfensan |
| 2C | 256169319 | G, A | sanfensan |
| 2C | 261833087 | G, A | sanfensan |
| 2C | 262787175 | A, T | sanfensan |
| 2C | 267745937 | G, A | sanfensan |
| 2C | 271735685 | G, A | sanfensan |
| 2C | 277919032 | G, A | sanfensan |
| 2C | 281899341 | C, T | sanfensan |
| 2C | 287576313 | C, T | sanfensan |
| 2C | 294884196 | G, A | sanfensan |
| 2C | 298708412 | T, A | sanfensan |
| 2C | 304070232 | A, G | sanfensan |
| 2C | 305356521 | G, T | sanfensan |
| 2C | 312681417 | G, A | sanfensan |
| 2C | 317317802 | T, C | sanfensan |
| 2C | 324693110 | G, T | sanfensan |
| 2C | 329639427 | C, G | sanfensan |
| 2C | 334559533 | C, A | sanfensan |
| 2C | 335512805 | C, T | sanfensan |
| 2C | 342249614 | G, A | sanfensan |
| 2C | 349065985 | C, T | sanfensan |
| 2C | 354184855 | C, T | sanfensan |
| 2C | 358792181 | C, T | sanfensan |
| 2C | 361602927 | A, C | sanfensan |
| 2C | 365574905 | T, C | sanfensan |
| 2C | 372578823 | A, T | sanfensan |
| 2C | 377682611 | C, T | sanfensan |
| 2C | 383773493 | G, A | sanfensan |
| 2C | 388264545 | G, T | sanfensan |
| 2C | 392647080 | T, A | sanfensan |
| 2C | 399910470 | G, A | sanfensan |
| 2C | 402251417 | T, G | sanfensan |
| 2C | 405239508 | A, G | sanfensan |
| 2C | 412396338 | A, G | sanfensan |
| 2C | 416260936 | G, A | sanfensan |
| 2C | 423413408 | G, A | sanfensan |
| 2C | 429152113 | C, T | sanfensan |
| 2C | 433743437 | G, A | sanfensan |
| 2C | 438694947 | G, A | sanfensan |
| 2C | 441372926 | G, A | sanfensan |
| 2C | 446094456 | T, G | sanfensan |
| 2C | 451391070 | A, G | sanfensan |
| 2C | 456022723 | A, C | sanfensan |
| 2C | 462451470 | G, A | sanfensan |
| 2C | 469973109 | G, A | sanfensan |
| 2C | 473490736 | G, A | sanfensan |
| 2C | 476541629 | C, T | sanfensan |
| 2C | 482708999 | T, G | sanfensan |
| 2C | 488486085 | C, G | sanfensan |
| 2C | 492299240 | T, G | sanfensan |
| 2C | 497502793 | C, T | sanfensan |
| 2C | 504463768 | G, A | sanfensan |
| 2C | 506288307 | G, A | sanfensan |
| 2C | 511067660 | T, C | sanfensan |
| 2C | 515199747 | G, T | sanfensan |
| 2C | 520028290 | C, G | sanfensan |
| 2C | 526639651 | C, T | sanfensan |
| 2C | 530779807 | G, A | sanfensan |
| 2C | 537336195 | A, G | sanfensan |
| 2C | 540940586 | T, C | sanfensan |
| 2C | 547202871 | G, A | sanfensan |
| 2C | 552060518 | G, C | sanfensan |
| 2D | 4090001 | G, A | sanfensan |
| 2D | 8884387 | G, A | sanfensan |
| 2D | 13578443 | C, G | sanfensan |
| 2D | 17675396 | A, G | sanfensan |
| 2D | 23696567 | G, C | sanfensan |
| 2D | 29926609 | C, A | sanfensan |
| 2D | 31795322 | C, G | sanfensan |
| 2D | 38891629 | C, T | sanfensan |
| 2D | 43755471 | A, C | sanfensan |
| 2D | 49839859 | T, C | sanfensan |
| 2D | 52853593 | T, G | sanfensan |
| 2D | 59369523 | A, G | sanfensan |
| 2D | 64844533 | C, T | sanfensan |
| 2D | 67973342 | G, C | sanfensan |
| 2D | 72762945 | G, T | sanfensan |
| 2D | 77924703 | G, A | sanfensan |
| 2D | 82246788 | C, G | sanfensan |
| 2D | 85545627 | A, G | sanfensan |
| 2D | 93391163 | T, C | sanfensan |
| 2D | 97511713 | T, C | sanfensan |
| 2D | 103508833 | A, G | sanfensan |
| 2D | 105773491 | C, T | sanfensan |
| 2D | 112039326 | T, C | sanfensan |
| 2D | 118313919 | A, G | sanfensan |
| 2D | 124707738 | G, A | sanfensan |
| 2D | 127418722 | T, C | sanfensan |
| 2D | 134051671 | A, G | sanfensan |
| 2D | 139923233 | T, C | sanfensan |
| 2D | 141313127 | G, T | sanfensan |
| 2D | 147473787 | C, A | sanfensan |
| 2D | 153131701 | A, G | sanfensan |
| 2D | 155782705 | A, C | sanfensan |
| 2D | 161543833 | T, G | sanfensan |
| 2D | 168209065 | T, C | sanfensan |
| 2D | 171707336 | G, A | sanfensan |
| 2D | 175227041 | C, G | sanfensan |
| 2D | 183727141 | C, A | sanfensan |
| 2D | 186245952 | T, C | sanfensan |
| 2D | 191571350 | T, C | sanfensan |
| 2D | 197886074 | C, A | sanfensan |
| 2D | 201218813 | A, G | sanfensan |
| 2D | 206194560 | G, T | sanfensan |
| 2D | 209315382 | T, C | sanfensan |
| 2D | 214621383 | T, G | sanfensan |
| 2D | 217094666 | T, C | sanfensan |
| 2D | 224088866 | C, T | sanfensan |
| 2D | 228951287 | A, G | sanfensan |
| 2D | 233694297 | A, C | sanfensan |
| 2D | 237373003 | T, C | sanfensan |
| 2D | 240374430 | G, T | sanfensan |
| 2D | 247507939 | G, A | sanfensan |
| 2D | 253371162 | C, T | sanfensan |
| 2D | 259426340 | T, C | sanfensan |
| 2D | 262144878 | T, C | sanfensan |
| 2D | 268152175 | C, T | sanfensan |
| 2D | 271878835 | A, C | sanfensan |
| 2D | 278388739 | G, A | sanfensan |
| 2D | 281916083 | G, A | sanfensan |
| 2D | 289003292 | T, C | sanfensan |
| 2D | 293861011 | T, C | sanfensan |
| 2D | 297746679 | G, A | sanfensan |
| 2D | 301635985 | T, C | sanfensan |
| 2D | 307441210 | A, G | sanfensan |
| 2D | 313997317 | C, T | sanfensan |
| 2D | 317780757 | T, C | sanfensan |
| 2D | 324471320 | G, C | sanfensan |
| 2D | 325394234 | C, T | sanfensan |
| 2D | 332243555 | G, A | sanfensan |
| 2D | 336723088 | C, T | sanfensan |
| 2D | 344502614 | T, C | sanfensan |
| 2D | 348558097 | G, A | sanfensan |
| 2D | 354230586 | G, A | sanfensan |
| 2D | 356506435 | C, T | sanfensan |
| 2D | 361372636 | C, A | sanfensan |
| 2D | 367508960 | T, C | sanfensan |
| 2D | 374623643 | C, T | sanfensan |
| 2D | 376574690 | G, A | sanfensan |
| 2D | 381350972 | A, G | sanfensan |
| 2D | 385713492 | C, T | sanfensan |
| 2D | 389191706 | C, A | sanfensan |
| 2D | 393186784 | T, C | sanfensan |
| 2D | 398053878 | C, T | sanfensan |
| 2D | 402113963 | G, A | sanfensan |
| 2D | 407714293 | C, T | sanfensan |
| 2D | 413300157 | C, G | sanfensan |
| 2D | 416994083 | C, T | sanfensan |
| 2D | 422486122 | A, G | sanfensan |
| 2D | 429086006 | C, T | sanfensan |
| 2D | 430932257 | T, G | sanfensan |
| 2D | 435517110 | T, C | sanfensan |
| 2D | 440681525 | G, T | sanfensan |
| 2D | 446278114 | T, C | sanfensan |
| 2D | 452744585 | A, G | sanfensan |
| 2D | 458588987 | G, A | sanfensan |
| 2D | 460269576 | T, G | sanfensan |
| 2D | 466552364 | C, T | sanfensan |
| 2D | 473536427 | A, C | sanfensan |
| 2D | 478638656 | T, G | sanfensan |
| 2D | 484074145 | G, A | sanfensan |
| 2D | 486599496 | C, T | sanfensan |
| 2D | 492020467 | C, T | sanfensan |
| 2D | 498876308 | C, T | sanfensan |
| 2D | 499971233 | T, C | sanfensan |
| 2D | 501118019 | C, T | sanfensan |
| 2D | 509719761 | G, T | sanfensan |
| 2D | 511708692 | G, A | sanfensan |
| 2D | 515022757 | T, A | sanfensan |
| 2D | 523356028 | C, A | sanfensan |
| 2D | 525941172 | A, G | sanfensan |
| 2D | 529724172 | A, C | sanfensan |
| 2D | 530568058 | G, A | sanfensan |
| 3A | 349403 | C, T | sanfensan |
| 3A | 5686447 | A, G | sanfensan |
| 3A | 14820154 | G, C | sanfensan |
| 3A | 19103892 | A, G | sanfensan |
| 3A | 24307017 | G, A | sanfensan |
| 3A | 28596998 | G, A | sanfensan |
| 3A | 34850179 | A, G | sanfensan |
| 3A | 39937020 | C, T | sanfensan |
| 3A | 43520691 | A, G | sanfensan |
| 3A | 49770087 | T, C | sanfensan |
| 3A | 54743914 | A, G | sanfensan |
| 3A | 56540660 | A, C | sanfensan |
| 3A | 60015652 | C, T | sanfensan |
| 3A | 68312753 | T, G | sanfensan |
| 3A | 70210131 | A, G | sanfensan |
| 3A | 75548656 | C, T | sanfensan |
| 3A | 81960668 | G, A | sanfensan |
| 3A | 87385909 | G, A | sanfensan |
| 3A | 91466974 | T, C | sanfensan |
| 3A | 99324808 | G, A | sanfensan |
| 3A | 103702261 | A, T | sanfensan |
| 3A | 106902222 | A, G | sanfensan |
| 3A | 114734802 | C, T | sanfensan |
| 3A | 117018962 | T, G | sanfensan |
| 3A | 123014541 | G, T | sanfensan |
| 3A | 125235584 | G, A | sanfensan |
| 3A | 132736923 | G, T | sanfensan |
| 3A | 133400727 | T, C | sanfensan |
| 3A | 135881496 | A, G | sanfensan |
| 3A | 140132188 | A, C | sanfensan |
| 3A | 149216557 | G, T | sanfensan |
| 3A | 153688728 | T, C | sanfensan |
| 3A | 157365828 | T, G | sanfensan |
| 3A | 162629353 | G, A | sanfensan |
| 3A | 163249925 | C, T | sanfensan |
| 3A | 165620860 | A, G | sanfensan |
| 3A | 170114512 | T, C | sanfensan |
| 3A | 175321928 | G, A | sanfensan |
| 3A | 183395685 | C, T | sanfensan |
| 3A | 188224937 | A, C | sanfensan |
| 3A | 194044731 | G, A | sanfensan |
| 3A | 197334481 | G, A | sanfensan |
| 3A | 204024961 | T, C | sanfensan |
| 3A | 209320496 | A, G | sanfensan |
| 3A | 214269791 | G, A | sanfensan |
| 3A | 215870959 | A, G | sanfensan |
| 3A | 220058696 | A, C | sanfensan |
| 3A | 228807812 | G, A | sanfensan |
| 3A | 234527749 | A, G | sanfensan |
| 3A | 237989422 | G, A | sanfensan |
| 3A | 241256197 | C, T | sanfensan |
| 3A | 248608009 | C, T | sanfensan |
| 3A | 254861238 | T, C | sanfensan |
| 3A | 255490400 | A, G | sanfensan |
| 3A | 261499403 | T, C | sanfensan |
| 3A | 268848139 | T, G | sanfensan |
| 3A | 273512863 | T, C | sanfensan |
| 3A | 279998619 | A, G | sanfensan |
| 3A | 280635220 | A, C | sanfensan |
| 3A | 289497088 | C, T | sanfensan |
| 3A | 291617575 | G, A | sanfensan |
| 3A | 296690651 | T, C | sanfensan |
| 3A | 303858433 | G, A | sanfensan |
| 3A | 308390029 | A, G | sanfensan |
| 3A | 313163176 | C, T | sanfensan |
| 3A | 315321758 | G, T | sanfensan |
| 3A | 323449820 | C, T | sanfensan |
| 3A | 326487847 | G, T | sanfensan |
| 3A | 334646062 | C, G | sanfensan |
| 3A | 339385657 | T, G | sanfensan |
| 3A | 344905994 | C, T | sanfensan |
| 3A | 346539460 | G, A | sanfensan |
| 3A | 354806237 | C, T | sanfensan |
| 3A | 358217253 | C, T | sanfensan |
| 3A | 363057750 | C, T | sanfensan |
| 3A | 368774140 | C, T | sanfensan |
| 3A | 370439246 | C, T | sanfensan |
| 3A | 379158179 | A, G | sanfensan |
| 3A | 380572844 | T, C | sanfensan |
| 3A | 385843902 | C, T | sanfensan |
| 3A | 393548401 | C, T | sanfensan |
| 3A | 396511248 | A, G | sanfensan |
| 3A | 400231496 | G, A | sanfensan |
| 3A | 407861434 | T, C | sanfensan |
| 3A | 410745937 | A, G | sanfensan |
| 3A | 417480860 | A, C | sanfensan |
| 3C | 4934705 | T, C | sanfensan |
| 3C | 9789448 | T, C | sanfensan |
| 3C | 14114292 | A, G | sanfensan |
| 3C | 16111717 | T, C | sanfensan |
| 3C | 20060240 | T, C | sanfensan |
| 3C | 29738599 | G, T | sanfensan |
| 3C | 34251466 | G, A | sanfensan |
| 3C | 39605336 | A, C | sanfensan |
| 3C | 41571752 | T, C | sanfensan |
| 3C | 48463788 | G, T | sanfensan |
| 3C | 50619185 | G, A | sanfensan |
| 3C | 59812660 | G, A | sanfensan |
| 3C | 63059450 | T, C | sanfensan |
| 3C | 69756699 | C, T | sanfensan |
| 3C | 72007306 | T, C | sanfensan |
| 3C | 75582282 | G, C | sanfensan |
| 3C | 81453337 | T, C | sanfensan |
| 3C | 89524634 | T, C | sanfensan |
| 3C | 94729319 | C, A | sanfensan |
| 3C | 96052025 | C, T | sanfensan |
| 3C | 100397477 | A, G | sanfensan |
| 3C | 108020814 | A, G | sanfensan |
| 3C | 113507812 | A, C | sanfensan |
| 3C | 119056248 | C, T | sanfensan |
| 3C | 121539032 | G, T | sanfensan |
| 3C | 127564610 | C, T | sanfensan |
| 3C | 130874626 | G, A | sanfensan |
| 3C | 137072359 | G, A | sanfensan |
| 3C | 142064744 | G, A | sanfensan |
| 3C | 147139993 | T, C | sanfensan |
| 3C | 152287508 | G, A | sanfensan |
| 3C | 158231130 | C, T | sanfensan |
| 3C | 161849544 | C, T | sanfensan |
| 3C | 167803980 | A, G | sanfensan |
| 3C | 171441935 | T, C | sanfensan |
| 3C | 178608408 | T, G | sanfensan |
| 3C | 180422132 | T, C | sanfensan |
| 3C | 186181001 | G, A | sanfensan |
| 3C | 192589566 | T, C | sanfensan |
| 3C | 195188213 | G, A | sanfensan |
| 3C | 204778810 | C, T | sanfensan |
| 3C | 207904801 | T, C | sanfensan |
| 3C | 214065619 | T, G | sanfensan |
| 3C | 217226719 | T, C | sanfensan |
| 3C | 221780018 | G, C | sanfensan |
| 3C | 228014218 | G, A | sanfensan |
| 3C | 233526022 | G, A | sanfensan |
| 3C | 235353093 | A, C | sanfensan |
| 3C | 244162351 | G, A | sanfensan |
| 3C | 248122047 | C, T | sanfensan |
| 3C | 253823594 | C, T | sanfensan |
| 3C | 259462643 | A, G | sanfensan |
| 3C | 260199510 | T, C | sanfensan |
| 3C | 269465277 | G, T | sanfensan |
| 3C | 274931211 | G, A | sanfensan |
| 3C | 277523171 | C, A | sanfensan |
| 3C | 284109639 | G, C | sanfensan |
| 3C | 285346116 | A, G | sanfensan |
| 3C | 294939142 | A, C | sanfensan |
| 3C | 296525393 | A, G | sanfensan |
| 3C | 304406734 | T, C | sanfensan |
| 3C | 309329884 | C, T | sanfensan |
| 3C | 310447021 | T, G | sanfensan |
| 3C | 319961145 | T, C | sanfensan |
| 3C | 321458383 | T, C | sanfensan |
| 3C | 325121366 | A, G | sanfensan |
| 3C | 330206369 | A, G | sanfensan |
| 3C | 332130807 | C, T | sanfensan |
| 3C | 338543178 | T, C | sanfensan |
| 3C | 343287383 | T, C | sanfensan |
| 3C | 349175645 | T, C | sanfensan |
| 3C | 353910191 | C, T | sanfensan |
| 3C | 358917955 | T, C | sanfensan |
| 3C | 363942089 | G, A | sanfensan |
| 3C | 366028325 | A, C | sanfensan |
| 3C | 372945120 | C, T | sanfensan |
| 3C | 375489817 | C, G | sanfensan |
| 3C | 384457117 | A, G | sanfensan |
| 3C | 388958566 | A, G | sanfensan |
| 3C | 394935493 | A, T | sanfensan |
| 3C | 395752355 | G, A | sanfensan |
| 3C | 403204424 | G, A | sanfensan |
| 3C | 408346907 | A, C | sanfensan |
| 3C | 412555939 | T, C | sanfensan |
| 3C | 418959428 | A, G | sanfensan |
| 3C | 424637971 | G, A | sanfensan |
| 3C | 425426160 | G, A | sanfensan |
| 3C | 431869178 | A, C | sanfensan |
| 3C | 437116797 | T, G | sanfensan |
| 3C | 442997546 | A, G | sanfensan |
| 3C | 445451564 | C, A | sanfensan |
| 3C | 449848039 | C, T | sanfensan |
| 3C | 450156513 | C, T | sanfensan |
| 3C | 458930099 | T, C | sanfensan |
| 3C | 460938762 | C, T | sanfensan |
| 3C | 465773933 | A, G | sanfensan |
| 3C | 466127904 | C, A | sanfensan |
| 3C | 472480324 | G, T | sanfensan |
| 3C | 476852155 | T, C | sanfensan |
| 3C | 482946601 | C, T | sanfensan |
| 3C | 488844690 | A, C | sanfensan |
| 3C | 494056611 | C, T | sanfensan |
| 3C | 499140906 | G, A | sanfensan |
| 3C | 504946938 | C, A | sanfensan |
| 3C | 505015277 | T, G | sanfensan |
| 3C | 513611369 | G, A | sanfensan |
| 3C | 515368248 | C, T | sanfensan |
| 3C | 520707894 | G, A | sanfensan |
| 3C | 529897647 | C, T | sanfensan |
| 3C | 534373018 | A, G | sanfensan |
| 3C | 536903652 | C, T | sanfensan |
| 3C | 540735781 | A, C | sanfensan |
| 3C | 545800871 | T, C | sanfensan |
| 3C | 551522255 | A, C | sanfensan |
| 3C | 556937242 | T, C | sanfensan |
| 3C | 561945091 | C, A | sanfensan |
| 3C | 565260776 | C, T | sanfensan |
| 3C | 572949143 | C, G | sanfensan |
| 3C | 577651667 | G, A | sanfensan |
| 3C | 580350958 | C, T | sanfensan |
| 3C | 589499247 | A, G | sanfensan |
| 3C | 593401324 | A, G | sanfensan |
| 3C | 596059950 | G, A | sanfensan |
| 3C | 602749897 | C, G | sanfensan |
| 3C | 606686603 | A, G | sanfensan |
| 3C | 612645249 | A, G | sanfensan |
| 3C | 615205024 | T, A | sanfensan |
| 3D | 3451014 | A, G | sanfensan |
| 3D | 6304975 | C, G | sanfensan |
| 3D | 8431856 | G, A | sanfensan |
| 3D | 12739908 | G, A | sanfensan |
| 3D | 17626366 | T, C | sanfensan |
| 3D | 22243586 | T, C | sanfensan |
| 3D | 29901822 | T, C | sanfensan |
| 3D | 34199181 | T, A | sanfensan |
| 3D | 38215402 | C, T | sanfensan |
| 3D | 40736696 | C, T | sanfensan |
| 3D | 45922953 | C, T | sanfensan |
| 3D | 54465933 | T, C | sanfensan |
| 3D | 59996227 | A, G | sanfensan |
| 3D | 63704529 | G, A | sanfensan |
| 3D | 66789700 | C, G | sanfensan |
| 3D | 70216974 | C, T | sanfensan |
| 3D | 78492763 | A, G | sanfensan |
| 3D | 82560649 | G, A | sanfensan |
| 3D | 89278480 | C, T | sanfensan |
| 3D | 94976535 | T, A | sanfensan |
| 3D | 99870793 | C, A | sanfensan |
| 3D | 102284469 | G, A | sanfensan |
| 3D | 105085292 | G, T | sanfensan |
| 3D | 112272049 | A, G | sanfensan |
| 3D | 117725865 | G, A | sanfensan |
| 3D | 124920499 | T, C | sanfensan |
| 3D | 125085251 | G, A | sanfensan |
| 3D | 130241097 | G, T | sanfensan |
| 3D | 136299812 | G, A | sanfensan |
| 3D | 140453087 | G, T | sanfensan |
| 3D | 149490075 | G, A | sanfensan |
| 3D | 154494846 | C, T | sanfensan |
| 3D | 155736382 | A, G | sanfensan |
| 3D | 160258419 | T, G | sanfensan |
| 3D | 165382934 | G, C | sanfensan |
| 3D | 174233842 | A, G | sanfensan |
| 3D | 178210807 | A, G | sanfensan |
| 3D | 183045672 | C, T | sanfensan |
| 3D | 185605470 | A, G | sanfensan |
| 3D | 191181967 | T, C | sanfensan |
| 3D | 199127188 | C, A | sanfensan |
| 3D | 201408393 | T, C | sanfensan |
| 3D | 208325818 | C, A | sanfensan |
| 3D | 213296950 | G, T | sanfensan |
| 3D | 217423350 | T, C | sanfensan |
| 3D | 223809923 | G, C | sanfensan |
| 3D | 226483167 | G, A | sanfensan |
| 3D | 231132075 | C, T | sanfensan |
| 3D | 237287427 | T, A | sanfensan |
| 3D | 241417679 | G, A | sanfensan |
| 3D | 248327064 | G, A | sanfensan |
| 3D | 250955048 | C, T | sanfensan |
| 3D | 257222395 | C, T | sanfensan |
| 3D | 263680149 | G, A | sanfensan |
| 3D | 268114489 | C, A | sanfensan |
| 3D | 273619731 | A, C | sanfensan |
| 3D | 276710058 | C, T | sanfensan |
| 3D | 284403130 | G, C | sanfensan |
| 3D | 288065151 | G, A | sanfensan |
| 3D | 294630726 | C, T | sanfensan |
| 3D | 297227408 | C, T | sanfensan |
| 3D | 300199691 | T, C | sanfensan |
| 3D | 306425893 | C, A | sanfensan |
| 3D | 314722897 | A, G | sanfensan |
| 3D | 315890257 | A, T | sanfensan |
| 3D | 323402379 | G, A | sanfensan |
| 3D | 325861485 | A, G | sanfensan |
| 3D | 330563924 | A, G | sanfensan |
| 3D | 335625837 | G, A | sanfensan |
| 3D | 340772061 | G, A | sanfensan |
| 3D | 348100481 | T, G | sanfensan |
| 3D | 352819049 | C, T | sanfensan |
| 3D | 358682575 | T, C | sanfensan |
| 3D | 361158177 | G, A | sanfensan |
| 3D | 369050731 | T, C | sanfensan |
| 3D | 373598604 | C, T | sanfensan |
| 3D | 378481165 | T, C | sanfensan |
| 3D | 381350490 | G, A | sanfensan |
| 3D | 388861217 | T, C | sanfensan |
| 3D | 389878476 | T, G | sanfensan |
| 3D | 391163814 | T, C | sanfensan |
| 3D | 397016298 | C, T | sanfensan |
| 3D | 403496100 | A, G | sanfensan |
| 3D | 406924921 | A, G | sanfensan |
| 3D | 410226093 | G, A | sanfensan |
| 3D | 417099879 | A, G | sanfensan |
| 3D | 419164521 | C, G | sanfensan |
| 3D | 423904272 | T, C | sanfensan |
| 3D | 426092994 | A, G | sanfensan |
| 3D | 433821318 | G, A | sanfensan |
| 3D | 436973559 | T, G | sanfensan |
| 3D | 439655087 | A, G | sanfensan |
| 3D | 441290663 | T, C | sanfensan |
| 3D | 448791478 | T, C | sanfensan |
| 3D | 451012282 | C, G | sanfensan |
| 3D | 456635851 | G, A | sanfensan |
| 3D | 460647997 | A, T | sanfensan |
| 4A | 167665 | A, G | sanfensan |
| 4A | 5170696 | A, G | sanfensan |
| 4A | 12321972 | G, A | sanfensan |
| 4A | 19482740 | T, C | sanfensan |
| 4A | 22870941 | T, C | sanfensan |
| 4A | 27380296 | T, C | sanfensan |
| 4A | 30862932 | G, T | sanfensan |
| 4A | 36420099 | T, G | sanfensan |
| 4A | 38087452 | T, G | sanfensan |
| 4A | 44284137 | A, C | sanfensan |
| 4A | 45337009 | T, C | sanfensan |
| 4A | 50385302 | T, C | sanfensan |
| 4A | 58613149 | T, C | sanfensan |
| 4A | 62632618 | T, C | sanfensan |
| 4A | 69360237 | A, C | sanfensan |
| 4A | 72548515 | T, C | sanfensan |
| 4A | 78860501 | A, G | sanfensan |
| 4A | 83477002 | A, G | sanfensan |
| 4A | 86718114 | A, C | sanfensan |
| 4A | 90186259 | T, C | sanfensan |
| 4A | 99717884 | C, G | sanfensan |
| 4A | 103226101 | C, T | sanfensan |
| 4A | 108841089 | C, A | sanfensan |
| 4A | 109097229 | G, A | sanfensan |
| 4A | 110362403 | T, C | sanfensan |
| 4A | 119524477 | G, C | sanfensan |
| 4A | 124335960 | C, T | sanfensan |
| 4A | 129987141 | C, T | sanfensan |
| 4A | 132188846 | A, G | sanfensan |
| 4A | 138010184 | A, G | sanfensan |
| 4A | 141248665 | T, C | sanfensan |
| 4A | 141974369 | A, G | sanfensan |
| 4A | 146462646 | G, A | sanfensan |
| 4A | 151979768 | T, C | sanfensan |
| 4A | 158451505 | G, A | sanfensan |
| 4A | 163350428 | A, G | sanfensan |
| 4A | 167565220 | A, G | sanfensan |
| 4A | 173953101 | T, G | sanfensan |
| 4A | 178883204 | G, C | sanfensan |
| 4A | 184596923 | C, T | sanfensan |
| 4A | 189201669 | A, C | sanfensan |
| 4A | 193547898 | G, A | sanfensan |
| 4A | 199508298 | A, G | sanfensan |
| 4A | 201195563 | C, T | sanfensan |
| 4A | 207185396 | C, A | sanfensan |
| 4A | 213373498 | T, C | sanfensan |
| 4A | 216222538 | A, G | sanfensan |
| 4A | 221795376 | C, T | sanfensan |
| 4A | 228345642 | A, T | sanfensan |
| 4A | 231330723 | A, C | sanfensan |
| 4A | 235514032 | G, A | sanfensan |
| 4A | 242193423 | C, T | sanfensan |
| 4A | 247945600 | C, T | sanfensan |
| 4A | 252369025 | T, C | sanfensan |
| 4A | 259286784 | G, C | sanfensan |
| 4A | 261531070 | A, G | sanfensan |
| 4A | 268127413 | G, T | sanfensan |
| 4A | 273296067 | A, G | sanfensan |
| 4A | 276596131 | T, C | sanfensan |
| 4A | 280307793 | C, T | sanfensan |
| 4A | 289509128 | C, T | sanfensan |
| 4A | 293451571 | G, A | sanfensan |
| 4A | 299169983 | T, G | sanfensan |
| 4A | 300515091 | T, C | sanfensan |
| 4A | 305599904 | G, A | sanfensan |
| 4A | 311085314 | A, C | sanfensan |
| 4A | 316042196 | C, T | sanfensan |
| 4A | 322946253 | T, C | sanfensan |
| 4A | 325478648 | G, C | sanfensan |
| 4A | 330347981 | A, G | sanfensan |
| 4A | 337443775 | C, T | sanfensan |
| 4A | 340462080 | A, G | sanfensan |
| 4A | 347382275 | A, G | sanfensan |
| 4A | 354497490 | T, G | sanfensan |
| 4A | 359050714 | C, A | sanfensan |
| 4A | 362340167 | C, T | sanfensan |
| 4A | 367751846 | G, A | sanfensan |
| 4A | 370622530 | G, T | sanfensan |
| 4A | 379177676 | T, C | sanfensan |
| 4A | 381166365 | T, C | sanfensan |
| 4A | 385406079 | C, G | sanfensan |
| 4A | 398015147 | A, C | sanfensan |
| 4A | 401416507 | G, A | sanfensan |
| 4A | 406634471 | A, G | sanfensan |
| 4A | 413012139 | A, G | sanfensan |
| 4A | 418384264 | C, T | sanfensan |
| 4A | 424141863 | C, T | sanfensan |
| 4A | 426462945 | A, T | sanfensan |
| 4A | 434286106 | G, T | sanfensan |
| 4A | 435134503 | C, T | sanfensan |
| 4A | 440807368 | C, G | sanfensan |
| 4A | 445324027 | C, T | sanfensan |
| 4A | 450745492 | T, C | sanfensan |
| 4A | 458822974 | A, G | sanfensan |
| 4A | 462418830 | A, C | sanfensan |
| 4A | 466738641 | T, C | sanfensan |
| 4C | 3595818 | G, A | sanfensan |
| 4C | 6028133 | A, G | sanfensan |
| 4C | 13049345 | T, A | sanfensan |
| 4C | 19972099 | A, G | sanfensan |
| 4C | 20771307 | T, C | sanfensan |
| 4C | 25886686 | G, C | sanfensan |
| 4C | 32211660 | C, A | sanfensan |
| 4C | 36873418 | G, C | sanfensan |
| 4C | 40449202 | C, G | sanfensan |
| 4C | 47158151 | C, A | sanfensan |
| 4C | 51926597 | T, C | sanfensan |
| 4C | 57722231 | G, A | sanfensan |
| 4C | 61365967 | G, A | sanfensan |
| 4C | 66290570 | C, T | sanfensan |
| 4C | 69406750 | G, A | sanfensan |
| 4C | 71656189 | G, A | sanfensan |
| 4C | 75202185 | A, G | sanfensan |
| 4C | 80202847 | G, T | sanfensan |
| 4C | 86069619 | A, G | sanfensan |
| 4C | 92181840 | C, T | sanfensan |
| 4C | 95153438 | G, A | sanfensan |
| 4C | 101779057 | T, C | sanfensan |
| 4C | 106362411 | C, T | sanfensan |
| 4C | 113373499 | T, G | sanfensan |
| 4C | 116319458 | C, T | sanfensan |
| 4C | 122530727 | A, G | sanfensan |
| 4C | 128925968 | T, G | sanfensan |
| 4C | 130658845 | T, C | sanfensan |
| 4C | 139094233 | A, G | sanfensan |
| 4C | 141439434 | G, T | sanfensan |
| 4C | 149617745 | A, C | sanfensan |
| 4C | 151871212 | T, C | sanfensan |
| 4C | 159944864 | C, T | sanfensan |
| 4C | 164691088 | T, C | sanfensan |
| 4C | 169970304 | C, A | sanfensan |
| 4C | 170331200 | A, C | sanfensan |
| 4C | 178692748 | C, T | sanfensan |
| 4C | 184841316 | C, T | sanfensan |
| 4C | 185887772 | T, C | sanfensan |
| 4C | 193734961 | C, T | sanfensan |
| 4C | 197024216 | G, A | sanfensan |
| 4C | 202625381 | G, A | sanfensan |
| 4C | 209016901 | A, G | sanfensan |
| 4C | 213200350 | C, T | sanfensan |
| 4C | 215000144 | C, T | sanfensan |
| 4C | 224645115 | C, T | sanfensan |
| 4C | 229625246 | A, G | sanfensan |
| 4C | 234547399 | T, C | sanfensan |
| 4C | 236119582 | A, C | sanfensan |
| 4C | 241687009 | G, A | sanfensan |
| 4C | 245369143 | G, T | sanfensan |
| 4C | 252253086 | T, A | sanfensan |
| 4C | 255164714 | C, T | sanfensan |
| 4C | 260860063 | G, A | sanfensan |
| 4C | 267013553 | G, A | sanfensan |
| 4C | 273797546 | G, A | sanfensan |
| 4C | 277862984 | C, T | sanfensan |
| 4C | 282087726 | G, A | sanfensan |
| 4C | 287337582 | G, A | sanfensan |
| 4C | 292238812 | C, T | sanfensan |
| 4C | 296562693 | C, T | sanfensan |
| 4C | 301499481 | T, C | sanfensan |
| 4C | 308695483 | G, A | sanfensan |
| 4C | 311224923 | G, A | sanfensan |
| 4C | 316095744 | G, A | sanfensan |
| 4C | 324951331 | C, T | sanfensan |
| 4C | 325766510 | T, G | sanfensan |
| 4C | 331060999 | G, A | sanfensan |
| 4C | 339044922 | C, T | sanfensan |
| 4C | 342159259 | G, A | sanfensan |
| 4C | 345139072 | G, A | sanfensan |
| 4C | 350531907 | G, T | sanfensan |
| 4C | 357426076 | G, A | sanfensan |
| 4C | 362889043 | G, A | sanfensan |
| 4C | 369798229 | G, A | sanfensan |
| 4C | 372472310 | C, T | sanfensan |
| 4C | 378265862 | T, C | sanfensan |
| 4C | 381319655 | G, A | sanfensan |
| 4C | 385095261 | T, G | sanfensan |
| 4C | 387723697 | C, T | sanfensan |
| 4C | 392326058 | T, C | sanfensan |
| 4C | 395186922 | T, C | sanfensan |
| 4C | 404320541 | A, C | sanfensan |
| 4C | 407798339 | C, G | sanfensan |
| 4C | 414198946 | A, G | sanfensan |
| 4C | 418786637 | C, T | sanfensan |
| 4C | 420987303 | G, C | sanfensan |
| 4C | 425274444 | A, T | sanfensan |
| 4C | 437039125 | T, C | sanfensan |
| 4C | 442594181 | C, T | sanfensan |
| 4C | 446643052 | G, T | sanfensan |
| 4C | 451884828 | C, A | sanfensan |
| 4C | 455850824 | G, A | sanfensan |
| 4C | 463494104 | C, T | sanfensan |
| 4C | 467165660 | C, T | sanfensan |
| 4C | 472240988 | T, G | sanfensan |
| 4C | 475026617 | T, C | sanfensan |
| 4C | 481585885 | G, A | sanfensan |
| 4C | 487240271 | G, T | sanfensan |
| 4C | 493243160 | C, T | sanfensan |
| 4C | 499531487 | C, A | sanfensan |
| 4C | 504518607 | G, A | sanfensan |
| 4C | 509955623 | G, A | sanfensan |
| 4C | 511514837 | A, G | sanfensan |
| 4C | 514138096 | G, A | sanfensan |
| 4C | 519111243 | A, T | sanfensan |
| 4C | 520487689 | C, T | sanfensan |
| 4C | 526516206 | A, G | sanfensan |
| 4C | 530500162 | C, T | sanfensan |
| 4C | 537644622 | T, C | sanfensan |
| 4C | 540730312 | G, A | sanfensan |
| 4C | 545836115 | G, T | sanfensan |
| 4C | 549932188 | A, G | sanfensan |
| 4C | 550915489 | A, T | sanfensan |
| 4C | 558054888 | C, T | sanfensan |
| 4C | 562963997 | A, G | sanfensan |
| 4C | 569467277 | A, G | sanfensan |
| 4C | 572657475 | T, C | sanfensan |
| 4C | 575327801 | A, G | sanfensan |
| 4C | 584368593 | T, G | sanfensan |
| 4C | 588543504 | A, G | sanfensan |
| 4C | 589445451 | A, G | sanfensan |
| 4C | 593669777 | A, C | sanfensan |
| 4C | 599151537 | A, G | sanfensan |
| 4C | 600053663 | T, C | sanfensan |
| 4C | 607995668 | A, G | sanfensan |
| 4C | 610178903 | T, C | sanfensan |
| 4C | 614566248 | G, C | sanfensan |
| 4C | 616610794 | C, T | sanfensan |
| 4C | 624701501 | T, C | sanfensan |
| 4C | 626629588 | G, A | sanfensan |
| 4C | 631097435 | A, C | sanfensan |
| 4C | 635505874 | C, A | sanfensan |
| 4C | 642622496 | T, C | sanfensan |
| 4C | 645881564 | A, C | sanfensan |
| 4C | 653135330 | G, T | sanfensan |
| 4C | 655135466 | A, G | sanfensan |
| 4C | 660684760 | G, A | sanfensan |
| 4C | 665124776 | G, C | sanfensan |
| 4C | 674949846 | A, G | sanfensan |
| 4C | 675185316 | A, G | sanfensan |
| 4C | 683318163 | T, C | sanfensan |
| 4C | 689664776 | T, C | sanfensan |
| 4C | 694624379 | C, T | sanfensan |
| 4C | 695265876 | A, G | sanfensan |
| 4C | 701273830 | A, G | sanfensan |
| 4D | 3022923 | A, G | sanfensan |
| 4D | 7849878 | C, T | sanfensan |
| 4D | 10672344 | A, G | sanfensan |
| 4D | 16331251 | T, G | sanfensan |
| 4D | 23073905 | A, G | sanfensan |
| 4D | 28279824 | T, G | sanfensan |
| 4D | 32649688 | A, G | sanfensan |
| 4D | 37038379 | A, C | sanfensan |
| 4D | 43180451 | T, C | sanfensan |
| 4D | 45472604 | A, G | sanfensan |
| 4D | 50482410 | A, G | sanfensan |
| 4D | 58490879 | G, A | sanfensan |
| 4D | 62917919 | G, A | sanfensan |
| 4D | 65612418 | G, A | sanfensan |
| 4D | 71637400 | C, T | sanfensan |
| 4D | 76187635 | C, T | sanfensan |
| 4D | 82058789 | T, C | sanfensan |
| 4D | 88887772 | G, A | sanfensan |
| 4D | 92323177 | A, G | sanfensan |
| 4D | 97430755 | A, G | sanfensan |
| 4D | 102821671 | C, G | sanfensan |
| 4D | 106018930 | T, G | sanfensan |
| 4D | 110865436 | A, G | sanfensan |
| 4D | 116039043 | C, T | sanfensan |
| 4D | 118774001 | C, G | sanfensan |
| 4D | 121609230 | A, G | sanfensan |
| 4D | 128046905 | T, C | sanfensan |
| 4D | 134025258 | T, C | sanfensan |
| 4D | 137854184 | C, G | sanfensan |
| 4D | 141417395 | A, G | sanfensan |
| 4D | 148636319 | A, G | sanfensan |
| 4D | 151219343 | C, T | sanfensan |
| 4D | 153032013 | T, C | sanfensan |
| 4D | 159949170 | T, C | sanfensan |
| 4D | 163913258 | A, G | sanfensan |
| 4D | 165337682 | T, A | sanfensan |
| 4D | 173510202 | T, G | sanfensan |
| 4D | 179301600 | C, G | sanfensan |
| 4D | 184426060 | G, A | sanfensan |
| 4D | 187340496 | G, T | sanfensan |
| 4D | 193603755 | A, G | sanfensan |
| 4D | 196382892 | T, C | sanfensan |
| 4D | 202074844 | C, T | sanfensan |
| 4D | 206447702 | G, A | sanfensan |
| 4D | 212380635 | C, T | sanfensan |
| 4D | 215885690 | T, C | sanfensan |
| 4D | 220033123 | T, G | sanfensan |
| 4D | 227048389 | A, G | sanfensan |
| 4D | 232039750 | C, T | sanfensan |
| 4D | 236815937 | T, A | sanfensan |
| 4D | 242091618 | C, T | sanfensan |
| 4D | 245101303 | C, A | sanfensan |
| 4D | 250407222 | A, G | sanfensan |
| 4D | 256019130 | T, C | sanfensan |
| 4D | 260909900 | T, C | sanfensan |
| 4D | 267887871 | A, G | sanfensan |
| 4D | 271126060 | C, A | sanfensan |
| 4D | 277162174 | T, G | sanfensan |
| 4D | 283660950 | G, A | sanfensan |
| 4D | 285824135 | G, T | sanfensan |
| 4D | 294845699 | G, A | sanfensan |
| 4D | 299632398 | G, A | sanfensan |
| 4D | 303350038 | T, C | sanfensan |
| 4D | 307597358 | T, A | sanfensan |
| 4D | 314228425 | C, T | sanfensan |
| 4D | 314253165 | G, A | sanfensan |
| 4D | 315607825 | A, C | sanfensan |
| 4D | 321533893 | G, T | sanfensan |
| 4D | 325136857 | A, T | sanfensan |
| 4D | 326786150 | A, T | sanfensan |
| 4D | 332125743 | A, G | sanfensan |
| 4D | 335136720 | A, G | sanfensan |
| 4D | 340382269 | C, T | sanfensan |
| 4D | 348383382 | A, G | sanfensan |
| 4D | 352477479 | T, C | sanfensan |
| 4D | 356651761 | A, G | sanfensan |
| 4D | 363724413 | C, G | sanfensan |
| 4D | 366480345 | A, G | sanfensan |
| 4D | 374238192 | T, C | sanfensan |
| 4D | 378149215 | T, C | sanfensan |
| 4D | 384366440 | G, A | sanfensan |
| 4D | 389105351 | A, C | sanfensan |
| 4D | 390762487 | A, G | sanfensan |
| 4D | 398109909 | C, G | sanfensan |
| 4D | 402374421 | T, C | sanfensan |
| 4D | 407356318 | G, A | sanfensan |
| 4D | 413268611 | T, C | sanfensan |
| 4D | 418031647 | G, A | sanfensan |
| 4D | 420702529 | G, A | sanfensan |
| 4D | 426669740 | A, G | sanfensan |
| 4D | 432669116 | G, A | sanfensan |
| 4D | 437532533 | G, T | sanfensan |
| 4D | 443034471 | C, T | sanfensan |
| 4D | 445235008 | A, C | sanfensan |
| 4D | 451513413 | A, G | sanfensan |
| 5A | 2255953 | A, C | sanfensan |
| 5A | 6260213 | C, A | sanfensan |
| 5A | 13803621 | C, T | sanfensan |
| 5A | 15561310 | G, C | sanfensan |
| 5A | 24452734 | T, C | sanfensan |
| 5A | 27224463 | G, A | sanfensan |
| 5A | 34502392 | A, G | sanfensan |
| 5A | 36710142 | G, A | sanfensan |
| 5A | 44344522 | G, A | sanfensan |
| 5A | 49562544 | T, C | sanfensan |
| 5A | 53433795 | T, C | sanfensan |
| 5A | 56109353 | G, A | sanfensan |
| 5A | 64589225 | C, T | sanfensan |
| 5A | 68491563 | T, C | sanfensan |
| 5A | 70531911 | C, T | sanfensan |
| 5A | 79896986 | T, C | sanfensan |
| 5A | 83113930 | T, C | sanfensan |
| 5A | 86401636 | T, C | sanfensan |
| 5A | 94264887 | T, C | sanfensan |
| 5A | 95056890 | T, C | sanfensan |
| 5A | 102064390 | A, G | sanfensan |
| 5A | 106428331 | G, A | sanfensan |
| 5A | 110419074 | G, A | sanfensan |
| 5A | 116006562 | G, A | sanfensan |
| 5A | 121002541 | A, T | sanfensan |
| 5A | 127363246 | T, C | sanfensan |
| 5A | 132453075 | T, C | sanfensan |
| 5A | 137069112 | A, C | sanfensan |
| 5A | 142368295 | C, T | sanfensan |
| 5A | 147275003 | T, G | sanfensan |
| 5A | 152777713 | C, T | sanfensan |
| 5A | 156493283 | C, T | sanfensan |
| 5A | 160244662 | A, G | sanfensan |
| 5A | 166708997 | C, G | sanfensan |
| 5A | 174872733 | T, C | sanfensan |
| 5A | 176197051 | A, G | sanfensan |
| 5A | 181213909 | T, C | sanfensan |
| 5A | 188859714 | T, C | sanfensan |
| 5A | 190215196 | C, T | sanfensan |
| 5A | 195549254 | T, C | sanfensan |
| 5A | 198785103 | A, G | sanfensan |
| 5A | 202377152 | T, C | sanfensan |
| 5A | 207723478 | G, A | sanfensan |
| 5A | 212325208 | A, G | sanfensan |
| 5A | 216205965 | A, G | sanfensan |
| 5A | 221258102 | C, T | sanfensan |
| 5A | 225234818 | G, T | sanfensan |
| 5A | 230456063 | A, T | sanfensan |
| 5A | 238470416 | T, C | sanfensan |
| 5A | 240660477 | G, C | sanfensan |
| 5A | 245072572 | A, G | sanfensan |
| 5A | 250830642 | G, C | sanfensan |
| 5A | 259737745 | A, G | sanfensan |
| 5A | 261185047 | G, A | sanfensan |
| 5A | 269804753 | A, G | sanfensan |
| 5A | 273021787 | T, C | sanfensan |
| 5A | 276143054 | T, C | sanfensan |
| 5A | 282716721 | A, C | sanfensan |
| 5A | 289601566 | A, C | sanfensan |
| 5A | 290520143 | A, T | sanfensan |
| 5A | 297192869 | T, G | sanfensan |
| 5A | 301832004 | T, C | sanfensan |
| 5A | 308399750 | G, T | sanfensan |
| 5A | 311247256 | G, A | sanfensan |
| 5A | 317923031 | C, A | sanfensan |
| 5A | 321801333 | T, C | sanfensan |
| 5A | 327241850 | C, T | sanfensan |
| 5A | 333717258 | A, G | sanfensan |
| 5A | 335041321 | G, A | sanfensan |
| 5A | 341600328 | C, T | sanfensan |
| 5A | 347629159 | C, T | sanfensan |
| 5A | 350824983 | T, C | sanfensan |
| 5A | 356271802 | A, G | sanfensan |
| 5A | 363526707 | C, T | sanfensan |
| 5A | 369477353 | T, C | sanfensan |
| 5A | 370529828 | C, G | sanfensan |
| 5A | 375579309 | T, C | sanfensan |
| 5A | 383503810 | C, T | sanfensan |
| 5A | 387182267 | G, C | sanfensan |
| 5A | 390102471 | A, G | sanfensan |
| 5A | 398649985 | C, A | sanfensan |
| 5A | 404206934 | A, G | sanfensan |
| 5A | 409217956 | G, A | sanfensan |
| 5A | 411203550 | C, T | sanfensan |
| 5A | 415301859 | A, G | sanfensan |
| 5A | 416065809 | T, G | sanfensan |
| 5A | 421891077 | A, G | sanfensan |
| 5A | 428858871 | C, T | sanfensan |
| 5A | 434243983 | A, G | sanfensan |
| 5A | 436126819 | A, G | sanfensan |
| 5A | 441185565 | C, T | sanfensan |
| 5A | 447327633 | G, T | sanfensan |
| 5A | 452417280 | C, G | sanfensan |
| 5A | 458701937 | C, G | sanfensan |
| 5A | 460085569 | A, G | sanfensan |
| 5A | 468161069 | T, G | sanfensan |
| 5A | 474226937 | C, G | sanfensan |
| 5A | 478302848 | A, G | sanfensan |
| 5A | 483995877 | C, A | sanfensan |
| 5A | 488177553 | G, T | sanfensan |
| 5A | 488401160 | A, T | sanfensan |
| 5C | 3489579 | C, T | sanfensan |
| 5C | 6268971 | C, T | sanfensan |
| 5C | 12203702 | G, A | sanfensan |
| 5C | 16396954 | A, G | sanfensan |
| 5C | 22928378 | C, A | sanfensan |
| 5C | 26060685 | T, C | sanfensan |
| 5C | 31503941 | C, T | sanfensan |
| 5C | 39150034 | T, C | sanfensan |
| 5C | 44163319 | T, G | sanfensan |
| 5C | 47044843 | C, T | sanfensan |
| 5C | 50558717 | C, A | sanfensan |
| 5C | 53047612 | G, A | sanfensan |
| 5C | 56146722 | T, G | sanfensan |
| 5C | 63376645 | G, A | sanfensan |
| 5C | 69905284 | T, C | sanfensan |
| 5C | 74387265 | T, C | sanfensan |
| 5C | 77896048 | C, T | sanfensan |
| 5C | 81012623 | C, T | sanfensan |
| 5C | 86566325 | A, G | sanfensan |
| 5C | 94254331 | G, C | sanfensan |
| 5C | 95909941 | T, C | sanfensan |
| 5C | 101827763 | A, G | sanfensan |
| 5C | 109797422 | C, A | sanfensan |
| 5C | 114362886 | G, T | sanfensan |
| 5C | 119134297 | A, C | sanfensan |
| 5C | 121784178 | G, A | sanfensan |
| 5C | 125633452 | C, A | sanfensan |
| 5C | 132720505 | G, A | sanfensan |
| 5C | 138374966 | A, G | sanfensan |
| 5C | 143890607 | C, T | sanfensan |
| 5C | 145866226 | G, A | sanfensan |
| 5C | 150225588 | G, A | sanfensan |
| 5C | 156966699 | A, G | sanfensan |
| 5C | 160214876 | G, C | sanfensan |
| 5C | 168811131 | T, C | sanfensan |
| 5C | 174809389 | G, C | sanfensan |
| 5C | 177125115 | A, C | sanfensan |
| 5C | 182633756 | G, A | sanfensan |
| 5C | 189642953 | T, C | sanfensan |
| 5C | 193221965 | A, G | sanfensan |
| 5C | 199654787 | C, T | sanfensan |
| 5C | 201058413 | C, T | sanfensan |
| 5C | 208411390 | T, C | sanfensan |
| 5C | 214681134 | T, C | sanfensan |
| 5C | 216349665 | C, T | sanfensan |
| 5C | 220308120 | C, T | sanfensan |
| 5C | 223909826 | T, C | sanfensan |
| 5C | 227213415 | T, C | sanfensan |
| 5C | 234663041 | C, T | sanfensan |
| 5C | 236099260 | A, C | sanfensan |
| 5C | 244246397 | C, A | sanfensan |
| 5C | 246386706 | C, A | sanfensan |
| 5C | 252990985 | A, T | sanfensan |
| 5C | 255616974 | C, A | sanfensan |
| 5C | 261629346 | G, A | sanfensan |
| 5C | 266182002 | C, T | sanfensan |
| 5C | 271236736 | C, A | sanfensan |
| 5C | 275627914 | A, G | sanfensan |
| 5C | 283728720 | C, T | sanfensan |
| 5C | 285163996 | T, C | sanfensan |
| 5C | 292163090 | G, A | sanfensan |
| 5C | 297500311 | T, C | sanfensan |
| 5C | 304040309 | T, C | sanfensan |
| 5C | 306535191 | C, T | sanfensan |
| 5C | 313032349 | T, G | sanfensan |
| 5C | 318243088 | T, G | sanfensan |
| 5C | 320862543 | G, A | sanfensan |
| 5C | 325755346 | C, T | sanfensan |
| 5C | 333233541 | T, C | sanfensan |
| 5C | 338139569 | T, C | sanfensan |
| 5C | 342626437 | A, G | sanfensan |
| 5C | 347361434 | G, A | sanfensan |
| 5C | 354441762 | C, T | sanfensan |
| 5C | 358341050 | C, G | sanfensan |
| 5C | 363432176 | T, C | sanfensan |
| 5C | 369584563 | T, C | sanfensan |
| 5C | 373117738 | G, A | sanfensan |
| 5C | 377549147 | G, A | sanfensan |
| 5C | 384699195 | G, A | sanfensan |
| 5C | 385128549 | C, T | sanfensan |
| 5C | 391966210 | C, T | sanfensan |
| 5C | 399070404 | C, T | sanfensan |
| 5C | 402179080 | A, G | sanfensan |
| 5C | 405507477 | T, C | sanfensan |
| 5C | 411038650 | A, G | sanfensan |
| 5C | 416614236 | G, C | sanfensan |
| 5C | 421435620 | T, G | sanfensan |
| 5C | 427497128 | A, G | sanfensan |
| 5C | 432532568 | A, C | sanfensan |
| 5C | 435770963 | C, T | sanfensan |
| 5C | 440258824 | A, G | sanfensan |
| 5C | 447827826 | T, C | sanfensan |
| 5C | 451307256 | C, A | sanfensan |
| 5C | 458701577 | A, C | sanfensan |
| 5C | 462771476 | G, T | sanfensan |
| 5C | 468438848 | G, A | sanfensan |
| 5C | 473285230 | T, C | sanfensan |
| 5C | 477766211 | T, C | sanfensan |
| 5C | 480911717 | C, G | sanfensan |
| 5C | 489509201 | C, G | sanfensan |
| 5C | 490176188 | G, C | sanfensan |
| 5C | 498023277 | A, G | sanfensan |
| 5C | 503467351 | T, C | sanfensan |
| 5C | 509322035 | T, C | sanfensan |
| 5C | 512872294 | A, G | sanfensan |
| 5C | 515412167 | A, G | sanfensan |
| 5C | 520108101 | T, C | sanfensan |
| 5C | 526886988 | G, A | sanfensan |
| 5C | 533665172 | G, A | sanfensan |
| 5C | 536150519 | G, A | sanfensan |
| 5C | 542459731 | T, C | sanfensan |
| 5C | 548440305 | T, C | sanfensan |
| 5C | 551331730 | A, G | sanfensan |
| 5C | 555077838 | C, G | sanfensan |
| 5C | 560097889 | T, C | sanfensan |
| 5C | 569206294 | C, T | sanfensan |
| 5C | 572903980 | A, G | sanfensan |
| 5C | 579001353 | A, G | sanfensan |
| 5C | 582680547 | C, T | sanfensan |
| 5C | 585411392 | C, T | sanfensan |
| 5C | 593970148 | C, T | sanfensan |
| 5C | 598395218 | T, C | sanfensan |
| 5C | 601401624 | C, A | sanfensan |
| 5D | 4994611 | T, G | sanfensan |
| 5D | 6622542 | G, C | sanfensan |
| 5D | 10884878 | C, G | sanfensan |
| 5D | 16839171 | G, T | sanfensan |
| 5D | 21309845 | G, A | sanfensan |
| 5D | 27535507 | T, A | sanfensan |
| 5D | 34803208 | G, A | sanfensan |
| 5D | 36778603 | T, G | sanfensan |
| 5D | 41959313 | T, C | sanfensan |
| 5D | 47178435 | T, C | sanfensan |
| 5D | 52127682 | C, T | sanfensan |
| 5D | 54096343 | T, G | sanfensan |
| 5D | 56457835 | G, A | sanfensan |
| 5D | 63243306 | A, C | sanfensan |
| 5D | 64721781 | C, T | sanfensan |
| 5D | 65173806 | A, C | sanfensan |
| 5D | 66436115 | C, T | sanfensan |
| 5D | 74642065 | T, C | sanfensan |
| 5D | 79567830 | G, A | sanfensan |
| 5D | 83382549 | G, A | sanfensan |
| 5D | 87296427 | T, C | sanfensan |
| 5D | 93053910 | T, C | sanfensan |
| 5D | 99261576 | C, T | sanfensan |
| 5D | 101623688 | C, T | sanfensan |
| 5D | 109259133 | T, C | sanfensan |
| 5D | 110187772 | C, T | sanfensan |
| 5D | 116420976 | T, C | sanfensan |
| 5D | 120360110 | G, A | sanfensan |
| 5D | 128589324 | T, C | sanfensan |
| 5D | 134480766 | G, A | sanfensan |
| 5D | 136403599 | C, T | sanfensan |
| 5D | 142232990 | C, T | sanfensan |
| 5D | 145960007 | A, G | sanfensan |
| 5D | 153395376 | G, A | sanfensan |
| 5D | 155066422 | T, C | sanfensan |
| 5D | 162991627 | T, C | sanfensan |
| 5D | 167232512 | C, T | sanfensan |
| 5D | 172000670 | T, C | sanfensan |
| 5D | 177160582 | G, A | sanfensan |
| 5D | 182553299 | C, T | sanfensan |
| 5D | 187357553 | C, T | sanfensan |
| 5D | 191107356 | A, G | sanfensan |
| 5D | 199208574 | C, T | sanfensan |
| 5D | 203029590 | A, G | sanfensan |
| 5D | 209809131 | T, G | sanfensan |
| 5D | 211555734 | A, T | sanfensan |
| 5D | 216140070 | T, G | sanfensan |
| 5D | 221499002 | A, G | sanfensan |
| 5D | 228576676 | A, G | sanfensan |
| 5D | 230582553 | G, C | sanfensan |
| 5D | 238425107 | C, T | sanfensan |
| 5D | 242649305 | G, A | sanfensan |
| 5D | 245087633 | T, C | sanfensan |
| 5D | 250750865 | G, T | sanfensan |
| 5D | 258642655 | G, A | sanfensan |
| 5D | 262006477 | A, G | sanfensan |
| 5D | 266745740 | G, A | sanfensan |
| 5D | 271633260 | G, A | sanfensan |
| 5D | 278837964 | C, T | sanfensan |
| 5D | 280282084 | T, C | sanfensan |
| 5D | 289610610 | A, T | sanfensan |
| 5D | 293820840 | C, T | sanfensan |
| 5D | 298575083 | A, T | sanfensan |
| 5D | 302837192 | T, C | sanfensan |
| 5D | 305502791 | T, C | sanfensan |
| 5D | 314628778 | A, G | sanfensan |
| 5D | 319756146 | G, A | sanfensan |
| 5D | 322226424 | C, A | sanfensan |
| 5D | 328035262 | C, T | sanfensan |
| 5D | 332326959 | C, T | sanfensan |
| 5D | 339720781 | T, A | sanfensan |
| 5D | 344363610 | A, C | sanfensan |
| 5D | 345547743 | T, C | sanfensan |
| 5D | 353592498 | G, A | sanfensan |
| 5D | 357027031 | C, T | sanfensan |
| 5D | 363095927 | T, G | sanfensan |
| 5D | 365082132 | T, C | sanfensan |
| 5D | 372087578 | A, C | sanfensan |
| 5D | 375075448 | A, G | sanfensan |
| 5D | 382109948 | T, C | sanfensan |
| 5D | 385044778 | A, G | sanfensan |
| 5D | 394858589 | C, T | sanfensan |
| 5D | 399910298 | C, T | sanfensan |
| 5D | 402227007 | G, A | sanfensan |
| 5D | 407502206 | T, C | sanfensan |
| 5D | 410635861 | A, G | sanfensan |
| 5D | 419664992 | T, C | sanfensan |
| 5D | 421256053 | A, G | sanfensan |
| 5D | 427332097 | C, G | sanfensan |
| 5D | 433626893 | G, T | sanfensan |
| 5D | 437055110 | C, G | sanfensan |
| 5D | 440529761 | C, T | sanfensan |
| 5D | 446386566 | C, T | sanfensan |
| 5D | 454896966 | T, C | sanfensan |
| 5D | 458673318 | G, A | sanfensan |
| 5D | 462992703 | G, A | sanfensan |
| 5D | 466900163 | C, T | sanfensan |
| 5D | 474924404 | C, T | sanfensan |
| 5D | 478407877 | T, C | sanfensan |
| 5D | 482793426 | T, C | sanfensan |
| 5D | 488236543 | A, C | sanfensan |
| 5D | 492418351 | A, G | sanfensan |
| 5D | 497166345 | G, T | sanfensan |
| 5D | 500430897 | T, G | sanfensan |
| 6A | 4492157 | T, C | sanfensan |
| 6A | 8762221 | G, T | sanfensan |
| 6A | 11190798 | T, C | sanfensan |
| 6A | 17691406 | C, T | sanfensan |
| 6A | 20852081 | T, C | sanfensan |
| 6A | 26524896 | C, T | sanfensan |
| 6A | 33584644 | C, T | sanfensan |
| 6A | 36710498 | A, G | sanfensan |
| 6A | 42587073 | A, G | sanfensan |
| 6A | 48196250 | G, C | sanfensan |
| 6A | 54285408 | C, G | sanfensan |
| 6A | 57328665 | T, A | sanfensan |
| 6A | 64386015 | C, A | sanfensan |
| 6A | 67285702 | C, T | sanfensan |
| 6A | 71815074 | C, A | sanfensan |
| 6A | 77438434 | C, A | sanfensan |
| 6A | 83659654 | G, A | sanfensan |
| 6A | 85235001 | C, A | sanfensan |
| 6A | 94561841 | T, G | sanfensan |
| 6A | 95323662 | A, C | sanfensan |
| 6A | 103614490 | T, C | sanfensan |
| 6A | 108982250 | C, T | sanfensan |
| 6A | 109794291 | G, A | sanfensan |
| 6A | 113440903 | T, G | sanfensan |
| 6A | 118377169 | C, G | sanfensan |
| 6A | 124172048 | A, G | sanfensan |
| 6A | 125350773 | A, C | sanfensan |
| 6A | 130843545 | T, C | sanfensan |
| 6A | 135683529 | T, C | sanfensan |
| 6A | 143547400 | A, T | sanfensan |
| 6A | 147670247 | A, G | sanfensan |
| 6A | 151304761 | T, C | sanfensan |
| 6A | 157973297 | G, C | sanfensan |
| 6A | 158294119 | A, C | sanfensan |
| 6A | 163354468 | G, C | sanfensan |
| 6A | 165693464 | T, C | sanfensan |
| 6A | 173487971 | G, A | sanfensan |
| 6A | 176286584 | G, A | sanfensan |
| 6A | 183958426 | C, T | sanfensan |
| 6A | 186274335 | G, A | sanfensan |
| 6A | 194312081 | C, T | sanfensan |
| 6A | 198052953 | G, A | sanfensan |
| 6A | 203626460 | T, G | sanfensan |
| 6A | 208551969 | G, A | sanfensan |
| 6A | 212325078 | A, G | sanfensan |
| 6A | 216393559 | G, A | sanfensan |
| 6A | 222443511 | C, G | sanfensan |
| 6A | 227146163 | G, A | sanfensan |
| 6A | 232355131 | C, A | sanfensan |
| 6A | 236306595 | G, A | sanfensan |
| 6A | 242551165 | C, T | sanfensan |
| 6A | 248683146 | T, A | sanfensan |
| 6A | 254218064 | C, T | sanfensan |
| 6A | 256311166 | G, A | sanfensan |
| 6A | 263945630 | T, C | sanfensan |
| 6A | 265291617 | G, A | sanfensan |
| 6A | 272551424 | T, C | sanfensan |
| 6A | 276146901 | A, C | sanfensan |
| 6A | 283068354 | T, C | sanfensan |
| 6A | 287654071 | G, A | sanfensan |
| 6A | 291439784 | A, C | sanfensan |
| 6A | 296813413 | T, A | sanfensan |
| 6A | 304331839 | T, C | sanfensan |
| 6A | 309571759 | G, C | sanfensan |
| 6A | 311843254 | A, C | sanfensan |
| 6A | 318306732 | G, A | sanfensan |
| 6A | 324491687 | A, C | sanfensan |
| 6A | 326644360 | G, A | sanfensan |
| 6A | 330034066 | C, T | sanfensan |
| 6A | 335513362 | C, T | sanfensan |
| 6A | 339809705 | C, G | sanfensan |
| 6A | 343191906 | T, C | sanfensan |
| 6A | 349219222 | G, A | sanfensan |
| 6A | 351759840 | A, G | sanfensan |
| 6A | 358242236 | G, T | sanfensan |
| 6A | 362754991 | T, C | sanfensan |
| 6A | 367473345 | G, A | sanfensan |
| 6A | 371831728 | G, A | sanfensan |
| 6A | 377260484 | T, C | sanfensan |
| 6A | 382654181 | C, T | sanfensan |
| 6A | 389448736 | A, G | sanfensan |
| 6A | 394354567 | T, C | sanfensan |
| 6A | 395689953 | A, G | sanfensan |
| 6A | 402143190 | A, G | sanfensan |
| 6A | 409084859 | A, C | sanfensan |
| 6A | 414438357 | A, G | sanfensan |
| 6A | 415355947 | T, C | sanfensan |
| 6A | 421445718 | A, C | sanfensan |
| 6A | 422458226 | G, A | sanfensan |
| 6A | 425035666 | C, A | sanfensan |
| 6A | 433725430 | A, G | sanfensan |
| 6A | 437585163 | T, C | sanfensan |
| 6A | 444959115 | G, C | sanfensan |
| 6A | 445547963 | C, T | sanfensan |
| 6A | 454994988 | A, G | sanfensan |
| 6C | 4740123 | A, C | sanfensan |
| 6C | 7818111 | T, G | sanfensan |
| 6C | 10608939 | A, T | sanfensan |
| 6C | 17502954 | A, C | sanfensan |
| 6C | 21919209 | C, T | sanfensan |
| 6C | 27489398 | T, C | sanfensan |
| 6C | 32883642 | T, G | sanfensan |
| 6C | 35200287 | C, A | sanfensan |
| 6C | 38472337 | T, C | sanfensan |
| 6C | 43196331 | A, G | sanfensan |
| 6C | 47589408 | A, G | sanfensan |
| 6C | 51088171 | A, G | sanfensan |
| 6C | 59096246 | G, A | sanfensan |
| 6C | 60407986 | C, T | sanfensan |
| 6C | 65757372 | C, T | sanfensan |
| 6C | 66609935 | G, A | sanfensan |
| 6C | 72292296 | A, G | sanfensan |
| 6C | 75973462 | A, T | sanfensan |
| 6C | 81974276 | C, T | sanfensan |
| 6C | 88129794 | G, C | sanfensan |
| 6C | 94193475 | T, C | sanfensan |
| 6C | 95536874 | T, C | sanfensan |
| 6C | 101717822 | C, T | sanfensan |
| 6C | 107891612 | C, T | sanfensan |
| 6C | 112851715 | A, T | sanfensan |
| 6C | 116496712 | C, T | sanfensan |
| 6C | 121111029 | T, C | sanfensan |
| 6C | 125320836 | G, A | sanfensan |
| 6C | 132900758 | T, A | sanfensan |
| 6C | 135785484 | G, C | sanfensan |
| 6C | 143030880 | C, A | sanfensan |
| 6C | 148851945 | G, A | sanfensan |
| 6C | 154103608 | A, C | sanfensan |
| 6C | 156034908 | T, G | sanfensan |
| 6C | 162091462 | A, C | sanfensan |
| 6C | 165806162 | A, T | sanfensan |
| 6C | 172155825 | G, A | sanfensan |
| 6C | 175590346 | C, T | sanfensan |
| 6C | 180731171 | G, A | sanfensan |
| 6C | 185475790 | A, G | sanfensan |
| 6C | 194315523 | G, A | sanfensan |
| 6C | 196088194 | G, A | sanfensan |
| 6C | 196851358 | C, T | sanfensan |
| 6C | 200122712 | A, C | sanfensan |
| 6C | 207092401 | A, G | sanfensan |
| 6C | 213333184 | T, G | sanfensan |
| 6C | 217526046 | G, A | sanfensan |
| 6C | 222420550 | C, T | sanfensan |
| 6C | 227353579 | G, A | sanfensan |
| 6C | 234060684 | A, G | sanfensan |
| 6C | 235857775 | G, A | sanfensan |
| 6C | 244931366 | T, C | sanfensan |
| 6C | 246070168 | G, A | sanfensan |
| 6C | 253789587 | C, T | sanfensan |
| 6C | 257684248 | G, C | sanfensan |
| 6C | 260070968 | C, T | sanfensan |
| 6C | 266510278 | C, T | sanfensan |
| 6C | 272777330 | C, T | sanfensan |
| 6C | 275890891 | G, A | sanfensan |
| 6C | 284003530 | C, T | sanfensan |
| 6C | 289057124 | C, T | sanfensan |
| 6C | 292273925 | G, A | sanfensan |
| 6C | 295852267 | A, C | sanfensan |
| 6C | 301581063 | G, A | sanfensan |
| 6C | 308002832 | T, C | sanfensan |
| 6C | 310533434 | G, A | sanfensan |
| 6C | 316651599 | G, C | sanfensan |
| 6C | 320349680 | G, A | sanfensan |
| 6C | 325082414 | T, C | sanfensan |
| 6C | 331834200 | C, T | sanfensan |
| 6C | 335435627 | T, C | sanfensan |
| 6C | 341601920 | C, A | sanfensan |
| 6C | 348384420 | T, C | sanfensan |
| 6C | 354770875 | T, C | sanfensan |
| 6C | 355787593 | T, C | sanfensan |
| 6C | 363981785 | C, T | sanfensan |
| 6C | 367279098 | C, G | sanfensan |
| 6C | 372097294 | G, T | sanfensan |
| 6C | 376086340 | G, A | sanfensan |
| 6C | 382987219 | C, T | sanfensan |
| 6C | 385017932 | A, G | sanfensan |
| 6C | 393722890 | C, T | sanfensan |
| 6C | 396489334 | T, C | sanfensan |
| 6C | 402999061 | G, A | sanfensan |
| 6C | 404034553 | G, A | sanfensan |
| 6C | 407439238 | G, C | sanfensan |
| 6C | 412317336 | C, T | sanfensan |
| 6C | 416156258 | T, C | sanfensan |
| 6C | 422586122 | G, C | sanfensan |
| 6C | 429700897 | C, T | sanfensan |
| 6C | 430532023 | A, G | sanfensan |
| 6C | 436366975 | G, A | sanfensan |
| 6C | 440291570 | T, G | sanfensan |
| 6C | 447368142 | A, G | sanfensan |
| 6C | 454158229 | C, T | sanfensan |
| 6C | 457431878 | G, A | sanfensan |
| 6C | 464408570 | A, G | sanfensan |
| 6C | 466196657 | G, A | sanfensan |
| 6C | 470467140 | G, T | sanfensan |
| 6C | 475226773 | G, A | sanfensan |
| 6C | 484622983 | G, A | sanfensan |
| 6C | 489733584 | G, T | sanfensan |
| 6C | 491124804 | T, C | sanfensan |
| 6C | 496637243 | G, A | sanfensan |
| 6C | 502828531 | C, T | sanfensan |
| 6C | 505002174 | G, A | sanfensan |
| 6C | 507736024 | C, T | sanfensan |
| 6C | 512237599 | C, T | sanfensan |
| 6C | 516058707 | C, T | sanfensan |
| 6C | 522649701 | C, G | sanfensan |
| 6C | 529463088 | G, T | sanfensan |
| 6C | 532838127 | C, T | sanfensan |
| 6C | 538744714 | C, T | sanfensan |
| 6C | 541894652 | T, C | sanfensan |
| 6C | 548890274 | A, G | sanfensan |
| 6C | 550597198 | C, T | sanfensan |
| 6C | 555553384 | T, C | sanfensan |
| 6C | 563333574 | G, T | sanfensan |
| 6C | 566938058 | T, C | sanfensan |
| 6C | 574826495 | A, C | sanfensan |
| 6C | 576805876 | G, A | sanfensan |
| 6C | 581584319 | G, C | sanfensan |
| 6C | 585343852 | C, T | sanfensan |
| 6C | 590741273 | T, C | sanfensan |
| 6C | 599908901 | G, A | sanfensan |
| 6C | 601571029 | C, T | sanfensan |
| 6C | 605547418 | C, T | sanfensan |
| 6D | 2777295 | C, T | sanfensan |
| 6D | 6267655 | C, A | sanfensan |
| 6D | 13107420 | C, T | sanfensan |
| 6D | 16132609 | C, A | sanfensan |
| 6D | 23982220 | A, C | sanfensan |
| 6D | 26754468 | T, G | sanfensan |
| 6D | 32172782 | C, T | sanfensan |
| 6D | 35628869 | T, C | sanfensan |
| 6D | 44217561 | A, G | sanfensan |
| 6D | 45240610 | T, G | sanfensan |
| 6D | 50128337 | G, C | sanfensan |
| 6D | 55030113 | A, G | sanfensan |
| 6D | 64553482 | T, C | sanfensan |
| 6D | 66247035 | T, C | sanfensan |
| 6D | 73309196 | A, G | sanfensan |
| 6D | 77363272 | A, G | sanfensan |
| 6D | 84655953 | T, C | sanfensan |
| 6D | 89015326 | T, C | sanfensan |
| 6D | 91516090 | A, G | sanfensan |
| 6D | 98584233 | A, G | sanfensan |
| 6D | 104170504 | T, G | sanfensan |
| 6D | 108654428 | T, C | sanfensan |
| 6D | 114351697 | C, G | sanfensan |
| 6D | 118475182 | G, A | sanfensan |
| 6D | 120657246 | A, G | sanfensan |
| 6D | 128950046 | A, G | sanfensan |
| 6D | 132670154 | T, C | sanfensan |
| 6D | 139598945 | A, G | sanfensan |
| 6D | 142833751 | A, C | sanfensan |
| 6D | 146878955 | C, T | sanfensan |
| 6D | 150804995 | G, A | sanfensan |
| 6D | 159442646 | T, C | sanfensan |
| 6D | 161979848 | G, A | sanfensan |
| 6D | 166007399 | C, T | sanfensan |
| 6D | 171110144 | T, C | sanfensan |
| 6D | 178157758 | C, T | sanfensan |
| 6D | 181144200 | A, C | sanfensan |
| 6D | 181398145 | T, G | sanfensan |
| 6D | 188963826 | T, C | sanfensan |
| 6D | 190320583 | G, A | sanfensan |
| 6D | 198738426 | G, A | sanfensan |
| 6D | 204695478 | C, T | sanfensan |
| 6D | 209111889 | A, C | sanfensan |
| 6D | 213388210 | T, C | sanfensan |
| 6D | 217365606 | G, A | sanfensan |
| 6D | 220497638 | C, T | sanfensan |
| 6D | 226248655 | G, T | sanfensan |
| 6D | 230357776 | T, C | sanfensan |
| 6D | 239771210 | A, G | sanfensan |
| 6D | 240712867 | T, A | sanfensan |
| 6D | 245616215 | G, A | sanfensan |
| 6D | 254910351 | T, C | sanfensan |
| 6D | 257401569 | T, C | sanfensan |
| 6D | 263701170 | T, G | sanfensan |
| 6D | 268768437 | A, G | sanfensan |
| 6D | 270928208 | C, A | sanfensan |
| 6D | 278371196 | C, T | sanfensan |
| 6D | 284570630 | A, G | sanfensan |
| 6D | 285173852 | A, G | sanfensan |
| 6D | 290592914 | T, A | sanfensan |
| 7A | 556988 | G, A | sanfensan |
| 7A | 9387452 | T, C | sanfensan |
| 7A | 10624667 | T, C | sanfensan |
| 7A | 16247028 | G, A | sanfensan |
| 7A | 24258432 | T, A | sanfensan |
| 7A | 29212189 | T, C | sanfensan |
| 7A | 32277248 | T, G | sanfensan |
| 7A | 38743380 | T, C | sanfensan |
| 7A | 42755867 | A, G | sanfensan |
| 7A | 46387061 | C, T | sanfensan |
| 7A | 51855350 | A, G | sanfensan |
| 7A | 57885922 | C, A | sanfensan |
| 7A | 63281807 | T, C | sanfensan |
| 7A | 65480794 | G, A | sanfensan |
| 7A | 71137163 | T, C | sanfensan |
| 7A | 75733143 | T, A | sanfensan |
| 7A | 82158130 | G, C | sanfensan |
| 7A | 85385227 | C, T | sanfensan |
| 7A | 91146020 | C, T | sanfensan |
| 7A | 99518702 | T, C | sanfensan |
| 7A | 100100809 | C, A | sanfensan |
| 7A | 108559177 | C, T | sanfensan |
| 7A | 110159568 | G, T | sanfensan |
| 7A | 119859092 | T, C | sanfensan |
| 7A | 120329198 | C, A | sanfensan |
| 7A | 125074054 | T, C | sanfensan |
| 7A | 131168556 | C, T | sanfensan |
| 7A | 139918960 | A, G | sanfensan |
| 7A | 143266476 | T, C | sanfensan |
| 7A | 149300335 | G, A | sanfensan |
| 7A | 154734171 | T, C | sanfensan |
| 7A | 159313052 | C, T | sanfensan |
| 7A | 160075815 | C, G | sanfensan |
| 7A | 167272883 | C, T | sanfensan |
| 7A | 171591003 | T, G | sanfensan |
| 7A | 177018083 | G, T | sanfensan |
| 7A | 184807238 | C, A | sanfensan |
| 7A | 186333580 | A, G | sanfensan |
| 7A | 190961467 | T, C | sanfensan |
| 7A | 197600313 | G, A | sanfensan |
| 7A | 201074764 | C, A | sanfensan |
| 7A | 208272139 | T, G | sanfensan |
| 7A | 212498087 | C, T | sanfensan |
| 7A | 219185506 | G, T | sanfensan |
| 7A | 222607909 | A, G | sanfensan |
| 7A | 226206685 | T, C | sanfensan |
| 7A | 234382860 | A, T | sanfensan |
| 7A | 235670912 | C, T | sanfensan |
| 7A | 242252774 | A, G | sanfensan |
| 7A | 246670561 | A, C | sanfensan |
| 7A | 250206642 | T, C | sanfensan |
| 7A | 259731844 | C, T | sanfensan |
| 7A | 261123685 | T, C | sanfensan |
| 7A | 267061512 | G, C | sanfensan |
| 7A | 273875056 | A, T | sanfensan |
| 7A | 279451537 | C, G | sanfensan |
| 7A | 283880271 | G, A | sanfensan |
| 7A | 289470204 | T, C | sanfensan |
| 7A | 294405482 | T, C | sanfensan |
| 7A | 296729065 | G, T | sanfensan |
| 7A | 304733749 | T, C | sanfensan |
| 7A | 306515921 | T, C | sanfensan |
| 7A | 310678817 | G, A | sanfensan |
| 7A | 317998480 | C, T | sanfensan |
| 7A | 324083519 | A, G | sanfensan |
| 7A | 326015574 | A, G | sanfensan |
| 7A | 334096551 | G, A | sanfensan |
| 7A | 337503568 | G, A | sanfensan |
| 7A | 342389640 | T, C | sanfensan |
| 7A | 349080210 | A, G | sanfensan |
| 7A | 352615237 | T, C | sanfensan |
| 7A | 355460008 | G, A | sanfensan |
| 7A | 361858462 | T, C | sanfensan |
| 7A | 363873233 | C, T | sanfensan |
| 7A | 367416741 | A, G | sanfensan |
| 7A | 370900705 | C, A | sanfensan |
| 7A | 377494585 | C, T | sanfensan |
| 7A | 380620357 | T, C | sanfensan |
| 7A | 387542205 | A, G | sanfensan |
| 7A | 393193897 | T, C | sanfensan |
| 7A | 397890567 | A, G | sanfensan |
| 7A | 402356276 | A, T | sanfensan |
| 7A | 408034177 | T, G | sanfensan |
| 7A | 411938390 | C, T | sanfensan |
| 7A | 415820845 | C, T | sanfensan |
| 7A | 421650338 | A, G | sanfensan |
| 7A | 429369869 | A, G | sanfensan |
| 7A | 433335698 | G, A | sanfensan |
| 7A | 438899194 | T, C | sanfensan |
| 7A | 442020982 | T, C | sanfensan |
| 7A | 449206870 | A, G | sanfensan |
| 7A | 452644138 | A, C | sanfensan |
| 7A | 455480446 | T, G | sanfensan |
| 7A | 463118331 | A, G | sanfensan |
| 7A | 467735097 | C, T | sanfensan |
| 7A | 471902717 | A, G | sanfensan |
| 7A | 476107323 | A, G | sanfensan |
| 7A | 482021904 | C, T | sanfensan |
| 7A | 485131125 | A, G | sanfensan |
| 7A | 493482291 | G, A | sanfensan |
| 7A | 495121727 | T, C | sanfensan |
| 7C | 4614858 | T, G | sanfensan |
| 7C | 5317219 | C, T | sanfensan |
| 7C | 14340861 | A, G | sanfensan |
| 7C | 19478034 | G, A | sanfensan |
| 7C | 24565676 | G, A | sanfensan |
| 7C | 29930105 | T, C | sanfensan |
| 7C | 32494696 | G, A | sanfensan |
| 7C | 39005353 | C, T | sanfensan |
| 7C | 41317427 | A, G | sanfensan |
| 7C | 47961168 | A, C | sanfensan |
| 7C | 50182911 | G, C | sanfensan |
| 7C | 55925952 | A, G | sanfensan |
| 7C | 61239403 | C, G | sanfensan |
| 7C | 67047337 | T, G | sanfensan |
| 7C | 71975946 | G, T | sanfensan |
| 7C | 72522696 | C, T | sanfensan |
| 7C | 79287234 | A, C | sanfensan |
| 7C | 84848962 | G, A | sanfensan |
| 7C | 88219749 | C, T | sanfensan |
| 7C | 94995046 | A, G | sanfensan |
| 7C | 97771126 | T, C | sanfensan |
| 7C | 104891291 | G, A | sanfensan |
| 7C | 108552236 | A, G | sanfensan |
| 7C | 113384152 | G, T | sanfensan |
| 7C | 118141656 | A, G | sanfensan |
| 7C | 123007101 | G, C | sanfensan |
| 7C | 129029468 | T, C | sanfensan |
| 7C | 134291990 | T, C | sanfensan |
| 7C | 137256580 | T, C | sanfensan |
| 7C | 140877690 | G, A | sanfensan |
| 7C | 145949568 | G, C | sanfensan |
| 7C | 151007290 | G, A | sanfensan |
| 7C | 157054447 | C, T | sanfensan |
| 7C | 163909424 | G, A | sanfensan |
| 7C | 166612341 | A, C | sanfensan |
| 7C | 171906105 | A, G | sanfensan |
| 7C | 177293559 | T, C | sanfensan |
| 7C | 179283192 | T, C | sanfensan |
| 7C | 180628837 | C, T | sanfensan |
| 7C | 185998111 | A, G | sanfensan |
| 7C | 191666515 | A, G | sanfensan |
| 7C | 197044982 | A, G | sanfensan |
| 7C | 204791800 | A, C | sanfensan |
| 7C | 206297454 | T, C | sanfensan |
| 7C | 214665049 | T, C | sanfensan |
| 7C | 216739159 | A, G | sanfensan |
| 7C | 220606602 | T, C | sanfensan |
| 7C | 225604579 | C, A | sanfensan |
| 7C | 232971786 | G, C | sanfensan |
| 7C | 236226644 | C, T | sanfensan |
| 7C | 242089348 | C, G | sanfensan |
| 7C | 247003405 | C, T | sanfensan |
| 7C | 249178004 | T, G | sanfensan |
| 7C | 250146710 | C, T | sanfensan |
| 7C | 257100796 | G, A | sanfensan |
| 7C | 261648943 | C, A | sanfensan |
| 7C | 268903706 | T, C | sanfensan |
| 7C | 273820692 | C, G | sanfensan |
| 7C | 278686128 | T, C | sanfensan |
| 7C | 280947265 | G, A | sanfensan |
| 7C | 286809973 | T, C | sanfensan |
| 7C | 291012394 | G, C | sanfensan |
| 7C | 296841339 | A, C | sanfensan |
| 7C | 301552328 | T, C | sanfensan |
| 7C | 304378578 | A, G | sanfensan |
| 7C | 306040659 | G, A | sanfensan |
| 7C | 314412301 | G, T | sanfensan |
| 7C | 318527191 | C, T | sanfensan |
| 7C | 320205502 | A, G | sanfensan |
| 7C | 320293039 | G, T | sanfensan |
| 7C | 327857134 | C, A | sanfensan |
| 7C | 331712162 | T, C | sanfensan |
| 7C | 339499335 | T, C | sanfensan |
| 7C | 340372302 | C, T | sanfensan |
| 7C | 349292850 | C, T | sanfensan |
| 7C | 350022239 | G, A | sanfensan |
| 7C | 350993553 | A, G | sanfensan |
| 7C | 358188563 | T, C | sanfensan |
| 7C | 363492217 | C, T | sanfensan |
| 7C | 369432993 | G, A | sanfensan |
| 7C | 371369521 | T, G | sanfensan |
| 7C | 377634589 | A, G | sanfensan |
| 7C | 384171632 | C, T | sanfensan |
| 7C | 386369415 | G, A | sanfensan |
| 7C | 392363537 | C, T | sanfensan |
| 7C | 396114953 | A, G | sanfensan |
| 7C | 400812310 | C, T | sanfensan |
| 7C | 405139125 | A, G | sanfensan |
| 7C | 413833654 | A, C | sanfensan |
| 7C | 414551856 | G, T | sanfensan |
| 7C | 416262975 | C, T | sanfensan |
| 7C | 420923703 | T, C | sanfensan |
| 7C | 426911439 | C, A | sanfensan |
| 7C | 430613225 | G, A | sanfensan |
| 7C | 432461759 | G, A | sanfensan |
| 7C | 435550002 | C, T | sanfensan |
| 7C | 444070312 | G, T | sanfensan |
| 7C | 445489155 | G, A | sanfensan |
| 7C | 452005889 | A, T | sanfensan |
| 7C | 457711724 | T, C | sanfensan |
| 7C | 462688367 | G, A | sanfensan |
| 7C | 464956118 | A, G | sanfensan |
| 7C | 465254207 | G, T | sanfensan |
| 7C | 471154727 | G, A | sanfensan |
| 7C | 476266406 | C, A | sanfensan |
| 7C | 480020947 | C, T | sanfensan |
| 7C | 485814933 | A, G | sanfensan |
| 7C | 491712274 | T, C | sanfensan |
| 7C | 496402779 | G, C | sanfensan |
| 7C | 500349440 | G, A | sanfensan |
| 7C | 505836662 | C, T | sanfensan |
| 7C | 514393934 | A, G | sanfensan |
| 7C | 518087154 | C, G | sanfensan |
| 7C | 520313196 | A, G | sanfensan |
| 7C | 528181463 | G, C | sanfensan |
| 7C | 530421340 | G, C | sanfensan |
| 7D | 2445778 | G, A | sanfensan |
| 7D | 9742047 | A, G | sanfensan |
| 7D | 9847518 | A, G | sanfensan |
| 7D | 13815389 | A, T | sanfensan |
| 7D | 18736551 | G, T | sanfensan |
| 7D | 20505929 | C, T | sanfensan |
| 7D | 25511353 | G, A | sanfensan |
| 7D | 30499092 | G, A | sanfensan |
| 7D | 36614804 | T, G | sanfensan |
| 7D | 40700604 | G, C | sanfensan |
| 7D | 46604719 | C, T | sanfensan |
| 7D | 50727705 | C, G | sanfensan |
| 7D | 57700439 | C, T | sanfensan |
| 7D | 60169262 | C, A | sanfensan |
| 7D | 69063876 | A, C | sanfensan |
| 7D | 71993254 | T, C | sanfensan |
| 7D | 79211559 | A, G | sanfensan |
| 7D | 82904994 | C, T | sanfensan |
| 7D | 85615821 | A, G | sanfensan |
| 7D | 91224657 | T, C | sanfensan |
| 7D | 98952743 | G, A | sanfensan |
| 7D | 100841898 | C, T | sanfensan |
| 7D | 108477777 | G, A | sanfensan |
| 7D | 110334702 | C, T | sanfensan |
| 7D | 116643732 | C, T | sanfensan |
| 7D | 123307950 | T, C | sanfensan |
| 7D | 127161912 | A, G | sanfensan |
| 7D | 133410614 | T, G | sanfensan |
| 7D | 138641756 | C, T | sanfensan |
| 7D | 141652553 | T, G | sanfensan |
| 7D | 146207944 | C, T | sanfensan |
| 7D | 151573471 | G, A | sanfensan |
| 7D | 156040797 | C, A | sanfensan |
| 7D | 161660385 | A, G | sanfensan |
| 7D | 167061192 | A, G | sanfensan |
| 7D | 170724248 | A, G | sanfensan |
| 7D | 178681077 | A, C | sanfensan |
| 7D | 183800740 | A, C | sanfensan |
| 7D | 186691354 | A, G | sanfensan |
| 7D | 192032150 | G, A | sanfensan |
| 7D | 197240877 | G, A | sanfensan |
| 7D | 203331302 | A, G | sanfensan |
| 7D | 206895614 | A, G | sanfensan |
| 7D | 214500259 | A, T | sanfensan |
| 7D | 216095702 | C, A | sanfensan |
| 7D | 223545306 | G, A | sanfensan |
| 7D | 226229762 | G, A | sanfensan |
| 7D | 232450357 | G, C | sanfensan |
| 7D | 237413151 | T, C | sanfensan |
| 7D | 243355850 | T, C | sanfensan |
| 7D | 247929240 | G, T | sanfensan |
| 7D | 254107993 | C, G | sanfensan |
| 7D | 255422386 | T, C | sanfensan |
| 7D | 264369755 | A, C | sanfensan |
| 7D | 267080055 | G, C | sanfensan |
| 7D | 272102782 | T, C | sanfensan |
| 7D | 276724318 | T, C | sanfensan |
| 7D | 278615324 | G, C | sanfensan |
| 7D | 283701259 | T, G | sanfensan |
| 7D | 285490224 | C, T | sanfensan |
| 7D | 290974761 | C, T | sanfensan |
| 7D | 299205902 | T, C | sanfensan |
| 7D | 302081398 | G, A | sanfensan |
| 7D | 305799487 | A, G | sanfensan |
| 7D | 310083219 | A, G | sanfensan |
| 7D | 318265612 | C, T | sanfensan |
| 7D | 324554035 | C, A | sanfensan |
| 7D | 328162737 | A, G | sanfensan |
| 7D | 332476238 | C, T | sanfensan |
| 7D | 336554421 | T, C | sanfensan |
| 7D | 342546157 | T, C | sanfensan |
| 7D | 348921802 | A, G | sanfensan |
| 7D | 352024567 | A, C | sanfensan |
| 7D | 358950077 | C, T | sanfensan |
| 7D | 362820207 | G, A | sanfensan |
| 7D | 368623531 | T, C | sanfensan |
| 7D | 371406914 | G, T | sanfensan |
| 7D | 379681421 | T, C | sanfensan |
| 7D | 382815770 | C, T | sanfensan |
| 7D | 389570780 | G, A | sanfensan |
| 7D | 390101972 | G, A | sanfensan |
| 7D | 399143078 | C, T | sanfensan |
| 7D | 400807921 | C, T | sanfensan |
| 7D | 406112763 | T, G | sanfensan |
| 7D | 411104681 | A, G | sanfensan |
| 7D | 415490064 | G, A | sanfensan |
| 7D | 423220521 | A, G | sanfensan |
| 7D | 426049443 | T, C | sanfensan |
| 7D | 432545626 | A, G | sanfensan |
| 7D | 438677802 | A, T | sanfensan |
| 7D | 442595402 | T, C | sanfensan |
| 7D | 447536505 | C, T | sanfensan |
| 7D | 453173144 | T, A | sanfensan |
| 7D | 456025113 | C, T | sanfensan |
| 7D | 463196054 | A, T | sanfensan |
| 7D | 466703278 | A, G | sanfensan |
| 7D | 474383321 | G, T | sanfensan |
| 7D | 478751163 | T, C | sanfensan |
| 7D | 481249084 | C, G | sanfensan |
| 7D | 486113092 | A, T | sanfensan |
| 7D | 491181988 | G, T | sanfensan |
| 7D | 497047184 | A, G | sanfensan |
| 1A | 106635350 | G, C | OT3098 |
| 1A | 304418445 | G, A | OT3098 |
| 1A | 338092374 | C, T | OT3098 |
| 1A | 348499239 | C, T | OT3098 |
| 1A | 369374878 | A, G | OT3098 |
| 1A | 383912262 | C, T | OT3098 |
| 1A | 397750385 | A, G | OT3098 |
| 1A | 512105703 | G, A | OT3098 |
| 1C | 40349872 | G, T | OT3098 |
| 1C | 80742591 | G, T | OT3098 |
| 1C | 104538708 | A, G | OT3098 |
| 1C | 119650459 | G, T | OT3098 |
| 1C | 120468329 | T, G | OT3098 |
| 1C | 181195253 | G, A | OT3098 |
| 1C | 242119623 | C, T | OT3098 |
| 1C | 265897428 | C, T | OT3098 |
| 1C | 280066160 | G, A | OT3098 |
| 1C | 291282749 | A, T | OT3098 |
| 1C | 319005689 | C, A | OT3098 |
| 1C | 342223334 | G, A | OT3098 |
| 1C | 362511327 | C, T | OT3098 |
| 1C | 403231603 | G, T | OT3098 |
| 1D | 28155193 | G, A | OT3098 |
| 1D | 105612570 | C, T | OT3098 |
| 1D | 114754382 | T, A | OT3098 |
| 1D | 153648523 | C, T | OT3098 |
| 1D | 168181978 | G, A | OT3098 |
| 1D | 178507460 | T, C | OT3098 |
| 1D | 186530691 | C, G | OT3098 |
| 1D | 199381843 | C, T | OT3098 |
| 1D | 213294278 | C, A | OT3098 |
| 1D | 234770750 | A, G | OT3098 |
| 1D | 249534977 | G, A | OT3098 |
| 1D | 336367249 | T, C | OT3098 |
| 1D | 458302223 | A, G | OT3098 |
| 2A | 288229046 | G, T | OT3098 |
| 2A | 298965546 | A, G | OT3098 |
| 2A | 312180949 | G, A | OT3098 |
| 2A | 348182068 | C, T | OT3098 |
| 2A | 374638766 | A, G | OT3098 |
| 2A | 399111341 | G, A | OT3098 |
| 2C | 28799948 | T, C | OT3098 |
| 2C | 42159271 | T, G | OT3098 |
| 2C | 61574030 | G, T | OT3098 |
| 2C | 87111306 | T, C | OT3098 |
| 2C | 163497553 | G, A | OT3098 |
| 2C | 384573213 | A, G | OT3098 |
| 2C | 526110948 | A, G | OT3098 |
| 2C | 530336382 | G, A | OT3098 |
| 2D | 25489315 | A, G | OT3098 |
| 2D | 48295932 | C, T | OT3098 |
| 2D | 136287278 | G, A | OT3098 |
| 2D | 257949893 | T, C | OT3098 |
| 2D | 374246423 | T, G | OT3098 |
| 2D | 439808678 | A, G | OT3098 |
| 2D | 464345033 | G, A | OT3098 |
| 3A | 13251626 | G, A | OT3098 |
| 3A | 65224422 | C, T | OT3098 |
| 3A | 80331258 | G, A | OT3098 |
| 3A | 102356888 | G, A | OT3098 |
| 3A | 235151032 | C, T | OT3098 |
| 3A | 290609726 | A, G | OT3098 |
| 3A | 342060468 | A, G | OT3098 |
| 3A | 375362853 | G, T | OT3098 |
| 3C | 83743224 | C, T | OT3098 |
| 3C | 105875209 | T, A | OT3098 |
| 3C | 153371948 | G, A | OT3098 |
| 3C | 160415743 | A, G | OT3098 |
| 3C | 188382442 | C, T | OT3098 |
| 3C | 199311115 | T, C | OT3098 |
| 3C | 215249361 | C, T | OT3098 |
| 3C | 242293326 | A, C | OT3098 |
| 3C | 253359904 | T, C | OT3098 |
| 3C | 272308234 | G, T | OT3098 |
| 3C | 302216223 | G, A | OT3098 |
| 3C | 312338539 | T, C | OT3098 |
| 3C | 327416798 | T, C | OT3098 |
| 3C | 349649824 | A, C | OT3098 |
| 3C | 351983098 | G, A | OT3098 |
| 3C | 416217578 | T, A | OT3098 |
| 3C | 533296338 | T, C | OT3098 |
| 3C | 543025252 | A, G | OT3098 |
| 3C | 551825075 | C, T | OT3098 |
| 3C | 571627195 | T, C | OT3098 |
| 3C | 606797452 | C, A | OT3098 |
| 3C | 620635579 | G, A | OT3098 |
| 3D | 471246706 | C, T | OT3098 |
| 4A | 4383966 | T, C | OT3098 |
| 4A | 91672697 | A, G | OT3098 |
| 4A | 176172514 | T, C | OT3098 |
| 4A | 262281263 | T, G | OT3098 |
| 4A | 281599989 | C, T | OT3098 |
| 4A | 315467166 | T, C | OT3098 |
| 4A | 330624554 | C, T | OT3098 |
| 4A | 344502223 | G, A | OT3098 |
| 4A | 370035053 | A, G | OT3098 |
| 4A | 409954087 | G, A | OT3098 |
| 4A | 438257237 | C, G | OT3098 |
| 4A | 452257049 | G, A | OT3098 |
| 4C | 228404747 | C, T | OT3098 |
| 4C | 326801689 | T, C | OT3098 |
| 4C | 372872071 | G, A | OT3098 |
| 4C | 405897221 | T, C | OT3098 |
| 4C | 415253727 | C, G | OT3098 |
| 4C | 501843966 | T, G | OT3098 |
| 4C | 589184920 | A, T | OT3098 |
| 4C | 629827712 | C, A | OT3098 |
| 4C | 646042521 | A, T | OT3098 |
| 4C | 660379855 | G, A | OT3098 |
| 4C | 680363280 | G, A | OT3098 |
| 4C | 690482618 | G, A | OT3098 |
| 4D | 323386091 | T, C | OT3098 |
| 4D | 348557099 | G, A | OT3098 |
| 4D | 377370815 | A, C | OT3098 |
| 4D | 386553314 | C, T | OT3098 |
| 4D | 434004607 | A, G | OT3098 |
| 4D | 455273707 | C, T | OT3098 |
| 5A | 12250117 | C, T | OT3098 |
| 5A | 66713969 | G, T | OT3098 |
| 5A | 163771340 | C, T | OT3098 |
| 5A | 229347037 | A, G | OT3098 |
| 5A | 247731935 | C, T | OT3098 |
| 5A | 259020640 | C, T | OT3098 |
| 5A | 275554651 | C, T | OT3098 |
| 5A | 450812604 | G, A | OT3098 |
| 5A | 464203725 | C, T | OT3098 |
| 5C | 19684941 | G, A | OT3098 |
| 5C | 34175263 | G, T | OT3098 |
| 5C | 56717315 | T, G | OT3098 |
| 5C | 103289073 | G, T | OT3098 |
| 5C | 162396640 | T, C | OT3098 |
| 5C | 276244815 | G, A | OT3098 |
| 5C | 313968741 | G, A | OT3098 |
| 5C | 330901610 | A, G | OT3098 |
| 5C | 491868915 | T, C | OT3098 |
| 5C | 501340024 | G, A | OT3098 |
| 5C | 537051000 | T, C | OT3098 |
| 5C | 567644495 | G, A | OT3098 |
| 5C | 570184840 | A, C | OT3098 |
| 5D | 94959246 | G, A | OT3098 |
| 5D | 100600265 | G, A | OT3098 |
| 5D | 124321696 | T, C | OT3098 |
| 5D | 149532876 | G, A | OT3098 |
| 5D | 193714037 | A, G | OT3098 |
| 5D | 220790821 | A, G | OT3098 |
| 5D | 247323372 | T, C | OT3098 |
| 5D | 341143271 | A, G | OT3098 |
| 5D | 392150519 | A, C | OT3098 |
| 5D | 487408616 | A, G | OT3098 |
| 5D | 496068151 | G, T | OT3098 |
| 6A | 47261549 | G, C | OT3098 |
| 6A | 79634441 | C, A | OT3098 |
| 6A | 90176068 | T, C | OT3098 |
| 6A | 103858240 | T, C | OT3098 |
| 6A | 143659802 | T, C | OT3098 |
| 6A | 159101934 | A, T | OT3098 |
| 6A | 171391185 | C, A | OT3098 |
| 6A | 184973728 | A, G | OT3098 |
| 6A | 192005904 | G, A | OT3098 |
| 6A | 263939310 | C, T | OT3098 |
| 6A | 300620942 | A, G | OT3098 |
| 6A | 419205117 | T, A | OT3098 |
| 6C | 59229976 | C, T | OT3098 |
| 6C | 84591374 | C, A | OT3098 |
| 6C | 87587254 | G, C | OT3098 |
| 6C | 144536268 | C, A | OT3098 |
| 6C | 238362770 | C, A | OT3098 |
| 6C | 246928523 | C, T | OT3098 |
| 6C | 292396236 | T, C | OT3098 |
| 6C | 342467150 | A, G | OT3098 |
| 6C | 366301685 | G, A | OT3098 |
| 6C | 467269710 | C, T | OT3098 |
| 6C | 485083612 | C, T | OT3098 |
| 6C | 574716448 | C, T | OT3098 |
| 6C | 602967220 | T, C | OT3098 |
| 6C | 611213471 | T, C | OT3098 |
| 6D | 67717241 | G, A | OT3098 |
| 6D | 87582596 | A, G | OT3098 |
| 6D | 130251011 | A, G | OT3098 |
| 6D | 143238944 | C, T | OT3098 |
| 6D | 161653963 | A, G | OT3098 |
| 6D | 193391379 | C, T | OT3098 |
| 6D | 202580655 | G, A | OT3098 |
| 6D | 240523148 | G, C | OT3098 |
| 6D | 256237708 | G, T | OT3098 |
| 6D | 265237433 | T, C | OT3098 |
| 6D | 283670697 | G, A | OT3098 |
| 7A | 6894726 | A, G | OT3098 |
| 7A | 93976095 | A, C | OT3098 |
| 7A | 212399895 | C, G | OT3098 |
| 7A | 226861026 | A, C | OT3098 |
| 7A | 295965773 | A, G | OT3098 |
| 7A | 423933210 | T, C | OT3098 |
| 7A | 440302050 | T, C | OT3098 |
| 7A | 457458508 | A, G | OT3098 |
| 7A | 488323283 | A, G | OT3098 |
| 7C | 2133869 | C, T | OT3098 |
| 7C | 6687935 | A, G | OT3098 |
| 7C | 40731489 | G, A | OT3098 |
| 7C | 135716079 | C, A | OT3098 |
| 7C | 192742152 | C, T | OT3098 |
| 7C | 213883268 | A, G | OT3098 |
| 7C | 225224321 | T, C | OT3098 |
| 7C | 271932318 | G, A | OT3098 |
| 7C | 303692756 | A, G | OT3098 |
| 7C | 314575954 | T, C | OT3098 |
| 7C | 433548395 | T, C | OT3098 |
| 7C | 461822950 | C, T | OT3098 |
| 7C | 478807736 | A, G | OT3098 |
| 7D | 43006316 | G, A | OT3098 |
| 7D | 53808953 | G, A | OT3098 |
| 7D | 72525182 | T, A | OT3098 |
| 7D | 100996318 | C, T | OT3098 |
| 7D | 119049740 | A, G | OT3098 |
| 7D | 123197785 | C, T | OT3098 |
| 7D | 131225276 | A, G | OT3098 |
| 7D | 196173654 | T, G | OT3098 |
| 7D | 227132197 | T, C | OT3098 |
| 7D | 237053803 | T, G | OT3098 |
| 7D | 248735393 | T, C | OT3098 |
| 7D | 256172681 | C, T | OT3098 |
| 7D | 291312060 | C, T | OT3098 |
| 7D | 363340392 | T, C | OT3098 |
| 7D | 370273697 | A, G | OT3098 |
| 7D | 382112487 | G, A | OT3098 |
| 7D | 391806334 | A, G | OT3098 |
| 7D | 416042498 | C, T | OT3098 |
| 7D | 430774655 | T, C | OT3098 |
| 7D | 466485714 | C, T | OT3098 |

4. An application of the 2K SNP liquid chip of Avena sativa according to claim 3 in variety identifying, seed purity analysis, hybrid breeding and fingerprint construction of the Avena sativa.

5. A variety identifying method of Avena sativa, comprising following steps:
(1) obtaining genomic DNAs of samples to be detected;
(2) constructing a sequencing library based on the genomic DNAs;
(3) performing a probe hybridization reaction on the sequencing library and the 2K SNP liquid chip of the Avena sativa according to claim 3;
(4) extracting genotyping information after sequence sequencing captured by the liquid chip to form a genotyping file; and
(5) constructing fingerprint data of 2429 SNP loci of the samples to be detected; comparing the fingerprints of the samples to be detected in pairs, and performing statistical comparison on a total number of loci and a number of difference loci to calculate similarity of loci of the samples to be detected.
